(19) United States
(12) Patent Application Publication
(10) Pub. No.: US 2023/0167424 A1
(43) Pub. Date: Jun. 1, 2023

(54) COMPOSITIONS AND METHODS FOR THE TARGETING OF PCSK9

(71) Applicant: Scribe Therapeutics Inc., Alameda, CA (US)

(72) Inventors: Benjamin OAKES, El Cerrito, CA (US); Sean HIGGINS, Alameda, CA (US); Hannah SPINNER, Boston, MA (US); Sarah DENNY, San Francisco, CA (US); Brett T. STAAHL, Tiburon, CA (US); Kian TAYLOR, Atlanta, GA (US); Katherine BANEY, Berkeley, CA (US); Isabel COLIN, Oakland, CA (US); Maroof ADIL, Davis, CA (US); Cole URNES, Los Angeles, CA (US)

(21) Appl. No.: 17/791,130

(22) PCT Filed: Jan. 8, 2021
(86) PCT No.: PCT/US21/12804

§ 371 Date: Jul. 6, 2022

Related U.S. Application Data
(60) Provisional application No. 62/959,685, filed on Jan. 10, 2020.

(51) Int. Cl. C12N 9/22; C12N 15/11; C12N 15/90; A61P 3/06

(52) U.S. Cl. CPC C12N 9/22; C12N 15/11; C12N 15/111; C12N 15/907; C12Y 304/21061; A61P 3/06; C12N 2310/20; C12N 2800/80; A61K 38/00

(57) ABSTRACT

Provided herein are systems comprising Class2, Type V CRISPR polypeptides, guide nucleic acids (gNA), and optionally donor template nucleic acids useful in the modification of a PCSK9 gene. The systems are also useful for introduction into cells, for example eukaryotic cells having mutations in the PCSK9 gene. Also provided are methods of using such CasX:gNA systems to modify cells having such mutations.

### CROSS REFERENCE TO RELATED APPLICATIONS

This application claims priority to U.S. provisional patent application No. 62/959,685, filed on Jan. 10, 2020, the contents of which are incorporated herein by reference in their entirety.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Jan. 6, 2021 is named SCRB_017_01WO_SeqList_ST25.txt and is 4.07 MB in size.

### BACKGROUND

In mammals, cholesterol is transported within lipoproteins via emulsification. The lipoprotein particles are classified based on their density: low-density lipoproteins (LDL), very low-density lipoproteins (VLDL), high-density lipoproteins (HDL), and chylomicrons. Surface LDL receptors are internalized during cholesterol absorption. A cell with abundant cholesterol will have its LDL receptor synthesis blocked to prevent new cholesterol in LDL particles from being taken up. Conversely, LDL receptor synthesis is promoted when a cell is deficient in cholesterol. When the process is unregulated, excess LDL particles will travel in the blood without uptake by an LDL receptor. LDL particles in the blood are oxidized and taken up by macrophages, which then become engorged and form foam cells. These foam cells can become trapped in the walls of blood vessels and contribute to atherosclerotic plaque formation, which is one of the main causes of heart attacks, strokes, and other serious medical problems.

The liver protein proprotein convertase subtilisin/kexin Type 9 (PCSK9) is a secreted, globular, auto-activating serine protease that binds to the low-density lipoprotein receptor (LDL-R) during endocytosis of LDL particles, preventing recycling of the LDL-R to the cell surface and leading to reduction of LDL-cholesterol clearance. PCSK9 binds to the LDL-R (through the EGF-A domain), preventing the conformational change of the receptor-ligand complex, which redirects the LDL-R to the lysosome instead. As the receptor for low-density lipoprotein particles (LDL) typically transports thousands of fat molecules (including cholesterol) per particle within extracellular fluid, blocking or inhibiting the function of PCSK9 to boost LDL-R-mediated clearance of LDL cholesterol can lower LDL particle concentrations. PCSK9 is expressed mainly in the liver, the intestine, the kidney, and the central nervous system, but is also highly expressed in arterial walls such as endothelium, smooth muscle cells, and macrophages, with a local effect that can regulate vascular homeostasis and atherosclerosis.

PCSK9 is a member of the proprotein convertase (PC) family and its gene is mutated in ˜2% to 3% of individuals with familial hypercholesterolemia (FH) (Sepideh Mikaeeli, S., et al. Functional analysis of natural PCSK9 mutants in modern and archaic humans. FEBS J. 2019 Aug. 6. doi: 10.1111/febs.15036). Researchers have identified several PCSK9 mutations that cause an inherited form of high cholesterol (hypercholesterolemia). These mutations change a single protein building block (amino acid) in the PCSK9 protein. Researchers describe the mutations responsible for hypercholesterolemia as “gain-of-function” because they appear to enhance the activity of the PCSK9 protein or give the protein a new, atypical function (Blesa, S., et al. A New PCSK9 Gene Promoter Variant Affects Gene Expression and Causes Autosomal Dominant Hypercholesterolemia. J. Clin. Endocrinol. & Metab. 93:3577 (2008)). The overactive PCSK9 protein substantially reduces the number of low-density lipoprotein receptors on the surface of liver cells. With fewer receptors to remove low-density lipoproteins from the blood, people with gain-of-function mutations in the PCSK9 gene have very high blood cholesterol levels. Autosomal dominant hypercholesterolemia (ADH) is a genetic disorder characterized by increased low-density lipoprotein (LDL)-cholesterol levels, leading to high risk of premature cardiovascular disease. Approximately 10 mutations in PCSK9 have been identified as a cause of the disease in different populations. All known mutations in PCSK9 causing hypercholesterolemia produce an increase in the enzymatic activity of this protease (Bleasa, S., 2008). In addition, mutations in PCSK9 can lead to autosomal dominant familial hypobetalipoproteinemia, which can lead to hepatic steatosis, cirrhosis, and other disorders.

The advent of CRISPR/Cas systems and the programmable nature of these minimal systems has facilitated their use as a versatile technology for genomic manipulation and engineering. However, current methods of generating PCSK9 protective variants and loss-of-function mutants in vivo have been ineffective due to the large number of cells that need to be modified to modulate cholesterol levels. Other concerns involve off-target effects, genome instability, or oncogenic modifications that may be caused by genome editing, as well as a lack of safe delivery modalities for gene-editing systems. Thus, there remains a need for improved compositions and methods to regulate PCSK9. Provided herein are compositions and methods for targeting PCSK9, as well as delivery vectors, to the address this need.

### SUMMARY

The present disclosure relates to modified Class 2, Type V CRISPR proteins and guide nucleic acids used in the editing of proprotein convertase subtilisin/kexin Type 9 (PCSK9) gene target nucleic acid sequences having one or more mutations. The Class 2, Type V CRISPR proteins and guide nucleic acids can be modified for passive entry into target cells. The Class 2, Type V CRISPR proteins and guide nucleic acids are useful in a variety of methods for target nucleic acid modification, which methods are also provided.

In one aspect, the present disclosure relates to Class 2 Type V CRISPR protein and guide nucleic acid systems (e.g. CasX:gNA system) and methods used to alter a target nucleic acid comprising the gene encoding the PCSK9 protein (PCSK9 gene) in cells. In some embodiments of the disclosure, the CasX:gNA system has utility in knocking-down or knocking-out a PCSK9 gene with one or more mutations, which may be a gain of function mutation, in order to reduce or eliminate expression of the mutant PCSK9 gene product and resulting elevated hypercholesterolemia in subjects having a PCSK9 disorder. In other embodiments, the CasX:gNA system has utility in correcting the sequence of a PCSK9 gene with a gain of function mutation.

In some embodiments, the Class 2 Type V:gNA system gNA is a gRNA, or a gDNA, or a chimera of RNA and DNA, and may be a single-molecule gNA or a dual-molecule gNA. In other embodiments, the system gNA has a targeting sequence comprising a sequence having at least about 50%, at least about 60%, at least about 70%, at least about 80%, at least about 85%, at least about 90%, or at least about 95%, or 100% sequence identity to a sequence of SEQ ID NOS: 247-303, 315-436, 612-2100, or 2286-13861. In some embodiments, the gNA has a targeting sequence consisting of a sequence selected from the group consisting of SEQ ID NOS: 247-303, 315-436, 612-2100, and 2286-13861. In some embodiments, the targeting sequence of the gNA is complementary to a sequence within or proximal to an exon of the PCSK9 gene. In another embodiment, the targeting sequence of the gNA is complementary to a sequence within or proximal to an intron of the PCSK9 gene. In another embodiment, the targeting sequence of the gNA is complementary to a sequence within or proximal to an intron-exon junction of the PCSK9 gene. In another embodiment, the targeting sequence of the gNA is complementary to a sequence within or proximal to a regulatory element of the PCSK9 gene. In another embodiment, the targeting sequence of the gNA is complementary to a sequence within or proximal to an intergenic region of the PCSK9 gene. The gNA can comprise a targeting sequence comprising 14 to 30 consecutive nucleotides. In other embodiments, the targeting sequence of the gNA consists of 20 nucleotides. In other embodiments, the targeting sequence consists of 19 nucleotides. In other embodiments, the targeting sequence consists of 18 nucleotides. In other embodiments, the targeting sequence consists of 17 nucleotides. In other embodiments, the targeting sequence consists of 16 nucleotides. In other embodiments, the targeting sequence consists of 15 nucleotides.

In some embodiments, the gNA has a scaffold comprising a sequence selected from the group consisting of SEQ ID NOS: 4-16 as set forth in Table 1, or a sequence having at least about 50%, at least about 60%, at least about 70%, at least about 80%, at least about 90%, at least about 95%, at least about 96%, at least about 97%, at least about 98%, or at least about 99% sequence identity thereto. In other embodiments, the CasX:gNA system gNA has a scaffold comprising a sequence selected from the group consisting of SEQ ID NOS: 2101-2286 as set forth in Table 2, or a sequence having at least about 50%, at least about 60%, at least about 70%, at least about 80%, at least about 90%, at least about 95%, at least about 96%, at least about 97%, at least about 98%, at least about 99% sequence identity thereto.

In some embodiments, the CasX:gNA systems comprise a reference CasX sequence comprising any one of SEQ ID NOS: 1-3 or a CasX variant comprising a sequence of SEQ ID NOS: 49-160, 439, 441, 443, 445, 447-460, 472, 474, 476, 478, 480, 482, 484, 486, or 488, or 490 as set forth in Tables 3, 5-7 and 9, or a sequence having at least about 50%, at least about 60%, at least about 70%, at least about 80%, or at least about 90%, or at least about 95%, or at least about 96%, or at least about 97%, or at least about 98%, or at least about 99% sequence identity thereto. In these embodiments, a CasX variant exhibits one or more improved characteristics relative to the reference CasX protein. In some embodiments, the CasX protein has binding affinity for a protospacer adjacent motif (PAM) sequence selected from the group consisting of TTC, ATC, GTC, and CTC. In some embodiments, the CasX protein has binding affinity for the PAM sequence that is at least 1.5-fold greater compared to the binding affinity of any one of the CasX proteins of SEQ ID NOS: 1-3 for the PAM sequences selected from the group consisting of TTC, ATC, GTC, and CTC.

In some embodiments of the Class 2 Type V CRISPR:gNA system of the disclosure, the CRISPR molecule and the gNA molecule are associated together in a ribonuclear protein complex (RNP). In a particular embodiment, the RNP comprising a CasX variant and the gNA variant exhibits greater editing efficiency and/or binding of a target sequence in the target DNA when any one of the PAM sequences TTC, ATC, GTC, or CTC is located 1 nucleotide 5′ to the non-target strand sequence having identity with the targeting sequence of the gNA in a cellular assay system compared to the editing efficiency and/or binding of an RNP comprising a reference CasX protein and a reference gNA in a comparable assay system.

In some embodiments, the system further comprises a donor template comprising a nucleic acid comprising at least a portion of a gene encoding a PCSK9 protein or RNA sequence, a PCSK9 regulatory region, or both the encoding and the regulatory regions, and wherein the PCSK9 encoding gene portion is selected from the group consisting of a PCSK9 exon, a PCSK9 intron, and a PCSK9 intron-exon junction, wherein the donor template is used to knock down or knock out the PCSK9 gene or is used to correct the mutation in the PCSK9 gene. In some embodiments, the system further comprises a donor template comprising a nucleic acid comprising a sequence encoding at least a portion of SEQ ID NO: 33. In other embodiments, the system further comprises a donor template comprising a nucleic acid comprising a nucleic acid sequence having one or more mutations relative to the wild-type PCSK9 gene sequence of SEQ ID NO: 33. In some cases the donor sequence is a single-stranded DNA template or a single stranded RNA template. In other cases, the donor template is a double-stranded DNA template.

In other embodiments, the disclosure provides nucleic acids encoding the systems of any of the embodiments described herein, as well as vectors comprising the nucleic acids. In some embodiments, the vector is selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, and an RNA vector. In other embodiments, the vector is a virus-like particle (VLP) comprising an RNP of a CasX and gNA of any of the embodiments described herein and, optionally, a donor template nucleic acid.

In other embodiments, the disclosure provides a method of modifying a PCSK9 target nucleic acid sequence of a cell wherein the PCSK9 gene comprises one or more mutations, wherein said method comprises introducing into the cell: a) a composition comprising the Class 2 Type V:gNA system of any of the embodiments disclosed herein comprising a first gNA; b) the nucleic acid of any of the embodiments disclosed herein; c) the vector of any of the embodiments disclosed herein; d) the VLP of any of the embodiments disclosed herein; or e) a combination of two or more of the foregoing wherein the PCSK9 target nucleic acid sequence of the cells targeted by the first gNA is modified by the Class 2 Type V CRISPR protein (e.g. CasX). In some embodiments of the method, the method comprises introducing into the cells of the population a second gNA or a nucleic acid encoding the second gNA, wherein the second gNA has a targeting sequence complementary to a different or overlapping portion of the PCSK9 target nucleic acid compared to the first gNA, resulting in an additional break in the PCSK9 target nucleic acid of the cells of the population. In some embodiments of the method, the modifying comprises introducing an insertion, deletion, substitution, duplication, or inversion of one or more nucleotides in the target nucleic acid sequence as compared to the wild-type sequence. In some cases, the method further comprises contacting the target nucleic acid with a donor template nucleic acid of any of the embodiments disclosed herein. In some embodiments of the method, the donor template comprises a nucleic acid comprising at least a portion of a PCSK9 gene for correcting (by knocking in) the mutation of the PCSK9 gene, or comprises a sequence comprising a mutation or heterologous sequence for knocking out the mutant PCSK9. In those cases where the modification results in a knock-down of the PCSK9 gene, expression of the non-functional PCSK9 protein is reduced by at least about 10%, at least about 20%, at least about 30%, at least about 40%, at least about 50%, at least about 60%, at least about 70%, at least about 80%, or at least about 90% in comparison to cells that have not been modified. In other cases, wherein the modification results in a knock-out of the PCSK9 gene, the target nucleic acid of the cells of the population is modified such that at least about 10%, at least about 20%, at least about 30%, at least about 40%, at least about 50%, at least about 60%, at least about 70%, at least about 80%, or at least about 90% of the cells do not express a detectable level of non-functional PCSK9 protein. Expression of PCSK9 protein can be measured by flow cytometry, ELISA, cell-based assays, Western blot or other methods known in the art or as described in the Examples.

In some cases, the modifying of the target nucleic acid sequence in a cell occurs in vivo. In some embodiments, the cell is a eukaryotic cell selected from the group consisting of a rodent cell, a mouse cell, a rat cell, a primate cell, a non-human primate cell, and a human cell. In some embodiments, the cell is a hepatocyte, or a cell of the intestine, the kidney, the central nervous system, a smooth muscle cell, a macrophage, a retinal cell, or a cell of arterial walls such as the endothelium. In some embodiments, the cell is an eye cell. In other embodiments, the disclosure provides methods of modifying a target nucleic acid sequence wherein the target cells are contacted using vectors encoding the CasX protein and one or more gNAs, and optionally further comprising a donor template. In some cases, the vector is an Adeno-Associated Viral (AAV) vector selected from AAV1, AAV2, AAV3, AAV4, AAV5, AAV6, AAV7, AAV8, AAV9, AAV10, AAV11, AAV12, AAV44.9, AAV-Rh74, or AAVRh10. In other cases, the vector is a lentiviral vector. In other embodiments, the disclosure provides methods wherein the target cells are contacted using a vector wherein the vector is a virus-like particle comprising an RNP of a CasX and gNA of any of the embodiments described herein and, optionally, a donor template nucleic acid. In some embodiments of the method, the vector is administered to a subject at a therapeutically effective dose. The subject can be a mouse, rat, pig, non-human primate, or a human. The dose can be administered by a route of administration selected from the group consisting of intravenous, intraportal vein injection, intraperitoneal, intramuscular, subcutaneous, intraocular, and oral routes.

In other embodiments, the disclosure provides a method of treating a PCSK9 or related disorder in a subject in need thereof, comprising modifying a gene encoding PCSK9 gene in a cell of the subject, the modifying comprising contacting said cells with a therapeutically effective dose of: i) a composition comprising a CasX and gNA of any of the embodiments disclosed herein comprising a first gNA, and, optionally, a donor template; ii) a nucleic acid encoding the composition of (i); a vector selected from the group consisting of a retroviral vector, a lentiviral vector, an adenoviral vector, an adeno-associated viral (AAV) vector, a herpes simplex virus (HSV) vector, a plasmid, a minicircle, a nanoplasmid, a DNA vector, and an RNA vector, and comprising a nucleic acid of (ii); iii) a VLP comprising the composition of (i); or iv) combinations of two or more of (i)-(iii), wherein the PCSK9 gene of the cells targeted by the first gNA is modified by the CasX protein (and, optionally, the donor template) such that the mutation of the PCSK9 gene is corrected or compensated for and a functional PCSK9 protein is expressed. In other embodiments of the foregoing method of treating a PCSK9-related disease in a subject, the PCSK9 gene is knocked-down or knocked-out such that the expression of non-functional PCSK9 protein is reduced or eliminated. In some embodiments, the subject is selected from the group consisting of a rodent, a mouse, a rat, a non-human primate, and a human. In the foregoing, the vector or VLP is administered to the subject by a route of administration selected from intravenous, intraportal vein injection, intraperitoneal, intramuscular, subcutaneous, intraocular, or oral routes. In some embodiments, the PCSK9-related disorder is selected from the group consisting of autosomal dominant hypercholesterolemia (ADH), hypercholesterolemia, elevated total cholesterol levels, hyperlipidemia, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, age-related macular degeneration (AMD) or a combination thereof.

In some cases, the method results in improvement in at least one clinically-relevant endpoint selected from the group consisting of percent change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, visual acuity, and unstable angina. In other cases, the method results in improvement in at least two clinically-relevant endpoints.

In another aspect, the present disclosure provides pharmaceutical compositions and kits comprising the nucleic acids, vectors, Class 2, Type V CRISPR proteins, gNAs and gene editing pairs described herein.

In another aspect, provided herein are compositions comprising gene editing pairs, or compositions of vectors comprising or encoding gene editing pairs for use as a medicament for the treatment of a subject having a PCSK9-related disease.

In another aspect, provided herein are Class 2, Type V CRISPR:gNA systems, compositions comprising Class 2, Type V CRISPR:gNA systems, vectors comprising or encoding Class 2, Type V CRISPR:gNA systems, VLP comprising Class 2, Type V CRISPR:gNA systems, or populations of cells edited using the Class 2,Type V CRISPR:gNA systems for use as a medicament for the treatment of a PCSK9-related disease.

In another aspect, provided herein are Class 2, Type V CRISPR:gNA systems, composition comprising Class 2, Type V CRISPR:gNA systems, or vectors comprising or encoding Class 2, Type V CRISPR:gNA systems, VLP comprising Class 2,Type V CRISPR:gNA systems, populations of cells edited using the Class 2, Type V CRISPR:gNA systems, for use in a method of treatment of a PCSK9-related disease in a subject in need thereof.

In another aspect, provided herein are Class 2, Type V CRISPR:gNA systems, composition comprising Class 2, Type V CRISPR:gNA systems, or vectors comprising or encoding Class 2, Type V CRISPR:gNA systems, VLP comprising Class 2,Type V CRISPR:gNA systems, populations of cells edited using the Class 2, Type V CRISPR:gNA systems, for use in the manufacture of a medicament for the treatment of a PCSK9-related disease in a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The contents of PCT/US2020/036505, filed on Jun. 5, 2020, and the contents of U.S. Provisional Patent Application No. 63/121,196, filed on Dec. 3, 2020, both which disclose CasX variants and gNA variants, are hereby incorporated by reference in their entireties.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an SDS-PAGE gel of StX2 purification fractions visualized by colloidal Coomassie staining, as described in Example 1.

FIG. 2 shows the chromatogram from a size exclusion chromatography assay of the StX2, using of Superdex 200 16/600 pg Gel Filtration, as described in Example 1.

FIG. 3 shows an SDS-PAGE gel of StX2 purification fractions visualized by colloidal Coomassie staining, as described in Example 1.

FIG. 4 is a schematic showing the organization of the components in the pSTX34 plasmid used to assemble the CasX constructs, as described in Example 2.

FIG. 5 is a schematic showing the steps of generating the CasX 119 variant, as described in Example 2.

FIG. 6 shows an SDS-PAGE gel of purification samples, visualized on a Bio-Rad Stain-Free™ gel, as described in Example 2.

FIG. 7 shows the chromatogram of Superdex 200 16/600 pg Gel Filtration, as described in Example 2.

FIG. 8 shows an SDS-PAGE gel of gel filtration samples, stained with colloidal Coomassie, as described in Example 2.

FIG. 9 shows the results of an editing assay of 6 target genes in HEK293T cells, as described in Example 10. Each dot represents results using an individual spacer.

FIG. 10 shows the results of an editing assay of 6 target genes in HEK293T cells, with individual bars representing the results obtained with individual spacers, as described in Example 10.

FIG. 11 shows the results of an editing assay of 4 target genes in HEK293T cells, as described in Example 10. Each dot represents results using an individual spacer utilizing a CTC PAM.

FIG. 12 is a graph of the results of an assay for the quantification of active fractions of RNP formed by sgRNA174 and the CasX variants, as described in Example 12. Equimolar amounts of RNP and target were co-incubated and the amount of cleaved target was determined at the indicated timepoints. Mean and standard deviation of three independent replicates are shown for each timepoint. The biphasic fit of the combined replicates is shown. “2” refers to the reference CasX protein of SEQ ID NO:2.

FIG. 13 shows the quantification of active fractions of RNP formed by CasX2 (reference CasX protein of SEQ ID NO:2) and the modified sgRNAs, as described in Example 12. Equimolar amounts of RNP and target were co-incubated and the amount of cleaved target was determined at the indicated timepoints. Mean and standard deviation of three independent replicates are shown for each timepoint. The biphasic fit of the combined replicates is shown.

FIG. 14 shows the quantification of active fractions of RNP formed by CasX 491 and the modified sgRNAs under guide-limiting conditions, as described in Example 12. Equimolar amounts of RNP and target were co-incubated and the amount of cleaved target was determined at the indicated timepoints. The biphasic fit of the data is shown.

FIG. 15 shows the quantification of cleavage rates of RNP formed by sgRNA174 and the CasX variants, as described in Example 12. Target DNA was incubated with a 20-fold excess of the indicated RNP and the amount of cleaved target was determined at the indicated time points. Mean and standard deviation of three independent replicates are shown for each timepoint, except for 488 and 491 where a single replicate is shown. The monophasic fit of the combined replicates is shown.

FIG. 16 shows the quantification of cleavage rates of RNP formed by CasX2 and the sgRNA variants, as described in Example 12. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 17 shows the quantification of initial velocities of RNP formed by CasX2 and the sgRNA variants, as described in Example 12. The first two time-points of the previous cleavage experiment were fit with a linear model to determine the initial cleavage velocity.

FIG. 18 shows the quantification of cleavage rates of RNP formed by CasX491 and the sgRNA variants, as described in Example 12. Target DNA was incubated with a 20-fold excess of the indicated RNP at 10° C. and the amount of cleaved target was determined at the indicated time points. The monophasic fit of the timepoints is shown.

FIG. 19 is a diagram and an example fluorescence activated cell sorting (FACS) plot illustrating an exemplary method for assaying the effectiveness of a reference CasX protein or single guide RNA (sgRNA), or variants thereof, as described in Example 21. A reporter (e.g., GFP reporter) coupled to a gRNA target sequence, complementary to the gRNA spacer, is integrated into a reporter cell line. Cells are transformed or transfected with a CasX protein and/or sgRNA variant, with the spacer motif of the sgRNA complementary to and targeting the gRNA target sequence of the reporter. Ability of the CasX:sgRNA ribonucleoprotein complex to cleave the target sequence is assayed by FACS. Cells that lose reporter expression indicate occurrence of CasX:sgRNA ribonucleoprotein complex-mediated cleavage and indel formation.

FIG. 20 shows results of gene editing in an EGFP disruption assay, as described in Example 23. Editing was measured by indel formation and GFP disruption in HEK293 cells carrying a GFP reporter. The Figure shows the improvement in editing efficiency of a CasX sgRNA variant of SEQ ID NO:5 versus the reference of SEQ ID NO:4 across 10 targets. When averaged across 10 targets, the editing efficiency of sgRNA SEQ ID NO:5 improved 176% compared to SEQ ID NO:4.

FIG. 21 shows results of gene editing in an EGFP disruption assay where further editing improvements were obtained in the sgRNA scaffold of SEQ ID NO:5 by swapping the extended stem loop sequence (indicated in the X-axis) for additional sequences to generate the scaffolds whose sequences are shown in Table 2, as described in Example 24.

FIG. 22 is a graph showing the fold improvement of sgRNA variants generated by DME mutations normalized to SEQ ID NO:5 as the CasX reference sgRNA, as described in Example 24. ATTATCTCATTACT is provided as SEQ ID NO: 13862.

FIG. 23 is a graph showing the fold improvement normalized to the SEQ ID NO:5 reference CasX sgRNA of variants created by both combining (stacking) scaffold stem mutations showing improved cleavage, DME mutations showing improved cleavage, and using ribozyme appendages showing improved cleavage (the appendages and their sequences are listed in Table 16 in Example 24). The resulting sgRNA variants yield 2-fold or greater improvement in cleavage compared to SEQ ID NO:5 in this assay. EGFP editing assays were performed with spacer target sequences of E6 (TGTGGTCGGGGTAGCGGCTG (SEQ ID NO: 17)) and E7 (TCAAGTCCGCCATGCCCGAA (SEQ ID NO: 18)) described in Example 23.

FIG. 24 is a graph of results of editing assayed by NGS of CasX at the PCSK9 locus in HEK293T cells showing total editing percentage, as described in Example 25.

FIG. 25 is a graph of results of editing assayed by NGS of CasX at the PCSK9 locus in Hep2G cells showing total editing percentage as described in Example 26.

FIG. 26 is a graph of results of editing assayed by NGS of CasX at the PCSK9 locus in AML12 cells showing total editing percentage as described in Example 27.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid sequence to be specifically hybridizable; it can have at least about 70%, at least about 80%, or at least about 90%, or at least about 95% sequence identity and still hybridize to the target nucleic acid sequence. Moreover, a polynucleotide may hybridize over one or more segments such that intervening or adjacent segments are not involved in the hybridization event (e.g., a loop structure or hairpin structure, a ‘bulge’, ‘bubble’ and the like).

A “gene,” for the purposes of the present disclosure, includes a DNA region encoding a gene product (e.g., a protein, RNA), as well as all DNA regions which regulate the production of the gene product, whether or not such regulatory element sequences are adjacent to coding and/or transcribed sequences. Accordingly, a gene may include regulatory sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed, e.g. the strand containing the coding sequence, as well as the complementary strand.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “regulatory element” is used interchangeably herein with the term “regulatory sequence,” and is intended to include promoters, enhancers, and other expression regulatory elements (e.g. transcription termination signals, such as polyadenylation signals and poly-U sequences). Exemplary regulatory elements include transcription promoters such as, but not limited to, CMV, CMV+intron A, SV40, RSV, HIV-Ltr, elongation factor 1 alpha (EF1α), MMLV-ltr, as well as other regulatory elements such as internal ribosome entry site (IRES) or P2A peptide to permit translation of multiple genes from a single transcript, metallothionein, a transcription enhancer element, a transcription termination signal, polyadenylation sequences, sequences for optimization of initiation of translation, and translation termination sequences. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “promoter” refers to a DNA sequence that contains an RNA polymerase binding site, transcription start site, TATA box, and/or B recognition element and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue specific, inducible, etc.

The term “enhancer” refers to regulatory element DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such can be done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid sequence with a guide nucleic acid means that the target nucleic acid sequence and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L] [P]/[LP], where [P], [L] and [LP] represent molar concentrations of the protein, ligand and complex, respectively.

The disclosure provides compositions and methods useful for modifying a target nucleic acid sequence. As used herein “modifying” includes, but is not limited to, cleaving, nicking, editing, deleting, knocking in, knocking out, and the like.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor (e.g., such as the donor template) to the target. Homology-directed repair can result in an alteration of the sequence of the target nucleic acid sequence by insertion, deletion, or mutation if the donor template differs from the target DNA sequence and part or all of the sequence of the donor template is incorporated into the target DNA at the correct genomic locus.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in indels; the loss (deletion) or insertion of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide (or protein) has a certain percent “sequence similarity” or “sequence identity” to another polynucleotide or polypeptide, meaning that, when aligned, that percentage of bases or amino acids are the same, and in the same relative position, when comparing the two sequences. Sequence similarity (interchangeably referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, or cosmid, to which another DNA segment, i.e., an “insert”, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “low-density lipoprotein (LDL)” refers to one of the five major groups of lipoprotein, from least dense (lower weight-volume ratio particles) to most dense (larger weight-volume ratio particles): chylomicrons, very low-density lipoproteins (VLDL), low-density lipoproteins (LDL), intermediate-density lipoproteins (IDL), and high-density lipoproteins (HDL). Lipoproteins transfer lipids (fats) around the body in the extracellular fluid thereby facilitating the transfer of fats to the cells body via receptor-mediated endocytosis. An LDL particle is about 220-275 angstroms in diameter.

“Low-density lipoprotein (LDL) receptor” refers to a receptor protein of 839 amino acids (after removal of 21-amino acid signal peptide) that mediates the endocytosis of cholesterol-rich LDL particles. It is a cell-surface receptor that recognizes the apoprotein B100 and apoE protein found in chylomicron remnants and VLDL remnants (IDL) resulting in the binding and endocytosis of LDL-cholesterol. This process occurs in all nucleated cells, but mainly in the liver which removes approximately 70% of LDL from the circulation. The human LDLR gene is described in part in the NCBI database (ncbi.nlm.nih.gov) as Reference Sequence NG_009060.1, which is incorporated by reference herein.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” is meant as a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, rabbits, mice, rats and other rodents.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4^{th} Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Systems for Genetic Editing of PCSK9 Genes

In a first aspect, the present disclosure provides systems comprising a CRISPR nuclease protein and one or more guide nucleic acids (gNA), as well as nucleic acids encoding the CRISPR nuclease proteins and gNA, for use in modifying or editing a PCSK9 gene (referred to herein as the “target nucleic acid”) in order to modify expression of the PCSK9 gene product.

As used herein, a “system,” such as the systems comprising a CRISPR nuclease protein and one or more gNAs the disclosure, as well as nucleic acids encoding the CRISPR nuclease proteins and gNA, is used interchangeably with term “composition.”

The PCSK9 gene encodes proprotein convertase subtilisin/kexin Type 9 (“PCSK9”), a protein that binds to the receptor for low-density lipoprotein particles (LDL) for transport of LDL into the cell. The PCSK9 gene encompasses the sequence that spans chr1:55,039,476-55,064,853 of the human genome (GRCh38/hg38) (the notation refers to the chromosome 1 (chr1), starting at the 55,039,476 bp to 55,064,853 bp on chromosome 1 (Homo sapiens Updated Annotation Release 109.20190905, GRCh38.p13) (NCBI). The human PCSK9 gene is described in part in the NCBI database (ncbi.nlm.nih.gov) as Reference Sequence NG_009061.1, which is incorporated by reference herein. The PCSK9 locus has 12 exons that produces an mRNA of 3636 bp encoding a 692-amino acid protein that, following its synthesis, undergoes an autocatalytic cleavage reaction that clips off the prodomain, resulting in an activated protein having 540 amino acids. The prodomain remains attached to the catalytic and resistin-like domains, likely because the prodomain serves as a chaperone and facilitates folding and secretion (Seidah, N G et al., Proc Natl Acad Sci USA 100(3):928 (2003)). The secretory proprotein convertase neural apoptosis-regulated convertase 1 (NARC-1): liver regeneration and neuronal differentiation (Seidah N G, et al.). This protein, also called neural apoptosis regulated convertase, is a serine protease belonging to the protease K subfamily of subtilases.

The human PCSK9 gene (HGNC:20001) encodes a protein (Q8NBP7) having the sequence

(SEQ ID NO: 33)
MGTVSSRRSWWPLPLLLLLLLLLGPAGARAQEDEDGDYEELVLALRSEE
DGLAEAPEHGTTATFHRCAKDPWRLPGTYVVVLKEETHLSQSERTARRL
QAQAARRGYLTKILHVFHGLLPGFLVKMSGDLLELALKLPHVDYIEEDS
SVFAQSIPWNLERITPPRYRADEYQPPDGGSLVEVYLLDTSIQSDHREI
EGRVMVTDFENVPEEDGTRFHRQASKCDSHGTHLAGVVSGRDAGVAKGA
SMRSLRVLNCQGKGTVSGTLIGLEFIRKSQLVQPVGPLVVLLPLAGGYS
RVLNAACQRLARAGVVLVTAAGNFRDDACLYSPASAPEVITVGATNAQD
QPVTLGTLGTNFGRCVDLFAPGEDIIGASSDCSTCFVSQSGTSQAAAHV
AGIAAMMLSAEPELTLAELRQRLIHFSAKDVINEAWFPEDQRVLTPNLV
AALPPSTHGAGWQLFCRTVWSAHSGPTRMATAVARCAPDEELLSCSSFS
RSGKRRGERMEAQGGKLVCRAHNAFGGEGVYAIARCCLLPQANCSVHTA
PPAEASMGTRVHCHQQGHVLTGCSSHWEVEDLGTHKPPVLRPRGQPNQC
VGHREASIHASCCHAPGLECKVKEHGIPAPQEQVTVACEEGWTLTGCSA
LPGTSHVLGAYAVDNTCVVRSRDVSTTGSTSEGAVTAVAICCRSRHLAQ
ASQELQ.

In some embodiments, the disclosure provides systems specifically designed to modify the PCSK9 gene in eukaryotic cells having a gain of function mutation. In some cases, the CRISPR systems are designed to knock-down or knock-out the PCSK9 gene. In other cases, the CRISPR systems are designed to correct one or more mutations in the PCSK9 gene. Generally, any portion of the PCSK9 gene can be targeted using the programmable compositions and methods provided herein, described more fully, herein.

In some embodiments, the CRISPR nuclease is a Class 2, Type V nuclease. In some embodiments, the Class 2, Type V nuclease is selected from the group consisting of Cas12a, Cas12b, Cas12c, Cas12d (CasY), Cas12J, CasZ, and CasX. In some embodiments, the disclosure provides systems comprising one or more CasX proteins and one or more guide nucleic acids (gNA) as a CasX:gNA system.

In some embodiments, the CasX:gNA systems of the disclosure comprise one or more CasX proteins, one or more guide nucleic acids (gNA) and one or more donor template nucleic acids comprising a nucleic acid encoding a portion of a PCSK9 gene wherein the nucleic acid comprises a wild-type sequence, a cDNA sequence encoding a portion of a functional PCSK9 protein, a deletion, an insertion, or a mutation of one or more nucleotides in comparison to a genomic nucleic acid sequence encoding the mutant PCSK9. In some embodiments, the donor template comprises one or more mutations compared to a wild-type PCSK9 gene utilized for insertion for either knocking out or knocking down (described more fully, below) the target nucleic acid sequence with one or more mutations. In other cases, the CasX:gNA systems can optionally further comprise a donor template for the introduction (or knocking in) of all or a portion of gene encoding a sequence for the production of a wild-type PCSK9 protein (SEQ ID NO: 33) in the target cell.

In those cases where the PCSK9 mutation spans multiple exons, the disclosure contemplates a donor template of sufficient length that may also be optimized to contain synthetic intron sequences of shortened length (relative to the genomic intron) between the exons in the donor template to ensure proper expression and processing of the PCSK9 locus. In some embodiments, the donor polynucleotide comprises at least about 10, at least about 50, at least about 100, or at least about 200, or at least about 300, or at least about 400, or at least about 500, or at least about 600, or at least about 700, or at least about 800, or at least about 900, or at least about 1000, or at least about 10,000, or at least about 15,000, or at least about 30,000 nucleotides. In other embodiments, the donor polynucleotide comprises at least about 10 to about 30,000 nucleotides, or at least about 100 to about 15,000 nucleotides, or at least about 400 to about 10,000 nucleotides, or at least about 600 to about 5000 nucleotides, or at least about 1000 to about 2000 nucleotides, wherein the PCSK9 gene portion is selected from the group consisting of a PCSK9 exon, a PCSK9 intron, a PCSK9 intron-exon junction, a PCSK9 regulatory region, a PCSK9 coding region, a PCSK9 non-coding region, a combination of any of the preceding portions of the PCSK9 gene, or the entirety of the PCSK9 gene. In some embodiments, the PCSK9 gene portion comprises a combination of any of a PCSK9 exon sequence, a PCSK9 intron sequence, a PCSK9 intron-exon junction sequence, or a PCSK9 regulatory region sequence. In some embodiments, the donor template is a single stranded DNA template or a single stranded RNA template. In other embodiments, the donor template is a double stranded DNA template.

In some embodiments, the disclosure provides gene editing pairs of a CasX and a gNA of any of the embodiments described herein that are capable of being bound together prior to their use for gene editing and, thus, are “pre-complexed” as a ribonuclear protein complex (RNP). The use of a pre-complexed RNP confers advantages in the delivery of the system components to a cell or target nucleic acid sequence for editing of the target nucleic acid sequence. In some embodiments, the functional RNP can be delivered ex vivo to a cell by electrophoresis or by chemical means. In other embodiments, the functional RNP can be delivered either ex vivo or in vivo by a vector in their functional form. The gNA can provide target specificity to the complex by including a targeting sequence (or “spacer”) having a nucleotide sequence that is complementary to a sequence of the target nucleic acid sequence while the CasX protein of the pre-complexed CasX:gNA provides the site-specific activity such as cleavage or nicking of the target sequence that is guided to a target site (e.g., stabilized at a target site within the PCSK9 gene) within a target nucleic acid sequence by virtue of its association with the gNA. The CasX proteins and gNA components of the CasX:gNA systems and their sequences, features and functions and their use in the editing of the PCSK9 gene are described more fully, below.

### III. Guide Nucleic Acids of the Systems for Genetic Editing

In another aspect, the disclosure relates to guide nucleic acids (gNA) comprising a targeting sequence complementary to a target nucleic acid sequence of a PCSK9 gene, wherein the gNA is capable of forming a complex with a CRISPR protein that has specificity to a protospacer adjacent motif (PAM) sequence comprising a TC motif in the complementary non-target strand, and wherein the PAM sequence is located 1 nucleotide 5′ of the sequence in the non-target strand that is complementary to the target nucleic acid sequence in the target strand of the target nucleic acid. In some embodiments, the gNA is capable of forming a complex with a Class 2, Type V CRISPR nuclease. In a particular embodiment, the gNA is capable of forming a complex with a CasX nuclease.

In some embodiments, the disclosure relates to guide nucleic acids (gNA) utilized in the CasX:gNA systems that have utility in genome editing of a PCSK9 gene in a cell. The present disclosure provides specifically-designed guide nucleic acids (“gNAs”) with targeting sequences that are complementary to (and are therefore able to hybridize with) the PCSK9 gene as a component of the gene editing CasX:gNA systems. Representative, but non-limiting examples of targeting sequences to the PCSK9 target nucleic acid that can be utilized in the gNA of the embodiments are presented as SEQ ID NOS: 247-303, 315-436, 612-2100, and 2286-13861. In some embodiments, the gNA is a deoxyribonucleic acid molecule (“gDNA”); in some embodiments, the gNA is a ribonucleic acid molecule (“gRNA”); and in other embodiments, the gNA is a chimera, and comprises both DNA and RNA. As used herein, the terms gNA, gRNA, and gDNA cover naturally-occurring molecules, as well as sequence variants.

It is envisioned that in some embodiments, multiple gNAs (e.g., two or more) are delivered in the methods for the modification of a target nucleic acid sequence by use of the CasX:gNA systems which is then edited by host cell repair mechanisms such as non-homologous end joining (NHEJ), homology-directed repair (HDR, which can include, for example, insertion of a donor template to replace all or a portion of the PCSK9 exon), homology-independent targeted integration (HITI), micro-homology mediated end joining (MMEJ), single strand annealing (SSA) or base excision repair (BER). For example, when an editing event designed to delete one or more mutant exons of the PCSK9 gene is desired, a pair of gNAs can be used in order to bind and cleave at two different sites 5′ and 3′ of the exon(s) bearing the mutation(s) within the PCSK9 gene. In the context of nucleic acids, cleavage refers to the breakage of the covalent backbone of a nucleic acid molecule; either DNA or RNA, by the nuclease. Both single-stranded cleavage and double-stranded cleavage are possible, and double-stranded cleavage can occur as a result of two distinct single-stranded cleavage events. In some embodiments, small indels introduced by the CasX:gNA systems of the embodiments described herein and cellular repair systems can restore the protein reading frame of the mutant PCSK9 gene (“reframing” strategy). When the reframing strategy is used, the cells may be contacted with a single gNA. Reference gNA and gNA variants.

In some embodiments, a gNA of the present disclosure comprises a sequence of a naturally-occurring gNA (a “reference gNA”). In other cases, a reference gNA of the disclosure may be subjected to one or more mutagenesis methods, such as the mutagenesis methods described herein, which may include Deep Mutational Evolution (DME), deep mutational scanning (DMS), error prone PCR, cassette mutagenesis, random mutagenesis, staggered extension PCR, gene shuffling, or domain swapping, in order to generate one or more gNA variants with a modified sequence relative to the reference gNA, wherein the gNA variant exhibits enhanced or varied properties relative to the reference gNA. gNA variants also include variants comprising one or more exogenous sequences, for example fused to either the 5′ or 3′ end, or inserted internally. The activity of reference gNAs may be used as a benchmark against which the activity of gNA variants are compared, thereby measuring improvements in function or other characteristics of the gNA variants. In other embodiments, a reference gNA may be subjected to one or more deliberate, specifically-targeted mutations in order to produce a gNA variant, for example a rationally designed variant.

The gNAs of the disclosure comprise two segments: a targeting sequence and a protein-binding segment. The targeting segment of a gNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a target ssRNA, a target ssDNA, a strand of a double stranded target DNA, etc.), described more fully below. The targeting sequence of a gNA is capable of binding to a target nucleic acid sequence, including a coding sequence, a complement of a coding sequence, a non-coding sequence, and to regulatory elements. The protein-binding segment (or “activator” or “protein-binding sequence”) interacts with (e.g., binds to) a CasX protein as a complex, forming an RNP (described more fully, below). The protein-binding segment is alternatively referred to herein as a “scaffold”, which is comprised of several regions, described more fully, below.

In the case of a dual guide RNA (dgRNA), the targeter and the activator portions each have a duplex-forming segment, where the duplex forming segment of the targeter and the duplex-forming segment of the activator have complementarity with one another and hybridize to one another to form a double stranded duplex (dsRNA duplex for a gRNA). When the gNA is a gRNA, the term “targeter” or “targeter RNA” is used herein to refer to a crRNA-like molecule (crRNA: “CRISPR RNA”) of a CasX dual guide RNA (and therefore of a CasX single guide RNA when the “activator” and the “targeter” are linked together, e.g., by intervening nucleotides). The crRNA has a 5′ region that anneals with the tracrRNA followed by the nucleotides of the targeting sequence. Thus, for example, a guide RNA (dgRNA or sgRNA) comprises a guide sequence and a duplex-forming segment of a crRNA, which can also be referred to as a crRNA repeat. A corresponding tracrRNA-like molecule (activator) also comprises a duplex-forming stretch of nucleotides that forms the other half of the dsRNA duplex of the protein-binding segment of the guide RNA. Thus, a targeter and an activator, as a corresponding pair, hybridize to form a dual guide NA, referred to herein as a “dual guide NA”, a “dual-molecule gNA”, a “dgNA”, a “double-molecule guide NA”, or a “two-molecule guide NA”. Site-specific binding and/or cleavage of a target nucleic acid sequence (e.g., genomic DNA) by the CasX protein can occur at one or more locations (e.g., a sequence of a target nucleic acid) determined by base-pairing complementarity between the targeting sequence of the gNA and the target nucleic acid sequence. Thus, for example, the gNA of the disclosure have sequences complementarity to and therefore can hybridize with the target nucleic acid that is adjacent to a sequence complementary to a TC PAM motif or a PAM sequence, such as ATC, CTC, GTC, or TTC. Because the targeting sequence of a guide sequence hybridizes with a sequence of a target nucleic acid sequence, a targeter can be modified by a user to hybridize with a specific target nucleic acid sequence, so long as the location of the PAM sequence is considered. Thus, in some cases, the sequence of a targeter may be a non-naturally occurring sequence. In other cases, the sequence of a targeter may be a naturally-occurring sequence, derived from the gene to be edited. In other embodiments, the activator and targeter of the gNA are covalently linked to one another (rather than hybridizing to one another) and comprise a single molecule, referred to herein as a “single-molecule gNA,” “one-molecule guide NA,” “single guide NA”, “single guide RNA”, a “single-molecule guide RNA,” a “one-molecule guide RNA”, a “single guide DNA”, a “single-molecule DNA”, or a “one-molecule guide DNA”, (“sgNA”, “sgRNA”, or a “sgDNA”). In some embodiments, the sgNA includes an “activator” or a “targeter” and thus can be an “activator-RNA” and a “targeter-RNA,” respectively.

Collectively, the assembled gNAs of the disclosure comprise four distinct regions, or domains: the RNA triplex, the scaffold stem, the extended stem, and the targeting sequence that, in the embodiments of the disclosure, is specific for a target nucleic acid and is located on the 3′ end of the gNA. The RNA triplex, the scaffold stem, and the extended stem, together, are referred to as the “scaffold” of the gNA,

a. RNA Triplex

In some embodiments of the guide RNAs provided herein (including reference sgRNAs), there is a RNA triplex, and the RNA triplex comprises the sequence of a UUU-nX(˜4-15)-UUU stem loop (SEQ ID NO: 19) that ends with an AAAG after 2 intervening stem loops (the scaffold stem loop and the extended stem loop), forming a pseudoknot that may also extend past the triplex into a duplex pseudoknot. The UU-UUU-AAA sequence of the triplex forms as a nexus between the targeting sequence, scaffold stem, and extended stem. In exemplary CasX sgRNAs, the UUU-loop-UUU region is coded for first, then the scaffold stem loop, and then the extended stem loop, which is linked by the tetraloop, and then an AAAG closes off the triplex before becoming the targeting sequence.

b. Scaffold Stem Loop

In some embodiments of sgNAs of the disclosure, the triplex region is followed by the scaffold stem loop. The scaffold stem loop is a region of the gNA that is bound by CasX protein (such as a CasX variant protein). In some embodiments, the scaffold stem loop is a fairly short and stable stem loop. In some cases, the scaffold stem loop does not tolerate many changes, and requires some form of an RNA bubble. In some embodiments, the scaffold stem is necessary for CasX sgNA function. While it is perhaps analogous to the nexus stem of Cas9 as being a critical stem loop, the scaffold stem of a CasX sgNA, in some embodiments, has a necessary bulge (RNA bubble) that is different from many other stem loops found in CRISPR/Cas systems. In some embodiments, the presence of this bulge is conserved across sgNA that interact with different CasX proteins. An exemplary sequence of a scaffold stem loop sequence of a gNA comprises the sequence CCAGCGACUAUGUCGUAUGG (SEQ ID NO: 20). In other embodiments, the disclosure provides gNA variants wherein the scaffold stem loop is replaced with an RNA stem loop sequence from a heterologous RNA source with proximal 5′ and 3′ ends, such as, but not limited to stem loop sequences selected from MS2, Q β, U1 hairpin II, Uvsx, or PP7 stem loops. In some cases, the heterologous RNA stem loop of the gNA is capable of binding a protein, an RNA structure, a DNA sequence, or a small molecule.

c. Extended Stem Loop

In some embodiments of the CasX sgNAs of the disclosure, the scaffold stem loop is followed by the extended stem loop. In some embodiments, the extended stem comprises a synthetic tracr and crRNA fusion that is largely unbound by the CasX protein. In some embodiments, the extended stem loop can be highly malleable. In some embodiments, a single guide gRNA is made with a GAAA tetraloop linker or a GAGAAA linker between the tracr and crRNA in the extended stem loop. In some cases, the targeter and activator of a CasX sgNA are linked to one another by intervening nucleotides and the linker can have a length of from 3 to 20 nucleotides. In some embodiments of the CasX sgNAs of the disclosure, the extended stem is a large 32-bp loop that sits outside of the CasX protein in the ribonucleoprotein complex. An exemplary sequence of an extended stem loop sequence of a sgNA comprises the sequence GCGCUUAUUUAUCGGAGAGAAAUCCGAUAAAUAAGAAGC (SEQ ID NO: 21). In some embodiments, the extended stem loop comprises a GAGAAA spacer sequence. In some embodiments, the disclosure provides gNA variants wherein the extended stem loop is replaced with an RNA stem loop sequence from a heterologous RNA source with proximal 5′ and 3′ ends, such as, but not limited to stem loop sequences selected from MS2, Qβ, U1 hairpin II, Uvsx, or PP7 stem loops. In such cases, the heterologous RNA stem loop increases the stability of the gNA. In other embodiments, the disclosure provides gNA variants having an extended stem loop region comprising at least 10, at least 100, at least 500, at least 1000, or at least 10,000 nucleotides, or at least 10-10,000, at least 10-1000, or at least 10-100 nucleotides.

d. Targeting Sequence

In some embodiments of the gNAs of the disclosure, the extended stem loop is followed by a region that forms part of the triplex, and then the targeting sequence (or “spacer”) at the 3′ end of the gNA. The targeting sequence targets the CasX ribonucleoprotein holo complex (i.e., the RNP) to a specific region of the target nucleic acid sequence of the gene to be modified. Thus, for example, gNA targeting sequences of the disclosure have sequences complementarity to, and therefore can hybridize to, a portion of the PCSK9 gene in a nucleic acid in a eukaryotic cell (e.g., a eukaryotic chromosome, chromosomal sequence, a eukaryotic RNA, etc.) as a component of the RNP when the TC PAM motif or any one of the PAM sequences TTC, ATC, GTC, or CTC is located 1 nucleotide 5′ to the non-target strand sequence complementary to the target sequence. The targeting sequence of a gNA can be modified so that the gNA can target a desired sequence of any desired target nucleic acid sequence, so long as the PAM sequence location is taken into consideration. In some embodiments, the gNA scaffold is 5′ of the targeting sequence, with the targeting sequence on the 3′ end of the gNA. In some embodiments, the PAM motif sequence recognized by the nuclease of the RNP is TC. In other embodiments, the PAM sequence recognized by the nuclease of the RNP is NTC.

In some embodiments, the targeting sequence of the gNA is complementary to a portion of a gene encoding a PCSK9 protein, which may comprise one or more mutations. In some embodiments, the targeting sequence of a gNA is complementary to a PCSK9 exon selected from the group consisting of exon 1, exon 2, exon 3, exon 4, exon 5, exon 6, exon 7, exon 8, exon 9, exon 10, exon 11, and exon 12. In some embodiments, the targeting sequence of a gNA is specific for a PCSK9 intron. In some embodiments, the targeting sequence of the gNA is specific for a PCSK9 intron-exon junction. In some embodiments, the targeting sequence of the gNA is complementary to a sequence comprising one or more single nucleotide polymorphisms (SNPs) of the PCSK9 gene or its complement. SNPs that are within PCSK9 coding sequence or within PCSK9 non-coding sequence are both within the scope of the instant disclosure. In other embodiments, the targeting sequence of the gNA is complementary to a sequence of an intergenic region of the PCSK9 gene or a sequence complementary to an intergenic region of the PCSK9 gene. In other embodiments, the targeting sequence of a gNA is complementary to a regulatory element of the PCSK9 gene. In those cases where the targeting sequence is specific for a regulatory element, such regulatory elements include, but are not limited to promoter regions, enhancer regions, intergenic regions, 5′ untranslated regions (5′ UTR), 3′ untranslated regions (3′ UTR), conserved elements, and regions comprising cis-regulatory elements. The promoter region is intended to encompass nucleotides within 5 kb of the initiation point of the encoding sequence or, in the case of gene enhancer elements or conserved elements, can be thousands of bp, hundreds of thousands of bp, or even millions of bp away from the encoding sequence of the gene of the target nucleic acid. In the foregoing, the targets are those in which the encoding gene of the target is intended to be knocked out or knocked down such that the targeted protein is not expressed or is expressed at a lower level in a cell.

In some embodiments, the targeting sequence has between 14 and 35 consecutive nucleotides. In some embodiments, the targeting sequence has 14, 15, 16, 18, 18, 19, 20, 21, 22, 23 24, 25, 26, 27, 28, 29, 30, 31, 32, 33, 34 or 35 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides and the targeting sequence can comprise 0 to 5, 0 to 4, 0 to 3, or 0 to 2 mismatches relative to the target nucleic acid sequence and retain sufficient binding specificity such that the RNP comprising the gNA comprising the targeting sequence can form a complementary bond with respect to the target nucleic acid.

Representative, but non-limiting examples of targeting sequences to wild-type PCSK9 nucleic acid are presented as SEQ ID NOS: 315-436, 612-2100, and 2286-13861, and are shown below as Table A, representing targeting sequences for PCSK9 target nucleic acid. In one embodiment, the targeting sequence of the gNA comprises a sequence having at least about 65%, at least about 75%, at least about 85%, or at least about 95% identity to a sequence selected from the group consisting of SEQ ID NOS: 315-436, 612-2100, and 2286-13861. In another embodiment, the targeting sequence of the gNA consists of a sequence selected from the group consisting of SEQ ID NOS: 315-436, 612-2100, and 2286-13861. In the foregoing embodiments, thymine (T) nucleotides can be substituted for one or more or all of the uracil (U) nucleotides in any of the targeting sequences such that the gNA can be a gDNA or a gRNA, or a chimera of RNA and DNA. In some embodiments, a targeting sequence selected from the group consisting of SEQ ID NOS: 315-436, 612-2100, and 2286-13861 has at least 1, 2, 3, 4, 5, or 6 or more thymine nucleotides substituted for uracil nucleotides. In other embodiments, a gNA, gRNA, or gDNA of the disclosure comprises 1, 2, 3 or more targeting sequences selected from the group consisting of SEQ ID NOS: 315-436, 612-2100, and 2286-13861, or targeting sequences that are at least 50% identical, at least 55% identical, at least 60% identical, at least 65% identical, at least 70% identical, at least 75% identical, at least 80% identical, at least 85% identical, at least 90% identical, at least 95% identical to one or more sequences of SEQ ID NOS: 315-436, 612-2100, and 2286-13861. In some embodiments, the targeting sequence of the gNA comprises a sequence selected from the group consisting of SEQ ID NOS: 315-436, 612-2100, and 2286-13861 with a single nucleotide removed from the 3′ end of the sequence. In other embodiments, the targeting sequence of the gNA comprises a sequence o selected from the group consisting of SEQ ID NOS: 315-436, 612-2100, and 2286-13861 with two nucleotides removed from the 3′ end of the sequence. In other embodiments, the targeting sequence of the gNA comprises a sequence selected from the group consisting of SEQ ID NOS: 315-436, 612-2100, and 2286-13861 with three nucleotides removed from the 3′ end of the sequence. In other embodiments, the targeting sequence of the gNA comprises a sequence selected from the group consisting of SEQ ID NOS: 315-436, 612-2100, and 2286-13861 with four nucleotides removed from the 3′ end of the sequence. In other embodiments, the targeting sequence of the gNA comprises a sequence selected from the group consisting of SEQ ID NOS: 315-436, 612-2100, and 2286-13861 with five nucleotides removed from the 3′ end of the sequence.

TABLE A
Targeting Sequences Specific to PCSK9

| | PAM |
|---|---|
| SEQ ID NO: | Sequence |
| 315-436, 612-2, 100, | ATCN |
| 2,286-3,183 | |
| 3,184-7,251 | TTCN |
| 7,252-11,521 | CTCN |
| 11,522-13, 861 | GTCN |

In some embodiments, the targeting sequence is complementary to a nucleic acid sequence encoding a mutation of the PCSK9 protein of SEQ ID NO: 33 or mutations that disrupt the function or expression of the PCSK9 protein. Several missense mutations (S127R, D129G, F216L, D374H, and D374Y) are associated with hypercholesterolemia and premature atherosclerosis; hence are considered gain-of-function mutations (Shilpa Pandit, S., et al. Functional analysis of sites within PCSK9 responsible for hypercholesterolemias. J Lipid Res., 49:1333 (2008)), and the disclosure contemplates targeting sequences that are complementary to DNA sequences encoding these mutations in the PCSK9 gene, including a sequence selected from the group consisting of AGCAGGUCGCCUCUCAUCUU (SEQ ID NO: 272), CAUCUUCACCAGGAAGCCAG (SEQ ID NO: 273), CCUCUCAUCUUCACCAGGAA (SEQ ID NO: 274), UGGUGAAGAUGAGAGGCGAC (SEQ ID NO: 275), GUGGAGGCGGGUCCCGUCCU (SEQ ID NO: 281), AGCCACUGCAGCACCUGCUU (SEQ ID NO: 287), UUGGUGCCUCCAGCCACUGC (SEQ ID NO: 288), AGCUACUGCAGCACCUGCUU (SEQ ID NO: 289), and UUGGUGCCUCCAGCUACUGC (SEQ ID NO: 290).

Several mutations are considered loss of function mutations, including R46L, G106R, Y142X, N157K, R237W and C679X, and are associated with hypocholesterolemia (Berke, K., et al. Missense Mutations in the PCSK9 Gene Are Associated With Hypocholesterolemia and Possibly Increased Response to Statin Therapy. Arteriosclerosis Thrombosis and Vascular Biol. 26:1094 (2006)), and the disclosure contemplates targeting sequences that are complementary to DNA sequences encoding these mutations in the PCSK9 gene. Exemplary targeting sequences specific to PCSK9 mutations, and the ClinVar (/www.ncbi.nlm.nih.gov/clinvar/) identifiers of the PCSK9 mutations targeted by the spacers, are presented in Table B below.

TABLE B
Targeting Sequences for PCSK9 Mutations

| | | SEQ |
|---|---|---|
| | | ID |
| Name | Sequence | NO: |
| VCV000431555 | UGGUGGCCGCUGCCAUGCUG | 24 |
| VCV000440705 | CACGGGCGCCCGUGCGCAGG | 248 |
| VCV000440705 | UGCGCACGGGCGCCCGUGGG | 249 |
| VCV000440705 | UGGGUCCCACGGGCGCCCGU | 250 |
| VCV000496566 | CGCGGGCGCCGUGCGCAGGA | 251 |
| VCV000496566 | UCCUCCUGCGCACGGCGCCC | 252 |
| VCV000496566 | UCCUGCGCACGGCGCCCGCG | 253 |
| VCV000496566 | UGCGCACGGCGCCCGCGGGA | 254 |
| VCV000496566 | UGGGUCCCGCGGGCGCCGUG | 255 |
| VCV000440706 | CAACGCAAGGCUAGCACCAG | 256 |
| VCV000440706 | UCCUCGCAACGCAAGGCUAG | 257 |
| VCV000440706 | UCGCAACGCAAGGCUAGCAC | 258 |
| VCV000440708 | CCCAGGCCGUCCUCCUCGGA | 259 |
| VCV000440708 | GAGGAGGACGGCCUGGGCGA | 260 |
| VCV000440708 | GGUGCUUCGCCCAGGCCGUC | 261 |
| VCV000440708 | GUGCUCGGGUGCUUCGCCCA | 262 |
| VCV000265918 | GUGGUUCCGUGCUCGGGUGC | 263 |
| VCV000440709 | CAUCCCUACACCUGCACCUU | 264 |
| VCV000440709 | CUACACCUGCACCUUGGCGC | 265 |
| VCV000440712 | CCUCCGUCAGCACCACCACG | 266 |
| VCV000440712 | GUCAGCACCACCACGUAGGU | 267 |
| VCV000440712 | UCCGUCAGCACCACCACGUA | 268 |
| VCV000440714 | GAGCGCACUGCCUGCCGCCU | 269 |
| VCV000440715 | UGGUGGGGUAUCCCCGGCGG | 270 |
| VCV000375849 | UGGUGGGGUAUCCCCGGCGG | 271 |
| VCV000002873 | AGCAGGUCGCCUCUCAUCUU | 272 |
| VCV000002873 | CAUCUUCACCAGGAAGCCAG | 273 |
| VCV000002873 | CCUCUCAUCUUCACCAGGAA | 274 |
| VCV000002873 | UGGUGAAGAUGAGAGGCGAC | 275 |
| VCV000189308 | AAGUUGGUGACCAUGACCCU | 276 |
| VCV000189308 | CCAACUUCGAGAAUGUGCCC | 277 |
| VCV000189308 | CGAAGUUGGUGACCAUGACC | 278 |
| VCV000189308 | GGCACAUUCUCGAAGUUGGU | 279 |
| VCV000189308 | UGGUCACCAACUUCGAGAAU | 280 |
| VCV000002874 | GUGGAGGCGGGUCCCGUCCU | 281 |
| VCV000440717 | CACUUGCUGGCCUGCUAGAC | 282 |
| VCV000440717 | UCUAGCAGGCCAGCAAGUGU | 283 |
| VCV000440717 | UUGUGUUCGUCUAGCAGGCC | 284 |
| VCV000438337 | ACACAGCGGCCAAAGAUGGU | 285 |
| VCV000438337 | UUGGCCGCUGUGUGGACCUC | 286 |
| VCV000265939 | AGCCACUGCAGCACCUGCUU | 287 |
| VCV000265939 | UUGGUGCCUCCAGCCACUGC | 288 |
| VCV000002875 | AGCUACUGCAGCACCUGCUU | 289 |
| VCV000002875 | UUGGUGCCUCCAGCUACUGC | 290 |
| VCV000440720 | CAGGCUGCUGCCCACAUGGC | 291 |
| VCV000440721 | GCACACUCGGGGCCUGCACG | 292 |
| VCV000440721 | GGGCCUGCACGGAUGGCCAC | 293 |
| VCV000440721 | GUGCAGGCCCCGAGUGUGCU | 294 |
| VCV000440722 | ACAGCCGUCGCCCGCUGCGC | 295 |
| VCV000440722 | GGGCCUACACGGAUGUCCAC | 296 |
| VCV000440723 | CCUGCUGCGCCCCAGAUGAG | 297 |
| VCV000440723 | GGGGCGCAGCAGGCGACGGC | 298 |
| VCV000440723 | UCAUCUGGGGCGCAGCAGGC | 299 |
| VCV000440723 | UCUGGGGCGCAGCAGGCGAC | 300 |
| VCV000440724 | AUGCGCUCGCCCCGCCGCUU | 301 |
| VCV000440724 | CUUCCAUGCGCUCGCCCCGC | 302 |
| VCV000440725 | CGGCCCCUUGGCUGGUGCUG | 303 |

Representative, but non-limiting examples of targeting sequences to mutant PCSK9 nucleic acids are presented as SEQ ID NOS: 247-303, and are shown supra as Table B. In one embodiment, the targeting sequence of the gNA comprises a sequence having at least about 65%, at least about 75%, at least about 85%, or at least about 95% identity to a sequence selected from the group consisting of SEQ ID NOS: 247-303. In another embodiment, the targeting sequence of the gNA consists of a sequence selected from the group consisting of SEQ ID NOS: 247-303. In some embodiments, a targeting sequence selected from the group consisting of SEQ ID NOS: 247-303 has at least 1, 2, 3, 4, 5, or 6 or more thymine nucleotides substituted for uracil nucleotides. In other embodiments, the disclosure provides CasX:gNA systems comprising 1, 2, 3 or more gNA comprising targeting sequences selected from the group consisting of SEQ ID NOS: 247-303, or targeting sequences that are at least 50% identical, at least 55% identical, at least 60% identical, at least 65% identical, at least 70% identical, at least 75% identical, at least 80% identical, at least 85% identical, at least 90% identical, at least 95% identical to one or more sequences of SEQ ID NOs: 247-303. In some embodiments, the targeting sequence of the gNA comprises a sequence selected from the group consisting of SEQ ID NOS: 247-303 with a single nucleotide removed from the 3′ end of the sequence. In other embodiments, the targeting sequence of the gNA comprises a sequence o selected from the group consisting of SEQ ID NOS: 247-303 with two nucleotides removed from the 3′ end of the sequence. In other embodiments, the targeting sequence of the gNA comprises a sequence selected from the group consisting of SEQ ID NOS: 247-303 with three nucleotides removed from the 3′ end of the sequence. In other embodiments, the targeting sequence of the gNA comprises a sequence selected from the group consisting of SEQ ID NOS: 3247-303 with four nucleotides removed from the 3′ end of the sequence. In other embodiments, the targeting sequence of the gNA comprises a sequence selected from the group consisting of SEQ ID NOS: 247-303 with five nucleotides removed from the 3′ end of the sequence.

In some embodiments, the CasX:gNA system comprises a first gNA and further comprises a second (and optionally a third, fourth, fifth, or more) gNA, wherein the second gNA or additional gNA has a targeting sequence complementary to a different or overlapping portion of the target nucleic acid sequence compared to the targeting sequence of the first gNA such that multiple points in the target nucleic acid are targeted, and, for example, multiple breaks are introduced in the target nucleic acid by the CasX. It will be understood that in such cases, the second or additional gNA is complexed with an additional copy of the CasX protein. By selection of the targeting sequences of the gNA, defined regions of the target nucleic acid sequence bracketing a particular location within the target nucleic acid can be modified or edited using the CasX:gNA systems described herein, including facilitating the insertion of a donor template or excision of a region or exon comprising a mutation of the PCSK9 gene.

e. gNA Scaffolds

With the exception of the targeting sequence domain, the remaining components of the gNA are referred to herein as the scaffold. In some embodiments, the gNA scaffolds are derived from naturally-occurring sequences, described below as reference gNA. In other embodiments, the gNA scaffolds are variants of reference gNA wherein mutations, insertions, deletions or domain substitutions are introduced to confer desirable properties on the gNA.

In some embodiments, a CasX reference gRNA comprises a sequence isolated or derived from Deltaproteobacter. In some embodiments, the sequence is a CasX tracrRNA sequence. Exemplary CasX reference tracrRNA sequences isolated or derived from Deltaproteobacter may include: ACAUCUGGCGCGUUUAUUCCAUUACUUUGGAGCCAGUCCCAGCGACUAUGUCGU AUGGACGAAGCGCUUAUUUAUCGGAGA (SEQ ID NO: 22) and ACAUCUGGCGCGUUUAUUCCAUUACUUUGGAGCCAGUCCCAGCGACUAUGUCGU AUGGACGAAGCGCUUAUUUAUCGG (SEQ ID NO: 23). Exemplary crRNA sequences isolated or derived from Deltaproteobacter may comprise a sequence of CCGAUAAGUAAAACGCAUCAAAG (SEQ ID NO: 24). In some embodiments, a CasX reference gNA comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Deltaproteobacter.

In some embodiments, a CasX reference guide RNA comprises a sequence isolated or derived from Planctomycetes. In some embodiments, the sequence is a CasX tracrRNA sequence. Exemplary CasX reference tracrRNA sequences isolated or derived from Planctomycetes may include: UACUGGCGCUUUUAUCUCAUUACUUUGAGAGCCAUCACCAGCGACUAUGUCGUA UGGGUAAAGCGCUUAUUUAUCGGAGA (SEQ ID NO: 8) and UACUGGCGCUUUUAUCUCAUUACUUUGAGAGCCAUCACCAGCGACUAUGUCGUA UGGGUAAAGCGCUUAUUUAUCGG (SEQ ID NO: 9). Exemplary crRNA sequences isolated or derived from Planctomycetes may comprise a sequence of UCUCCGAUAAAUAAGAAGCAUCAAAG (SEQ ID NO: 27). In some embodiments, a CasX reference gNA comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Planctomycetes.

In some embodiments, a CasX reference gNA comprises a sequence isolated or derived from Candidatus Sungbacteria. In some embodiments, the sequence is a CasX tracrRNA sequence. Exemplary CasX reference tracrRNA sequences isolated or derived from Candidatus Sungbacteria may comprise sequences of: GUUUACACACUCCCUCUCAUAGGGU (SEQ ID NO: 10), GUUUACACACUCCCUCUCAUGAGGU (SEQ ID NO: 11), UUUUACAUACCCCCUCUCAUGGGAU (SEQ ID NO: 12) and GUUUACACACUCCCUCUCAUGGGGG (SEQ ID NO: 13). In some embodiments, a CasX reference guide RNA comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Candidatus Sungbacteria.

Table 1 provides the sequences of reference gRNAs tracr and scaffold sequences. In some embodiments, the disclosure provides gNA sequences wherein the gNA has a scaffold comprising a sequence having at least one nucleotide modification relative to a reference gNA sequence having a sequence of any one of SEQ ID NOS:4-16 of Table 1. It will be understood that in those embodiments wherein a vector comprises a DNA encoding sequence for a gNA, or where a gNA is a gDNA or a chimera of RNA and DNA, that thymine (T) bases can be substituted for the uracil (U) bases of any of the gNA sequence embodiments described herein, including the sequences of Table 1 and Table 2.

TABLE 1
Reference gRNA tracr and scaffold sequences

| SEQ | |
|---|---|
| ID | |
| NO. | Nucleotide Sequence |
| 4 | ACAUCUGGCGCGUUUAUUCCAUUACUUUGGAGCCAGUCCCAGCGACUAUGUCG |
| | UAUGGACGAAGCGCUUAUUUAUCGGAGAGAAACCGAUAAGUAAAACGCAUCAA |
| | AG |
| 5 | UACUGGCGCUUUUAUCUCAUUACUUUGAGAGCCAUCACCAGCGACUAUGUCGU |
| | AUGGGUAAAGCGCUUAUUUAUCGGAGAGAAAUCCGAUAAAUAAGAAGCAUCAA |
| | AG |
| 6 | ACAUCUGGCGCGUUUAUUCCAUUACUUUGGAGCCAGUCCCAGCGACUAUGUCG |
| | UAUGGACGAAGCGCUUAUUUAUCGGAGA |
| 7 | ACAUCUGGCGCGUUUAUUCCAUUACUUUGGAGCCAGUCCCAGCGACUAUGUCG |
| | UAUGGACGAAGCGCUUAUUUAUCGG |
| 8 | UACUGGCGCUUUUAUCUCAUUACUUUGAGAGCCAUCACCAGCGACUAUGUCGU |
| | AUGGGUAAAGCGCUUAUUUAUCGGAGA |
| 9 | UACUGGCGCUUUUAUCUCAUUACUUUGAGAGCCAUCACCAGCGACUAUGUCGU |
| | AUGGGUAAAGCGCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAUGUCGUAUGG |
| | GUAAAGCGCUUAUUUAUCGGA |

f. gNA Variants

In another aspect, the disclosure relates to guide nucleic acid variants (referred to herein alternatively as “gNA variant” or “gRNA variant”), which comprise one or more modifications relative to a reference gRNA scaffold. As used herein, “scaffold” refers to all parts to the gNA necessary for gNA function with the exception of the spacer sequence.

In some embodiments, the scaffold of the gNA variant is a variant comprising one or more additional changes to a sequence of a reference gRNA that comprises SEQ ID NO:4 or SEQ ID NO:5. In those embodiments where the scaffold of the reference gRNA is derived from SEQ ID NO:4 or SEQ ID NO:5, the one or more improved or added characteristics of the gNA variant are improved compared to the same characteristic in SEQ ID NO:4 or SEQ ID NO:5. In some embodiments, a gNA variant comprises one or more nucleotide substitutions, insertions, deletions, or swapped or replaced regions relative to a reference gRNA sequence of the disclosure. In some embodiments, a mutation can occur in any region of a reference gRNA to produce a gNA variant. In some embodiments, the scaffold of the gNA variant sequence has at least 20%, at least 30%, at least 40%, at least 50%, at least 60%, or at least 70%, at least 80%, at least 85%, at least about 90%, at least about 95%, at least about 96%, at least about 97%, at least about 98%, or at least about 99% identity to the sequence of SEQ ID NO:4 or SEQ ID NO:5.

In some embodiments, a gNA variant comprises one or more nucleotide changes within one or more regions of the reference gRNA that improve a characteristic of the reference gRNA. Exemplary regions include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In one embodiment, the gNA variant scaffold comprises a scaffold stem loop having at least 60% sequence identity to SEQ ID NO:14. In another embodiment, the gNA variant comprises a scaffold stem loop having the sequence of CCAGCGACUAUGUCGUAGUGG (SEQ ID NO: 32). In another embodiment, the disclosure provides a gNA scaffold comprising, relative to SEQ ID NO:5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G64U. In the foregoing embodiment, the gNA scaffold comprises the sequence

(SEQ ID NO: 2238)
ACUGGCGCUUUUAUCUGAUUACUUUGAGAGCCAUCACCAGCGAC
UAUGUCGUAGUGGGUAAAGCUCCCUCUUCGGAGGGAGCAUCAAAG.

All gNA variants that have one or more improved functions or characteristics, or add one or more new functions when the variant gNA is compared to a reference gRNA described herein, are envisaged as within the scope of the disclosure. A representative example of such a gNA variant is guide 174 (SEQ ID NO:2238), the design of which is described in the Examples. In some embodiments, the gNA variant adds a new function to the RNP comprising the gNA variant. In some embodiments, the gNA variant has an improved characteristic selected from: improved stability; improved solubility; improved transcription of the gNA; improved resistance to nuclease activity; increased folding rate of the gNA; decreased side product formation during folding; increased productive folding; improved binding affinity to a CasX protein; improved binding affinity to a target DNA when complexed with a CasX protein; improved gene editing when complexed with a CasX protein; improved specificity of editing when complexed with a CasX protein; and improved ability to utilize a greater spectrum of one or more PAM sequences, including ATC, CTC, GTC, or TTC (also referred to as ATCN, CTCN, GTCN and TTCN PAMs), in the editing of target DNA when complexed with a CasX protein, or any combination thereof. In some cases, the one or more of the improved characteristics of the gNA variant is at least about 1.1 to about 100,000-fold improved relative to the reference gNA of SEQ ID NO:4 or SEQ ID NO:5. In other cases, the one or more improved characteristics of the gNA variant is at least about 1.1, at least about 10, at least about 100, at least about 1000, at least about 10,000, at least about 100,000-fold or more improved relative to the reference gNA of SEQ ID NO:4 or SEQ ID NO:5. In other cases, the one or more of the improved characteristics of the gNA variant is about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to the reference gNA of SEQ ID NO:4 or SEQ ID NO:5. In other cases, the one or more improved characteristics of the gNA variant is about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to the reference gNA of SEQ ID NO:4 or SEQ ID NO:5.

In some embodiments, a gNA variant can be created by subjecting a reference gRNA to a one or more mutagenesis methods, such as the mutagenesis methods described herein, below, which may include Deep Mutational Evolution (DME), deep mutational scanning (DMS), error prone PCR, cassette mutagenesis, random mutagenesis, staggered extension PCR, gene shuffling, or domain swapping, in order to generate the gNA variants of the disclosure. The activity of reference gRNAs may be used as a benchmark against which the activity of gNA variants are compared, thereby measuring improvements in function of gNA variants. In other embodiments, a reference gRNA may be subjected to one or more deliberate, targeted mutations, substitutions, or domain swaps in order to produce a gNA variant, for example a rationally designed variant. Exemplary gRNA variants produced by such methods are described in the Examples and representative sequences of gNA scaffolds are presented in Table 2.

In some embodiments, the gNA variant comprises one or more modifications compared to a reference guide nucleic acid scaffold sequence, wherein the one or more modification is selected from: at least one nucleotide substitution in a region of the gNA variant; at least one nucleotide deletion in a region of the gNA variant; at least one nucleotide insertion in a region of the gNA variant; a substitution of all or a portion of a region of the gNA variant; a deletion of all or a portion of a region of the gNA variant; or any combination of the foregoing. In some cases, the modification is a substitution of 1 to 15 consecutive or non-consecutive nucleotides in the gNA variant in one or more regions. In other cases, the modification is a deletion of 1 to 10 consecutive or non-consecutive nucleotides in the gNA variant in one or more regions. In other cases, the modification is an insertion of 1 to 10 consecutive or non-consecutive nucleotides in the gNA variant in one or more regions. In other cases, the modification is a substitution of the scaffold stem loop or the extended stem loop with an RNA stem loop sequence from a heterologous RNA source with proximal 5′ and 3′ ends. In some cases, a gNA variant of the disclosure comprises two or more modifications in one region. In other cases, a gNA variant of the disclosure comprises modifications in two or more regions. In other cases, a gNA variant comprises any combination of the foregoing modifications described in this paragraph.

In some embodiments, a 5′ G is added to a gNA variant sequence for expression in vivo, as transcription from a U6 promoter is more efficient and more consistent with regard to the start site when the +1 nucleotide is a G. In other embodiments, two 5′ Gs are added to a gNA variant sequence for in vitro transcription to increase production efficiency, as T7 polymerase strongly prefers a G in the +1 position and a purine in the +2 position. In some cases, the 5′ G bases are added to the reference scaffolds of Table 1. In other cases, the 5′ G bases are added to the variant scaffolds of Table 2.

Table 2 provides exemplary gNA variant scaffold sequences. In Table 2, (−) indicates a deletion at the specified position(s) relative to the reference sequence of SEQ ID NO:5, (+) indicates an insertion of the specified base(s) at the position indicated relative to SEQ ID NO:5, (:) indicates the range of bases at the specified start:stop coordinates of a deletion or substitution relative to SEQ ID NO:5, and multiple insertions, deletions or substitutions are separated by commas; e.g., A14C, U17G. In some embodiments, the gNA variant scaffold comprises any one of SEQ ID NOS: 2101-2285 as listed in Table 2, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the gNA variant scaffold comprises or consists essentially of a sequence selected from the group consisting of SEQ ID NOS: 2101-2285 as set forth in Table 2. It will be understood that in those embodiments wherein a vector comprises a DNA encoding sequence for a gNA, or where a gNA is a gDNA or a chimera of RNA and DNA, that thymine (T) bases can be substituted for the uracil (U) bases of any of the gNA sequence embodiments described herein.

TABLE 2
Exemplary gNA Scaffold Sequences

| SEQ | | |
|---|---|---|
| ID | NAME or | |
| NO: | Modification | NUCLEOTIDE SEQUENCE |
| 2101 | phage | UACUGGCGCUUUUAUCUCAUUACUUUGAGAGCCAUCACCAGCGACUA |
| | replication | UGUCGUAUGGGUAAAGCGCAGGUGGGACGACCUCUCGGUCGUCCUAU |
| | stable | CUGAAGCAUCAAAG |
| 2102 | Kissing | UACUGGCGCUUUUAUCUCAUUACUUUGAGAGCCAUCACCAGCGACUA |
| | loop_b1 | UGUCGUAUGGGUAAAGCGCUGCUCGACGCGUCCUCGAGCAGAAGCAU |
| | | CAAAG |
| 2103 | Kissing | UACUGGCGCUUUUAUCUCAUUACUUUGAGAGCCAUCACCAGCGACUA |
| | loopa | UGUCGUAUGGGUAAAGCGCUGCUCGCUCCGUUCGAGCAGAAGCAUCA |
| | | AAG |
| 2104 | 32:uvsX | GUACUGGCGCUUUUAUCUCAUUACUUUGAGAGCCAUCACCAGCGACU |
| | hairpin | AUGUCGUAUGGGUAAAGCGCCCUCUUCGGAGGGAAGCAUCAAAG |
| 2105 | PP7 | UACUGGCGCUUUUAUCUCAUUACUUUGAGAGCCAUCACCAGCGACUA |
| | | UGUCGUAUGGGUAAAGCGCAGGAGUUUCUAUGGAAACCCUGAAGCAU |
| | | CAAAG |
| 2106 | 64:trip mut, | GUACUGGCGCCUUUAUCUCAUUACUUUGAGAGCCAUCACCAGCGACU |
| | extended stem | AUGUCGUAUGGGUAAAGCGCUUACGGACUUCGGUCCGUAAGAAGCAU |
| | truncation | CAAAG |
| 2107 | hyperstable | UACUGGCGCUUUUAUCUCAUUACUUUGAGAGCCAUCACCAGCGACUA |
| | tetraloop | UGUCGUAUGGGUAAAGCGCUGCGCUUGCGCAGAAGCAUCAAAG |
| 2108 | C18G | UACUGGCGCUUUUAUCUGAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2109 | U17G | UACUGGCGCUUUUAUCGCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2110 | CUUCGG | UACUGGCGCUUUUAUCUCAUUACUUUGAGAGCCAUCACCAGCGACUA |
| | loop | UGUCGUAUGGGUAAAGCGCUUAUUUAUCGGAGACUUCGGUCCGAUAA |
| | | AUAAGAAGCAUCAAAG |
| 2111 | MS2 | UACUGGCGCUUUUAUCUCAUUACUUUGAGAGCCAUCACCAGCGACUA |
| | | UGUCGUAUGGGUAAAGCGCACAUGAGGAUUACCCAUGUGAAGCAUCA |
| | | AAG |
| 2112 | −1, A2G, −78, | GCUGGCGCUUUUAUCUCAUUACUUUGAGAGCCAUCACCAGCGACUAU |
| | G77U | GUCGUAUGGGUAAAGCGCUUAUUUAUCGUGAGAAAUCCGAUAAAUAA |
| | | GAAGCAUCAAAG |
| 2113 | QB | UACUGGCGCUUUUAUCUCAUUACUUUGAGAGCCAUCACCAGCGACUA |
| | | UGUCGUAUGGGUAAAGCGCUGCAUGUCUAAGACAGCAGAAGCAUCAA |
| | | AG |
| 2114 | 45, 44 hairpin | UACUGGCGCUUUUAUCUCAUUACUUUGAGAGCCAUCACCAGCGACUA |
| | | UGUCGUAUGGGUAAAGCGCAGGGCUUCGGCCGAAGCAUCAAAG |
| 2115 | UIA | UACUGGCGCUUUUAUCUCAUUACUUUGAGAGCCAUCACCAGCGACUA |
| | | UGUCGUAUGGGUAAAGCGCAAUCCAUUGCACUCCGGAUUGAAGCAUC |
| | | AAAG |
| 2116 | A14C, U17G | UACUGGCGCUUUUCUCGCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2117 | CUUCGG | UACUGGCGCUUUUAUCUCAUUACUUUGAGAGCCAUCACCAGCGACUA |
| | loop modified | UGUCGUAUGGGUAAAGCGCUUAUUUAUCGGACUUCGGUCCGAUAAAU |
| | | AAGAAGCAUCAAAG |
| 2118 | Kissing | UACUGGCGCUUUUAUCUCAUUACUUUGAGAGCCAUCACCAGCGACUA |
| | loop_b2 | UGUCGUAUGGGUAAAGCGCUGCUCGUUUGCGGCUACGAGCAGAAGCA |
| | | UCAAAG |
| 2119 | −76:78, −83:87 | UACUGGCGCUUUUAUCUCAUUACUUUGAGAGCCAUCACCAGCGACUA |
| | | UGUCGUAUGGGUAAAGCGCUUAUUUAUCGAGAGAUAAAUAAGAAGCA |
| | | UCAAAG |
| 2120 | −4 | UACGGCGCUUUUAUCUCAUUACUUUGAGAGCCAUCACCAGCGACUAU |
| | | GUCGUAUGGGUAAAGCGCUUAUUUAUCGGAGAGAAAUCCGAUAAAUA |
| | | AGAAGCAUCAAAG |
| 2121 | extended stem | UACUGGCGCCUUUUAUCUCAUUACUUUGAGAGCCAUCACCAGCGACU |
| | truncation | AUGUCGUAUGGGUAAAGCGCUUACGGACUUCGGUCCGUAAGAAGCAU |
| | | CAAAG |
| 2122 | C55 | UACUGGCGCUUUUAUCUCAUUACUUUGAGAGCCAUCACCAGCGACUA |
| | | UGUCGUAUCGGUAAAGCGCUUAUUUAUCGGAGAGAAAUCCGAUAAAU |
| | | AAGAAGCAUCAAAG |
| 2123 | trip mut | UACUGGCGCCUUUAUCUCAUUACUUUGAGAGCCAUCACCAGCGACUA |
| | | UGUCGUAUGGGUAAAGCGCUUAUUUAUCGGACUUCGGUCCGAUAAAU |
| | | AAGAAGCAUCAAAG |
| 2124 | −76:78 | UACUGGCGCUUUUAUCUCAUUACUUUGAGAGCCAUCACCAGCGACUA |
| | | UGUCGUAUGGGUAAAGCGCUUAUUUAUCGAGAAAUCCGAUAAAUAAG |
| | | AAGCAUCAAAG |
| 2125 | −1:5 | GCGCUUUUAUCUCAUUACUUUGAGAGCCAUCACCAGCGACUAUGUCG |
| | | UAUGGGUAAAGCGCUUAUUUAUCGGAGAGAAAUCCGAUAAAUAAGAA |
| | | GCAUCAAAG |
| 2126 | −83:87 | UACUGGCGCUUUUAUCUCAUUACUUUGAGAGCCAUCACCAGCGACUA |
| | | UGUCGUAUGGGUAAAGCGCUUAUUUAUCGGAGAGAGAUAAAUAAGAA |
| | | GCAUCAAAG |
| 2127 | = +G28, | UACUGGCGCUUUUAUCUCAUUACUUUGGAGAGCCAUCACCAGCGACU |
| | A82U, −84, | AUGUCGUAUGGGUAAAGCGCUUAUUUAUCGGAGAGUAUCCGAUAAAU |
| | | AAGAAGCAUCAAAG |
| 2128 | = +51U | UACUGGCGCUUUUAUCUCAUUACUUUGAGAGCCAUCACCAGCGACUA |
| | | UGUUCGUAUGGGUAAAGCGCUUAUUUAUCGGAGAGAAAUCCGAUAAA |
| | | UAAGAAGCAUCAAAG |
| 2129 | −1:4, +G5A, | AGCGCUUUUAUCUCAUUACUUUGAGAGCCAUCACCAGCGACUAUGUC |
| | +G86, | GUAUGGGUAAAGCGCUUAUUUAUCGGAGAGAAAUGCCGAUAAAUAAG |
| | | AAGCAUCAAAG |
| 2130 | = +A94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | UAAGAAGCAUCAAAG |
| 2131 | = +G72 | UACUGGCGCUUUUAUCUCAUUACUUUGAGAGCCAUCACCAGCGACUA |
| | | UGUCGUAUGGGUAAAGCGCUUAUUGUAUCGGAGAGAAAUCCGAUAAA |
| | | UAAGAAGCAUCAAAG |
| 2132 | shorten front, | GCGCUUUUAUCUCAUUACUUUGAGAGCCAUCACCAGCGACUAUGUCG |
| | CUUCGG | UAUGGGUAAAGCGCUUAUUUAUCGGACUUCGGUCCGAUAAAUAAGCG |
| | loop modified, | CAUCAAAG |
| | extend | |
| | extended | |
| 2133 | A14C | UACUGGCGCUUUUCUCUCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2134 | −1:3, +G3 | GUGGCGCUUUUAUCUCAUUACUUUGAGAGCCAUCACCAGCGACUAUG |
| | | UCGUAUGGGUAAAGCGCUUAUUUAUCGGAGAGAAAUCCGAUAAAUAA |
| | | GAAGCAUCAAAG |
| 2135 | = +C45, +U46 | UACUGGCGCUUUUAUCUCAUUACUUUGAGAGCCAUCACCAGCGACCU |
| | | UAUGUCGUAUGGGUAAAGCGCUUAUUUAUCGGAGAGAAAUCCGAUAA |
| | | AUAAGAAGCAUCAAAG |
| 2136 | CUUCGG | GAUGGCGCUUUUAUCUCAUUACUUUGAGAGCCAUCACCAGCGACUAU |
| | loop modified, | GUCGUAUGGGUAAAGCGCUUAUUUAUCGGACUUCGGUCCGAUAAAUA |
| | fun start | AGAAGCAUCAAAG |
| 2137 | −93:94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | GAAGCAUCAAAG |
| 2138 | = +U45 | UACUGGCGCUUUUAUCUCAUUACUUUGAGAGCCAUCACCAGCGAUCU |
| | | AUGUCGUAUGGGUAAAGCGCUUAUUUAUCGGAGAGAAAUCCGAUAAA |
| | | UAAGAAGCAUCAAAG |
| 2139 | −69, −94 | UACUGGCGCUUUUAUCUCAUUACUUUGAGAGCCAUCACCAGCGACUA |
| | | UGUCGUAUGGGUAAAGGCUUAUUUAUCGGAGAGAAAUCCGAUAAAAA |
| | | GAAGCAUCAAAG |
| 2140 | −94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | AGAAGCAUCAAAG |
| 2141 | modified | UACUGGCGCUUUAUCUCAUUACUUUGAGAGCCAUCACCAGCGACUAU |
| | CUUCGG, | GUCGUAUGGGUAAAGCGCUUAUUUAUCGGACUUCGGUCCGAUAAAUA |
| | minus U in 1st | AGAAGCAUCAAAG |
| | triplex | |
| 2142 | −1:4, +C4, | CGGCGCUUUUCUCGCAUUACUUUGAGAGCCAUCACCAGCGACUAUGU |
| | A14C, U17G, | CGUAUGGGUAAAGCGCUUAUUGUAUCGAGAGAUAAAUAAGAAGCAUC |
| | +G72, −76:78, | AAAG |
| | −83:87 | |
| 2143 | U1C, −73 | CACUGGCGCUUUUAUCUCAUUACUUUGAGAGCCAUCACCAGCGACUA |
| | | UGUCGUAUGGGUAAAGCGCUUAUUUUCGGAGAGAAAUCCGAUAAAUA |
| | | AGAAGCAUCAAAG |
| 2144 | Scaffold | UACUGGCGCUUUAUCUCAUUACUUUGAGAGCCAUCACCAGCGACUUC |
| | uuCG, stem | GGUCGUAUGGGUAAAGCGCUUAUGUAUCGGCUUCGGCCGAUACAUAA |
| | uuCG. Stem | GAAGCAUCAAAG |
| | swap, t | |
| | shorten | |
| 2145 | Scaffold | UACUGGCGCUUUUAUCUCAUUACUUUGAGAGCCAUCACCAGCGACUU |
| | uuCG, stem | CGGUCGUAUGGGUAAAGCGCUUAUGUAUCGGCUUCGGCCGAUACAUA |
| | uuCG. Stem | AGAAGCAUCAAAG |
| | swap | |
| 2146 | = +G60 | UACUGGCGCUUUUAUCUCAUUACUUUGAGAGCCAUCACCAGCGACUA |
| | | UGUCGUAUGGGUGAAAGCGCUUAUUUAUCGGAGAGAAAUCCGAUAAA |
| | | UAAGAAGCAUCAAAG |
| 2147 | no stem | UACUGGCGCUUUUAUCUCAUUACUUUGAGAGCCAUCACCAGCGACUU |
| | Scaffold | CGGUCGUAUGGGUAAAG |
| | uuCG | |
| 2148 | no stem | GAUGGGCUUUUAUCUCAUUACUUUGAGAGCCAUCACCAGCGACUUCG |
| | Scaffold | GUCGUAUGGGUAAAG |
| | uuCG, fun | |
| | start | |
| 2149 | Scaffold | GAUGGGCUUUUAUCUCAUUACUUUGAGAGCCAUCACCAGCGACUUCG |
| | uuCG, stem | GUCGUAUGGGUAAAGCGCUUAUUUAUCGGCUUCGGCCGAUAAAUAAG |
| | uuCG, fun | AAGCAUCAAAG |
| | start | |
| 2150 | Pseudoknots | UACUGGCGCUUUUAUCUCAUUACUUUGAGAGCCAUCACCAGCGACUA |
| | | UGUCGUAUGGGUAAAGCGCUACACUGGGAUCGCUGAAUUAGAGAUCG |
| | | GCGUCCUUUCAUUCUAUAUACUUUGGAGUUUUAAAAUGUCUCUAAGU |
| | | ACAGAAGCAUCAAAG |
| 2151 | Scaffold | GGCGCUUUUAUCUCAUUACUUUGAGAGCCAUCACCAGCGACUUCGGU |
| | uuCG, stem | CGUAUGGGUAAAGCGCUUAUUUAUCGGCUUCGGCCGAUAAAUAAGAA |
| | uuCG | GCAUCAAAG |
| 2152 | Scaffold | GCUGGCGCUUUUAUCUCAUUACUUUGAGAGCCAUCACCAGCGACUUC |
| | uuCG, stem | GGUCGUAUGGGUAAAGCGCUUAUUUAUCGGCUUCGGCCGAUAAAUAA |
| | uuCG, no start | GAAGCAUCAAAG |
| 2153 | Scaffold | UACUGGCGCUUUUAUCUCAUUACUUUGAGAGCCAUCACCAGCGACUU |
| | uuCG | CGGUCGUAUGGGUAAAGCGCUUAUUUAUCGGAGAGAAAUCCGAUAAA |
| | | UAAGAAGCAUCAAAG |
| 2154 | = +GCUC36 | UACUGGCGCUUUUAUCUCAUUACUUUGAGAGCCAUGCUCCACCAGCG |
| | | ACUAUGUCGUAUGGGUAAAGCGCUUAUUUAUCGGAGAGAAAUCCGAU |
| | | AAAUAAGAAGCAUCAAAG |
| 2155 | G quadriplex | UACUGGCGCUUUUAUCUCAUUACUUUGAGAGCCAUCACCAGCGACUA |
| | telomere | UGUCGUAUGGGUAAAGCGGGGUUAGGGUUAGGGUUAGGGAAGCAUCA |
| | basket + ends | AAG |
| 2156 | G quadriplex | UACUGGCGCUUUUAUCUCAUUACUUUGAGAGCCAUCACCAGCGACUA |
| | M3q | UGUCGUAUGGGUAAAGCGGAGGGAGGGAGGGAGAGGGAAAGCAUCAA |
| | | AG |
| 2157 | G quadriplex | UACUGGCGCUUUUAUCUCAUUACUUUGAGAGCCAUCACCAGCGACUA |
| | telomere | UGUCGUAUGGGUAAAGCGUUGGGUUAGGGUUAGGGUUAGGGAAAAGC |
| | basket no ends | AUCAAAG |
| 2158 | 45, 44 hairpin | UACUGGCGCUUUUAUCUCAUUACUUUGAGAGCCAUCACCAGCGACUA |
| | (old version) | UGUCGUAUGGGUAAAGCGC----------AGGGCUUCGGCCG----- |
| | | --GAAGCAUCAAAG |
| 2159 | Sarcin−ricin | UACUGGCGCUUUUAUCUCAUUACUUUGAGAGCCAUCACCAGCGACUA |
| | loop | UGUCGUAUGGGUAAAGCGCCUGCUCAGUACGAGAGGAACCGCAGGAA |
| | | GCAUCAAAG |
| 2160 | uvsX, C18G | UACUGGCGCUUUUAUCUGAUUACUUUGAGAGCCAUCACCAGCGACUA |
| | | UGUCGUAUGGGUAAAGCGCCCUCUUCGGAGGGAAGCAUCAAAG |
| 2161 | truncated stem | UACUGGCGCCUUUAUCUGAUUACUUUGAGAGCCAUCACCAGCGACUA |
| | loop, C18G, | UGUCGUAUGGGUAAAGCGCUUACGGACUUCGGUCCGUAAGAAGCAUC |
| | trip mut | AAAG |
| | (U10C) | |
| 2162 | short phage | UACUGGCGCUUUUAUCUGAUUACUUUGAGAGCCAUCACCAGCGACUA |
| | rep, C18G | UGUCGUAUGGGUAAAGCGCGGACGACCUCUCGGUCGUCCGAAGCAUC |
| | | AAAG |
| 2163 | phage rep | UACUGGCGCUUUUAUCUGAUUACUUUGAGAGCCAUCACCAGCGACUA |
| | loop, C18G | UGUCGUAUGGGUAAAGCGCAGGUGGGACGACCUCUCGGUCGUCCUAU |
| | | CUGAAGCAUCAAAG |
| 2164 | = +G18, | UACUGGCGCCUUUAUCUGCAUUACUUUGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2165 | truncated stem | GCUGGCGCUUUUAUCUGAUUACUUUGAGAGCCAUCACCAGCGACUAU |
| | loop, C18G, | GUCGUAUGGGUAAAGCGCUUACGGACUUCGGUCCGUAAGAAGCAUCA |
| | −1 A2G | AAG |
| 2166 | phage rep | UACUGGCGCCUUUAUCUGAUUACUUUGAGAGCCAUCACCAGCGACUA |
| | loop, C18G, | UGUCGUAUGGGUAAAGCGCAGGUGGGACGACCUCUCGGUCGUCCUAU |
| | trip mut | CUGAAGCAUCAAAG |
| | (U10C) | |
| 2167 | short phage | UACUGGCGCCUUUAUCUGAUUACUUUGAGAGCCAUCACCAGCGACUA |
| | rep, C18G, | UGUCGUAUGGGUAAAGCGCGGACGACCUCUCGGUCGUCCGAAGCAUC |
| | trip mut | AAAG |
| | (U10C) | |
| 2168 | uvsX, trip mut | UACUGGCGCCUUUAUCUCAUUACUUUGAGAGCCAUCACCAGCGACUA |
| | (U10C) | UGUCGUAUGGGUAAAGCGCCCUCUUCGGAGGGAAGCAUCAAAG |
| 2169 | truncated stem | UACUGGCGCUUUUAUCUCAUUACUUUGAGAGCCAUCACCAGCGACUA |
| | loop | UGUCGUAUGGGUAAAGCGCUUACGGACUUCGGUCCGUAAGAAGCAUC |
| | | AAAG |
| 2170 | = +A17, | UACUGGCGCCUUUAUCAUCAUUACUUUGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2171 | 3′ HDV | UACUGGCGCUUUUAUCUCAUUACUUUGAGAGCCAUCACCAGCGACUA |
| | genomic | UGUCGUAUGGGUAAAGCGCUUAUUUAUCGGAGAGAAAUCCGAUAAAU |
| | ribozyme | AAGAAGCAUCAAAGGGCCGGCAUGGUCCCAGCCUCCUCGCUGGCGCC |
| | | GGCUGGGCAACAUUCCGAGGGGACCGUCCCCUCGGUAAUGGCGAAUG |
| | | GGACCC |
| 2172 | phage rep | UACUGGCGCCUUUAUCUCAUUACUUUGAGAGCCAUCACCAGCGACUA |
| | loop, trip mut | UGUCGUAUGGGUAAAGCGCAGGUGGGACGACCUCUCGGUCGUCCUAU |
| | (U10C) | CUGAAGCAUCAAAG |
| 2173 | −79:80 | UACUGGCGCUUUUAUCUCAUUACUUUGAGAGCCAUCACCAGCGACUA |
| | | UGUCGUAUGGGUAAAGCGCUUAUUUAUCGGAGAAAUCCGAUAAAUAA |
| | | GAAGCAUCAAAG |
| 2174 | short phage | UACUGGCGCCUUUAUCUCAUUACUUUGAGAGCCAUCACCAGCGACUA |
| | rep, trip mut | UGUCGUAUGGGUAAAGCGCGGACGACCUCUCGGUCGUCCGAAGCAUC |
| | (U10C) | AAAG |
| 2175 | extra | UACUGGCGCUUUUAUCUCAUUACUUUGAGAGCCAUCACCAGCGACUA |
| | truncated stem | UGUCGUAUGGGUAAAGCGCCGGACUUCGGUCCGGAAGCAUCAAAG |
| | loop | |
| 2176 | U17G, C18G | UACUGGCGCUUUUAUCGGAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2177 | short phage | UACUGGCGCUUUUAUCUCAUUACUUUGAGAGCCAUCACCAGCGACUA |
| | rep | UGUCGUAUGGGUAAAGCGCGGACGACCUCUCGGUCGUCCGAAGCAUC |
| | | AAAG |
| 2178 | uvsX, C18G, | GCUGGCGCUUUUAUCUGAUUACUUUGAGAGCCAUCACCAGCGACUAU |
| | −1 A2G | GUCGUAUGGGUAAAGCGCCCUCUUCGGAGGGAAGCAUCAAAG |
| 2179 | uvsX, C18G, | GCUGGCGCCUUUAUCUGAUUACUUUGAGAGCCAUCACCAGCGACUAU |
| | trip mut | GUCGUAUGGGUAAAGCUCCCUCUUCGGAGGGAGCAUCAAAG |
| | (U10C), −1 | |
| | A2G, HDV | |
| | −99 G65U | |
| 2180 | 3′ HDV | UACUGGCGCUUUUAUCUCAUUACUUUGAGAGCCAUCACCAGCGACUA |
| | antigenomic | UGUCGUAUGGGUAAAGCGCUUAUUUAUCGGAGAGAAAUCCGAUAAAU |
| | ribozyme | AAGAAGCAUCAAAGGGGUCGGCAUGGCAUCUCCACCUCCUCGCGGUC |
| | | CGACCUGGGCAUCCGAAGGAGGACGCACGUCCACUCGGAUGGCUAAG |
| | | GGAGAGCCA |
| 2181 | uvsX, C18G, | GCUGGCGCCUUUAUCUGAUUACUUUGAGAGCCAUCACCAGCGACUAU |
| | trip mut | GUCGUAUGGGUAAAGCGCCCUCUUCGGAGGGCGCAUCAAAG |
| | (U10C), −1 | |
| | A2G, HDV | |
| | AA(98:99)C | |
| 2182 | 3′ HDV | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (Lior Nissim, | AAGAAGCAUCAAAGUUUUGGCCGGCAUGGUCCCAGCCUCCUCGCUGG |
| | Timothy Lu) | CGCCGGCUGGGCAACAUGCUUCGGCAUGGCGAAUGGGACCCCGGG |
| 2183 | TAC(1:3)GA, | GAUGGCGCCUUUAUCUCAUUACUUUGAGAGCCAUCACCAGCGACUAU |
| | stacked onto | GUCGUAUGGGUAAAGCGCUUACGGACUUCGGUCCGUAAGAAGCAUCA |
| | 64 | AAG |
| 2184 | uvsX, −1 A2G | GCUGGCGCUUUUAUCUCAUUACUUUGAGAGCCAUCACCAGCGACUAU |
| | | GUCGUAUGGGUAAAGCGCCCUCUUCGGAGGGAAGCAUCAAAG |
| 2185 | truncated stem | GCUGGCGCCUUUAUCUGAUUACUUUGAGAGCCAUCACCAGCGACUAU |
| | loop, C18G, | GUCGUAUGGGUAAAGCUCUUACGGACUUCGGUCCGUAAGAGCAUCAA |
| | trip mut | AG |
| | (U10C), −1 | |
| | A2G, HDV | |
| | −99 G65U | |
| 2186 | short phage | GCUGGCGCCUUUAUCUGAUUACUUUGAGAGCCAUCACCAGCGACUAU |
| | rep, C18G, | GUCGUAUGGGUAAAGCUCGGACGACCUCUCGGUCGUCCGAGCAUCAA |
| | trip mut | AG |
| | (U10C), −1 | |
| | A2G, HDV − | |
| | 99 G65U | |
| 2187 | 3′ sTRSV WT | UACUGGCGCUUUUAUCUCAUUACUUUGAGAGCCAUCACCAGCGACUA |
| | viral | UGUCGUAUGGGUAAAGCGCUUAUUUAUCGGAGAGAAAUCCGAUAAAU |
| | Hammerhead | AAGAAGCAUCAAAGCCUGUCACCGGAUGUGCUUUCCGGUCUGAUGAG |
| | ribozyme | UCCGUGAGGACGAAACAGG |
| 2188 | short phage | GCUGGCGCUUUUAUCUGAUUACUUUGAGAGCCAUCACCAGCGACUAU |
| | rep, C18G, −1 | GUCGUAUGGGUAAAGCGCGGACGACCUCUCGGUCGUCCGAAGCAUCA |
| | A2G | AAG |
| 2189 | short phage | GCUGGCGCCUUUAUCUGAUUACUUUGAGAGCCAUCACCAGCGACUAU |
| | rep, C18G, | GUCGUAUGGGUAAAGCGCGGACGACCUCUCGGUCGUCCGAAGCAUCA |
| | trip mut | AAG |
| | (U10C), −1 | |
| | A2G, 3′ | |
| | genomic HDV | |
| 2190 | phage rep | GCUGGCGCCUUUAUCUGAUUACUUUGAGAGCCAUCACCAGCGACUAU |
| | loop, C18G, | GUCGUAUGGGUAAAGCUCAGGUGGGACGACCUCUCGGUCGUCCUAUC |
| | trip mut | UGAGCAUCAAAG |
| | (U10C), −1 | |
| | A2G, HDV | |
| | −99 G65U | |
| 2191 | 3′ HDV | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (Owen Ryan, | AAGAAGCAUCAAAGGAUGGCCGGCAUGGUCCCAGCCUCCUCGCUGGC |
| | Jamie Cate) | GCCGGCUGGGCAACACCUUCGGGUGGCGAAUGGGAC |
| 2192 | phage rep | GCUGGCGCUUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | −1 A2G | UGAAGCAUCAAAG |
| 2193 | 0.14 | UACUGGCGCUUUUAUCUCAUUACUUUGAGAGCCAUCACCAGCGACUA |
| | | UGUCGUACUGGGUAAAGCGCUUAUUUAUCGGAGAGAAAUCCGAUAAA |
| | | UAAGAAGCAUCAAAG |
| 2194 | −78, G77U | UACUGGCGCUUUUAUCUCAUUACUUUGAGAGCCAUCACCAGCGACUA |
| | | UGUCGUAUGGGUAAAGCGCUUAUUUAUCGUGAGAAAUCCGAUAAAUA |
| | | AGAAGCAUCAAAG |
| 2195 | | GUACUGGCGCUUUUAUCUCAUUACUUUGAGAGCCAUCACCAGCGACU |
| | | AUGUCGUAUGGGUAAAGCGCUUAUUUAUCGGAGAGAAAUCCGAUAAA |
| | | UAAGAAGCAUCAAAG |
| 2196 | short phage | GCUGGCGCUUUUAUCUCAUUACUUUGAGAGCCAUCACCAGCGACUAU |
| | rep, −1 A2G | GUCGUAUGGGUAAAGCGCGGACGACCUCUCGGUCGUCCGAAGCAUCA |
| | | AAG |
| 2197 | truncated stem | GCUGGCGCCUUUAUCUGAUUACUUUGAGAGCCAUCACCAGCGACUAU |
| | loop, C18G, | GUCGUAUGGGUAAAGCGCUUACGGACUUCGGUCCGUAAGAAGCAUCA |
| | trip mut | AAG |
| | (U10C), −1 | |
| | A2G | |
| 2198 | −1, A2G | GCUGGCGCUUUUAUCUCAUUACUUUGAGAGCCAUCACCAGCGACUAU |
| | | GUCGUAUGGGUAAAGCGCUUAUUUAUCGGAGAGAAAUCCGAUAAAUA |
| | | AGAAGCAUCAAAG |
| 2199 | truncated stem | GCUGGCGCCUUUAUCUCAUUACUUUGAGAGCCAUCACCAGCGACUAU |
| | loop, trip mut | GUCGUAUGGGUAAAGCGCUUACGGACUUCGGUCCGUAAGAAGCAUCA |
| | (U10C), −1 | AAG |
| | A2G | |
| 2200 | uvsX, C18G, | GCUGGCGCCUUUAUCUGAUUACUUUGAGAGCCAUCACCAGCGACUAU |
| | trip mut | GUCGUAUGGGUAAAGCGCCCUCUUCGGAGGGAAGCAUCAAAG |
| | (U10C), −1 | |
| | A2G | |
| 2201 | phage rep | GCUGGCGCUUUUAUCUCAUUACUUUGAGAGCCAUCACCAGCGACUAU |
| | loop, −1 A2G | GUCGUAUGGGUAAAGCGCAGGUGGGACGACCUCUCGGUCGUCCUAUC |
| | | UGAAGCAUCAAAG |
| 2202 | phage rep | GCUGGCGCCUUUAUCUCAUUACUUUGAGAGCCAUCACCAGCGACUAU |
| | loop, trip mut | GUCGUAUGGGUAAAGCGCAGGUGGGACGACCUCUCGGUCGUCCUAUC |
| | (U10C), −1 | UGAAGCAUCAAAG |
| | A2G | |
| 2203 | phage rep | GCUGGCGCCUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | trip mut | UGAAGCAUCAAAG |
| | (U10C), −1 | |
| | A2G | |
| 2204 | truncated stem | UACUGGCGCUUUUAUCUGAUUACUUUGAGAGCCAUCACCAGCGACUA |
| | loop, C18G | UGUCGUAUGGGUAAAGCGCUUACGGACUUCGGUCCGUAAGAAGCAUC |
| | | AAAG |
| 2205 | uvsX, trip mut | GCUGGCGCCUUUAUCUCAUUACUUUGAGAGCCAUCACCAGCGACUAU |
| | (U10C), −1 | GUCGUAUGGGUAAAGCGCCCUCUUCGGAGGGAAGCAUCAAAG |
| | A2G | |
| 2206 | truncated stem | GCUGGCGCUUUUAUCUCAUUACUUUGAGAGCCAUCACCAGCGACUAU |
| | loop, −1 A2G | GUCGUAUGGGUAAAGCGCUUACGGACUUCGGUCCGUAAGAAGCAUCA |
| | | AAG |
| 2207 | short phage | GCUGGCGCCUUUAUCUCAUUACUUUGAGAGCCAUCACCAGCGACUAU |
| | rep, trip mut | GUCGUAUGGGUAAAGCGCGGACGACCUCUCGGUCGUCCGAAGCAUCA |
| | (U10C), −1 | AAG |
| | A2G | |
| 2208 | 5′ HDV | GAUGGCCGGCAUGGUCCCAGCCUCCUCGCUGGCGCCGGCUGGGCAAC |
| | ribozyme | ACCUUCGGGUGGCGAAUGGGACUACUGGCGCUUUUAUCUCAUUACUU |
| | (Owen Ryan, | UGAGAGCCAUCACCAGCGACUAUGUCGUAUGGGUAAAGCGCUUAUUU |
| | Jamie Cate) | AUGGGAGAGAAAUCCGAUAAAUAAGAAGCAUCAAAG |
| 2209 | 5′ HDV | GGCCGGCAUGGUCCCAGCCUCCUCGCUGGCGCCGGCUGGGCAACAUU |
| | genomic | CCGAGGGGACCGUCCCCUCGGUAAUGGCGAAUGGGACCCUACUGGCG |
| | ribozyme | CUUUUAUCUCAUUACUUUGAGAGCCAUCACCAGCGACUAUGUCGUAU |
| | | GGGUAAAGCGCUUAUUUAUCGGAGAGAAAUCCGAUAAAUAAGAAGCA |
| | | UCAAAG |
| 2210 | truncated stem | GCUGGCGCCUUUAUCUGAUUACUUUGAGAGCCAUCACCAGCGACUAU |
| | loop, C18G, | GUCGUAUGGGUAAAGCGCUUACGGACUUCGGUCCGUAAGCGCAUCAA |
| | trip mut | AG |
| | (U10C), −1 | |
| | A2G, HDV | |
| | AA(98:99)C | |
| 2211 | 5′ env25 pistol | CGUGGUUAGGGCCACGUUAAAUAGUUGCUUAAGCCCUAAGCGUUGAU |
| | ribozyme | CUUCGGAUCAGGUGCAAUACUGGCGCUUUUAUCUCAUUACUUUGAGA |
| | (with an added | GCCAUCACCAGCGACUAUGUCGUAUGGGUAAAGCGCUUAUUUAUCGG |
| | CUUCGG | AGAGAAAUCCGAUAAAUAAGAAGCAUCAAAG |
| | loop) | |
| 2212 | 5′ HDV | GGGUCGGCAUGGCAUCUCCACCUCCUCGCGGUCCGACCUGGGCAUCC |
| | antigenomic | GAAGGAGGACGCACGUCCACUCGGAUGGCUAAGGGAGAGCCAUACUG |
| | ribozyme | GCGCUUUUAUCUCAUUACUUUGAGAGCCAUCACCAGCGACUAUGUCG |
| | | UAUGGGUAAAGCGCUUAUUUAUCGGAGAGAAAUCCGAUAAAUAAGAA |
| | | GCAUCAAAG |
| 2213 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCCAGUACUGAUGAGUCCGUGAGGACGAAACGAG |
| | (Lior Nissim, | UAAGCUCGUCUACUGGCGCUUUUAUCUCAU |
| | Timothy Lu) | |
| | guide scaffold | |
| | scar | |
| 2214 | = +A27, | UACUGGCGCCUUUAUCUCAUUACUUUAGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2215 | 5′ Hammerhea | CGACUACUGAUGAGUCCGUGAGGACGAAACGAGUAAGCUCGUCUAGU |
| | d ribozyme | CGUACUGGCGCUUUUAUCUCAUUACUUUGAGAGCCAUCACCAGCGAC |
| | (Lior Nissim, | UAUGUCGUAUGGGUAAAGCGCUUAUUUAUCGGAGAGAAAUCCGAUAA |
| | Timothy Lu) | AUAAGAAGCAUCAAAG |
| | smaller scar | |
| 2216 | phage rep | GCUGGCGCCUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | trip mut | UGCGCAUCAAAG |
| | (U10C), −1 | |
| | A2G, HDV | |
| | AA(98:99)C | |
| 2217 | −27, stacked | UACUGGCGCCUUUAUCUCAUUACUUUAGAGCCAUCACCAGCGACUAU |
| | onto 64 | GUCGUAUGGGUAAAGCGCUUACGGACUUCGGUCCGUAAGAAGCAUCA |
| | | AAG |
| 2218 | 3′ Hatchet | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAGCAUUCCUCAGAAAAUGACAAACCUGUGGGGCGU |
| | | AAGUAGAUCUUCGGAUCUAUGAUCGUGCAGACGUUAAAAUCAGGU |
| 2219 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCGACUACUGAUGAGUCCGUGAGGACGAAACGAG |
| | (Lior Nissim, | UAAGCUCGUCUAGUCGCGUGUAGCGAAGCA |
| | Timothy Lu) | |
| 2220 | 5′ Hatchet | CAUUCCUCAGAAAAUGACAAACCUGUGGGGCGUAAGUAGAUCUUCGG |
| | | AUCUAUGAUCGUGCAGACGUUAAAAUCAGGUUACUGGCGCUUUUAUC |
| | | UCAUUACUUUGAGAGCCAUCACCAGCGACUAUGUCGUAUGGGUAAAG |
| | | CGCUUAUUUAUCGGAGAGAAAUCCGAUAAAUAAGAAGCAUCAAAG |
| 2221 | 5′ HDV | UUUUGGCCGGCAUGGUCCCAGCCUCCUCGCUGGCGCCGGCUGGGCAA |
| | ribozyme | CAUGCUUCGGCAUGGCGAAUGGGACCCCGGGUACUGGCGCUUUUAUC |
| | (Lior Nissim, | UCAUUACUUUGAGAGCCAUCACCAGCGACUAUGUCGUAUGGGUAAAG |
| | Timothy Lu) | CGCUUAUUUAUCGGAGAGAAAUCCGAUAAAUAAGAAGCAUCAAAG |
| 2222 | 5′ | CGACUACUGAUGAGUCCGUGAGGACGAAACGAGUAAGCUCGUCUAGU |
| | Hammerhead | CGCGUGUAGCGAAGCAUACUGGCGCUUUUAUCUCAUUACUUUGAGAG |
| | ribozyme | CCAUCACCAGCGACUAUGUCGUAUGGGUAAAGCGCUUAUUUAUCGGA |
| | (Lior Nissim, | GAGAAAUCCGAUAAAUAAGAAGCAUCAAAG |
| | Timothy Lu) | |
| 2223 | 3′ HH15 | UACUGGCGCUUUUAUCUCAUUACUUUGAGAGCCAUCACCAGCGACUA |
| | Minimal | UGUCGUAUGGGUAAAGCGCUUAUUUAUCGGAGAGAAAUCCGAUAAAU |
| | Hammerhead | AAGAAGCAUCAAAGGGGAGCCCCGCUGAUGAGGUCGGGGAGACCGAA |
| | ribozyme | AGGGACUUCGGUCCCUACGGGGCUCCC |
| 2224 | 5′ RBMX | CCACCCCCACCACCACCCCCACCCCCACCACCACCCUACUGGCGCUU |
| | recruiting | UUAUCUCAUUACUUUGAGAGCCAUCACCAGCGACUAUGUCGUAUGGG |
| | motif | UAAAGCGCUUAUUUAUCGGAGAGAAAUCCGAUAAAUAAGAAGCAUCA |
| | | AAG |
| 2225 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCGACUACUGAUGAGUCCGUGAGGACGAAACGAG |
| | (Lior Nissim, | UAAGCUCGUCUAGUCG |
| | Timothy Lu) | |
| | smaller scar | |
| 2226 | 3′ env25 pistol | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (with an added | AAGAAGCAUCAAAGCGUGGUUAGGGCCACGUUAAAUAGUUGCUUAAG |
| | CUUCGG | CCCUAAGCGUUGAUCUUCGGAUCAGGUGCAA |
| | loop) | |
| 2227 | 3′ Env−9 | UACUGGCGCUUUUAUCUCAUUACUUUGAGAGCCAUCACCAGCGACUA |
| | Twister | UGUCGUAUGGGUAAAGCGCUUAUUUAUCGGAGAGAAAUCCGAUAAAU |
| | | AAGAAGCAUCAAAGGGCAAUAAAGCGGUUACAAGCCCGCAAAAAUAG |
| | | CAGAGUAAUGUCGCGAUAGCGCGGCAUUAAUGCAGCUUUAUUG |
| 2228 | = +AUUAUC | UACUGGCGCUUUUAUCUCAUUACUAUUAUCUCAUUACUUUGAGAGCC |
| | UCAUUACU | AUCACCAGCGACUAUGUCGUAUGGGUAAAGCGCUUAUUUAUCGGAGA |
| | 25 | GAAAUCCGAUAAAUAAGAAGCAUCAAAG |
| 2229 | 5′ Env−9 | GGCAAUAAAGCGGUUACAAGCCCGCAAAAAUAGCAGAGUAAUGUCGC |
| | Twister | GAUAGCGCGGCAUUAAUGCAGCUUUAUUGUACUGGCGCUUUUAUCUC |
| | | AUUACUUUGAGAGCCAUCACCAGCGACUAUGUCGUAUGGGUAAAGCG |
| | | CUUAUUUAUCGGAGAGAAAUCCGAUAAAUAAGAAGCAUCAAAG |
| 2230 | 3′ Twisted | UACUGGCGCUUUUAUCUCAUUACUUUGAGAGCCAUCACCAGCGACUA |
| | Sister 1 | UGUCGUAUGGGUAAAGCGCUUAUUUAUCGGAGAGAAAUCCGAUAAAU |
| | | AAGAAGCAUCAAAGACCCGCAAGGCCGACGGCAUCCGCCGCCGCUGG |
| | | UGCAAGUCCAGCCGCCCCUUCGGGGGCGGGCGCUCAUGGGUAAC |
| 2231 | no stem | UACUGGCGCUUUUAUCUCAUUACUUUGAGAGCCAUCACCAGCGACUA |
| | | UGUCGUAUGGGUAAAG |
| 2232 | 5′ HH15 | GGGAGCCCCGCUGAUGAGGUCGGGGAGACCGAAAGGGACUUCGGUCC |
| | Minimal | CUACGGGGCUCCCUACUGGCGCUUUUAUCUCAUUACUUUGAGAGCCA |
| | Hammerhead | UCACCAGCGACUAUGUCGUAUGGGUAAAGCGCUUAUUUAUCGGAGAG |
| | ribozyme | AAAUCCGAUAAAUAAGAAGCAUCAAAG |
| 2233 | 5′ | CCAGUACUGAUGAGUCCGUGAGGACGAAACGAGUAAGCUCGUCUACU |
| | Hammerhead | GGCGCUUUUAUCUCAUUACUGGCGCUUUUAUCUCAUUACUUUGAGAG |
| | ribozyme | CCAUCACCAGCGACUAUGUCGUAUGGGUAAAGCGCUUAUUUAUCGGA |
| | (Lior Nissim, | GAGAAAUCCGAUAAAUAAGAAGCAUCAAAG |
| | Timothy Lu) | |
| | guide scaffold | |
| | scar | |
| 2234 | 5′ Twisted | ACCCGCAAGGCCGACGGCAUCCGCCGCCGCUGGUGCAAGUCCAGCCG |
| | Sister 1 | CCCCUUCGGGGGCGGGCGCUCAUGGGUAACUACUGGCGCUUUUAUCU |
| | | CAUUACUUUGAGAGCCAUCACCAGCGACUAUGUCGUAUGGGUAAAGC |
| | | GCUUAUUUAUCGGAGAGAAAUCCGAUAAAUAAGAAGCAUCAAAG |
| 2235 | 5′ sTRSV WT | CCUGUCACCGGAUGUGCUUUCCGGUCUGAUGAGUCCGUGAGGACGAA |
| | viral | ACAGGUACUGGCGCUUUUAUCUCAUUACUUUGAGAGCCAUCACCAGC |
| | Hammerhead | GACUAUGUCGUAUGGGUAAAGCGCUUAUUUAUCGGAGAGAAAUCCGA |
| | ribozyme | UAAAUAAGAAGCAUCAAAG |
| 2236 | 148:= +G55, | GUACUGGCGCCUUUAUCUCAUUACUUUGAGAGCCAUCACCAGCGACU |
| | stacked onto | AUGUCGUAGUGGGUAAAGCGCUUACGGACUUCGGUCCGUAAGAAGCA |
| | 64 | UCAAAG |
| 2237 | 158: | GUACUGGCGCUUUUAUCUGAUUACUUUGAGAGCCAUCACCAGCGACU |
| | 103 + 148(+G5 | AUGUCGUAGUGGGUAAAGCUCCCUCUUCGGAGGGAGCAUCAAAG |
| | 5) −99, G65U | |
| 2238 | 174:Uvsx | ACUGGCGCUUUUAUCUGAUUACUUUGAGAGCCAUCACCAGCGACUAU |
| | Extended stem | GUCGUAGUGGGUAAAGCUCCCUCUUCGGAGGGAGCAUCAAAG |
| | with [A99] | |
| | G65U), | |
| | C18G, \hat{ }G55, | |
| | [GU−1] | |
| 2239 | 175:extended | ACUGGCGCCUUUAUCUCAUUACUUUGAGAGCCAUCACCAGCGACUAU |
| | stem | GUCGUAUGGGUAAAGCGCUUACGGACUUCGGUCCGUAAGAAGCAUCA |
| | truncation, | AAG |
| | U10C, [GU−1] | |
| 2240 | 176:174 with | GCUGGCGCUUUUAUCUGAUUACUUUGAGAGCCAUCACCAGCGACUAU |
| | AIG | GUCGUAGUGGGUAAAGCUCCCUCUUCGGAGGGAGCAUCAAAG |
| | substitution | |
| | for T7 | |
| | transcription | |
| 2241 | 177:174 with | ACUGGCGCUUUUAUCUGAUUACUUUGAGAGCCAUCACCAGCGACUAU |
| | bubble (+G55) | GUCGUAUGGGUAAAGCUCCCUCUUCGGAGGGAGCAUCAAAG |
| | removed | |
| 2242 | 181:stem 42 | ACUGGCGCCUUUAUCUGAUUACUUUGAGAGCCAUCACCAGCGACUAU |
| | (truncated | GUCGUAUGGGUAAAGCGCUUACGGACUUCGGUCCGUAAGAAGCAUCA |
| | stem loop); | AAG |
| | U10C, C18G, | |
| | [GU−1] | |
| | (95 + [GU−1]) | |
| 2243 | 182:stem 42 | ACUGGCGCUUUUAUCUGAUUACUUUGAGAGCCAUCACCAGCGACUAU |
| | (truncated | GUCGUAUGGGUAAAGCGCUUACGGACUUCGGUCCGUAAGAAGCAUCA |
| | stem loop); | AAG |
| | C18G, [GU−1] | |
| 2244 | 183:stem 42 | ACUGGCGCUUUUAUCUGAUUACUUUGAGAGCCAUCACCAGCGACUAU |
| | (truncated | GUCGUAGUGGGUAAAGCGCUUACGGACUUCGGUCCGUAAGAAGCAUC |
| | stem loop); | AAAG |
| | C18G,\hat{ }G55, | |
| | [GU−1] | |
| 2245 | 184:stem 48 | ACUGGCGCUUUUAUCUGAUUACUUUGAGAGCCAUCACCAGCGACUAU |
| | (uvsx, −99 | GUCGUAUUGGGUAAAGCUCCCUCUUCGGAGGGAGCAUCAAAG |
| | g65t); | |
| | C18G,\hat{ }T55, | |
| | [GU−1] | |
| 2246 | 185:stem 42 | ACUGGCGCUUUUAUCUGAUUACUUUGAGAGCCAUCACCAGCGACUAU |
| | (truncated | GUCGUAUUGGGUAAAGCGCUUACGGACUUCGGUCCGUAAGAAGCAUC |
| | stem loop); | AAAG |
| | C18G,\hat{ }U55, | |
| | [GU−1] | |
| 2247 | 186:stem 42 | ACUGGCGCCUUUAUCAUCAUUACUUUGAGAGCCAUCACCAGCGACUA |
| | (truncated | UGUCGUAUGGGUAAAGCGCUUACGGACUUCGGUCCGUAAGAAGCAUC |
| | stem loop); | AAAG |
| | U10C ,\hat{ }A17, | |
| | [GU−1] | |
| 2248 | 187:stem 46 | ACUGGCGCUUUUAUCUGAUUACUUUGAGAGCCAUCACCAGCGACUAU |
| | (uvsx); | GUCGUAGUGGGUAAAGCGCCCUCUUCGGAGGGAAGCAUCAAAG |
| | C18G,\hat{ }G55, | |
| | [GU−1] | |
| 2249 | 188:stem 50 | ACUGGCGCUUUUAUCUGAUUACUUUGAGAGCCAUCACCAGCGACUAU |
| | (ms2U15C, | GUCGUAGUGGGUAAAGCUCACAUGAGGAUCACCCAUGUGAGCAUCAA |
| | −99, g65t); | AG |
| | C18G,\hat{ }G55, | |
| | [GU−1] | |
| 2250 | 189:174 + | ACUGGCACUUUUACCUGAUUACUUUGAGAGCCAACACCAGCGACUAU |
| | G8A; U15C; | GUCGUAGUGGGUAAAGCUCCCUCUUCGGAGGGAGCAUCAAAG |
| | U35A | |
| 2251 | 190:174 + | ACUGGCACUUUUAUCUGAUUACUUUGAGAGCCAUCACCAGCGACUAU |
| | G8A | GUCGUAGUGGGUAAAGCUCCCUCUUCGGAGGGAGCAUCAAAG |
| 2252 | 191:174 + | ACUGGCCCUUUUAUCUGAUUACUUUGAGAGCCAUCACCAGCGACUAU |
| | G8C | GUCGUAGUGGGUAAAGCUCCCUCUUCGGAGGGAGCAUCAAAG |
| 2253 | 192:174 + | ACUGGCGCUUUUACCUGAUUACUUUGAGAGCCAUCACCAGCGACUAU |
| | U15C | GUCGUAGUGGGUAAAGCUCCCUCUUCGGAGGGAGCAUCAAAG |
| 2254 | 193, 174 + | ACUGGCGCUUUUAUCUGAUUACUUUGAGAGCCAACACCAGCGACUAU |
| | U35A | GUCGUAGUGGGUAAAGCUCCCUCUUCGGAGGGAGCAUCAAAG |
| 2255 | 195:175 + | ACUGGCACCUUUACCUGAUUACUUUGAGAGCCAACACCAGGGACUAU |
| | C18G + | GUCGUAUGGGUAAAGCGCUUACGGACUUCGGUCCGUAAGAAGCAUCA |
| | G8A; U15C; | AAG |
| | U35A | |
| 2256 | 196:175 + | ACUGGCACCUUUAUCUGAUUACUUUGAGAGCCAUCACCAGCGACUAU |
| | C18G + G8A | GUCGUAUGGGUAAAGCGCUUACGGACUUCGGUCCGUAAGAAGCAUCA |
| | | AAG |
| 2257 | 197:175 + | ACUGGCCCCUUUAUCUGAUUACUUUGAGAGCCAUCACCAGCGACUAU |
| | C18G + G8C | GUCGUAUGGGUAAAGCGCUUACGGACUUCGGUCCGUAAGAAGCAUCA |
| | | AAG |
| 2258 | 198:175 + | ACUGGCGCCUUUAUCUGAUUACUUUGAGAGCCAACACCAGCGACUAU |
| | C18G + U35A | GUCGUAUGGGUAAAGCGCUUACGGACUUCGGUCCGUAAGAAGCAUCA |
| | | AAG |
| 2259 | 199:174 + | GCUGGCGCUUUUAUCUGAUUACUUUGAGAGCCAUCACCAGCGACUAU |
| | A2G (test G | GUCGUAGUGGGUAAAGCUCCCUCUUCGGAGGGAGCAUCAAAG |
| | transcription | |
| | at start; | |
| | ccGCT...) | |
| 2260 | 200:174 + | GACUGGCGCUUUUAUCUGAUUACUUUGAGAGCCAUCACCAGCGACUA |
| | \hat{ }G1 | UGUCGUAGUGGGUAAAGCUCCCUCUUCGGAGGGAGCAUCAAAG |
| | (ccGACU...) | |
| 2261 | 201:174 + | ACUGGCGCCUUUAUCUGAUUACUUUGGAGAGCCAUCACCAGCGACUA |
| | U10C; \hat{ }G28 | UGUCGUAGUGGGUAAAGCUCCCUCUUCGGAGGGAGCAUCAAAG |
| 2262 | 202:174 + | ACUGGCGCAUUUAUCUGAUUACUUUGUGAGCCAUCACCAGCGACUAU |
| | U10A; A28U | GUCGUAGUGGGUAAAGCUCCCUCUUCGGAGGGAGCAUCAAAG |
| 2263 | 203:174 + | ACUGGCGCCUUUAUCUGAUUACUUUGAGAGCCAUCACCAGCGACUAU |
| | U10C | GUCGUAGUGGGUAAAGCUCCCUCUUCGGAGGGAGCAUCAAAG |
| 2264 | 204:174 + | ACUGGCGCUUUUAUCUGAUUACUUUGGAGAGCCAUCACCAGCGACUA |
| | \hat{ }G28 | UGUCGUAGUGGGUAAAGCUCCCUCUUCGGAGGGAGCAUCAAAG |
| 2265 | 205:174 + | ACUGGCGCAUUUAUCUGAUUACUUUGAGAGCCAUCACCAGCGACUAU |
| | U10A | GUCGUAGUGGGUAAAGCUCCCUCUUCGGAGGGAGCAUCAAAG |
| 2266 | 206, 174 + | ACUGGCGCUUUUAUCUGAUUACUUUGUGAGCCAUCACCAGCGACUAU |
| | A28U | GUCGUAGUGGGUAAAGCUCCCUCUUCGGAGGGAGCAUCAAAG |
| 2267 | 207:174 + | ACUGGCGCUUUUAUUCUGAUUACUUUGAGAGCCAUCACCAGCGACUA |
| | \hat{ }U15 | UGUCGUAGUGGGUAAAGCUCCCUCUUCGGAGGGAGCAUCAAAG |
| 2268 | 208:174 + | ACGGCGCUUUUAUCUGAUUACUUUGAGAGCCAUCACCAGCGACUAUG |
| | [U4] | UCGUAGUGGGUAAAGCUCCCUCUUCGGAGGGAGCAUCAAAG |
| 2269 | 209:174 + | ACUGGCGCUUUUAUAUGAUUACUUUGAGAGCCAUCACCAGCGACUAU |
| | C16A | GUCGUAGUGGGUAAAGCUCCCUCUUCGGAGGGAGCAUCAAAG |
| 2270 | 210:174 + | ACUGGCGCUUUUAUCUUGAUUACUUUGAGAGCCAUCACCAGCGACUA |
| | \hat{ }U17 | UGUCGUAGUGGGUAAAGCUCCCUCUUCGGAGGGAGCAUCAAAG |
| 2271 | 211:174 + | ACUGGCGCUUUUAUCUGAUUACUUUGAGAGCCAGCACCAGCGACUAU |
| | U35G | GUCGUAGUGGGUAAAGCUCCCUCUUCGGAGGGAGCAUCAAAG |
| | (compare with | |
| | 174 + U35A | |
| | above) | |
| 2272 | 212:174 | ACUGGCGCUGUUAUCUGAUUACUUCGAGAGCCAUCACCAGCGACUAU |
| | + U11G, | GUCGUAGUGGGUAAAGCUCCCUCUUCGGAGGGAGCAUCGAAG |
| | A105G | |
| | (A86G), | |
| | U26C | |
| 2273 | 213:174 | ACUGGCGCUCUUAUCUGAUUACUUCGAGAGCCAUCACCAGCGACUAU |
| | + U11C, | GUCGUAGUGGGUAAAGCUCCCUCUUCGGAGGGAGCAUCGAAG |
| | A105G | |
| | (A86G), | |
| | U26C | |
| 2274 | 214: | ACUGGCGCUUGUAUCUGAUUACUCUGAGAGCCAUCACCAGCGACUAU |
| | 174 + U12G; | GUCGUAGUGGGUAAAGCUCCCUCUUCGGAGGGAGCAUCAGAG |
| | A106G | |
| | (A87G), | |
| | U25C | |
| 2275 | 215: | ACUGGCGCUUCUAUCUGAUUACUCUGAGAGCCAUCACCAGCGACUAU |
| | 174 + U12C; | GUCGUAGUGGGUAAAGCUCCCUCUUCGGAGGGAGCAUCAGAG |
| | A106G | |
| | (A87G), | |
| | U25C | |
| 2276 | 216: | ACUGGCGCUUUGAUCUGAUUACCUUGAGAGCCAUCACCAGCGACUAU |
| | 174_tx_11.G, | GUCGUAGUGGGUAAAGCUCCCUCUUCGGAGGGAGCAUCAAGG |
| | 87.G, 22.C | |
| 2277 | 217: | ACUGGCGCUUUCAUCUGAUUACCUUGAGAGCCAUCACCAGCGACUAU |
| | 174_tx_11.C, | GUCGUAGUGGGUAAAGCUCCCUCUUCGGAGGGAGCAUCAAGG |
| | 78.G, 22.C | |
| 2278 | 218:174 | ACUGGCGCUGUUAUCUGAUUACUUUGAGAGCCAUCACCAGCGACUAU |
| | + U11G | GUCGUAGUGGGUAAAGCUCCCUCUUCGGAGGGAGCAUCAAAG |
| 2279 | 219:174 | ACUGGCGCUUUUAUCUGAUUACUUUGAGAGCCAUCACCAGCGACUAU |
| | + A105G | GUCGUAGUGGGUAAAGCUCCCUCUUCGGAGGGAGCAUCGAAG |
| | (A86G) | |
| 2280 | 220:174 | ACUGGCGCUUUUAUCUGAUUACUUCGAGAGCCAUCACCAGCGACUAU |
| | + U26C | GUCGUAGUGGGUAAAGCUCCCUCUUCGGAGGGAGCAUCAAAG |
| 2281 | 221:182 + | ACUGGCACUUCUAUCUGAUUACUCUGAGAGCCAUCACCAGCGACUAU |
| | G8A(196) | GUCGUAUGGGUAAAGCCGCUUACGGACUUCGGUCCGUAAGAGGCAUC |
| | + 215 | AGAG |
| | mutations + | |
| | AC63, A88G | |
| 2282 | 222:174 + | ACUGGCACUUCUAUCUGAUUACUCUGAGAGCCAUCACCAGCGACUAU |
| | G8A(196) | GUCGUAGUGGGUAAAGCUCCCUCUUCGGAGGGAGCAUCAGAG |
| | + 215 | |
| | mutations | |
| 2283 | 223:181 + | ACUGGCACCUUUAUCUGAUUACUUUGAGAGCCAUCACCAGCGACUAU |
| | G8A(196) + | GUCGUAUGGGUAAAGCCGCUUACGGACUUCGGUCCGUAAGAGGCAUC |
| | AC63, A88G | AAAG |
| 2284 | 224:182 + | ACUGGCACUUGUAUCUGAUUACUCUGAGAGCCAUCACCAGCGACUAU |
| | G8A(196) | GUCGUAUGGGUAAAGCCGCUUACGGACUUCGGUCCGUAAGAGGCAUC |
| | + 214 | AGAG |
| | mutations + | |
| | AC63, A88G | |
| 2285 | 225:174 + | ACUGGCACUUGUAUCUGAUUACUCUGAGAGCCAUCACCAGCGACUAU |
| | G8A(196) | GUCGUAGUGGGUAAAGCUCCCUCUUCGGAGGGAGCAUCAGAG |
| | + 214 | |
| | mutations | |

In some embodiments, the gNA variant comprises a tracrRNA stem loop comprising the sequence -UUU-N4-25-UUU- (SEQ ID NO: 34). For example, the gNA variant comprises a scaffold stem loop or a replacement thereof, flanked by two triplet U motifs that contribute to the triplex region. In some embodiments, the scaffold stem loop or replacement thereof comprises at least 4 nucleotides, at least 5 nucleotides, at least 6 nucleotides, at least 7 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides.

In some embodiments, the gNA variant comprises a crRNA sequence with -AAAG- in a location 5′ to the spacer region. In some embodiments, the -AAAG- sequence is immediately 5′ to the spacer region.

In some embodiments, the at least one nucleotide modification to a reference gNA to produce a gNA variant comprises at least one nucleotide deletion in the CasX variant gNA relative to the reference gRNA. In some embodiments, a gNA variant comprises a deletion of 1, 2, 3, 4, 5, 6, 7, 8, 9, 10, 11, 12, 13, 14, 15, 16, 17, 18, 19 or 20 consecutive or non-consecutive nucleotides relative to a reference gNA. In some embodiments, the at least one deletion comprises a deletion of 1, 2, 3, 4, 5, 6, 7, 8, 9, 10, 11, 12, 13, 14, 15, 16, 17, 18, 19 or 20 or more consecutive nucleotides relative to a reference gNA. In some embodiments, the gNA variant comprises 2, 3, 4, 5, 6, 7, 8, 9, 10, 11, 12, 13, 14, 15, 16, 17, 18, 19 or 20 or more nucleotide deletions relative to the reference gNA, and the deletions are not in consecutive nucleotides. In those embodiments where there are two or more non-consecutive deletions in the gNA variant relative to the reference gRNA, any length of deletions, and any combination of lengths of deletions, as described herein, are contemplated as within the scope of the disclosure. For example, in some embodiments, a gNA variant may comprise a first deletion of one nucleotide, and a second deletion of two nucleotides and the two deletions are not consecutive. In some embodiments, a gNA variant comprises at least two deletions in different regions of the reference gRNA. In some embodiments, a gNA variant comprises at least two deletions in the same region of the reference gRNA. For example, the regions may be the extended stem loop, scaffold stem loop, scaffold stem bubble, triplex loop, pseudoknot, triplex, or a 5′ end of the gNA variant. The deletion of any nucleotide in a reference gRNA is contemplated as within the scope of the disclosure.

In some embodiments, the at least one nucleotide modification of a reference gRNA to generate a gNA variant comprises at least one nucleotide insertion. In some embodiments, a gNA variant comprises an insertion of 1, 2, 3, 4, 5, 6, 7, 8, 9 or 10 consecutive or non-consecutive nucleotides relative to a reference gRNA. In some embodiments, the at least one nucleotide insertion comprises an insertion of 1, 2, 3, 4, 5, 6, 7, 8, 9, 10, 11, 12, 13, 14, 15, 16, 17, 18, 19 or 20 or more consecutive nucleotides relative to a reference gRNA. In some embodiments, the gNA variant comprises 2 or more insertions relative to the reference gRNA, and the insertions are not consecutive. In those embodiments where there are two or more non-consecutive insertions in the gNA variant relative to the reference gRNA, any length of insertions, and any combination of lengths of insertions, as described herein, are contemplated as within the scope of the disclosure. For example, in some embodiments, a gNA variant may comprise a first insertion of one nucleotide, and a second insertion of two nucleotides and the two insertions are not consecutive. In some embodiments, a gNA variant comprises at least two insertions in different regions of the reference gRNA. In some embodiments, a gNA variant comprises at least two insertions in the same region of the reference gRNA. For example, the regions may be the extended stem loop, scaffold stem loop, scaffold stem bubble, triplex loop, pseudoknot, triplex, or a 5′ end of the gNA variant. Any insertion of A, G, C, U (or T, in the corresponding DNA) or combinations thereof at any location in the reference gRNA is contemplated as within the scope of the disclosure.

In some embodiments, the at least one nucleotide modification of a reference gRNA to generate a gNA variant comprises at least one nucleic acid substitution. In some embodiments, a gNA variant comprises 1, 2, 3, 4, 5, 6, 7, 8, 9, 10, 11, 12, 13, 14, 15, 16, 17, 18, 19 or 20 or more consecutive or non-consecutive substituted nucleotides relative to a reference gRNA. In some embodiments, a gNA variant comprises 1-4 nucleotide substitutions relative to a reference gRNA. In some embodiments, the at least one substitution comprises a substitution of 1, 2, 3, 4, 5, 6, 7, 8, 9, 10, 11, 12, 13, 14, 15, 16, 17, 18, 19 or 20 or more consecutive nucleotides relative to a reference gRNA. In some embodiments, the gNA variant comprises 2 or more substitutions relative to the reference gRNA, and the substitutions are not consecutive. In those embodiments where there are two or more non-consecutive substitutions in the gNA variant relative to the reference gRNA, any length of substituted nucleotides, and any combination of lengths of substituted nucleotides, as described herein, are contemplated as within the scope of the disclosure. For example, in some embodiments, a gNA variant may comprise a first substitution of one nucleotide, and a second substitution of two nucleotides and the two substitutions are not consecutive. In some embodiments, a gNA variant comprises at least two substitutions in different regions of the reference gRNA. In some embodiments, a gNA variant comprises at least two substitutions in the same region of the reference gRNA. For example, the regions may be the triplex, the extended stem loop, scaffold stem loop, scaffold stem bubble, triplex loop, pseudoknot, triplex, or a 5′ end of the gNA variant. Any substitution of A, G, C, U (or T, in the corresponding DNA) or combinations thereof at any location in the reference gRNA is contemplated as within the scope of the disclosure.

Any of the substitutions, insertions and deletions described herein can be combined to generate a gNA variant of the disclosure. For example, a gNA variant can comprise at least one substitution and at least one deletion relative to a reference gRNA, at least one substitution and at least one insertion relative to a reference gRNA, at least one insertion and at least one deletion relative to a reference gRNA, or at least one substitution, one insertion and one deletion relative to a reference gRNA.

In some embodiments, the gNA variant comprises a scaffold region at least 20% identical, at least 30% identical, at least 40% identical, at least 50% identical, at least 60% identical, at least 65% identical, at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to any one of SEQ ID NOS: 4-16. In some embodiments, the gNA variant comprises a scaffold region at least 60% homologous (or identical) to any one of SEQ ID NOS: 4-16.

In some embodiments, the gNA variant comprises a tracr stem loop at least 60% identical, at least 65% identical, at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NO: 14. In some embodiments, the gNA variant comprises a tracr stem loop at least 60% homologous (or identical) to SEQ ID NO: 14.

In some embodiments, the gNA variant comprises an extended stem loop at least 60% identical, at least 65% identical, at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NO: 15. In some embodiments, the gNA variant comprises an extended stem loop at least 60% homologous (or identical) to SEQ ID NO: 15.

In some embodiments, the gNA variant comprises an exogenous extended stem loop, with such differences from a reference gNA described as follows. In some embodiments, an exogenous extended stem loop has little or no identity to the reference stem loop regions disclosed herein (e.g., SEQ ID NO:15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, at least 500 bp, at least 600 bp, at least 700 bp, at least 800 bp, at least 900 bp, at least 1,000 bp, at least 2,000 bp, at least 3,000 bp, at least 4,000 bp, at least 5,000 bp, at least 6,000 bp, at least 7,000 bp, at least 8,000 bp, at least 9,000 bp, at least 10,000 bp, at least 12,000 bp, at least 15,000 bp or at least 20,000 bp. In some embodiments, the gNA variant comprises an extended stem loop region comprising at least 10, at least 100, at least 500, at least 1000, or at least 10,000 nucleotides. In some embodiments, the heterologous stem loop increases the stability of the gNA. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting gNA has increased stability and, depending on the choice of loop, can interact with certain cellular proteins or RNA. Such exogenous extended stem loops can comprise, for example a thermostable RNA such as MS2 (ACAUGAGGAUUACCCAUGU (SEQ ID NO: 35)), Qβ (UGCAUGUCUAAGACAGCA (SEQ ID NO: 36)), U1 hairpin II (AAUCCAUUGCACUCCGGAUU (SEQ ID NO: 37)), Uvsx (CCUCUUCGGAGG (SEQ ID NO: 38)), PP7 (AGGAGUUUCUAUGGAAACCCU (SEQ ID NO: 39)), Phage replication loop (AGGUGGGACGACCUCUCGGUCGUCCUAUCU (SEQ ID NO: 40)), Kissing loop_a (UGCUCGCUCCGUUCGAGCA (SEQ ID NO: 41)), Kissing loop_b1 (UGCUCGACGCGUCCUCGAGCA (SEQ ID NO: 42)), Kissing loop_b2 (UGCUCGUUUGCGGCUACGAGCA (SEQ ID NO: 43)), G quadriplex M3q (AGGGAGGGAGGGAGAGG (SEQ ID NO: 44)), G quadriplex telomere basket (GGUUAGGGUUAGGGUUAGG (SEQ ID NO: 45)), Sarcin-ricin loop (CUGCUCAGUACGAGAGGAACCGCAG (SEQ ID NO: 46)) or Pseudoknots (UACACUGGGAUCGCUGAAUUAGAGAUCGGCGUCCUUUCAUUCUAUAUACUUUGG AGUUUUAAAAUGUCUCUAAGUACA (SEQ ID NO: 47)). In some embodiments, an exogenous stem loop comprises a long non-coding RNA (lncRNA). As used herein, a lncRNA refers to a non-coding RNA that is longer than approximately 200 bp in length. In some embodiments, the 5′ and 3′ ends of the exogenous stem loop are base paired; i.e., interact to form a region of duplex RNA. In some embodiments, the 5′ and 3′ ends of the exogenous stem loop are base paired, and one or more regions between the 5′ and 3′ ends of the exogenous stem loop are not base paired. In some embodiments, the at least one nucleotide modification comprises: (a) substitution of 1 to 15 consecutive or non-consecutive nucleotides in the gNA variant in one or more regions; (b) a deletion of 1 to 10 consecutive or non-consecutive nucleotides in the gNA variant in one or more regions; (c) an insertion of 1 to 10 consecutive or non-consecutive nucleotides in the gNA variant in one or more regions; (d) a substitution of the scaffold stem loop or the extended stem loop with an RNA stem loop sequence from a heterologous RNA source with proximal 5′ and 3′ ends; or any combination of (a)-(d).

In some embodiments, the gNA variant comprises a scaffold stem loop sequence of CCAGCGACUAUGUCGUAGUGG (SEQ ID NO: 32). In some embodiments, the gNA variant comprises a scaffold stem loop sequence of CCAGCGACUAUGUCGUAGUGG (SEQ ID NO: 32) with at least 1, 2, 3, 4, or 5 mismatches thereto.

In some embodiments, the gNA variant comprises an extended stem loop region comprising less than 32 nucleotides, less than 31 nucleotides, less than 30 nucleotides, less than 29 nucleotides, less than 28 nucleotides, less than 27 nucleotides, less than 26 nucleotides, less than 25 nucleotides, less than 24 nucleotides, less than 23 nucleotides, less than 22 nucleotides, less than 21 nucleotides, or less than 20 nucleotides. In some embodiments, the gNA variant comprises an extended stem loop region comprising less than 32 nucleotides. In some embodiments, the gNA variant further comprises a thermostable stem loop.

In some embodiments, a gNA variant comprises a sequence of any one of SEQ ID NOS: 2201-2285, or having at least about 80%, at least about 90%, at least about 95%, at least about 96%, at least about 97%, at least about 98%, at least about 99% identity thereto. In some embodiments, a gNA variant comprises a sequence selected from the group consisting of SEQ ID NOS: 2106, 2237, 2238, 2239, 2241, 2244, 2275, 2279, 2280, and 2285.

In some embodiments of the gNA variants of the disclosure, the gNA variant comprises at least one modification, wherein the at least one modification compared to the reference guide scaffold of SEQ ID NO: 5 is selected from one or more of: (a) a C18G substitution in the triplex loop; (b) a G55 insertion in the stem bubble; (c) a U1 deletion; (d) a modification of the extended stem loop wherein (i) a 6 nt loop and 13 loop-proximal base pairs are replaced by a Uvsx hairpin; and (ii) a deletion of A99 and a substitution of G65U that results in a loop-distal base that is fully base-paired. In such embodiments, the gNA variant comprises the sequence of any one of SEQ ID NOS: 2236, 2237, 2238, 2241, 2244, 2248, 2249, or 2259-2285.

In exemplary embodiments, a sgRNA variant comprises one or more additional changes to a sequence of SEQ ID NO: 2238 (Variant Scaffold 174, referencing Table 2).

In exemplary embodiments, a sgRNA variant comprises one or more additional changes to a sequence of SEQ ID NO: 2239 (Variant Scaffold 175, referencing Table 2).

In some embodiments, the gNA variant further comprises a spacer (or targeting sequence) region located at the 3′ end of the gNA, described more fully, supra, which comprises at least 14 to about 35 nucleotides wherein the spacer is designed with a sequence that is complementary to a target DNA. In some embodiments, the gNA variant comprises a targeting sequence of at least 10 to 30 nucleotides complementary to a target DNA. In some embodiments, the targeting sequence has 14, 15, 16, 17, 18, 19, 20, 21, 22, 23, 24, 25, 26, 27, 28, 29, 30, 31, 32, 33, 34 or 35 nucleotides. In some embodiments, the gNA variant comprises a targeting sequence having 20 nucleotides. In some embodiments, the targeting sequence has 25 nucleotides. In some embodiments, the targeting sequence has 24 nucleotides. In some embodiments, the targeting sequence has 23 nucleotides. In some embodiments, the targeting sequence has 22 nucleotides. In some embodiments, the targeting sequence has 21 nucleotides. In some embodiments, the targeting sequence has 20 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides.

In some embodiments, the scaffold of the gNA variant is part of an RNP with a CasX variant protein comprising a sequence of any one of SEQ ID NOS: 49-160, 439, 441, 443, 445, 447-460, 472, 474, 476, 478, 480, 482, 484, 486, 488, or 490 as set forth in Tables 3, 5, 6, 7 and 9, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto. In the foregoing embodiments, the gNA further comprises a spacer sequence.

In the embodiments of the gNA variants, the gNA variant further comprises a spacer (or targeting sequence) region located at the 3′ end of the gNA, described more fully, supra, which comprises at least 14 to about 35 nucleotides wherein the spacer is designed with a sequence that is complementary to a target nucleic acid. In some embodiments, the gNA variant comprises a targeting sequence of at least 10 to 30 nucleotides complementary to a target nucleic acid. In some embodiments, the targeting sequence has 14, 15, 16, 17, 18, 19, 20, 21, 22, 23, 24, 25, 26, 27, 28, 29, 30, 31, 32, 33, 34 or 35 nucleotides. In some embodiments, the gNA variant comprises a targeting sequence having 20 nucleotides. In some embodiments, the targeting sequence has 25 nucleotides. In some embodiments, the targeting sequence has 24 nucleotides. In some embodiments, the targeting sequence has 23 nucleotides. In some embodiments, the targeting sequence has 22 nucleotides. In some embodiments, the targeting sequence has 21 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides. In some embodiments, the disclosure provides targeting sequences for inclusion in the gNA variants of the disclosure comprising a sequence selected from the group consisting of SEQ ID NOS: 247-303, 315-436, 612-2100, or 2286-13861, or a sequence that is at least 50% identical, at least 55% identical, at least 60% identical, at least 65% identical, at least 70% identical, at least 75% identical, at least 80% identical, at least 85% identical, at least 90% identical, at least 95% identical thereto. In some embodiments, the targeting sequence of the gNA variant comprises a sequence a sequence of SEQ ID NOS: 247-303, 315-436, 612-2100, or 2286-13861 with a single nucleotide removed from the 3′ end of the sequence. In other embodiments, the targeting sequence of the gNA variant comprises a sequence a sequence of SEQ ID NOS: 247-303, 315-436, 612-2100, or 2286-13861 with two nucleotides removed from the 3′ end of the sequence. In other embodiments, the targeting sequence of the gNA variant comprises a sequence a sequence of SEQ ID NOS: 247-303, 315-436, 612-2100, or 2286-13861 with three nucleotides removed from the 3′ end of the sequence. In other embodiments, the targeting sequence of the gNA variant comprises a sequence a sequence of SEQ ID NOS: 247-303, 315-436, 612-2100, or 2286-13861 with four nucleotides removed from the 3′ end of the sequence. In other embodiments, the targeting sequence of the gNA variant comprises a sequence a sequence of SEQ ID NOS: 247-303, 315-436, 612-2100, or 2286-13861 with five nucleotides removed from the 3′ end of the sequence.

In some embodiments, the gNA variant further comprises a spacer (targeting) region located at the 3′ end of the gNA, wherein the spacer is designed with a sequence that is complementary to a target nucleic acid. In some embodiments, the target nucleic acid comprises a PAM sequence located 5′ of the spacer with at least a single nucleotide separating the PAM from the first nucleotide of the spacer. In some embodiments, the PAM is located on the non-targeted strand of the target region, i.e. the strand that is complementary to the target nucleic acid. In some embodiments, the PAM sequence is ATC. In some embodiments, the targeting sequence for an ATC PAM comprises a sequence selected from the group consisting of SEQ ID NOS: 315-436, 612-2100, and 2286-3183, or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 315-436, 612-2100, and 2286-3183. In some embodiments, the targeting sequence for an ATC PAM is selected from the group consisting of SEQ ID NOS: 315-436, 612-2100, and 2286-3183. In some embodiments, the PAM sequence is CTC. In some embodiments, the targeting sequence for a CTC PAM comprises a sequence selected from the group consisting of SEQ ID NOS: 7252-11521, or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 7252-11521. In some embodiments, the targeting sequence for a CTC PAM is selected from the group consisting of SEQ ID NOS: 7252-11521. In some embodiments, the PAM sequence is GTC. In some embodiments, the targeting sequences for a GTC PAM comprises a sequence selected from the group consisting of SEQ ID NOS: 11522-13861 or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 11522-13861. In some embodiments, the targeting sequence for a GTC PAM is selected from the group consisting of SEQ ID NOS: 11522-13861. In some embodiments, the PAM sequence is TTC. In some embodiments, a targeting sequences for a TTC PAM comprises a sequence selected from the group consisting of SEQ ID NOS: 3184-7251, or a sequence that is at least 50% identical, at least 55% identical, at least 60% identical, at least 65% identical, at least 70% identical, at least 75% identical, at least 80% identical, at least 85% identical, at least 90% identical, at least 95% identical to SEQ ID NOS: 3184-7251. In some embodiments, a targeting sequence for a TTC PAM is selected from the group consisting of SEQ ID NOS: 3184-7251.

In some embodiments, the gNA variant comprises a targeting sequence located at the 3′ end of the gNA wherein the targeting sequence is complementary to a target nucleic acid sequence comprising a mutation, wherein the mutation is a gain of function mutation. In a particular embodiment of the foregoing, the mutation comprises an amino acid substitution selected from the group consisting of S127R, D129G, F216L, D374H, and D374Y relative to the sequence of SEQ ID NO: 33 In another particular embodiment of the foregoing, the targeting sequence comprises a sequence selected from the group consisting of SEQ ID NOS: 247-303 as set forth in Table B. In another particular embodiment of the foregoing, the targeting sequence comprises a sequence selected from the group consisting of AGCAGGUCGCCUCUCAUCUU (SEQ ID NO: 272), CAUCUUCACCAGGAAGCCAG (SEQ ID NO: 273), CCUCUCAUCUUCACCAGGAA (SEQ ID NO: 274), UGGUGAAGAUGAGAGGCGAC (SEQ ID NO: 275), GUGGAGGCGGGUCCCGUCCU (SEQ ID NO: 281), AGCCACUGCAGCACCUGCUU (SEQ ID NO: 287), UUGGUGCCUCCAGCCACUGC (SEQ ID NO: 288), AGCUACUGCAGCACCUGCUU (SEQ ID NO: 289), and UUGGUGCCUCCAGCUACUGC (SEQ ID NO: 290). In another particular embodiment of the foregoing, the targeting sequence consists of a sequence selected from the group consisting of AGCAGGUCGCCUCUCAUCUU (SEQ ID NO: 272), CAUCUUCACCAGGAAGCCAG (SEQ ID NO: 273), CCUCUCAUCUUCACCAGGAA (SEQ ID NO: 274), UGGUGAAGAUGAGAGGCGAC (SEQ ID NO: 275), GUGGAGGCGGGUCCCGUCCU (SEQ ID NO: 281), AGCCACUGCAGCACCUGCUU (SEQ ID NO: 287), UUGGUGCCUCCAGCCACUGC (SEQ ID NO: 288), AGCUACUGCAGCACCUGCUU (SEQ ID NO: 289), and UUGGUGCCUCCAGCUACUGC (SEQ ID NO: 290). In other embodiments, the gNA variant comprises a targeting sequence located at the 3′ end of the gNA wherein the targeting sequence is complementary to a target nucleic acid sequence comprising a mutation, wherein the mutation is a loss of function mutation. In a particular embodiment of the foregoing, the mutation comprises an amino acid substitution selected from the group consisting of R46L, G106R, Y142X, N157K, R237W or C679X relative to the sequence of SEQ ID NO: 33.

g. Complex Formation with CasX Protein

In some embodiments, a gNA variant has an improved ability to form a complex with a CasX protein (such as a reference CasX or a CasX variant protein) when compared to a reference gRNA. In some embodiments, a gNA variant has an improved affinity for a CasX protein (such as a reference or variant protein) when compared to a reference gRNA, thereby improving its ability to form a ribonucleoprotein (RNP) complex with the CasX protein, as described in the Examples. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising a gNA variant scaffold of the disclosure and its spacer are competent for gene editing of a target nucleic acid.

Exemplary nucleotide changes that can improve the ability of gNA variants to form a complex with CasX protein may, in some embodiments, include replacing the scaffold stem with a thermostable stem loop. Without wishing to be bound by any theory, replacing the scaffold stem with a thermostable stem loop could increase the overall binding stability of the gNA variant with the CasX protein. Alternatively, or in addition, removing a large section of the stem loop could change the gNA variant folding kinetics and make a functional folded gNA easier and quicker to structurally-assemble, for example by lessening the degree to which the gNA variant can get “tangled” in itself. In some embodiments, choice of scaffold stem loop sequence could change with different spacers that are utilized for the gNA. In some embodiments, scaffold sequence can be tailored to the spacer and therefore the target sequence. Biochemical assays can be used to evaluate the binding affinity of CasX protein for the gNA variant to form the RNP, including the assays of the Examples. For example, a person of ordinary skill can measure changes in the amount of a fluorescently tagged gNA that is bound to an immobilized CasX protein, as a response to increasing concentrations of an additional unlabeled “cold competitor” gNA. Alternatively, or in addition, fluorescence signal can be monitored to or seeing how it changes as different amounts of fluorescently labeled gNA are flowed over immobilized CasX protein. Alternatively, the ability to form an RNP can be assessed using in vitro cleavage assays against a defined target nucleic acid sequence.

h. gNA Stability

In some embodiments, a gNA variant has improved stability when compared to a reference gRNA. Increased stability and efficient folding may, in some embodiments, increase the extent to which a gNA variant persists inside a target cell, which may thereby increase the chance of forming a functional RNP capable of carrying out CasX functions such as gene editing. Increased stability of gNA variants may also, in some embodiments, allow for a similar outcome with a lower amount of gNA delivered to a cell, which may in turn reduce the chance of off-target effects during gene editing. Guide RNA stability can be assessed in a variety of ways, including for example in vitro by assembling the guide, incubating for varying periods of time in a solution that mimics the intracellular environment, and then measuring functional activity via the in vitro cleavage assays described herein. Alternatively, or in addition, gNAs can be harvested from cells at varying time points after initial transfection/transduction of the gNA to determine how long gNA variants persist relative to reference gRNAs.

i. Solubility

In some embodiments, a gNA variant has improved solubility when compared to a reference gRNA. In some embodiments, a gNA variant has improved solubility of the CasX protein:gNA RNP when compared to a reference gRNA. In some embodiments, solubility of the CasX protein:gNA RNP is improved by the addition of a ribozyme sequence to a 5′ or 3′ end of the gNA variant, for example the 5′ or 3′ of a reference sgRNA. Some ribozymes, such as the M1 ribozyme, can increase solubility of proteins through RNA mediated protein folding. Increased solubility of CasX RNPs comprising a gNA variant as described herein can be evaluated through a variety of means known to one of skill in the art, such as by taking densitometry readings on a gel of the soluble fraction of lysed E. coli in which the CasX and gNA variants are expressed.

j. Resistance to Nuclease Activity

In some embodiments, a gNA variant has improved resistance to nuclease activity compared to a reference gRNA that may, for example, increase the persistence of a variant gNA in an intracellular environment, thereby improving gene editing. Resistance to nuclease activity may be evaluated through a variety of methods known to one of skill in the art. For example, in vitro methods of measuring resistance to nuclease activity may include for example contacting reference gNA and variants with one or more exemplary RNA nucleases and measuring degradation. Alternatively, or in addition, measuring persistence of a gNA variant in a cellular environment using the methods described herein can indicate the degree to which the gNA variant is nuclease resistant.

k. Binding Affinity to a Target DNA

In some embodiments, a gNA variant has improved affinity for the target DNA relative to a reference gRNA. In certain embodiments, a ribonucleoprotein complex comprising a gNA variant has improved affinity for the target DNA, relative to the affinity of an RNP comprising a reference gRNA. In some embodiments, the improved affinity of the RNP for the target DNA comprises improved affinity for the target sequence, improved affinity for the PAM sequence, improved ability of the RNP to search DNA for the target sequence, or any combinations thereof. In some embodiments, the improved affinity for the target DNA is the result of increased overall DNA binding affinity.

Without wishing to be bound by theory, it is possible that nucleotide changes in the gNA variant that affect the function of the OBD in the CasX protein may increase the affinity of CasX variant protein binding to the protospacer adjacent motif (PAM), as well as the ability to bind or utilize an increased spectrum of PAM sequences other than the canonical TTC PAM recognized by the reference CasX protein of SEQ ID NO:2, including PAM sequences selected from the group consisting of TTC, ATC, GTC, and CTC, thereby increasing the affinity and diversity of the CasX variant protein for target DNA sequences, resulting in a substantial increase in the target nucleic acid sequences that can be edited and/or bound, compared to a reference CasX. As described more fully, below, increasing the sequences of the target nucleic acid that can be edited, compared to a reference CasX, refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with at least a single nucleotide separating the PAM from the first nucleotide of the protospacer. Alternatively, or in addition, changes in the gNA that affect function of the helical I and/or helical II domains that increase the affinity of the CasX variant protein for the target DNA strand can increase the affinity of the CasX RNP comprising the variant gNA for target DNA. Without being bound to theory or mechanism, the enhanced binding to target DNA can lead to enhanced cleavage rate of the target DNA by the RNP, wherein the RNP has at least a 5-fold, at least a 10-fold, or at least a 30-fold increased cleavage rate in an in vitro assay compared to an RNP of the reference CasX and the gNA of SEQ ID NO: 4 or SEQ ID NO:5.

l. Adding or Changing gNA Function

In some embodiments, gNA variants can comprise larger structural changes that change the topology of the gNA variant with respect to the reference gRNA, thereby allowing for different gNA functionality. For example, in some embodiments a gNA variant has swapped an endogenous stem loop of the reference gRNA scaffold with a previously identified stable RNA structure or a stem loop that can interact with a protein or RNA binding partner to recruit additional moieties to the CasX or to recruit CasX to a specific location, such as the inside of a viral capsid, that has the binding partner to the said RNA structure. In other scenarios the RNAs may be recruited to each other, as in Kissing loops, such that two CasX proteins can be co-localized for more effective gene editing at the target DNA sequence. Such RNA structures may include MS2, Qβ, U1 hairpin II, Uvsx, PP7, Phage replication loop, Kissing loop_a, Kissing loop_b1, Kissing loop_b2, G quadriplex M3q, G quadriplex telomere basket, Sarcin-ricin loop, or a Pseudoknot.

In some embodiments, a gNA variant comprises a terminal fusion partner. Exemplary terminal fusions may include fusion of the gRNA to a self-cleaving ribozyme or protein binding motif. As used herein, a “ribozyme” refers to an RNA or segment thereof with one or more catalytic activities similar to a protein enzyme. Exemplary ribozyme catalytic activities may include, for example, cleavage and/or ligation of RNA, cleavage and/or ligation of DNA, or peptide bond formation. In some embodiments, such fusions could either improve scaffold folding or recruit DNA repair machinery. For example, a gRNA may in some embodiments be fused to a hepatitis delta virus (HDV) antigenomic ribozyme, HDV genomic ribozyme, hatchet ribozyme (from metagenomic data), env25 pistol ribozyme (representative from Aliistipes putredinis), HH15 Minimal Hammerhead ribozyme, tobacco ringspot virus (TRSV) ribozyme, WT viral Hammerhead ribozyme (and rational variants), or Twisted Sister 1 or RBMX recruiting motif. Hammerhead ribozymes are RNA motifs that catalyze reversible cleavage and ligation reactions at a specific site within an RNA molecule. Hammerhead ribozymes include type I, type II and type III hammerhead ribozymes. The HDV, pistol, and hatchet ribozymes have self-cleaving activities. gNA variants comprising one or more ribozymes may allow for expanded gNA function as compared to a gRNA reference. For example, gNAs comprising self-cleaving ribozymes can, in some embodiments, be transcribed and processed into mature gNAs as part of polycistronic transcripts. Such fusions may occur at either the 5′ or the 3′ end of the gNA. In some embodiments, a gNA variant comprises a fusion at both the 5′ and the 3′ end, wherein each fusion is independently as described herein. In some embodiments, a gNA variant comprises a phage replication loop or a tetraloop. In some embodiments, a gNA comprises a hairpin loop that is capable of binding a protein. For example, in some embodiments the hairpin loop is an MS2, Qβ, U1 hairpin II, Uvsx, or PP7 hairpin loop. Exemplary sequences encoding ribozymes are selected from the group consisting of SEQ ID NOS: 598-611, as described in Table 16.

In some embodiments, a gNA variant comprises one or more RNA aptamers. As used herein, an “RNA aptamer” refers to an RNA molecule that binds a target with high affinity and high specificity. In some embodiments, a gNA variant comprises one or more riboswitches. As used herein, a “riboswitch” refers to an RNA molecule that changes state upon binding a small molecule. In some embodiments, the gNA variant further comprises one or more protein binding motifs. Adding protein binding motifs to a reference gRNA or gNA variant of the disclosure may, in some embodiments, allow a CasX RNP to associate with additional proteins, which can, for example, add the functionality of those proteins to the CasX RNP.

m. Chemically Modified gNA

In some embodiments, the disclosure relates to chemically-modified gNA. In some embodiments, the present disclosure provides a chemically-modified gNA that has guide RNA functionality and has reduced susceptibility to cleavage by a nuclease. A gNA that comprises any nucleotide other than the four canonical ribonucleotides A, C, G, and U, or a deoxynucleotide, is a chemically modified gNA. In some cases, a chemically-modified gNA comprises any backbone or internucleotide linkage other than a natural phosphodiester internucleotide linkage. In certain embodiments, the retained functionality includes the ability of the modified gNA to bind to a CasX of any of the embodiments described herein. In certain embodiments, the retained functionality includes the ability of the modified gNA to bind to a PCSK9 target nucleic acid sequence. In certain embodiments, the retained functionality includes targeting a CasX protein or the ability of a pre-complexed CasX protein-gNA to bind to a target nucleic acid sequence. In certain embodiments, the retained functionality includes the ability to nick a target polynucleotide by a CasX-gNA. In certain embodiments, the retained functionality includes the ability to cleave a target nucleic acid sequence by a CasX-gNA. In certain embodiments, the retained functionality is any other known function of a gNA in a CasX system with a CasX protein of the embodiments of the disclosure.

In some embodiments, the disclosure provides a chemically-modified gNA in which a nucleotide sugar modification is incorporated into the gNA selected from the group consisting of 2′-O-C1-4alkyl such as 2′-O-methyl (2′-OMe), 2′-deoxy (2′-H), 2′-O-C1-3alkyl-O-C1-3alkyl such as 2′-methoxyethyl (“2′-MOE”), 2′-fluoro (“2′-F”), 2′-amino (“2′-NH₂”), 2′-arabinosyl (“2′-arabino”) nucleotide, 2′-F-arabinosyl (“2′-F-arabino”) nucleotide, 2′-locked nucleic acid (“LNA”) nucleotide, 2′-unlocked nucleic acid (“ULNA”) nucleotide, a sugar in L form (“L-sugar”), and 4′-thioribosyl nucleotide. In other embodiments, an internucleotide linkage modification incorporated into the guide RNA is selected from the group consisting of: phosphorothioate “P(S)” (P(S)), phosphonocarboxylate (P(CH₂)ₙCOOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂)ₙCOO⁻)), alkylphosphonate (P(C₁₋₃alkyl) such as methylphosphonate —P(CH₃), boranophosphonate (P(BH₃)), and phosphorodithioate (P(S)₂).

In certain embodiments, the disclosure provides a chemically-modified gNA in which a nucleobase (“base”) modification is incorporated into the gNA selected from the group consisting of: 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”), 5-methyl-2-pyrimidine, x(A,G,C,T) and y(A,G,C,T).

In other embodiments, the disclosure provides a chemically-modified gNA in which one or more isotopic modifications are introduced on the nucleotide sugar, the nucleobase, the phosphodiester linkage and/or the nucleotide phosphates, including nucleotides comprising one or more ¹⁵N, ¹³C, ¹⁴C, deuterium, ³H, ³²P, ¹²⁵I, ¹³¹I atoms or other atoms or elements used as tracers.

In some embodiments, an “end” modification incorporated into the gNA is selected from the group consisting of: PEG (polyethyleneglycol), hydrocarbon linkers (including: heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers), spermine linkers, dyes including fluorescent dyes (for example fluoresceins, rhodamines, cyanines) attached to linkers such as for example 6-fluorescein-hexyl, quenchers (for example dabcyl, BHQ) and other labels (for example biotin, digoxigenin, acridine, streptavidin, avidin, peptides and/or proteins). In some embodiments, an “end” modification comprises a conjugation (or ligation) of the gNA to another molecule comprising an oligonucleotide of deoxynucleotides and/or ribonucleotides, a peptide, a protein, a sugar, an oligosaccharide, a steroid, a lipid, a folic acid, a vitamin and/or other molecule. In certain embodiments, the disclosure provides a chemically-modified gNA in which an “end” modification (described above) is located internally in the gNA sequence via a linker such as, for example, a 2-(4-butylamidofluorescein)propane-1,3-diol bis(phosphodiester) linker, which is incorporated as a phosphodiester linkage and can be incorporated anywhere between two nucleotides in the gNA.

In some embodiments, the disclosure provides a chemically-modified gNA having an end modification comprising a terminal functional group such as an amine, a thiol (or sulfhydryl), a hydroxyl, a carboxyl, carbonyl, thionyl, thiocarbonyl, a carbamoyl, a thiocarbamoyl, a phoshoryl, an alkene, an alkyne, an halogen or a functional group-terminated linker that can be subsequently conjugated to a desired moiety selected from the group consisting of a fluorescent dye, a non-fluorescent label, a tag (for ¹⁴C, example biotin, avidin, streptavidin, or moiety containing an isotopic label such as ¹⁵N, ¹³C, deuterium, ³H, ³²P, ¹²⁵I and the like), an oligonucleotide (comprising deoxynucleotides and/or ribonucleotides, including an aptamer), an amino acid, a peptide, a protein, a sugar, an oligosaccharide, a steroid, a lipid, a folic acid, and a vitamin. The conjugation employs standard chemistry well-known in the art, including but not limited to coupling via N-hydroxysuccinimide, isothiocyanate, DCC (or DCI), and/or any other standard method as described in “Bioconjugate Techniques” by Greg T. Hermanson, Publisher Elsevier Science, 3^{rd} ed. (2013), the contents of which are incorporated herein by reference in its entirety.

### IV. Proteins for Modifying a Target Nucleic Acid

The present disclosure provides systems comprising a CRISPR nuclease that have utility in genome editing of eukaryotic cells. In some embodiments, the CRISPR nuclease employed in the genome editing systems is a Class 2, Type V nuclease. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the Type V nucleases of the embodiments recognize a 5′-TC PAM motif and produce staggered ends cleaved solely by the RuvC domain. In some embodiments, the Type V nuclease is selected from the group consisting of Cas12a, Cas12b, Cas12c, Cas12d (CasY), CasZ and CasX. In some embodiments, the present disclosure provides systems comprising a CasX protein and one or more gNA acids (CasX:gNA system) that are specifically designed to modify a target nucleic acid sequence in eukaryotic cells.

The term “CasX protein”, as used herein, refers to a family of proteins, and encompasses all naturally occurring CasX proteins (“reference CasX”), proteins that share at least 50% identity to naturally occurring CasX proteins, as well as CasX variants possessing one or more improved characteristics relative to a naturally occurring CasX protein, described more fully, below.

Exemplary improved characteristics of the CasX variant embodiments include, but are not limited to improved folding of the variant, improved binding affinity to the gNA, improved binding affinity to the target nucleic acid, improved ability to utilize a greater spectrum of PAM sequences in the editing and/or binding of target DNA, improved unwinding of the target DNA, increased editing activity, improved editing efficiency, improved editing specificity, increased percentage of a eukaryotic genome that can be efficiently edited, increased activity of the nuclease, increased target strand loading for double strand cleavage, decreased target strand loading for single strand nicking, decreased off-target cleavage, improved binding of the non-target strand of DNA, improved target nucleic acid sequence cleavage rate, improved protein stability, improved protein:gNA (RNP) complex stability, improved protein solubility, improved ribonuclear protein complex (RNP) formation, higher percentage of cleavage-competent RNP, improved protein:gNA (RNP) complex solubility, improved protein yield, improved protein expression, and improved fusion characteristics, as described more fully, below. In some embodiments, the RNP of the CasX variant and the gNA variant exhibit one or more of the improved characteristics that are at least about 1.1 to about 100,000-fold improved relative to an RNP of the reference CasX protein of SEQ ID NO:1, SEQ ID NO:2, or SEQ ID NO:3 and the gNA of Table 1, when assayed in a comparable fashion. In other cases, the one or more improved characteristics of an RNP of the CasX variant and the gNA variant are at least about 1.1, at least about 10, at least about 100, at least about 1000, at least about 10,000, at least about 100,000-fold or more improved relative to an RNP of the reference CasX protein of SEQ ID NO:1, SEQ ID NO:2, or SEQ ID NO:3 and the gNA of Table 1. In other cases, the one or more of the improved characteristics of an RNP of the CasX variant and the gNA variant are about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold, about 10 to 100,00-fold, about 10 to 10,00-fold, about 10 to 1,000-fold, about 10 to 500-fold, about 10 to 100-fold, about 10 to 50-fold, about 10 to 20-fold, about 2 to 70-fold, about 2 to 50-fold, about 2 to 30-fold, about 2 to 20-fold, about 2 to 10-fold, about 5 to 50-fold, about 5 to 30-fold, about 5 to 10-fold, about 100 to 100,00-fold, about 100 to 10,00-fold, about 100 to 1,000-fold, about 100 to 500-fold, about 500 to 100,00-fold, about 500 to 10,00-fold, about 500 to 1,000-fold, about 500 to 750-fold, about 1,000 to 100,00-fold, about 10,000 to 100,00-fold, about 20 to 500-fold, about 20 to 250-fold, about 20 to 200-fold, about 20 to 100-fold, about 20 to 50-fold, about 50 to 10,000-fold, about 50 to 1,000-fold, about 50 to 500-fold, about 50 to 200-fold, or about 50 to 100-fold, improved relative to an RNP of the reference CasX protein of SEQ ID NO:1, SEQ ID NO:2, or SEQ ID NO:3 and the gNA of Table 1, when assayed in a comparable fashion. In other cases, the one or more improved characteristics of an RNP of the CasX variant and the gNA variant are about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 40-fold, 45-fold, 50-fold, 55-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, 400-fold, 425-fold, 450-fold, 475-fold, or 500-fold improved relative to an RNP of the reference CasX protein of SEQ ID NO:1, SEQ ID NO:2, or SEQ ID NO:3 and the gNA of Table 1, when assayed in a comparable fashion. In a particular embodiment, an RNP of a CasX variant and gNA variant exhibits an increased cleavage rate of at least a 5-fold, at least a 10-fold, or at least a 30-fold increase in an in vitro assay compared to an RNP of the reference CasX proteins of SEQ ID NOS: 1-3 and reference gNAs of SEQ ID NO: 4 or SEQ ID NO: 5. Supportive data of such improvements are presented in the Examples, below.

The term “CasX variant” is inclusive of variants that are fusion proteins; i.e., the CasX is “fused to” a heterologous sequence. This includes CasX variants comprising CasX variant sequences and N-terminal, C-terminal, or internal fusions of the CasX to a heterologous protein or domain thereof.

CasX proteins of the disclosure comprise at least one of the following domains: a non-target strand binding (NTSB) domain, a target strand loading (TSL) domain, a helical I domain, a helical II domain, an oligonucleotide binding domain (OBD), and a RuvC DNA cleavage domain (the last of which may be modified or deleted in a catalytically dead CasX variant), described more fully, below. Additionally, the CasX variant proteins of the disclosure have an enhanced ability to efficiently edit and/or bind target DNA, when complexed with a gNA as an RNP, utilizing PAM TC motif, including PAM sequences selected from TTC, ATC, GTC, or CTC, compared to an RNP of a reference CasX protein and reference gNA. In the foregoing, the PAM sequence is located at least 1 nucleotide 5′ to the non-target strand of the protospacer having identity with the targeting sequence of the gNA in a assay system compared to the editing efficiency and/or binding of an RNP comprising a reference CasX protein and reference gNA in a comparable assay system. In one embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is TTC. In another embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is ATC. In another embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is CTC. In another embodiment, an RNP of a CasX variant and gNA variant exhibits greater editing efficiency and/or binding of a target sequence in the target DNA compared to an RNP comprising a reference CasX protein and a reference gNA in a comparable assay system, wherein the PAM sequence of the target DNA is GTC. In the foregoing embodiments, the increased editing efficiency and/or binding affinity for the one or more PAM sequences is at least 1.5-fold greater or more compared to the editing efficiency and/or binding affinity of an RNP of any one of the CasX proteins of SEQ ID NOS:1-3 and the gNA of SEQ ID NOS: 4 and 5 of Table 1 for the PAM sequences.

In some embodiments, a CasX protein can bind and/or modify (e.g., cleave, nick, methylate, demethylate, etc.) a target nucleic acid and/or a polypeptide associated with target nucleic acid (e.g., methylation or acetylation of a histone tail). In some embodiments, the CasX protein is catalytically dead (dCasX) but retains the ability to bind a target nucleic acid. An exemplary catalytically dead CasX protein comprises one or more mutations in the active site of the RuvC domain of the CasX protein. In some embodiments, a catalytically dead CasX protein comprises substitutions at residues 672, 769 and/or 935 of SEQ ID NO:1. In one embodiment, a catalytically dead CasX protein comprises substitutions of D672A, E769A and/or D935A in a reference CasX protein of SEQ ID NO:1. In other embodiments, a catalytically dead CasX protein comprises substitutions at amino acids 659, 756 and/or 922 in a reference CasX protein of SEQ ID NO:2. In some embodiments, a catalytically dead CasX protein comprises D659A, E756A and/or D922A substitutions in a reference CasX protein of SEQ ID NO:2. In further embodiments, a catalytically dead CasX protein comprises deletions of all or part of the RuvC domain of the CasX protein. It will be understood that the same foregoing substitutions can similarly be introduced into the CasX variants of the disclosure, resulting in a dCasX variant. In one embodiment, all or a portion of the RuvC domain is deleted from the CasX variant, resulting in a dCasX variant. Catalytically inactive dCasX variant proteins can, in some embodiments, be used for base editing or epigenetic modifications. With a higher affinity for DNA, in some embodiments, catalytically inactive dCasX variant proteins can, relative to catalytically active CasX, find their target nucleic acid faster, remain bound to target nucleic acid for longer periods of time, bind target nucleic acid in a more stable fashion, or a combination thereof, thereby improving these functions of the catalytically dead CasX variant protein compared to a CasX variant that retains its cleavage capability.

a. Non-Target Strand Binding Domain

The reference CasX proteins of the disclosure comprise a non-target strand binding domain (NTSBD). The NTSBD is a domain not previously found in any Cas proteins; for example this domain is not present in Cas proteins such as Cas9, Cas12a/Cpf1, Cas13, Cas14, CASCADE, CSM, or CSY. Without being bound to theory or mechanism, a NTSBD in a CasX allows for binding to the non-target DNA strand and may aid in unwinding of the non-target and target strands. The NTSBD is presumed to be responsible for the unwinding, or the capture, of a non-target DNA strand in the unwound state. The NTSBD is in direct contact with the non-target strand in CryoEM model structures derived to date and may contain a non-canonical zinc finger domain. The NTSBD may also play a role in stabilizing DNA during unwinding, guide RNA invasion and R-loop formation. In some embodiments, an exemplary NTSBD comprises amino acids 101-191 of SEQ ID NO:1 or amino acids 103-192 of SEQ ID NO:2. In some embodiments, the NTSBD of a reference CasX protein comprises a four-stranded beta sheet.

b. Target Strand Loading Domain

The reference CasX proteins of the disclosure comprise a Target Strand Loading (TSL) domain. The TSL domain is a domain not found in certain Cas proteins such as Cas9, CASCADE, CSM, or CSY. Without wishing to be bound by theory or mechanism, it is thought that the TSL domain is responsible for aiding the loading of the target DNA strand into the RuvC active site of a CasX protein. In some embodiments, the TSL acts to place or capture the target-strand in a folded state that places the scissile phosphate of the target strand DNA backbone in the RuvC active site. The TSL comprises a cys4 (CXXC, CXXC zinc finger/ribbon domain (SEQ ID NO: 48) that is separated by the bulk of the TSL. In some embodiments, an exemplary TSL comprises amino acids 825-934 of SEQ ID NO:1 or amino acids 813-921 of SEQ ID NO:2.

c. Helical I Domain

The reference CasX proteins of the disclosure comprise a helical I domain. Certain Cas proteins other than CasX have domains that may be named in a similar way. However, in some embodiments, the helical I domain of a CasX protein comprises one or more unique structural features, or comprises a unique sequence, or a combination thereof, compared to non-CasX proteins. For example, in some embodiments, the helical I domain of a CasX protein comprises one or more unique secondary structures compared to domains in other Cas proteins that may have a similar name. For example, in some embodiments the helical I domain in a CasX protein comprises one or more alpha helices of unique structure and sequence in arrangement, number and length compared to other CRISPR proteins. In certain embodiments, the helical I domain is responsible for interacting with the bound DNA and spacer of the guide RNA. Without wishing to be bound by theory, it is thought that in some cases the helical I domain may contribute to binding of the protospacer adjacent motif (PAM). In some embodiments, an exemplary helical I domain comprises amino acids 57-100 and 192-332 of SEQ ID NO:1, or amino acids 59-102 and 193-333 of SEQ ID NO:2. In some embodiments, the helical I domain of a reference CasX protein comprises one or more alpha helices.

d. Helical II Domain

The reference CasX proteins of the disclosure comprise a helical II domain. Certain Cas proteins other than CasX have domains that may be named in a similar way. However, in some embodiments, the helical II domain of a CasX protein comprises one or more unique structural features, or a unique sequence, or a combination thereof, compared to domains in other Cas proteins that may have a similar name. For example, in some embodiments, the helical II domain comprises one or more unique structural alpha helical bundles that align along the target DNA:guide RNA channel. In some embodiments, in a CasX comprising a helical II domain, the target strand and guide RNA interact with helical II (and the helical I domain, in some embodiments) to allow RuvC domain access to the target DNA. The helical II domain is responsible for binding to the guide RNA scaffold stem loop as well as the bound DNA. In some embodiments, an exemplary helical II domain comprises amino acids 333-509 of SEQ ID NO:1, or amino acids 334-501 of SEQ ID NO:2.

e. Oligonucleotide Binding Domain

The reference CasX proteins of the disclosure comprise an Oligonucleotide Binding Domain (OBD). Certain Cas proteins other than CasX have domains that may be named in a similar way. However, in some embodiments, the OBD comprises one or more unique functional features, or comprises a sequence unique to a CasX protein, or a combination thereof. For example, in some embodiments the bridged helix (BH), helical I domain, helical II domain, and Oligonucleotide Binding Domain (OBD) together are responsible for binding of a CasX protein to the guide RNA. Thus, for example, in some embodiments the OBD is unique to a CasX protein in that it interacts functionally with a helical I domain, or a helical II domain, or both, each of which may be unique to a CasX protein as described herein. Specifically, in CasX the OBD largely binds the RNA triplex of the guide RNA scaffold. The OBD may also be responsible for binding to the protospacer adjacent motif (PAM). An exemplary OBD domain comprises amino acids 1-56 and 510-660 of SEQ ID NO:1, or amino acids 1-58 and 502-647 of SEQ ID NO:2.

f. RuvC DNA Cleavage Domain

The reference CasX proteins of the disclosure comprise a RuvC domain, that includes 2 partial RuvC domains (RuvC-I and RuvC-II). The RuvC domain is the ancestral domain of all type 12 CRISPR proteins. The RuvC domain originates from a TNPB (transposase B) like transposase. Similar to other RuvC domains, the CasX RuvC domain has a DED catalytic triad that is responsible for coordinating a magnesium (Mg) ion and cleaving DNA. In some embodiments, the RuvC has a DED motif active site that is responsible for cleaving both strands of DNA (one by one, most likely the non-target strand first at 11-14 nucleotides (nt) into the targeted sequence and then the target strand next at 2-4 nucleotides after the target sequence). Specifically in CasX, the RuvC domain is unique in that it is also responsible for binding the guide RNA scaffold stem loop that is critical for CasX function. An exemplary RuvC domain comprises amino acids 661-824 and 935-986 of SEQ ID NO:1, or amino acids 648-812 and 922-978 of SEQ ID NO:2.

g. Reference CasX Proteins

The disclosure provides naturally-occurring CasX proteins (referred to herein as a “reference CasX protein”) that function as an endonuclease that catalyzes a double strand break at a specific sequence in a targeted double-stranded DNA (dsDNA). The sequence specificity is provided by the targeting sequence of the associated gNA to which it is complexed, which hybridizes to a target sequence within the target nucleic acid. For example, reference CasX proteins can be isolated from naturally occurring prokaryotes, such as Deltaproteobacteria, Planctomycetes, or Candidatus Sungbacteria species. A reference CasX protein (sometimes referred to herein as a reference CasX protein) is a Type V CRISPR/Cas endonuclease belonging to the CasX (sometimes referred to as Cas12e) family of proteins that is capable of interacting with a guide NA to form a ribonucleoprotein (RNP) complex. In some embodiments, the RNP complex comprising the reference CasX protein can be targeted to a particular site in a target nucleic acid via base pairing between the targeting sequence (or spacer) of the gNA and a target sequence in the target nucleic acid. In some embodiments, the RNP comprising the reference CasX protein is capable of cleaving target DNA. In some embodiments, the RNP comprising the reference CasX protein is capable of nicking target DNA. In some embodiments, the RNP comprising the reference CasX protein is capable of editing target DNA, for example in those embodiments where the reference CasX protein is capable of cleaving or nicking DNA, followed by non-homologous end joining (NHEJ), homology-directed repair (HDR), homology-independent targeted integration (HITI), micro-homology mediated end joining (MMEJ), single strand annealing (SSA) or base excision repair (BER). In some embodiments, the RNP comprising the CasX protein is a catalytically dead (is catalytically inactive or has substantially no cleavage activity) CasX protein (dCasX), but retains the ability to bind the target DNA, described more fully, supra.

In some cases, a Type V reference CasX protein is isolated or derived from Deltaproteobacteria. In some embodiments, a CasX protein comprises a sequence at least 50% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of:

```
(SEQ ID NO: 1)
  1 MEKRINKIRK KLSADNATKP VSRSGPMKTL
      LVRVMTDDLK KRLEKRRKKP EVMPQVISNN
  61 AANNLRMLLD DYTKMKEAIL QVYWQEFKDD
        HVGLMCKFAQ PASKKIDQNK LKPEMDEKGN
121 LTTAGFACSQ CGQPLFVYKL EQVSEKGKAY
        TNYFGRCNVA EHEKLILLAQ LKPEKDSDEA
181 VTYSLGKFGQ RALDFYSIHV TKESTHPVKP
        LAQIAGNRYA SGPVGKALSD ACMGTIASFL
241 SKYQDIIIEH QKVVKGNQKR LESLRELAGK
        ENLEYPSVTL PPQPHTKEGV DAYNEVIARV
301 RMWVNLNLWQ KLKLSRDDAK PLLRLKGFPS
        FPVVERRENE VDWWNTINEV KKLIDAKRDM
361 GRVFWSGVTA EKRNTILEGY NYLPNENDHK
        KREGSLENPK KPAKRQFGDL LLYLEKKYAG
421 DWGKVFDEAW ERIDKKIAGL TSHIEREEAR
        NAEDAQSKAV LTDWLRAKAS FVLERLKEMD
481 EKEFYACEIQ LQKWYGDLRG NPFAVEAENR
        VVDISGFSIG SDGHSIQYRN LLAWKYLENG
541 KREFYLLMNY GKKGRIRFTD GTDIKKSGKW
        QGLLYGGGKA KVIDLTFDPD DEQLIILPLA
601 FGTRQGREFI WNDLLSLETG LIKLANGRVI
        EKTIYNKKIG RDEPALFVAL TFERREVVDP
661 SNIKPVNLIG VDRGENIPAV IALTDPEGCP
        LPEFKDSSGG PTDILRIGEG YKEKQRAIQA
721 AKEVEQRRAG GYSRKFASKS RNLADDMVRN
        SARDLFYHAV THDAVLVFEN LSRGFGRQGK
781 RTFMTERQYT KMEDWLTAKL AYEGLTSKTY
        LSKTLAQYTS KTCSNCGFTI TTADYDGMLV
841 RLKKTSDGWA TTLNNKELKA EGQITYYNRY
        KRQTVEKELS AELDRLSEES GNNDISKWTK
901 GRRDEALFLL KKRFSHRPVQ EQFVCLDCGH
        EVHADEQAAL NIARSWLFLN SNSTEFKSYK
961 SGKQPFVGAW QAFYKRRLKE VWKPNA.
```

In some cases, a Type V reference CasX protein is isolated or derived from Planctomycetes. In some embodiments, a CasX protein comprises a sequence at least 50% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of:

```
(SEQ ID NO: 2)
  1 MQEIKRINKI RRRLVKDSNT KKAGKTGPMK
        TLLVRVMTPD LRERLENLRK KPENIPQPIS
  61 NTSRANLNKL LTDYTEMKKA ILHVYWEEFQ
        KDPVGLMSRV AQPAPKNIDQ RKLIPVKDGN
121 ERLTSSGFAC SQCCQPLYVY KLEQVNDKGK
        PHTNYFGRCN VSEHERLILL SPHKPEANDE
181 LVTYSLGKFG QRALDFYSIH VTRESNHPVK
        PLEQIGGNSC ASGPVGKALS DACMGAVASF
241 LTKYQDIIIE HQKVIKKNEK RLANLKDIAS
        ANGLAFPKIT LPPQPHTKEG IEAYNNVVAQ
301 IVIWVNLNLW QKLKIGRDEA KPLQRLKGFP
        SFPLVERQAN EVDWWDMVCN VKKLINEKKE
361 DGKVFWQNLA GYKRQEALLP YLSSEEDRKK
        GKKFARYQFG DLLLHLEKKH GEDWGKVYDE
421 AWERIDKKVE GLSKHIKLEE ERRSEDAQSK
        AALTDWLRAK ASFVIEGLKE ADKDEFORCE
481 LKLQKWYGDL RGKPFAIEAE NSILDISGFS
        KQYNCAFIWQ KDGVKKLNLY LIINYFKGGK
541 LRFKKIKPEA FEANRFYTVI NKKSGEIVPM
        EVNFNFDDPN LIILPLAFGK RQGREFIWND
601 LLSLETGSLK LANGRVIEKT LYNRRTRQDE
        PALFVALTFE RREVLDSSNI KPMNLIGIDR
661 GENIPAVIAL TDPEGCPLSR FKDSLGNPTH
        ILRIGESYKE KQRTIQAAKE VEQRRAGGYS
721 RKYASKAKNL ADDMVRNTAR DLLYYAVTQD
        AMLIFENLSR GFGRQGKRTF MAERQYTRME
781 DWLTAKLAYE GLPSKTYLSK TLAQYTSKTC
        SNCGFTITSA DYDRVLEKLK KTATGWMTTI
841 NGKELKVEGQ ITYYNRYKRQ NVVKDLSVEL
        DRLSEESVNN DISSWTKGRS GEALSLLKKR
901 FSHRPVQEKF VCLNCGFETH ADEQAALNIA
        RSWLFLRSQE YKKYQTNKTT GNTDKRAFVE
961 TWQSFYRKKL KEVWKPAV.
```

In some embodiments, the CasX protein comprises the sequence of SEQ ID NO: 2, or at least 60% similarity thereto. In some embodiments, the CasX protein comprises the sequence of SEQ ID NO: 2, or at least 80% similarity thereto. In some embodiments, the CasX protein comprises the sequence of SEQ ID NO: 2, or at least 90% similarity thereto. In some embodiments, the CasX protein comprises the sequence of SEQ ID NO: 2, or at least 95% similarity thereto. In some embodiments, the CasX protein consists of the sequence of SEQ ID NO: 2. In some embodiments, the CasX protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO: 2. These mutations can be insertions, deletions, amino acid substitutions, or any combinations thereof.

In some cases, a Type V reference CasX protein is isolated or derived from Candidatus Sungbacteria. In some embodiments, a CasX protein comprises a sequence at least 50% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of

```
(SEQ ID NO: 3)
  1 MDNANKPSTK SLVNTTRISD HFGVTPGQVT
        RVFSFGIIPT KRQYAIIERW FAAVEAARER
  61 LYGMLYAHFQ ENPPAYLKEK FSYETFFKGR
        PVLNGLRDID PTIMTSAVFT ALRHKAEGAM
121 AAFHTNHRRL FEEARKKMRE YAECLKANEA
        LLRGAADIDW DKIVNALRTR LNTCLAPEYD
181 AVIADFGALC AFRALIAETN ALKGAYNHAL
        NQMLPALVKV DEPEEAEESP RLRFFNGRIN
241 DLPKFPVAER ETPPDTETII RQLEDMARVI
        PDTAEILGYI HRIRHKAARR KPGSAVPLPQ
301 RVALYCAIRM ERNPEEDPST VAGHFLGEID
        RVCEKRRQGL VRTPFDSQIR ARYMDIISFR
361 ATLAHPDRWT EIQFLRSNAA SRRVRAETIS
        APFEGFSWTS NRTNPAPQYG MALAKDANAP
421 ADAPELCICL SPSSAAFSVR EKGGDLIYMR
        PTGGRRGKDN PGKEITWVPG SFDEYPASGV
481 ALKLRLYFGR SQARRMLTNK TWGLLSDNPR
        VFAANAELVG KKRNPQDRWK LFFHMVISGP
541 PPVEYLDFSS DVRSRARTVI GINRGEVNPL
        AYAVVSVEDG QVLEEGLLGK KEYIDQLIET
601 RRRISEYQSR EQTPPRDLRQ RVRHLQDTVL
        GSARAKIHSL IAFWKGILAI ERLDDQFHGR
661 EQKIIPKKTY LANKTGFMNA LSFSGAVRVD
        KKGNPWGGMI EIYPGGISRT CTQCGTVWLA
721 RRPKNPGHRD AMVVIPDIVD DAAATGFDNV
        DCDAGTVDYG ELFTLSREWV RLTPRYSRVM
781 RGTLGDLERA IRQGDDRKSR QMLELALEPQ
        PQWGQFFCHR CGFNGQSDVL AATNLARRAI
841 SLIRRLPDTD TPPTP.
```

In some embodiments, the CasX protein comprises the sequence of SEQ ID NO: 3, or at least 60% similarity thereto. In some embodiments, the CasX protein comprises the sequence of SEQ ID NO: 3, or at least 80% similarity thereto. In some embodiments, the CasX protein comprises the sequence of SEQ ID NO: 3, or at least 90% similarity thereto. In some embodiments, the CasX protein comprises the sequence of SEQ ID NO: 3, or at least 95% similarity thereto. In some embodiments, the CasX protein consists of the sequence of SEQ ID NO: 3. In some embodiments, the CasX protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO: 3. These mutations can be insertions, deletions, amino acid substitutions, or any combinations thereof.

h. CasX Variant Proteins

The present disclosure provides variants of a reference CasX protein (interchangeably referred to herein as “CasX variant” or “CasX variant protein”), wherein the CasX variants comprise at least one sequence modification in at least one domain relative to a reference CasX protein, including the sequences of SEQ ID NOS:1-3. In some embodiments, the CasX variant exhibits at least one improved characteristic compared to the reference CasX protein. All variants that improve one or more functions or characteristics of the CasX variant protein when compared to a reference CasX protein described herein are envisaged as being within the scope of the disclosure. In some embodiments, the modification is a mutation in one or more amino acids of the reference CasX. In other embodiments, the modification is a substitution of one or more domains of the reference CasX with one or more domains from a different CasX. In some embodiments, insertion includes the insertion of a part or all of a domain from a different CasX protein. Mutations can occur in any one or more domains of the reference CasX protein, and may include, for example, deletion of part or all of one or more domains, or one or more amino acid substitutions, deletions, or insertions in any domain of the reference CasX protein. The domains of CasX proteins include the non-target strand binding (NTSB) domain, the target strand loading (TSL) domain, the helical I domain, the helical II domain, the oligonucleotide binding domain (OBD), and the RuvC DNA cleavage domain. Any change in amino acid sequence of a reference CasX protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence. In a particular feature, the CasX variant proteins of the disclosure have advantages over reference CasX proteins in that they have binding affinity for a greater diversity of PAM sequences, selected from TTC, ATC, GTC, or CTC, which enables the CasX variants to edit a significantly greater portion of the target nucleic acid compared to reference CasX proteins.

In some embodiments, the CasX variant protein comprises at least one modification in at least each of two domains of the reference CasX protein, including the sequences of SEQ ID NOS: 1-3. In some embodiments, the CasX variant protein comprises at least one modification in at least 2 domains, in at least 3 domains, at least 4 domains or at least 5 domains of the reference CasX protein. In some embodiments, the CasX variant protein comprises two or more modifications in at least one domain of the reference CasX protein. In some embodiments, the CasX variant protein comprises at least two modifications in at least one domain of the reference CasX protein, at least three modifications in at least one domain of the reference CasX protein or at least four modifications in at least one domain of the reference CasX protein. In some embodiments, wherein the CasX variant comprises two or more modifications compared to a reference CasX protein, each modification is made in a domain independently selected from the group consisting of a NTSBD, TSLD, Helical I domain, Helical II domain, OBD, and RuvC DNA cleavage domain.

In some embodiments, the at least one modification of the CasX variant protein comprises a deletion of at least a portion of one domain of the reference CasX protein of SEQ ID NOS: 1-3. In some embodiments, the deletion is in the NTSBD, TSLD, Helical I domain, Helical II domain, OBD, or RuvC DNA cleavage domain.

Suitable mutagenesis methods for generating CasX variant proteins of the disclosure may include, for example, Deep Mutational Evolution (DME), deep mutational scanning (DMS), error prone PCR, cassette mutagenesis, random mutagenesis, staggered extension PCR, gene shuffling, or domain swapping. In some embodiments, the CasX variants are designed, for example by selecting one or more desired mutations in a reference CasX. In certain embodiments, the activity of a reference CasX protein is used as a benchmark against which the activity of one or more CasX variants are compared, thereby measuring improvements in function of the CasX variants. Exemplary improvements of CasX variants include, but are not limited to, improved folding of the variant, improved binding affinity to the gNA, improved binding affinity to the target DNA, altered binding affinity to one or more PAM sequences, improved unwinding of the target DNA, increased activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved target nucleic acid sequence cleavage rate, improved protein stability, improved protein:gNA complex stability, improved protein solubility, improved protein:gNA complex solubility, improved protein yield, improved protein expression, and improved fusion characteristics, as described more fully, below.

In some embodiments of the CasX variants described herein, the at least one modification comprises: (a) a substitution of 1 to 100 consecutive or non-consecutive amino acids in the CasX variant compared to a reference CasX of SEQ ID NO:1, SEQ ID NO:2, or SEQ ID NO:3; (b) a deletion of 1 to 100 consecutive or non-consecutive amino acids in the CasX variant compared to a reference CasX; (c) an insertion of 1 to 100 consecutive or non-consecutive amino acids in the CasX compared to a reference CasX; or (d) any combination of (a)-(c). In some embodiments, the at least one modification comprises: (a) a substitution of 5-10 consecutive or non-consecutive amino acids in the CasX variant compared to a reference CasX of SEQ ID NO:1, SEQ ID NO:2, or SEQ ID NO:3; (b) a deletion of 1-5 consecutive or non-consecutive amino acids in the CasX variant compared to a reference CasX; (c) an insertion of 1-5 consecutive or non-consecutive amino acids in the CasX compared to a reference CasX; or (d) any combination of (a)-(c).

In some embodiments, the CasX variant protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO:1, SEQ ID NO:2, or SEQ ID NO:3. These mutations can be insertions, deletions, amino acid substitutions, or any combinations thereof.

In some embodiments, the CasX variant protein comprises at least one amino acid substitution in at least one domain of a reference CasX protein. In some embodiments, the CasX variant protein comprises at least about 1-4 amino acid substitutions, 1-10 amino acid substitutions, 1-20 amino acid substitutions, 1-30 amino acid substitutions, 1-40 amino acid substitutions, 1-50 amino acid substitutions, 1-60 amino acid substitutions, 1-70 amino acid substitutions, 1-80 amino acid substitutions, 1-90 amino acid substitutions, 1-100 amino acid substitutions, 2-10 amino acid substitutions, 2-20 amino acid substitutions, 2-30 amino acid substitutions, 3-10 amino acid substitutions, 3-20 amino acid substitutions, 3-30 amino acid substitutions, 4-10 amino acid substitutions, 4-20 amino acid substitutions, 3-300 amino acid substitutions, 5-10 amino acid substitutions, 5-20 amino acid substitutions, 5-30 amino acid substitutions, 10-50 amino acid substitutions, or 20-50 amino acid substitutions, relative to a reference CasX protein, which can be consecutive or non-consecutive, or in different domains. As used herein “consecutive amino acids” refer to amino acids that are contiguous in the primary sequence of a polypeptide. In some embodiments, the CasX variant protein comprises at least about 100 or more amino acid substitutions relative to a reference CasX protein. In some embodiments, the amino acid substitutions are conservative substitutions. In other embodiments, the substitutions are non-conservative; e.g., a polar amino acid is substituted for a non-polar amino acid, or vice versa.

Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a reference CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate a CasX variant protein of the disclosure.

In some embodiments, a CasX variant protein comprises at least one amino acid deletion relative to a reference CasX protein. In some embodiments, a CasX variant protein comprises a deletion of 1-4 amino acids, 1-10 amino acids, 1-20 amino acids, 1-30 amino acids, 1-40 amino acids, 1-50 amino acids, 1-60 amino acids, 1-70 amino acids, 1-80 amino acids, 1-90 amino acids, 1-100 amino acids, 2-10 amino acids, 2-20 amino acids, 2-30 amino acids, 3-10 amino acids, 3-20 amino acids, 3-30 amino acids, 4-10 amino acids, 4-20 amino acids, 3-300 amino acids, 5-10 amino acids, 5-20 amino acids, 5-30 amino acids, 10-50 amino acids or 20-50 amino acids relative to a reference CasX protein. In some embodiments, a CasX protein comprises a deletion of at least about 100 consecutive amino acids relative to a reference CasX protein. In some embodiments, a CasX variant protein comprises a deletion of at least 1, 2, 3, 4, 5, 6, 7, 8, 9, 10, 20, 30, 40, 50 or 100 consecutive amino acids relative to a reference CasX protein. In some embodiments, a CasX variant protein comprises a deletion of 1, 2, 3, 4, 5, 6, 7, 8, 9 or 10 consecutive amino acids.

In some embodiments, a CasX variant protein comprises two or more deletions relative to a reference CasX protein, and the two or more deletions are not consecutive amino acids. For example, a first deletion may be in a first domain of the reference CasX protein, and a second deletion may be in a second domain of the reference CasX protein. In some embodiments, a CasX variant protein comprises 2, 3, 4, 5, 6, 7, 8, 9, 10, 11, 12, 13, 14, 15, 16, 17, 18, 19 or 20 non-consecutive deletions relative to a reference CasX protein. In some embodiments, a CasX variant protein comprises at least 20 non-consecutive deletions relative to a reference CasX protein. Each non-consecutive deletion may be of any length of amino acids described herein, e.g., 1-4 amino acids, 1-10 amino acids, and the like.

In some embodiments, the CasX variant protein comprises at least one amino acid insertion relative to the sequence of SEQ ID NO:1, SEQ ID NO: 2, or SEQ ID NO: 3. In some embodiments, a CasX variant protein comprises an insertion of 1 amino acid, an insertion of 2-3 consecutive amino acids, 2-4 consecutive amino acids, 2-5 consecutive amino acids, 2-6 consecutive amino acids, 2-7 consecutive amino acids, 2-8 consecutive amino acids, 2-9 consecutive amino acids, 2-10 consecutive amino acids, 2-20 consecutive amino acids, 2-30 consecutive amino acids, 2-40 consecutive amino acids, 2-50 consecutive amino acids, 2-60 consecutive amino acids, 2-70 consecutive amino acids, 2-80 consecutive amino acids, 2-90 consecutive amino acids, 2-100 consecutive amino acids, 3-10 consecutive amino acids, 3-20 consecutive amino acids, 3-30 consecutive amino acids, 4-10 consecutive amino acids, 4-20 consecutive amino acids, 3-300 consecutive amino acids, 5-10 consecutive amino acids, 5-20 consecutive amino acids, 5-30 consecutive amino acids, 10-50 consecutive amino acids or 20-50 consecutive amino acids relative to a reference CasX protein. In some embodiments, the CasX variant protein comprises an insertion of 2, 3, 4, 5, 6, 7, 8, 9, 10, 11, 12, 13, 14, 15, 16, 17, 18, 19 or 20 consecutive amino acids. In some embodiments, a CasX variant protein comprises an insertion of at least about 100 consecutive amino acids.

In some embodiments, a CasX variant protein comprises two or more insertions relative to a reference CasX protein, and the two or more insertions are not consecutive amino acids of the sequence. For example, a first insertion may be in a first domain of the reference CasX protein, and a second insertion may be in a second domain of the reference CasX protein. In some embodiments, a CasX variant protein comprises 2, 3, 4, 5, 6, 7, 8, 9, 10, 11, 12, 13, 14, 15, 16, 17, 18, 19 or 20 non-consecutive insertions relative to a reference CasX protein. In some embodiments, a CasX variant protein comprises at least 10 to about 20 or more non-consecutive insertions relative to a reference CasX protein. Each non-consecutive insertion may be of any length of amino acids described herein, e.g., 1-4 amino acids, 1-10 amino acids, and the like.

Any amino acid, or combination of amino acids, can be inserted in the insertions described herein. For example, a proline, arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine or any combination thereof can be inserted into a reference CasX protein of the disclosure to generate a CasX variant protein.

Any permutation of the substitution, insertion and deletion embodiments described herein can be combined to generate a CasX variant protein of the disclosure. For example, a CasX variant protein can comprise at least one substitution and at least one deletion relative to a reference CasX protein sequence, at least one substitution and at least one insertion relative to a reference CasX protein sequence, at least one insertion and at least one deletion relative to a reference CasX protein sequence, or at least one substitution, one insertion and one deletion relative to a reference CasX protein sequence.

In some embodiments, the CasX variant protein has at least about 60% sequence similarity, at least 70% similarity, at least 80% similarity, at least 85% similarity, at least 86% similarity, at least 87% similarity, at least 88% similarity, at least 89% similarity, at least 90% similarity, at least 91% similarity, at least 92% similarity, at least 93% similarity, at least 94% similarity, at least 95% similarity, at least 96% similarity, at least 97% similarity, at least 98% similarity, at least 99% similarity, at least 99.5% similarity, at least 99.6% similarity, at least 99.7% similarity, at least 99.8% similarity or at least 99.9% similarity to one of SEQ ID NO: 1, SEQ ID NO: 2, or SEQ ID NO: 3.

In some embodiments, the CasX variant protein has at least about 60% sequence similarity to SEQ ID NO: 2 or a portion thereof. In some embodiments, the CasX variant protein comprises a substitution of Y789T of SEQ ID NO: 2, a deletion of P793 of SEQ ID NO: 2, a substitution of Y789D of SEQ ID NO: 2, a substitution of T72S of SEQ ID NO: 2, a substitution of I546V of SEQ ID NO: 2, a substitution of E552A of SEQ ID NO: 2, a substitution of A636D of SEQ ID NO: 2, a substitution of F536S of SEQ ID NO:2, a substitution of A708K of SEQ ID NO: 2, a substitution of Y797L of SEQ ID NO: 2, a substitution of L792G SEQ ID NO: 2, a substitution of A739V of SEQ ID NO: 2, a substitution of G791M of SEQ ID NO: 2, an insertion of A at position 661 of SEQ ID NO: 2, a substitution of A788W of SEQ ID NO: 2, a substitution of K390R of SEQ ID NO: 2, a substitution of A751S of SEQ ID NO: 2, a substitution of E385A of SEQ ID NO: 2, an insertion of P at position 696 of SEQ ID NO: 2, an insertion of M at position 773 of SEQ ID NO: 2, a substitution of G695H of SEQ ID NO: 2, an insertion of AS at position 793 of SEQ ID NO: 2, an insertion of AS at position 795 of SEQ ID NO: 2, a substitution of C477R of SEQ ID NO: 2, a substitution of C477K of SEQ ID NO: 2, a substitution of C479A of SEQ ID NO: 2, a substitution of C479L of SEQ ID NO: 2, a substitution of I55F of SEQ ID NO: 2, a substitution of K210R of SEQ ID NO: 2, a substitution of C233S of SEQ ID NO: 2, a substitution of D231N of SEQ ID NO: 2, a substitution of Q338E of SEQ ID NO: 2, a substitution of Q338R of SEQ ID NO: 2, a substitution of L379R of SEQ ID NO: 2, a substitution of K390R of SEQ ID NO: 2, a substitution of L481Q of SEQ ID NO: 2, a substitution of F495S of SEQ ID NO:2, a substitution of D600N of SEQ ID NO: 2, a substitution of T886K of SEQ ID NO: 2, a substitution of A739V of SEQ ID NO: 2, a substitution of K460N of SEQ ID NO: 2, a substitution of I199F of SEQ ID NO: 2, a substitution of G492P of SEQ ID NO: 2, a substitution of T1531 of SEQ ID NO: 2, a substitution of R591I of SEQ ID NO: 2, an insertion of AS at position 795 of SEQ ID NO: 2, an insertion of AS at position 796 of SEQ ID NO:2, an insertion of L at position 889 of SEQ ID NO: 2, a substitution of E121D of SEQ ID NO: 2, a substitution of S270W of SEQ ID NO: 2, a substitution of E712Q of SEQ ID NO: 2, a substitution of K942Q of SEQ ID NO: 2, a substitution of E552K of SEQ ID NO:2, a substitution of K25Q of SEQ ID NO: 2, a substitution of N47D of SEQ ID NO: 2, an insertion of T at position 696 of SEQ ID NO: 2, a substitution of L685I of SEQ ID NO: 2, a substitution of N880D of SEQ ID NO: 2, a substitution of Q102R of SEQ ID NO: 2, a substitution of M734K of SEQ ID NO: 2, a substitution of A724S of SEQ ID NO: 2, a substitution of T704K of SEQ ID NO: 2, a substitution of P224K of SEQ ID NO: 2, a substitution of K25R of SEQ ID NO: 2, a substitution of M29E of SEQ ID NO: 2, a substitution of H152D of SEQ ID NO: 2, a substitution of S219R of SEQ ID NO: 2, a substitution of E475K of SEQ ID NO: 2, a substitution of G226R of SEQ ID NO: 2, a substitution of A377K of SEQ ID NO: 2, a substitution of E480K of SEQ ID NO: 2, a substitution of K416E of SEQ ID NO: 2, a substitution of H164R of SEQ ID NO: 2, a substitution of K767R of SEQ ID NO: 2, a substitution of I7F of SEQ ID NO: 2, a substitution of M29R of SEQ ID NO: 2, a substitution of H435R of SEQ ID NO: 2, a substitution of E385Q of SEQ ID NO: 2, a substitution of E385K of SEQ ID NO: 2, a substitution of I279F of SEQ ID NO: 2, a substitution of D489S of SEQ ID NO: 2, a substitution of D732N of SEQ ID NO: 2, a substitution of A739T of SEQ ID NO: 2, a substitution of W885R of SEQ ID NO: 2, a substitution of E53K of SEQ ID NO: 2, a substitution of A238T of SEQ ID NO: 2, a substitution of P283Q of SEQ ID NO: 2, a substitution of E292K of SEQ ID NO: 2, a substitution of Q628E of SEQ ID NO: 2, a substitution of R388Q of SEQ ID NO: 2, a substitution of G791M of SEQ ID NO: 2, a substitution of L792K of SEQ ID NO: 2, a substitution of L792E of SEQ ID NO: 2, a substitution of M779N of SEQ ID NO: 2, a substitution of G27D of SEQ ID NO: 2, a substitution of K955R of SEQ ID NO: 2, a substitution of S867R of SEQ ID NO: 2, a substitution of R693I of SEQ ID NO: 2, a substitution of F189Y of SEQ ID NO: 2, a substitution of V635M of SEQ ID NO: 2, a substitution of F399L of SEQ ID NO: 2, a substitution of E498K of SEQ ID NO: 2, a substitution of E386R of SEQ ID NO: 2, a substitution of V254G of SEQ ID NO: 2, a substitution of P793S of SEQ ID NO: 2, a substitution of K188E of SEQ ID NO: 2, a substitution of QT945KI of SEQ ID NO: 2, a substitution of T620P of SEQ ID NO: 2, a substitution of T946P of SEQ ID NO: 2, a substitution of TT949PP of SEQ ID NO: 2, a substitution of N952T of SEQ ID NO: 2, a substitution of K682E of SEQ ID NO: 2, a substitution of K975R of SEQ ID NO: 2, a substitution of L212P of SEQ ID NO: 2, a substitution of E292R of SEQ ID NO: 2, a substitution of 1303K of SEQ ID NO: 2, a substitution of C349E of SEQ ID NO: 2, a substitution of E385P of SEQ ID NO: 2, a substitution of E386N of SEQ ID NO: 2, a substitution of D387K of SEQ ID NO: 2, a substitution of L404K of SEQ ID NO: 2, a substitution of E466H of SEQ ID NO: 2, a substitution of C477Q of SEQ ID NO: 2, a substitution of C477H of SEQ ID NO: 2, a substitution of C479A of SEQ ID NO: 2, a substitution of D659H of SEQ ID NO: 2, a substitution of T806V of SEQ ID NO: 2, a substitution of K8085 of SEQ ID NO: 2, an insertion of AS at position 797 of SEQ ID NO: 2, a substitution of V959M of SEQ ID NO: 2, a substitution of K975Q of SEQ ID NO: 2, a substitution of W974G of SEQ ID NO: 2, a substitution of A708Q of SEQ ID NO: 2, a substitution of V711K of SEQ ID NO: 2, a substitution of D733T of SEQ ID NO: 2, a substitution of L742W of SEQ ID NO: 2, a substitution of V747K of SEQ ID NO: 2, a substitution of F755M of SEQ ID NO: 2, a substitution of M771A of SEQ ID NO: 2, a substitution of M771Q of SEQ ID NO: 2, a substitution of W782Q of SEQ ID NO: 2, a substitution of G791F, of SEQ ID NO: 2 a substitution of L792D of SEQ ID NO: 2, a substitution of L792K of SEQ ID NO: 2, a substitution of P793Q of SEQ ID NO: 2, a substitution of P793G of SEQ ID NO: 2, a substitution of Q804A of SEQ ID NO: 2, a substitution of Y966N of SEQ ID NO: 2, a substitution of Y723N of SEQ ID NO: 2, a substitution of Y857R of SEQ ID NO: 2, a substitution of 5890R of SEQ ID NO: 2, a substitution of S932M of SEQ ID NO: 2, a substitution of L897M of SEQ ID NO: 2, a substitution of R624G of SEQ ID NO: 2, a substitution of 5603G of SEQ ID NO: 2, a substitution of N737S of SEQ ID NO: 2, a substitution of L307K of SEQ ID NO: 2, a substitution of I658V of SEQ ID NO: 2, an insertion of PT at position 688 of SEQ ID NO: 2, an insertion of SA at position 794 of SEQ ID NO: 2, a substitution of S877R of SEQ ID NO: 2, a substitution of N580T of SEQ ID NO: 2, a substitution of V335G of SEQ ID NO: 2, a substitution of T620S of SEQ ID NO: 2, a substitution of W345G of SEQ ID NO: 2, a substitution of T280S of SEQ ID NO: 2, a substitution of L406P of SEQ ID NO: 2, a substitution of A612D of SEQ ID NO: 2, a substitution of A751S of SEQ ID NO: 2, a substitution of E386R of SEQ ID NO: 2, a substitution of V351M of SEQ ID NO: 2, a substitution of K210N of SEQ ID NO: 2, a substitution of D40A of SEQ ID NO: 2, a substitution of E773G of SEQ ID NO: 2, a substitution of H207L of SEQ ID NO: 2, a substitution of T62A SEQ ID NO: 2, a substitution of T287P of SEQ ID NO: 2, a substitution of T832A of SEQ ID NO: 2, a substitution of A893S of SEQ ID NO: 2, an insertion of V at position 14 of SEQ ID NO: 2, an insertion of AG at position 13 of SEQ ID NO: 2, a substitution of R11V of SEQ ID NO: 2, a substitution of R12N of SEQ ID NO: 2, a substitution of R13H of SEQ ID NO: 2, an insertion of Y at position 13 of SEQ ID NO: 2, a substitution of R12L of SEQ ID NO: 2, an insertion of Q at position 13 of SEQ ID NO: 2, an substitution of V155 of SEQ ID NO: 2, an insertion of D at position 17 of SEQ ID NO: 2 or a combination thereof.

In some embodiments, a CasX variant protein comprises at least two amino acid changes to a reference CasX protein amino acid sequence. The at least two amino acid changes can be substitutions, insertions, or deletions of a reference CasX protein amino acid sequence, or any combination thereof. In some embodiments, the at least two amino acid changes to the sequence of a reference CasX variant protein are selected from the group consisting of: a substitution of Y789T of SEQ ID NO: 2, a deletion of P793 of SEQ ID NO: 2, a substitution of Y789D of SEQ ID NO: 2, a substitution of T72S of SEQ ID NO: 2, a substitution of I546V of SEQ ID NO: 2, a substitution of E552A of SEQ ID NO: 2, a substitution of A636D of SEQ ID NO: 2, a substitution of F536S of SEQ ID NO:2, a substitution of A708K of SEQ ID NO: 2, a substitution of Y797L of SEQ ID NO: 2, a substitution of L792G SEQ ID NO: 2, a substitution of A739V of SEQ ID NO: 2, a substitution of G791M of SEQ ID NO: 2, an insertion of A at position 661 of SEQ ID NO: 2, a substitution of A788W of SEQ ID NO: 2, a substitution of K390R of SEQ ID NO: 2, a substitution of A751S of SEQ ID NO: 2, a substitution of E385A of SEQ ID NO: 2, an insertion of P at position 696 of SEQ ID NO: 2, an insertion of M at position 773 of SEQ ID NO: 2, a substitution of G695H of SEQ ID NO: 2, an insertion of AS at position 793 of SEQ ID NO: 2, an insertion of AS at position 795 of SEQ ID NO: 2, a substitution of C477R of SEQ ID NO: 2, a substitution of C477K of SEQ ID NO: 2, a substitution of C479A of SEQ ID NO: 2, a substitution of C479L of SEQ ID NO: 2, a substitution of I55F of SEQ ID NO: 2, a substitution of K210R of SEQ ID NO: 2, a substitution of C233S of SEQ ID NO: 2, a substitution of D231N of SEQ ID NO: 2, a substitution of Q338E of SEQ ID NO: 2, a substitution of Q338R of SEQ ID NO: 2, a substitution of L379R of SEQ ID NO: 2, a substitution of K390R of SEQ ID NO: 2, a substitution of L481Q of SEQ ID NO: 2, a substitution of F495S of SEQ ID NO:2, a substitution of D600N of SEQ ID NO: 2, a substitution of T886K of SEQ ID NO: 2, a substitution of A739V of SEQ ID NO: 2, a substitution of K460N of SEQ ID NO: 2, a substitution of I199F of SEQ ID NO: 2, a substitution of G492P of SEQ ID NO: 2, a substitution of T1531 of SEQ ID NO: 2, a substitution of R591I of SEQ ID NO: 2, an insertion of AS at position 795 of SEQ ID NO: 2, an insertion of AS at position 796 of SEQ ID NO:2, an insertion of L at position 889 of SEQ ID NO: 2, a substitution of E121D of SEQ ID NO: 2, a substitution of S270W of SEQ ID NO: 2, a substitution of E712Q of SEQ ID NO: 2, a substitution of K942Q of SEQ ID NO: 2, a substitution of E552K of SEQ ID NO:2, a substitution of K25Q of SEQ ID NO: 2, a substitution of N47D of SEQ ID NO: 2, an insertion of T at position 696 of SEQ ID NO: 2, a substitution of L685I of SEQ ID NO: 2, a substitution of N880D of SEQ ID NO: 2, a substitution of Q102R of SEQ ID NO: 2, a substitution of M734K of SEQ ID NO: 2, a substitution of A724S of SEQ ID NO: 2, a substitution of T704K of SEQ ID NO: 2, a substitution of P224K of SEQ ID NO: 2, a substitution of K25R of SEQ ID NO: 2, a substitution of M29E of SEQ ID NO: 2, a substitution of H152D of SEQ ID NO: 2, a substitution of S219R of SEQ ID NO: 2, a substitution of E475K of SEQ ID NO: 2, a substitution of G226R of SEQ ID NO: 2, a substitution of A377K of SEQ ID NO: 2, a substitution of E480K of SEQ ID NO: 2, a substitution of K416E of SEQ ID NO: 2, a substitution of H164R of SEQ ID NO: 2, a substitution of K767R of SEQ ID NO: 2, a substitution of I7F of SEQ ID NO: 2, a substitution of M29R of SEQ ID NO: 2, a substitution of H435R of SEQ ID NO: 2, a substitution of E385Q of SEQ ID NO: 2, a substitution of E385K of SEQ ID NO: 2, a substitution of I279F of SEQ ID NO: 2, a substitution of D489S of SEQ ID NO: 2, a substitution of D732N of SEQ ID NO: 2, a substitution of A739T of SEQ ID NO: 2, a substitution of W885R of SEQ ID NO: 2, a substitution of E53K of SEQ ID NO: 2, a substitution of A238T of SEQ ID NO: 2, a substitution of P283Q of SEQ ID NO: 2, a substitution of E292K of SEQ ID NO: 2, a substitution of Q628E of SEQ ID NO: 2, a substitution of R388Q of SEQ ID NO: 2, a substitution of G791M of SEQ ID NO: 2, a substitution of L792K of SEQ ID NO: 2, a substitution of L792E of SEQ ID NO: 2, a substitution of M779N of SEQ ID NO: 2, a substitution of G27D of SEQ ID NO: 2, a substitution of K955R of SEQ ID NO: 2, a substitution of S867R of SEQ ID NO: 2, a substitution of R693I of SEQ ID NO: 2, a substitution of F189Y of SEQ ID NO: 2, a substitution of V635M of SEQ ID NO: 2, a substitution of F399L of SEQ ID NO: 2, a substitution of E498K of SEQ ID NO: 2, a substitution of E386R of SEQ ID NO: 2, a substitution of V254G of SEQ ID NO: 2, a substitution of P793S of SEQ ID NO: 2, a substitution of K188E of SEQ ID NO: 2, a substitution of QT945KI of SEQ ID NO: 2, a substitution of T620P of SEQ ID NO: 2, a substitution of T946P of SEQ ID NO: 2, a substitution of TT949PP of SEQ ID NO: 2, a substitution of N952T of SEQ ID NO: 2, a substitution of K682E of SEQ ID NO: 2, a substitution of K975R of SEQ ID NO: 2, a substitution of L212P of SEQ ID NO: 2, a substitution of E292R of SEQ ID NO: 2, a substitution of 1303K of SEQ ID NO: 2, a substitution of C349E of SEQ ID NO: 2, a substitution of E385P of SEQ ID NO: 2, a substitution of E386N of SEQ ID NO: 2, a substitution of D387K of SEQ ID NO: 2, a substitution of L404K of SEQ ID NO: 2, a substitution of E466H of SEQ ID NO: 2, a substitution of C477Q of SEQ ID NO: 2, a substitution of C477H of SEQ ID NO: 2, a substitution of C479A of SEQ ID NO: 2, a substitution of D659H of SEQ ID NO: 2, a substitution of T806V of SEQ ID NO: 2, a substitution of K8085 of SEQ ID NO: 2, an insertion of AS at position 797 of SEQ ID NO: 2, a substitution of V959M of SEQ ID NO: 2, a substitution of K975Q of SEQ ID NO: 2, a substitution of W974G of SEQ ID NO: 2, a substitution of A708Q of SEQ ID NO: 2, a substitution of V711K of SEQ ID NO: 2, a substitution of D733T of SEQ ID NO: 2, a substitution of L742W of SEQ ID NO: 2, a substitution of V747K of SEQ ID NO: 2, a substitution of F755M of SEQ ID NO: 2, a substitution of M771A of SEQ ID NO: 2, a substitution of M771Q of SEQ ID NO: 2, a substitution of W782Q of SEQ ID NO: 2, a substitution of G791F, of SEQ ID NO: 2 a substitution of L792D of SEQ ID NO: 2, a substitution of L792K of SEQ ID NO: 2, a substitution of P793Q of SEQ ID NO: 2, a substitution of P793G of SEQ ID NO: 2, a substitution of Q804A of SEQ ID NO: 2, a substitution of Y966N of SEQ ID NO: 2, a substitution of Y723N of SEQ ID NO: 2, a substitution of Y857R of SEQ ID NO: 2, a substitution of 5890R of SEQ ID NO: 2, a substitution of S932M of SEQ ID NO: 2, a substitution of L897M of SEQ ID NO: 2, a substitution of R624G of SEQ ID NO: 2, a substitution of 5603G of SEQ ID NO: 2, a substitution of N737S of SEQ ID NO: 2, a substitution of L307K of SEQ ID NO: 2, a substitution of I658V of SEQ ID NO: 2, an insertion of PT at position 688 of SEQ ID NO: 2, an insertion of SA at position 794 of SEQ ID NO: 2, a substitution of S877R of SEQ ID NO: 2, a substitution of N580T of SEQ ID NO: 2, a substitution of V335G of SEQ ID NO: 2, a substitution of T620S of SEQ ID NO: 2, a substitution of W345G of SEQ ID NO: 2, a substitution of T280S of SEQ ID NO: 2, a substitution of L406P of SEQ ID NO: 2, a substitution of A612D of SEQ ID NO: 2, a substitution of A751S of SEQ ID NO: 2, a substitution of E386R of SEQ ID NO: 2, a substitution of V351M of SEQ ID NO: 2, a substitution of K210N of SEQ ID NO: 2, a substitution of D40A of SEQ ID NO: 2, a substitution of E773G of SEQ ID NO: 2, a substitution of H207L of SEQ ID NO: 2, a substitution of T62A SEQ ID NO: 2, a substitution of T287P of SEQ ID NO: 2, a substitution of T832A of SEQ ID NO: 2, a substitution of A893S of SEQ ID NO: 2, an insertion of V at position 14 of SEQ ID NO: 2, an insertion of AG at position 13 of SEQ ID NO: 2, a substitution of R11V of SEQ ID NO: 2, a substitution of R12N of SEQ ID NO: 2, a substitution of R13H of SEQ ID NO: 2, an insertion of Y at position 13 of SEQ ID NO: 2, a substitution of R12L of SEQ ID NO: 2, an insertion of Q at position 13 of SEQ ID NO: 2, an substitution of V155 of SEQ ID NO: 2 and an insertion of D at position 17 of SEQ ID NO: 2. In some embodiments, the at least two amino acid changes to a reference CasX protein are selected from the amino acid changes disclosed in the sequences of Table 3.

In some embodiments, a CasX variant protein comprises more than one substitution, insertion and/or deletion of a reference CasX protein amino acid sequence. In some embodiments, the reference CasX protein comprises or consists essentially of SEQ ID NO: 2. In some embodiments, a CasX variant protein comprises a substitution of S794R and a substitution of Y797L of SEQ ID NO: 2. In some embodiments, a CasX variant protein comprises a substitution of K416E and a substitution of A708K of SEQ ID NO: 2. In some embodiments, a CasX variant protein comprises a substitution of A708K and a deletion of P793 of SEQ ID NO: 2. In some embodiments, a CasX variant protein comprises a deletion of P793 and a substitution of P793AS SEQ ID NO: 2. In some embodiments, a CasX variant protein comprises a substitution of Q367K and a substitution of I425S of SEQ ID NO: 2. In some embodiments, a CasX variant protein comprises a substitution of A708K, a deletion of P position 793 and a substitution A793V of SEQ ID NO: 2. In some embodiments, a CasX variant protein comprises a substitution of Q338R and a substitution of A339E of SEQ ID NO: 2. In some embodiments, a CasX variant protein comprises a substitution of Q338R and a substitution of A339K of SEQ ID NO: 2. In some embodiments, a CasX variant protein comprises a substitution of 5507G and a substitution of G508R of SEQ ID NO: 2. In some embodiments, a CasX variant protein comprises a substitution of L379R, a substitution of A708K and a deletion of P at position 793 of SEQ ID NO: 2. In some embodiments, a CasX variant protein comprises a substitution of C477K, a substitution of A708K and a deletion of P at position 793 of SEQ ID NO: 2. In some embodiments, a CasX variant protein comprises a substitution of L379R, a substitution of C477K, a substitution of A708K and a deletion of P at position of 793 of SEQ ID NO: 2. In some embodiments, a CasX variant protein comprises a substitution of L379R, a substitution of A708K, a deletion of P at position 793 and a substitution A739V of SEQ ID NO: 2. In some embodiments, a CasX variant protein comprises a substitution of C477K, a substitution of A708K, a deletion of P at position 793 and a substitution of A739V of SEQ ID NO: 2. In some embodiments, a CasX variant protein comprises a substitution of L379R, a substitution of C477K, a substitution of A708K, a deletion of P at position 793 and a substitution of A739V of SEQ ID NO: 2. In some embodiments, a CasX variant protein comprises a substitution of L379R, a substitution of A708K, a deletion of P at position 793 and a substitution of M779N of SEQ ID NO: 2. In some embodiments, a CasX variant protein comprises a substitution of L379R, a substitution of A708K, a deletion of P at position 793 and a substitution of M771N of SEQ ID NO: 2. In some embodiments, a CasX variant protein comprises a substitution of L379R, a substitution of 708K, a deletion of P at position 793 and a substitution of D489S of SEQ ID NO: 2. In some embodiments, a CasX variant protein comprises a substitution of L379R, a substitution of A708K, a deletion of P at position 793 and a substitution of A739T of SEQ ID NO: 2. In some embodiments, a CasX variant protein comprises a substitution of L379R, a substitution of A708K, a deletion of P at position 793 and a substitution of D732N of SEQ ID NO: 2. In some embodiments, a CasX variant protein comprises a substitution of L379R, a substitution of A708K, a deletion of P at position 793 and a substitution of G791M of SEQ ID NO: 2. In some embodiments, a CasX variant protein comprises a substitution of L379R, a substitution of 708K, a deletion of P at position 793 and a substitution of Y797L of SEQ ID NO: 2. In some embodiments, a CasX variant protein comprises a substitution of L379R, a substitution of C477K, a substitution of A708K, a deletion of P at position 793 and a substitution of M779N of SEQ ID NO: 2. In some embodiments, a CasX variant protein comprises a substitution of L379R, a substitution of C477K, a substitution of A708K, a deletion of P at position 793 and a substitution of M771N of SEQ ID NO: 2. In some embodiments, a CasX variant protein comprises a substitution of L379R, a substitution of C477K, a substitution of A708K, a deletion of P at position 793 and a substitution of D489S of SEQ ID NO: 2. In some embodiments, a CasX variant protein comprises a substitution of L379R, a substitution of C477K, a substitution of A708K, a deletion of P at position 793 and a substitution of A739T of SEQ ID NO: 2. In some embodiments, a CasX variant protein comprises a substitution of L379R, a substitution of C477K, a substitution of A708K, a deletion of P at position 793 and a substitution of D732N of SEQ ID NO: 2. In some embodiments, a CasX variant protein comprises a substitution of L379R, a substitution of C477K, a substitution of A708K, a deletion of P at position 793 and a substitution of G791M of SEQ ID NO: 2. In some embodiments, a CasX variant protein comprises a substitution of L379R, a substitution of C477K, a substitution of A708K, a deletion of P at position 793 and a substitution of Y797L of SEQ ID NO: 2. In some embodiments, a CasX variant protein comprises a substitution of L379R, a substitution of C477K, a substitution of A708K, a deletion of P at position 793 and a substitution of T620P of SEQ ID NO: 2. In some embodiments, a CasX variant protein comprises a substitution of A708K, a deletion of P at position 793 and a substitution of E386S of SEQ ID NO: 2. In some embodiments, a CasX variant protein comprises a substitution of E386R, a substitution of F399L and a deletion of P at position 793 of SEQ ID NO: 2. In some embodiments, a CasX variant protein comprises a substitution of R581I and A739V of SEQ ID NO: 2. In some embodiments, a CasX variant comprises any combination of the foregoing embodiments of this paragraph.

In some embodiments, a CasX variant protein comprises more than one substitution, insertion and/or deletion of a reference CasX protein amino acid sequence. In some embodiments, a CasX variant protein comprises a substitution of A708K, a deletion of P at position 793 and a substitution of A739V of SEQ ID NO: 2. In some embodiments, a CasX variant protein comprises a substitution of L379R, a substitution of A708K and a deletion of P at position 793 of SEQ ID NO: 2. In some embodiments, a CasX variant protein comprises a substitution of C477K, a substitution of A708K and a deletion of P at position 793 of SEQ ID NO: 2. In some embodiments, a CasX variant protein comprises a substitution of L379R, a substitution of C477K, a substitution of A708K and a deletion of P at position 793 of SEQ ID NO: 2. In some embodiments, a CasX variant protein comprises a substitution of L379R, a substitution of A708K, a deletion of P at position 793 and a substitution of A739V of SEQ ID NO: 2. In some embodiments, a CasX variant protein comprises a substitution of C477K, a substitution of A708K, a deletion of P at position 793 and a substitution of A739 of SEQ ID NO: 2. In some embodiments, a CasX variant protein comprises a substitution of L379R, a substitution of C477K, a substitution of A708K, a deletion of P at position 793 and a substitution of A739V of SEQ ID NO: 2. In some embodiments, a CasX variant protein comprises a substitution of L379R, a substitution of C477K, a substitution of A708K, a deletion of P at position 793 and a substitution of T620P of SEQ ID NO: 2. In some embodiments, a CasX variant protein comprises a substitution of M771A of SEQ ID NO: 2. In some embodiments, a CasX variant protein comprises a substitution of L379R, a substitution of A708K, a deletion of P at position 793 and a substitution of D732N of SEQ ID NO: 2. In some embodiments, a CasX variant protein comprises a substitution of W782Q of SEQ ID NO: 2. In some embodiments, a CasX variant protein comprises a substitution of M771Q of SEQ ID NO: 2. In some embodiments, a CasX variant protein comprises a substitution of R458I and a substitution of A739V of SEQ ID NO: 2. In some embodiments, a CasX variant protein comprises a substitution of L379R, a substitution of A708K, a deletion of P at position 793 and a substitution of M771N of SEQ ID NO: 2. In some embodiments, a CasX variant protein comprises a substitution of L379R, a substitution of A708K, a deletion of P at position 793 and a substitution of A739T of SEQ ID NO: 2. In some embodiments, a CasX variant protein comprises a substitution of L379R, a substitution of C477K, a substitution of A708K, a deletion of P at position 793 and a substitution of D489S of SEQ ID NO: 2. In some embodiments, a CasX variant protein comprises a substitution of L379R, a substitution of C477K, a substitution of A708K, a deletion of P at position 793 and a substitution of D732N of SEQ ID NO: 2. In some embodiments, a CasX variant comprises any combination of the foregoing embodiments of this paragraph.

In some embodiments, a CasX variant protein comprises a substitution of V711K of SEQ ID NO: 2. In some embodiments, a CasX variant protein comprises a substitution of L379R, a substitution of C477K, a substitution of A708K, a deletion of P at position 793 and a substitution of Y797L of SEQ ID NO: 2. In some embodiments, a CasX variant protein comprises a substitution of L379R, a substitution of A708K and a deletion of P at position 793 of SEQ ID NO: 2.

In some embodiments, a CasX variant protein comprises a substitution of L379R, a substitution of C477K, a substitution of A708K, a deletion of P at position 793 and a substitution of M771N of SEQ ID NO: 2. In some embodiments, a CasX variant protein comprises a substitution of A708K, a substitution of P at position 793 and a substitution of E386S of SEQ ID NO: 2.

In some embodiments, a CasX variant protein comprises a substitution of L379R, a substitution of C477K, a substitution of A708K and a deletion of P at position 793 of SEQ ID NO: 2. In some embodiments, a CasX variant protein comprises a substitution of L792D of SEQ ID NO: 2. In some embodiments, a CasX variant protein comprises a substitution of G791F of SEQ ID NO: 2. In some embodiments, a CasX variant protein comprises a substitution of A708K, a deletion of P at position 793 and a substitution of A739V of SEQ ID NO: 2. In some embodiments, a CasX variant protein comprises a substitution of L379R, a substitution of A708K, a deletion of P at position 793 and a substitution of A739V of SEQ ID NO: 2. In some embodiments, a CasX variant protein comprises a substitution of C477K, a substitution of A708K and a substitution of P at position 793 of SEQ ID NO: 2. In some embodiments, a CasX variant protein comprises a substitution of L249I and a substitution of M771N of SEQ ID NO: 2. In some embodiments, a CasX variant protein comprises a substitution of V747K of SEQ ID NO: 2. In some embodiments, a CasX variant protein comprises a substitution of L379R, a substitution of C477, a substitution of A708K, a deletion of P at position 793 and a substitution of M779N of SEQ ID NO: 2. In some embodiments, a CasX variant protein comprises a substitution of F755M.

In some embodiments, the CasX variant protein comprises between 400 and 2000 amino acids, between 500 and 1500 amino acids, between 700 and 1200 amino acids, between 800 and 1100 amino acids or between 900 and 1000 amino acids.

In some embodiments, the CasX variant protein comprises one or more modifications comprising a region of non-contiguous residues that form a channel in which gNA:target DNA complexing occurs. In some embodiments, the CasX variant protein comprises one or more modifications comprising a region of non-contiguous residues that form an interface which binds with the gNA. For example, in some embodiments of a reference CasX protein, the Helical I, Helical II and OBD domains all contact or are in proximity to the gNA:target DNA complex, and one or more modifications to non-contiguous residues within any of these domains may improve function of the CasX variant protein.

In some embodiments, the CasX variant protein comprises one or more modifications comprising a region of non-contiguous residues that form a channel which binds with the non-target strand DNA. For example, a CasX variant protein can comprise one or more modifications to non-contiguous residues of the NTSBD. In some embodiments, the CasX variant protein comprises one or more modifications comprising a region of non-contiguous residues that form an interface which binds with the PAM. For example, a CasX variant protein can comprise one or more modifications to non-contiguous residues of the Helical I domain or OBD. In some embodiments, the CasX variant protein comprises one or more modifications comprising a region of non-contiguous surface-exposed residues. As used herein, “surface-exposed residues” refers to amino acids on the surface of the CasX protein, or amino acids in which at least a portion of the amino acid, such as the backbone or a part of the side chain is on the surface of the protein. Surface exposed residues of cellular proteins such as CasX, which are exposed to an aqueous intracellular environment, are frequently selected from positively charged hydrophilic amino acids, for example arginine, asparagine, aspartate, glutamine, glutamate, histidine, lysine, serine, and threonine. Thus, for example, in some embodiments of the variants provided herein, a region of surface exposed residues comprises one or more insertions, deletions, or substitutions compared to a reference CasX protein. In some embodiments, one or more positively charged residues are substituted for one or more other positively charged residues, or negatively charged residues, or uncharged residues, or any combinations thereof. In some embodiments, one or more amino acids residues for substitution are near bound nucleic acid, for example residues in the RuvC domain or Helical I domain that contact target DNA, or residues in the OBD or Helical II domain that bind the gRNA, can be substituted for one or more positively charged or polar amino acids.

In some embodiments, the CasX variant protein comprises one or more modifications comprising a region of non-contiguous residues that form a core through hydrophobic packing in a domain of the reference CasX protein. Without wishing to be bound by any theory, regions that form cores through hydrophobic packing are rich in hydrophobic amino acids such as valine, isoleucine, leucine, methionine, phenylalanine, tryptophan, and cysteine. For example, in some reference CasX proteins, RuvC domains comprise a hydrophobic pocket adjacent to the active site. In some embodiments, between 2 to 15 residues of the region are charged, polar, or base-stacking. Charged amino acids (interchangeably referred to herein as residues) may include, for example, arginine, lysine, aspartic acid, and glutamic acid, and the side chains of these amino acids may form salt bridges provided a bridge partner is also present. Polar amino acids may include, for example, glutamine, asparagine, histidine, serine, threonine, tyrosine, and cysteine. Polar amino acids can, in some embodiments, form hydrogen bonds as proton donors or acceptors, depending on the identity of their side chains. As used herein, “base-stacking” includes the interaction of aromatic side chains of an amino acid residue (such as tryptophan, tyrosine, phenylalanine, or histidine) with stacked nucleotide bases in a nucleic acid. Any modification to a region of non-contiguous amino acids that are in close spatial proximity to form a functional part of the CasX variant protein is envisaged as within the scope of the disclosure.

i. CasX Variant Proteins with Domains from Multiple Source Proteins

In certain embodiments, the disclosure provides a chimeric CasX protein comprising protein domains from two or more different CasX proteins, such as two or more reference CasX proteins, or two or more CasX variant protein sequences as described herein. As used herein, a “chimeric CasX protein” refers to a CasX containing at least two domains isolated or derived from different sources, such as two naturally occurring proteins, which may, in some embodiments, be isolated from different species. For example, in some embodiments, a chimeric CasX protein comprises a first domain from a first CasX protein and a second domain from a second, different CasX protein. In some embodiments, the first domain can be selected from the group consisting of the NTSB, TSL, Helical I, Helical II, OBD and RuvC domains. In some embodiments, the second domain is selected from the group consisting of the NTSB, TSL, Helical I, Helical II, OBD and RuvC domains with the second domain being different from the foregoing first domain. For example, a chimeric CasX protein may comprise an NTSB, TSL, Helical I, Helical II, OBD domains from a CasX protein of SEQ ID NO: 2, and a RuvC domain from a CasX protein of SEQ ID NO: 1, or vice versa. As a further example, a chimeric CasX protein may comprise an NTSB, TSL, Helical II, OBD and RuvC domain from CasX protein of SEQ ID NO: 2, and a Helical I domain from a CasX protein of SEQ ID NO: 1, or vice versa. Thus, in certain embodiments, a chimeric CasX protein may comprise an NTSB, TSL, Helical II, OBD and RuvC domain from a first CasX protein, and a Helical I domain from a second CasX protein. In some embodiments of the chimeric CasX proteins, the domains of the first CasX protein are derived from the sequences of SEQ ID NO: 1, SEQ ID NO: 2 or SEQ ID NO: 3, and the domains of the second CasX protein are derived from the sequences of SEQ ID NO: 1, SEQ ID NO: 2 or SEQ ID NO: 3, and the first and second CasX proteins are not the same. In some embodiments, domains of the first CasX protein comprise sequences derived from SEQ ID NO: 1 and domains of the second CasX protein comprise sequences derived from SEQ ID NO: 2. In some embodiments, domains of the first CasX protein comprise sequences derived from SEQ ID NO: 1 and domains of the second CasX protein comprise sequences derived from SEQ ID NO: 3. In some embodiments, domains of the first CasX protein comprise sequences derived from SEQ ID NO: 2 and domains of the second CasX protein comprise sequences derived from SEQ ID NO: 3.

In some embodiments, a CasX variant protein comprises at least one chimeric domain comprising a first part from a first CasX protein and a second part from a second, different CasX protein. As used herein, a “chimeric domain” refers to a single domain containing at least two parts isolated or derived from different sources, such as two naturally occurring proteins or portions of domains from two reference CasX proteins. The at least one chimeric domain can be any of the NTSB, TSL, Helical I, Helical II, OBD or RuvC domains as described herein. In some embodiments, the first portion of a CasX domain comprises a sequence of SEQ ID NO: 1 and the second portion of a CasX domain comprises a sequence of SEQ ID NO: 2. In some embodiments, the first portion of the CasX domain comprises a sequence of SEQ ID NO: 1 and the second portion of the CasX domain comprises a sequence of SEQ ID NO: 3. In some embodiments, the first portion of the CasX domain comprises a sequence of SEQ ID NO: 2 and the second portion of the CasX domain comprises a sequence of SEQ ID NO: 3. In some embodiments, the at least one chimeric domain comprises a chimeric RuvC domain. As an example of the foregoing, the chimeric RuvC domain comprises amino acids 661 to 824 of SEQ ID NO: 1 and amino acids 922 to 978 of SEQ ID NO: 2. As an alternative example of the foregoing, a chimeric RuvC domain comprises amino acids 648 to 812 of SEQ ID NO: 2 and amino acids 935 to 986 of SEQ ID NO: 1. In some embodiments, a CasX protein comprises a first domain from a first CasX protein and a second domain from a second CasX protein, and at least one chimeric domain comprising at least two parts isolated from different CasX proteins using the approach of the embodiments described in this paragraph.

In some embodiments, a CasX variant protein comprises a sequence of SEQ ID NOS: 49-160 as set forth in Table 3. In some embodiments, a CasX variant protein consists of a sequence of SEQ ID NOS: 49-160 as set forth in Table 3. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence set forth in Table 3.

TABLE 3
CasX Variant Sequences
(*Where a number is indicated in the left column, itdesignates a CasX variant
identification number different than the SEQ ID NO assigned to it;
where indicated, are relative to SEQ ID NO: 2)

| Description* | Amino Acid Sequence |
|---|---|
| TSL, Helical | MQEIKRINKIRRRLVKDSNTKKAGKTGPMKTLLVRVMTPDLRERLENLRKKP |
| I, Helical II, | ENIPQPISNTSRANLNKLLTDYTEMKKAILHVYWEEFQKDPVGLMSRVAQPA |
| OBD and | SKKIDQNKLKPEMDEKGNLTTAGFACSQCGQPLFVYKLEQVSEKGKAYTNYF |
| RuvC | GRCNVAEHEKLILLAQLKPEKDSDEAVTYSLGKFGQRALDFYSIHVTRESNH |
| domains | PVKPLEQIGGNSCASGPVGKALSDACMGAVASFLTKYQDIIIEHQKVIKKNE |
| from SEQ ID | KRLANLKDIASANGLAFPKITLPPQPHTKEGIEAYNNVVAQIVIWVNLNLWQ |
| NO: 2 and an | KLKIGRDEAKPLQRLKGFPSFPLVERQANEVDWWDMVCNVKKLINEKKEDGK |
| NTSB | VFWQNLAGYKRQEALRPYLSSEEDRKKGKKFARYQFGDLLLHLEKKHGEDWG |
| domain from | KVYDEAWERIDKKVEGLSKHIKLEEERRSEDAQSKAALTDWLRAKASFVIEG |
| SEQ ID | LKEADKDEFCRCELKLQKWYGDLRGKPFAIEAENSILDISGFSKQYNCAFIW |
| NO: 1 | QKDGVKKLNLYLIINYFKGGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGIDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 49) |
| NTSB, | MQEIKRINKIRRRLVKDSNTKKAGKTGPMKTLLVRVMTPDLRERLENLRKKP |
| Helical I, | ENIPQPISNTSRANLNKLLTDYTEMKKAILHVYWEEFQKDPVGLMSRVAQPA |
| Helical II, | PKNIDQRKLIPVKDGNERLTSSGFACSQCCQPLYVYKLEQVNDKGKPHTNYF |
| OBD and | GRCNVSEHERLILLSPHKPEANDELVTYSLGKFGQRALDFYSIHVTRESNHP |
| RuvC | VKPLEQIGGNSCASGPVGKALSDACMGAVASFLTKYQDIIIEHQKVIKKNEK |
| domains | RLANLKDIASANGLAFPKITLPPQPHTKEGIEAYNNVVAQIVIWVNLNLWQK |
| from SEQ ID | LKIGRDEAKPLQRLKGFPSFPLVERQANEVDWWDMVCNVKKLINEKKEDGKV |
| NO: 2 and a | FWQNLAGYKRQEALRPYLSSEEDRKKGKKFARYQFGDLLLHLEKKHGEDWGK |
| TSL domain | VYDEAWERIDKKVEGLSKHIKLEEERRSEDAQSKAALTDWLRAKASFVIEGL |
| from SEQ ID | KEADKDEFCRCELKLQKWYGDLRGKPFAIEAENSILDISGFSKQYNCAFIWQ |
| NO: 1. | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITTADYDGMLVRLKKTS |
| | DGWATTLNNKELKAEGQITYYNRYKRQTVEKELSAELDRLSEESGNNDISKW |
| | TKGRRDEALFLLKKRFSHRPVQEQFVCLDCGHEVHADEQAALNIARSWLFLR |
| | SQEYKKYQTNKTTGNTDKRAFVETWQSFYRKKLKEVWKPAV (SEQ ID |
| | NO: 50) |
| TSL, Helical | MEKRINKIRKKLSADNATKPVSRSGPMKTLLVRVMTDDLKKRLEKRRKKPEV |
| I, Helical II, | MPQVISNNAANNLRMLLDDYTKMKEAILQVYWQEFKDDHVGLMCKFAQPAPK |
| OBD and | NIDQRKLIPVKDGNERLTSSGFACSQCCQPLYVYKLEQVNDKGKPHTNYFGR |
| RuvC | CNVSEHERLILLSPHKPEANDELVTYSLGKFGQRALDFYSIHVTKESTHPVK |
| domains | PLAQIAGNRYASGPVGKALSDACMGTIASFLSKYQDIIIEHQKVVKGNQKRL |
| from SEQ ID | ESLRELAGKENLEYPSVTLPPQPHTKEGVDAYNEVIARVRMWVNLNLWQKLK |
| NO: 1 and an | LSRDDAKPLLRLKGFPSFPVVERRENEVDWWNTINEVKKLIDAKRDMGRVFW |
| NTSB | SGVTAEKRNTILEGYNYLPNENDHKKREGSLENPKKPAKRQFGDLLLYLEKK |
| domain from | YAGDWGKVFDEAWERIDKKIAGLTSHIEREEARNAEDAQSKAVLTDWLRAKA |
| SEQ ID | SFVLERLKEMDEKEFYACEIQLQKWYGDLRGNPFAVEAENRVVDISGESIGS |
| NO: 2 | DGHSIQYRNLLAWKYLENGKREFYLLMNYGKKGRIRFTDGTDIKKSGKWQGL |
| | LYGGGKAKVIDLTFDPDDEQLIILPLAFGTRQGREFIWNDLLSLETGLIKLA |
| | NGRVIEKTIYNKKIGRDEPALFVALTFERREVVDPSNIKPVNLIGVDRGENI |
| | PAVIALTDPEGCPLPEFKDSSGGPTDILRIGEGYKEKQRAIQAAKEVEQRRA |
| | GGYSRKFASKSRNLADDMVRNSARDLFYHAVTHDAVLVFENLSRGFGRQGKR |
| | TFMTERQYTKMEDWLTAKLAYEGLTSKTYLSKTLAQYTSKTCSNCGFTITTA |
| | DYDGMLVRLKKTSDGWATTLNNKELKAEGQITYYNRYKRQTVEKELSAELDR |
| | LSEESGNNDISKWTKGRRDEALFLLKKRFSHRPVQEQFVCLDCGHEVHADEQ |
| | AALNIARSWLFLNSNSTEFKSYKSGKQPFVGAWQAFYKRRLKEVWKPNA |
| | (SEQ ID NO: 51) |
| NTSB, | MEKRINKIRKKLSADNATKPVSRSGPMKTLLVRVMTDDLKKRLEKRRKKPEV |
| Helical I, | MPQVISNNAANNLRMLLDDYTKMKEAILQVYWQEFKDDHVGLMCKFAQPASK |
| Helical II, | KIDQNKLKPEMDEKGNLTTAGFACSQCGQPLFVYKLEQVSEKGKAYTNYFGR |
| OBD and | CNVAEHEKLILLAQLKPEKDSDEAVTYSLGKFGQRALDFYSIHVTKESTHPV |
| RuvC | KPLAQIAGNRYASGPVGKALSDACMGTIASFLSKYQDIIIEHQKVVKGNQKR |
| domains | LESLRELAGKENLEYPSVTLPPQPHTKEGVDAYNEVIARVRMWVNLNLWQKL |
| from SEQ ID | KLSRDDAKPLLRLKGFPSFPVVERRENEVDWWNTINEVKKLIDAKRDMGRVF |
| NO: 1 and an | WSGVTAEKRNTILEGYNYLPNENDHKKREGSLENPKKPAKRQFGDLLLYLEK |
| TSL domain | KYAGDWGKVFDEAWERIDKKIAGLTSHIEREEARNAEDAQSKAVLTDWLRAK |
| from SEQ ID | ASFVLERLKEMDEKEFYACEIQLQKWYGDLRGNPFAVEAENRVVDISGESIG |
| NO: 2. | SDGHSIQYRNLLAWKYLENGKREFYLLMNYGKKGRIRFTDGTDIKKSGKWQG |
| | LLYGGGKAKVIDLTFDPDDEQLIILPLAFGTRQGREFIWNDLLSLETGLIKL |
| | ANGRVIEKTIYNKKIGRDEPALFVALTFERREVVDPSNIKPVNLIGVDRGEN |
| | IPAVIALTDPEGCPLPEFKDSSGGPTDILRIGEGYKEKQRAIQAAKEVEQRR |
| | AGGYSRKFASKSRNLADDMVRNSARDLFYHAVTHDAVLVFENLSRGFGRQGK |
| | RTFMTERQYTKMEDWLTAKLAYEGLTSKTYLSKTLAQYTSKTCSNCGFTITS |
| | ADYDRVLEKLKKTATGWMTTINGKELKVEGQITYYNRYKRQNVVKDLSVELD |
| | RLSEESVNNDISSWTKGRSGEALSLLKKRFSHRPVQEKFVCLNCGFETHADE |
| | QAALNIARSWLFLNSNSTEFKSYKSGKQPFVGAWQAFYKRRLKEVWKPNA |
| | (SEQ ID NO: 52) |
| NTSB, TSL, | MQEIKRINKIRRRLVKDSNTKKAGKTGPMKTLLVRVMTPDLRERLENLRKKP |
| Helical I, | ENIPQPISNTSRANLNKLLTDYTEMKKAILHVYWEEFQKDPVGLMSRVAQPA |
| Helical II and | PKNIDQRKLIPVKDGNERLTSSGFACSQCCQPLYVYKLEQVNDKGKPHTNYF |
| OBD | GRCNVSEHERLILLSPHKPEANDELVTYSLGKFGQRALDFYSIHVTRESNHP |
| domains SEQ | VKPLEQIGGNSCASGPVGKALSDACMGAVASFLTKYQDIIIEHQKVIKKNEK |
| IDNO: 2and | RLANLKDIASANGLAFPKITLPPQPHTKEGIEAYNNVVAQIVIWVNLNLWQK |
| an exogenous | LKIGRDEAKPLQRLKGFPSFPLVERQANEVDWWDMVCNVKKLINEKKEDGKV |
| RuvC | FWQNLAGYKRQEALRPYLSSEEDRKKGKKFARYQFGDLLLHLEKKHGEDWGK |
| domain or a | VYDEAWERIDKKVEGLSKHIKLEEERRSEDAQSKAALTDWLRAKASFVIEGL |
| portion | KEADKDEFCRCELKLQKWYGDLRGKPFAIEAENSILDISGFSKQYNCAFIWQ |
| thereof from | KDGVKKLNLYLIINYFKGGKLRFKKIKPEAFEANRFYTVINKKSGEIVPMEV |
| a second | NFNFDDPNLIILPLAFGKRQGREFIWNDLLSLETGSLKLANGRVIEKTLYNR |
| CasX | RTRQDEPALFVALTFERREVLDSSNIKPVNLIGVDRGENIPAVIALTDPEGC |
| protein. | PLPEFKDSSGGPTDILRIGEGYKEKQRAIQAAKEVEQRRAGGYSRKFASKSR |
| | NLADDMVRNSARDLFYHAVTHDAVLVFENLSRGFGRQGKRTFMTERQYTKME |
| | DWLTAKLAYEGLTSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHA (SEQ ID NO: |
| | 53) |
| | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHA (SEQ ID NO: 54) |
| NTSB, TSL, | MQEIKRINKIRRRLVKDSNTKKAGKTGPMKTLLVRVMTPDLRERLENLRKKP |
| Helical II, | ENIPQP1SNNAANNLRMLLDDYTKMKEAILQVYWQEFKDDHVGLMCKFAQPA |
| OBD and | PKNIDQRKLIPVKDGNERLTSSGFACSQCCQPLYVYKLEQVNDKGKPHTNYF |
| RuvC | GRCNVSEHERLILLSPHKPEANDELVTYSLGKFGQRALDFYSIHVTKESTHP |
| domains | VKPLAQIAGNRYASGPVGKALSDACMGTIASFLSKYQDIIIEHQKVVKGNQK |
| from SEQ ID | RLESLRELAGKENLEYPSVTLPPQPHTKEGVDAYNEVIARVRMWVNLNLWQK |
| NO: 2 and a | LKLSRDDAKPLLRLKGFPSFPLVERQANEVDWWDMVCNVKKLINEKKEDGKV |
| Helical I | FWQNLAGYKRQEALRPYLSSEEDRKKGKKFARYQFGDLLLHLEKKHGEDWGK |
| domain from | VYDEAWERIDKKVEGLSKHIKLEEERRSEDAQSKAALTDWLRAKASFVIEGL |
| SEQ ID | KEADKDEFCRCELKLQKWYGDLRGKPFAIEAENSILDISGFSKQYNCAFIWQ |
| NO: 1 | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 55) |
| NTSB, TSL, | MQEIKRINKIRRRLVKDSNTKKAGKTGPMKTLLVRVMTPDLRERLENLRKKP |
| Helical I, | ENIPQPISNTSRANLNKLLTDYTEMKKAILHVYWEEFQKDPVGLMSRVAQPA |
| OBD and | PKNIDQRKLIPVKDGNERLTSSGFACSQCCQPLYVYKLEQVNDKGKPHTNYF |
| RuvC | GRCNVSEHERLILLSPHKPEANDELVTYSLGKFGQRALDFYSIHVTRESNHP |
| domains | VKPLEQIGGNSCASGPVGKALSDACMGAVASFLTKYQDIIIEHQKVIKKNEK |
| from SEQ ID | RLANLKDIASANGLAFPKITLPPQPHTKEGIEAYNNVVAQIVIWVNLNLWQK |
| NO: 2 and a | LKIGRDEAKPLQRLKGFPSFPVVERRENEVDWWNTINEVKKLIDAKRDMGRV |
| Helical II | FWSGVTAEKRNTILEGYNYLPNENDHKKREGSLENPKKPAKRQFGDLLLYLE |
| domain from | KKYAGDWGKVFDEAWERIDKKIAGLTSHIEREEARNAEDAQSKAVLTDWLRA |
| SEQ ID | KASFVLERLKEMDEKEFYACEIQLQKWYGDLRGNPFAVEAENSILDISGFSK |
| NO: 1 | QYNCAFIWQKDGVKKLNLYLIINYFKGGKLRFKKIKPEAFEANRFYTVINKK |
| | SGEIVPMEVNFNFDDPNLIILPLAFGKRQGREFIWNDLLSLETGSLKLANGR |
| | VIEKTLYNRRTRQDEPALFVALTFERREVLDSSNIKPMNLIGIDRGENIPAV |
| | IALTDPEGCPLSRFKDSLGNPTHILRIGESYKEKQRTIQAKKEVEQRRAGGY |
| | SRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDR |
| | VLEKLKKTATGWMTTINGKELKVEGQITYYNRYKRQNVVKDLSVELDRLSEE |
| | SVNNDISSWTKGRSGEALSLLKKRFSHRPVQEKFVCLNCGFETHADEQAALN |
| | IARSWLFLRSQEYKKYQTNKTTGNTDKRAFVETWQSFYRKKLKEVWKPAV |
| | (SEQ ID NO: 56) |
| NTSB, TSL, | MISNTSRANLNKLLTDYTEMKKAILHVYWEEFQKDPVGLMSRVAQPAPKNID |
| Helical I, | QRKLIPVKDGNERLTSSGFACSQCCQPLYVYKLEQVNDKGKPHTNYFGRCNV |
| Helical II and | SEHERLILLSPHKPEANDELVTYSLGKFGQRALDFYSIHVTRESNHPVKPLE |
| RuvC | QIGGNSCASGPVGKALSDACMGAVASFLTKYQDIIIEHQKVIKKNEKRLANL |
| domains | KDIASANGLAFPKITLPPQPHTKEGIEAYNNVVAQIVIWVNLNLWQKLKIGR |
| from a first | DEAKPLQRLKGFPSFPLVERQANEVDWWDMVCNVKKLINEKKEDGKVFWQNL |
| CasX protein | AGYKRQEALRPYLSSEEDRKKGKKFARYQFGDLLLHLEKKHGEDWGKVYDEA |
| and an | WERIDKKVEGLSKHIKLEEERRSEDAQSKAALTDWLRAKASFVIEGLKEADK |
| exogenous | DEFCRCELKLQKWYGDLRGKPFAIEAENRVVDISGFSIGSDGHSIQYRNLLA |
| OBD or a | WKYLENGKREFYLLMNYGKKGRIRFTDGTDIKKSGKWQGLLYGGGKAKVIDL |
| part thereof | TFDPDDEQLIILPLAFGTRQGREFIWNDLLSLETGLIKLANGRVIEKTIYNK |
| from a | KIGRDEPALFVALTFERREVVDPSNIKPMNLIGIDRGENIPAVIALTDPEGC |
| second CasX | PLSRFKDSLGNPTHILRIGESYKEKQRTIQAKKEVEQRRAGGYSRKYASKAK |
| protein | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 57) |
| | MEKRINKIRKKLSADNATKPVSRSGPMKTLLVRVMTDDLKKRLEKRRKKPEV |
| | MPQVISNTSRANLNKLLTDYTEMKKAILHVYWEEFQKDPVGLMSRVAQPAPK |
| | NIDQRKLIPVKDGNERLTSSGFACSQCCQPLYVYKLEQVNDKGKPHTNYFGR |
| | CNVSEHERLILLSPHKPEANDELVTYSLGKFGQRALDFYSIHVTRESNHPVK |
| | PLEQIGGNSCASGPVGKALSDACMGAVASFLTKYQDIIIEHQKVIKKNEKRL |
| | ANLKDIASANGLAFPKITLPPQPHTKEGIEAYNNVVAQIVIWVNLNLWQKLK |
| | IGRDEAKPLQRLKGFPSFPLVERQANEVDWWDMVCNVKKLINEKKEDGKVFW |
| | QNLAGYKRQEALRPYLSSEEDRKKGKKFARYQFGDLLLHLEKKHGEDWGKVY |
| | DEAWERIDKKVEGLSKHIKLEEERRSEDAQSKAALTDWLRAKASFVIEGLKE |
| | ADKDEFCRCELKLQKWYGDLRGKPFAIEAENSILDISGFSKQYNCAFIWQKD |
| | GVKKLNLYLIINYFKGGKLRFKKIKPEAFEANRFYTVINKKSGEIVPMEVNF |
| | NFDDPNLIILPLAFGKRQGREFIWNDLLSLETGSLKLANGRVIEKTLYNRRT |
| | RQDEPALFVALTFERREVLDSSNIKPMNLIGIDRGENIPAVIALTDPEGCPL |
| | SRFKDSLGNPTHILRIGESYKEKQRTIQAKKEVEQRRAGGYSRKYASKAKNL |
| | ADDMVRNTARDLLYYAVTQDAMLIFENLSRGFGRQGKRTFMAERQYTRMEDW |
| | LTAKLAYEGLSKTYLSKTLAQYTSKTCSNCGFTITSADYDRVLEKLKKTATG |
| | WMTTINGKELKVEGQITYYNRYKRQNVVKDLSVELDRLSEESVNNDISSWTK |
| | GRSGEALSLLKKRFSHRPVQEKFVCLNCGFETHADEQAALNIARSWLFLRSQ |
| | EYKKYQTNKTTGNTDKRAFVETWQSFYRKKLKEVWKPAV (SEQ ID NO: |
| | 58) |
| | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENRVVDISGFSIGSDGHSIQY |
| | RNLLAWKYLENGKREFYLLMNYGKKGRIRFTDGTDIKKSGKWQGLLYGGGKA |
| | KVIDLTFDPDDEQLIILPLAFGTRQGREFIWNDLLSLETGLIKLANGRVIEK |
| | TIYNKKIGRDEPALEVALTFERREVVDPSNIKPMNLIGIDRGENIPAVIALT |
| | DPEGCPLSRFKDSLGNPTHILRIGESYKEKQRTIQAKKEVEQRRAGGYSRKY |
| | ASKAKNLADDMVRNTARDLLYYAVTQDAMLIFENLSRGFGRQGKRTFMAERQ |
| | YTRMEDWLTAKLAYEGLSKTYLSKTLAQYTSKTCSNCGFTITSADYDRVLEK |
| | LKKTATGWMTTINGKELKVEGQITYYNRYKRQNVVKDLSVELDRLSEESVNN |
| | DISSWTKGRSGEALSLLKKRFSHRPVQEKFVCLNCGFETHADEQAALNIARS |
| | WLFLRSQEYKKYQTNKTTGNTDKRAFVETWQSFYRKKLKEVWKPAV(SEQ |
| | ID NO: 59) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K, a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 and a | VYDEAWERIDKKVEGLSKHIKLEEERRSEDAQSKAALTDWLRAKASFVIEGL |
| substitution | KEADKDEFKRCELKLQKWYGDLRGKPFAIEAENSILDISGFSKQYNCAFIWQ |
| of T620P of | KDGVKKLNLYLIINYFKGGKLRFKKIKPEAFEANRFYTVINKKSGEIVPMEV |
| SEQ ID | NFNFDDPNLIILPLAFGKRQGREFIWNDLLSLETGSLKLANGRVIEKPLYNR |
| NO: 2 | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 60) |
| substitution | MQEIKRINKIRRRLVKDSNTKKAGKTGPMKTLLVRVMTPDLRERLENLRKKP |
| of M771A of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2. | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFAAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPA (SEQ ID |
| | NO: 61) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of A708K, a | GRCNVSEHERLILLSPHKPEANDELVTYSLGKFGQRALDFYSIHVTRESNHP |
| deletion of P | VKPLEQIGGNSCASGPVGKALSDACMGAVASFLTKYQDIIIEHQKVIKKNEK |
| at position | RLANLKDIASANGLAFPKITLPPQPHTKEGIEAYNNVVAQIVIWVNLNLWQK |
| 793 and a | LKIGRDEAKPLQRLKGFPSFPLVERQANEVDWWDMVCNVKKLINEKKEDGKV |
| substitution | FWQNLAGYKRQEALRPYLSSEEDRKKGKKFARYQFGDLLLHLEKKHGEDWGK |
| of D732N of | VYDEAWERIDKKVEGLSKHIKLEEERRSEDAQSKAALTDWLRAKASFVIEGL |
| SEQ ID | KEADKDEFCRCELKLQKWYGDLRGKPFAIEAENSILDISGFSKQYNCAFIWQ |
| NO: 2. | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 62) |
| substitution | MQEIKRINKIRRRLVKDSNTKKAGKTGPMKTLLVRVMTPDLRERLENLRKKP |
| of W782Q of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2. | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFMAERQYTRME |
| | DQLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 63) |
| substitution | MQEIKRINKIRRRLVKDSNTKKAGKTGPMKTLLVRVMTPDLRERLENLRKKP |
| of M771Q of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2 | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFQAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 64) |
| substitution | MQEIKRINKIRRRLVKDSNTKKAGKTGPMKTLLVRVMTPDLRERLENLRKKP |
| of R458I and | ENIPQPISNTSRANLNKLLTDYTEMKKAILHVYWEEFQKDPVGLMSRVAQPA |
| a substitution | PKNIDQRKLIPVKDGNERLTSSGFACSQCCQPLYVYKLEQVNDKGKPHTNYF |
| of A739V of | GRCNVSEHERLILLSPHKPEANDELVTYSLGKFGQRALDFYSIHVTRESNHP |
| SEQ ID | VKPLEQIGGNSCASGPVGKALSDACMGAVASFLTKYQDIIIEHQKVIKKNEK |
| NO: 2. | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLIAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTVRDLLYYAVTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 65) |
| L379R, a | MQEIKRINKIRRRLVKDSNTKKAGKTGPMKTLLVRVMTPDLRERLENLRKKP |
| substitution | ENIPQPISNTSRANLNKLLTDYTEMKKAILHVYWEEFQKDPVGLMSRVAQPA |
| of A708K, a | PKNIDQRKLIPVKDGNERLTSSGFACSQCCQPLYVYKLEQVNDKGKPHTNYF |
| deletion of P | GRCNVSEHERLILLSPHKPEANDELVTYSLGKFGQRALDFYSIHVTRESNHP |
| at position | VKPLEQIGGNSCASGPVGKALSDACMGAVASFLTKYQDIIIEHQKVIKKNEK |
| 793 and a | RLANLKDIASANGLAFPKITLPPQPHTKEGIEAYNNVVAQIVIWVNLNLWQK |
| substitution | LKIGRDEAKPLQRLKGFPSFPLVERQANEVDWWDMVCNVKKLINEKKEDGKV |
| of M771N of | FWQNLAGYKRQEALRPYLSSEEDRKKGKKFARYQFGDLLLHLEKKHGEDWGK |
| SEQ ID | VYDEAWERIDKKVEGLSKHIKLEEERRSEDAQSKAALTDWLRAKASFVIEGL |
| NO: 2 | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFNAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 66) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of A708K, a | GRCNVSEHERLILLSPHKPEANDELVTYSLGKFGQRALDFYSIHVTRESNHP |
| deletion of P | VKPLEQIGGNSCASGPVGKALSDACMGAVASFLTKYQDIIIEHQKVIKKNEK |
| at position | RLANLKDIASANGLAFPKITLPPQPHTKEGIEAYNNVVAQIVIWVNLNLWQK |
| 793 and a | LKIGRDEAKPLQRLKGFPSFPLVERQANEVDWWDMVCNVKKLINEKKEDGKV |
| substitution | FWQNLAGYKRQEALRPYLSSEEDRKKGKKFARYQFGDLLLHLEKKHGEDWGK |
| of A739T of | VYDEAWERIDKKVEGLSKHIKLEEERRSEDAQSKAALTDWLRAKASFVIEGL |
| SEQ ID | KEADKDEFCRCELKLQKWYGDLRGKPFAIEAENSILDISGFSKQYNCAFIWQ |
| NO: 2 | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTTRDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 67) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K, a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 and a | VYDEAWERIDKKVEGLSKHIKLEEERRSEDAQSKAALTDWLRAKASFVIEGL |
| substitution | KEADKDEFKRCELKLQKWYGSLRGKPFAIEAENSILDISGFSKQYNCAFIWQ |
| of D489S of | KDGVKKLNLYLIINYFKGGKLRFKKIKPEAFEANRFYTVINKKSGEIVPMEV |
| SEQ ID | NFNFDDPNLIILPLAFGKRQGREFIWNDLLSLETGSLKLANGRVIEKTLYNR |
| NO: 2. | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 68) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K, a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 and a | VYDEAWERIDKKVEGLSKHIKLEEERRSEDAQSKAALTDWLRAKASFVIEGL |
| substitution | KEADKDEFKRCELKLQKWYGDLRGKPFAIEAENSILDISGFSKQYNCAFIWQ |
| of D732N of | KDGVKKLNLYLIINYFKGGKLRFKKIKPEAFEANRFYTVINKKSGEIVPMEV |
| SEQ ID | NFNFDDPNLIILPLAFGKRQGREFIWNDLLSLETGSLKLANGRVIEKTLYNR |
| NO: 2. | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 69) |
| substitution | MQEIKRINKIRRRLVKDSNTKKAGKTGPMKTLLVRVMTPDLRERLENLRKKP |
| of V711K of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2. | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEKEQRRAGGYSRKYASKAK |
| | NLADDMVRNTARDLLYYAVTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 70) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K, a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 and a | VYDEAWERIDKKVEGLSKHIKLEEERRSEDAQSKAALTDWLRAKASFVIEGL |
| substitution | KEADKDEFKRCELKLQKWYGDLRGKPFAIEAENSILDISGFSKQYNCAFIWQ |
| of Y797L of | KDGVKKLNLYLIINYFKGGKLRFKKIKPEAFEANRFYTVINKKSGEIVPMEV |
| SEQ ID | NFNFDDPNLIILPLAFGKRQGREFIWNDLLSLETGSLKLANGRVIEKTLYNR |
| NO: 2. | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 71) |
| 119: | MQEIKRINKIRRRLVKDSNTKKAGKTGPMKTLLVRVMTPDLRERLENLRKKP |
| substitution | ENIPQPISNTSRANLNKLLTDYTEMKKAILHVYWEEFQKDPVGLMSRVAQPA |
| of L379R, a | PKNIDQRKLIPVKDGNERLTSSGFACSQCCQPLYVYKLEQVNDKGKPHTNYF |
| substitution | GRCNVSEHERLILLSPHKPEANDELVTYSLGKFGQRALDFYSIHVTRESNHP |
| of A708K | VKPLEQIGGNSCASGPVGKALSDACMGAVASFLTKYQDIIIEHQKVIKKNEK |
| and a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 of SEQ | VYDEAWERIDKKVEGLSKHIKLEEERRSEDAQSKAALTDWLRAKASFVIEGL |
| IDNO: 2. | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 72) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K, a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 and a | VYDEAWERIDKKVEGLSKHIKLEEERRSEDAQSKAALTDWLRAKASFVIEGL |
| substitution | KEADKDEFKRCELKLQKWYGDLRGKPFAIEAENSILDISGFSKQYNCAFIWQ |
| of M771N of | KDGVKKLNLYLIINYFKGGKLRFKKIKPEAFEANRFYTVINKKSGEIVPMEV |
| SEQ ID | NFNFDDPNLIILPLAFGKRQGREFIWNDLLSLETGSLKLANGRVIEKTLYNR |
| NO: 2. | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFNAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 73) |
| substitution | MQEIKRINKIRRRLVKDSNTKKAGKTGPMKTLLVRVMTPDLRERLENLRKKP |
| of A708K, a | ENIPQPISNTSRANLNKLLTDYTEMKKAILHVYWEEFQKDPVGLMSRVAQPA |
| deletion of P | PKNIDQRKLIPVKDGNERLTSSGFACSQCCQPLYVYKLEQVNDKGKPHTNYF |
| at position | GRCNVSEHERLILLSPHKPEANDELVTYSLGKFGQRALDFYSIHVTRESNHP |
| 793 and a | VKPLEQIGGNSCASGPVGKALSDACMGAVASFLTKYQDIIIEHQKVIKKNEK |
| substitution | RLANLKDIASANGLAFPKITLPPQPHTKEGIEAYNNVVAQIVIWVNLNLWQK |
| of E386S of | LKIGRDEAKPLQRLKGFPSFPLVERQANEVDWWDMVCNVKKLINEKKEDGKV |
| SEQ ID | FWQNLAGYKRQEALLPYLSSESDRKKGKKFARYQFGDLLLHLEKKHGEDWGK |
| NO: 2. | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 74) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K | RLANLKDIASANGLAFPKITLPPQPHTKEGIEAYNNVVAQIVIWVNLNLWQK |
| and a | LKIGRDEAKPLQRLKGFPSFPLVERQANEVDWWDMVCNVKKLINEKKEDGKV |
| deletion of P | FWQNLAGYKRQEALRPYLSSEEDRKKGKKFARYQFGDLLLHLEKKHGEDWGK |
| at position | VYDEAWERIDKKVEGLSKHIKLEEERRSEDAQSKAALTDWLRAKASFVIEGL |
| 793 of SEQ | KEADKDEFKRCELKLQKWYGDLRGKPFAIEAENSILDISGFSKQYNCAFIWQ |
| IDNO: 2. | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 75) |
| substitution | MQEIKRINKIRRRLVKDSNTKKAGKTGPMKTLLVRVMTPDLRERLENLRKKP |
| of L792D of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2. | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFMAERQYTRME |
| | DWLTAKLAYEGDPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 76) |
| substitution | MQEIKRINKIRRRLVKDSNTKKAGKTGPMKTLLVRVMTPDLRERLENLRKKP |
| of G791F of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2. | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFMAERQYTRME |
| | DWLTAKLAYEFLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 77) |
| substitution | MQEIKRINKIRRRLVKDSNTKKAGKTGPMKTLLVRVMTPDLRERLENLRKKP |
| of A708K, a | ENIPQPISNTSRANLNKLLTDYTEMKKAILHVYWEEFQKDPVGLMSRVAQPA |
| deletion of P | PKNIDQRKLIPVKDGNERLTSSGFACSQCCQPLYVYKLEQVNDKGKPHTNYF |
| at position | GRCNVSEHERLILLSPHKPEANDELVTYSLGKFGQRALDFYSIHVTRESNHP |
| 793 and a | VKPLEQIGGNSCASGPVGKALSDACMGAVASFLTKYQDIIIEHQKVIKKNEK |
| substitution | RLANLKDIASANGLAFPKITLPPQPHTKEGIEAYNNVVAQIVIWVNLNLWQK |
| of A739V of | LKIGRDEAKPLQRLKGFPSFPLVERQANEVDWWDMVCNVKKLINEKKEDGKV |
| SEQ ID | FWQNLAGYKRQEALLPYLSSEEDRKKGKKFARYQFGDLLLHLEKKHGEDWGK |
| NO: 2. | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTVRDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 78) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of A708K, a | GRCNVSEHERLILLSPHKPEANDELVTYSLGKFGQRALDFYSIHVTRESNHP |
| deletion of P | VKPLEQIGGNSCASGPVGKALSDACMGAVASFLTKYQDIIIEHQKVIKKNEK |
| at position | RLANLKDIASANGLAFPKITLPPQPHTKEGIEAYNNVVAQIVIWVNLNLWQK |
| 793 and a | LKIGRDEAKPLQRLKGFPSFPLVERQANEVDWWDMVCNVKKLINEKKEDGKV |
| substitution | FWQNLAGYKRQEALRPYLSSEEDRKKGKKFARYQFGDLLLHLEKKHGEDWGK |
| of A739V of | VYDEAWERIDKKVEGLSKHIKLEEERRSEDAQSKAALTDWLRAKASFVIEGL |
| SEQ ID | KEADKDEFCRCELKLQKWYGDLRGKPFAIEAENSILDISGFSKQYNCAFIWQ |
| NO: 2. | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTVRDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 79) |
| substitution | MQEIKRINKIRRRLVKDSNTKKAGKTGPMKTLLVRVMTPDLRERLENLRKKP |
| of C477K, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of A708K | GRCNVSEHERLILLSPHKPEANDELVTYSLGKFGQRALDFYSIHVTRESNHP |
| and a | VKPLEQIGGNSCASGPVGKALSDACMGAVASFLTKYQDIIIEHQKVIKKNEK |
| deletion of P | RLANLKDIASANGLAFPKITLPPQPHTKEGIEAYNNVVAQIVIWVNLNLWQK |
| at position | LKIGRDEAKPLQRLKGFPSFPLVERQANEVDWWDMVCNVKKLINEKKEDGKV |
| 793 of SEQ | FWQNLAGYKRQEALLPYLSSEEDRKKGKKFARYQFGDLLLHLEKKHGEDWGK |
| ID NO: 2. | VYDEAWERIDKKVEGLSKHIKLEEERRSEDAQSKAALTDWLRAKASFVIEGL |
| | KEADKDEFKRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 80) |
| substitution | MQEIKRINKIRRRLVKDSNTKKAGKTGPMKTLLVRVMTPDLRERLENLRKKP |
| of L249I and | ENIPQPISNTSRANLNKLLTDYTEMKKAILHVYWEEFQKDPVGLMSRVAQPA |
| a substitution | PKNIDQRKLIPVKDGNERLTSSGFACSQCCQPLYVYKLEQVNDKGKPHTNYF |
| of M771N of | GRCNVSEHERLILLSPHKPEANDELVTYSLGKFGQRALDFYSIHVTRESNHP |
| SEQ ID | VKPLEQIGGNSCASGPVGKALSDACMGAVASFLTKYQDIIIEHQKVIKKNEK |
| NO: 2. | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFNAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 81) |
| substitution | MQEIKRINKIRRRLVKDSNTKKAGKTGPMKTLLVRVMTPDLRERLENLRKKP |
| of V747K of | ENIPQPISNTSRANLNKLLTDYTEMKKAILHVYWEEFQKDPVGLMSRVAQPA |
| SEQ ID | PKNIDQRKLIPVKDGNERLTSSGFACSQCCQPLYVYKLEQVNDKGKPHTNYF |
| NO: 2. | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAKTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 82) |
| substitution | MQEIKRINKIRRRLVKDSNTKKAGKTGPMKTLLVRVMTPDLRERLENLRKKP |
| of L379R, a | ENIPQPISNTSRANLNKLLTDYTEMKKAILHVYWEEFQKDPVGLMSRVAQPA |
| substitution | PKNIDQRKLIPVKDGNERLTSSGFACSQCCQPLYVYKLEQVNDKGKPHTNYF |
| of C477K, a | GRCNVSEHERLILLSPHKPEANDELVTYSLGKFGQRALDFYSIHVTRESNHP |
| substitution | VKPLEQIGGNSCASGPVGKALSDACMGAVASFLTKYQDIIIEHQKVIKKNEK |
| of A708K, a | RLANLKDIASANGLAFPKITLPPQPHTKEGIEAYNNVVAQIVIWVNLNLWQK |
| deletion of P | LKIGRDEAKPLQRLKGFPSFPLVERQANEVDWWDMVCNVKKLINEKKEDGKV |
| at position | FWQNLAGYKRQEALRPYLSSEEDRKKGKKFARYQFGDLLLHLEKKHGEDWGK |
| 793 and a | VYDEAWERIDKKVEGLSKHIKLEEERRSEDAQSKAALTDWLRAKASFVIEGL |
| substitution | KEADKDEFKRCELKLQKWYGDLRGKPFAIEAENSILDISGFSKQYNCAFIWQ |
| of M779N of | KDGVKKLNLYLIINYFKGGKLRFKKIKPEAFEANRFYTVINKKSGEIVPMEV |
| SEQ ID | NFNFDDPNLIILPLAFGKRQGREFIWNDLLSLETGSLKLANGRVIEKTLYNR |
| NO: 2. | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRNE |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 83) |
| L379R, | MQEIKRINKIRRRLVKDSNTKKAGKTGPMKTLLVRVMTPDLRERLENLRKKP |
| F755M | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIMENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 84) |
| 429: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 85) |
| 430: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 86) |
| 431: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| E386N | LKIGRDEAKPLQRLKGFPSFPLVERQANEVDWWDMVCNVKKLINEKKEDGKV |
| | FWQNLAGYKRQEALRPYLSSENDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 87) |
| 432: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| L404K | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLKHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 88) |
| 433: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQVRALDFYSIHVTRESNH |
| Y857R, | PVKPLEQIGGNSCASGPVGKALSDACMGAVASFLTKYQDIIIEHQKVIKKNE |
| 1658V, | KRLANLKDIASANGLAFPKITLPPQPHTKEGIEAYNNVVAQIVIWVNLNLWQ |
| AV192 | KLKIGRDEAKPLQRLKGFPSFPLVERQANEVDWWDMVCNVKKLINEKKEDGK |
| | VFWQNLAGYKRQEALRPYLSSEEDRKKGKKFARYQFGDLLLHLEKKHGEDWG |
| | KVYDEAWERIDKKVEGLSKHIKLEEERRSEDAQSKAALTDWLRAKASFVIEG |
| | LKEADKDEFCRCELKLQKWYGDLRGKPFAIEAENSILDISGFSKQYNCAFIW |
| | QKDGVKKLNLYLIINYFKGGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGVDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLSKTYLSKTLAQYTSKTCSNCGFTITSADYDRVLEKLKKT |
| | ATGWMTTINGKELKVEGQITYYNRRKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 89) |
| 434: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| L404K, | LKIGRDEAKPLQRLKGFPSFPLVERQANEVDWWDMVCNVKKLINEKKEDGKV |
| E386N | FWQNLAGYKRQEALRPYLSSENDRKKGKKFARYQFGDLLKHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 90) |
| 435: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L | LKIGRDEAKPLQRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 91) |
| 436: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| E386N | FWQNLAGYKRQEALRPYLSSENDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 92) |
| 437: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| C477S | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFSRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 93) |
| 438: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| L404K | FWQNLAGYKRQEALRPYLSSEEDRKKGKKFARYQLGDLLKHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 94) |
| 439: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| E386N, | FWQNLAGYKRQEALRPYLSSENDRKKGKKFARYQLGDLLKHLEKKHGEDWGK |
| C477S, | VYDEAWERIDKKVEGLSKHIKLEEERRSEDAQSKAALTDWLRAKASFVIEGL |
| L404K | KEADKDEFSRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 95) |
| 440: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| Y797L | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 96) |
| 441: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| Y797L, | FWQNLAGYKRQEALRPYLSSENDRKKGKKFARYQLGDLLLHLEKKHGEDWGK |
| E386N | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 97) |
| 442: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| F399L, | LKIGRDEAKPLQRLKGFPSFPLVERQANEVDWWDMVCNVKKLINEKKEDGKV |
| Y797L, | FWQNLAGYKRQEALRPYLSSENDRKKGKKFARYQLGDLLKHLEKKHGEDWGK |
| E386N, | VYDEAWERIDKKVEGLSKHIKLEEERRSEDAQSKAALTDWLRAKASFVIEGL |
| C477S, | KEADKDEFSRCELKLQKWYGDLRGKPFAIEAENSILDISGFSKQYNCAFIWQ |
| L404K | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 98) |
| 443: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| Y797L | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 99) |
| 444: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| Y797L, | LKIGRDEAKPLQRLKGFPSFPLVERQANEVDWWDMVCNVKKLINEKKEDGKV |
| L404K | FWQNLAGYKRQEALRPYLSSEEDRKKGKKFARYQFGDLLKHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 100) |
| 445: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| Y797L, | LKIGRDEAKPLQRLKGFPSFPLVERQANEVDWWDMVCNVKKLINEKKEDGKV |
| E386N | FWQNLAGYKRQEALRPYLSSENDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 101) |
| 446: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| 1658V, | RLANLKDIASANGLAFPKITLPPQPHTKEGIEAYNNVVAQIVIWVNLNLWQK |
| Y797L, | LKIGRDEAKPLQRLKGFPSFPLVERQANEVDWWDMVCNVKKLINEKKEDGKV |
| E386N, | FWQNLAGYKRQEALRPYLSSENDRKKGKKFARYQFGDLLKHLEKKHGEDWGK |
| C477S, | VYDEAWERIDKKVEGLSKHIKLEEERRSEDAQSKAALTDWLRAKASFVIEGL |
| L404K | KEADKDEFSRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTLLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 102) |
| 447: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| E386N | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSENDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 103) |
| 448: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| Y857R, | VKPLEQIGGNSCASGPVGKALSDACMGAVASFLTKYQDIIIEHQKVIKKNEK |
| E386N, | RLANLKDIASANGLAFPKITLPPQPHTKEGIEAYNNVVAQIVIWVNLNLWQK |
| L404K | LKIGRDEAKPLQRLKGFPSFPLVERQANEVDWWDMVCNVKKLINEKKEDGKV |
| | FWQNLAGYKRQEALRPYLSSENDRKKGKKFARYQFGDLLKHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 104) |
| 449: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| D732N, | VKPLEQIGGNSCASGPVGKALSDACMGAVASFLTKYQDIIIEHQKVIKKNEK |
| E385P, | RLANLKDIASANGLAFPKITLPPQPHTKEGIEAYNNVVAQIVIWVNLNLWQK |
| Y857R | LKIGRDEAKPLQRLKGFPSFPLVERQANEVDWWDMVCNVKKLINEKKEDGKV |
| | FWQNLAGYKRQEALRPYLSSPEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 105) |
| 450: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| D732N, | VKPLEQIGGNSCASGPVGKALSDACMGAVASFLTKYQDIIIEHQKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V | FWQNLAGYKRQEALRPYLSSPEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 106) |
| 451: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| D732N, | VKPLEQIGGNSCASGPVGKALSDACMGAVASFLTKYQDIIIEHQKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPEDRKKGKKFARYQLGDLLLHLEKKHGEDWGK |
| F399L | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 107) |
| 452: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| D732N, | VKPLEQIGGNSCASGPVGKALSDACMGAVASFLTKYQDIIIEHQKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPNDRKKGKKFARYQFGDLLLHLEKKHGEDWGK |
| E386N | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 108) |
| 453: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| D732N, | VKPLEQIGGNSCASGPVGKALSDACMGAVASFLTKYQDIIIEHQKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPEDRKKGKKFARYQFGDLLKHLEKKHGEDWGK |
| L404K | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLANDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 109) |
| 454: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHKKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| Q252K | FWQNLAGYKRQEALRPYLSSPEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 110) |
| 455: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHKKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPEDRKKGKKFARYQFGDLLLHLEKKHGEDWGK |
| Q252K | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 111) |
| 456: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHKKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPNDRKKGKKFARYQFGDLLLHLEKKHGEDWGK |
| E386N, | VYDEAWERIDKKVEGLSKHIKLEEERRSEDAQSKAALTDWLRAKASFVIEGL |
| Q252K | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 112) |
| 457: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHKKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPEDRKKGKKFARYQLGDLLLHLEKKHGEDWGK |
| F399L, | VYDEAWERIDKKVEGLSKHIKLEEERRSEDAQSKAALTDWLRAKASFVIEGL |
| Q252K | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 113) |
| 458: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHKKVIKKNEK |
| E385P, | RLANLKDIASANGLAFPKITLPPQPHTKEGIEAYNNVVAQIVIWVNLNLWQK |
| Y857R, | LKIGRDEAKPLQRLKGFPSFPLVERQANEVDWWDMVCNVKKLINEKKEDGKV |
| 1658V, | FWQNLAGYKRQEALRPYLSSPEDRKKGKKFARYQFGDLLKHLEKKHGEDWGK |
| L404K, | VYDEAWERIDKKVEGLSKHIKLEEERRSEDAQSKAALTDWLRAKASFVIEGL |
| Q252K | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 114) |
| 459: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHQKVIKKNEK |
| Y857R, | RLANLKDIASANGLAFPKITLPPQPHTKEGIEAYNNVVAQIVIWVNLNLWQK |
| 1658V, | LKIGRDEAKPLQRLKGFPSFPLVERQANEVDWWDMVCNVKKLINEKKEDGKV |
| E386N | FWQNLAGYKRQEALRPYLSSENDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 115) |
| 460: | MQEIKRINKIRRRLVKDSNTKKAGKTGPMKTLLVRVMTPDLRERLENLRKKP |
| L379R, | ENIPQPISNTSRANLNKLLTDYTEMKKAILHVYWEEFQKDPVGLMSRVAQPA |
| A708K, | PKNIDQRKLIPVKDGNERLTSSGFACSQCCQPLYVYKLEQVNDKGKPHTNYF |
| P793_, | GRCNVSEHERLILLSPHKPEANDELVTYSLGKFGQRALDFYSIHVTRESNHP |
| T620P, | VKPLEQIGGNSCASGPVGKALSDACMGAVASFLTKYQDIIIEHKKVIKKNEK |
| E385P, | RLANLKDIASANGLAFPKITLPPQPHTKEGIEAYNNVVAQIVIWVNLNLWQK |
| Q252K | LKIGRDEAKPLQRLKGFPSFPLVERQANEVDWWDMVCNVKKLINEKKEDGKV |
| | FWQNLAGYKRQEALRPYLSSPEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKPLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 116) |
| 278 | QEIKRINKIRRRLVKDSNTKKAGKTGPMKTLLVRVMTPDLRERLENLRKKPE |
| | NIPQPISNTSRANLNKLLTDYTEMKKAILHVYWEEFQKDPVGLMSRVAQPAP |
| | KNIDQRKLIPVKDGNERLTSSGFACSQCCQPLYVYKLEQVNDKGKPHTNYFG |
| | RCNVSEHERLILLSPHKPEANDELVTYSLGKFGQRALDFYSIHVTRESNHPV |
| | KPLEQIGGNSCASGPVGKALSDACMGAVASFLTKYQDIIIEHQKVIKKNEKR |
| | LANLKDIASANGLAFPKITLPPQPHTKEGIEAYNNVVAQIVIWVNLNLWQKL |
| | KIGRDEAKPLQRLKGFPSFPLVERQANEVDWWDMVCNVKKLINEKKEDGKVF |
| | WQNLAGYKRQEALRPYLSSEEDRKKGKKFARYQFGDLLLHLEKKHGEDWGKV |
| | YDEAWERIDKKVEGLSKHIKLEEERRSEDAQSKAALTDWLRAKASFVIEGLK |
| | EADKDEFCRCELKLQKWYGDLRGKPFAIEAENSILDISGFSKQYNCAFIWQK |
| | DGVKKLNLYLIINYFKGGKLRFKKIKPEAFEANRFYTVINKKSGEIVPMEVN |
| | FNFDDPNLIILPLAFGKRQGREFIWNDLLSLETGSLKLANGRVIEKTLYNRR |
| | TRQDEPALFVALTFERREVLDSSNIKPMNLIGIDRGENIPAVIALTDPEGCP |
| | LSRFKDSLGNPTHILRIGESYKEKQRTIQAKKEVEQRRAGGYSRKYASKAKN |
| | LADDMVRNTARDLLYYAVTQDAMLIFENLSRGFGRQGKRTFMAERQYTRMED |
| | WLTAKLAYEGLSKTYLSKTLAQYTSKTCSNCGFTITSADYDRVLEKLKKTAT |
| | GWMTTINGKELKVEGQITYYNRYKRQNVVKDLSVELDRLSEESVNNDISSWT |
| | KGRSGEALSLLKKRFSHRPVQEKFVCLNCGFETHADEQAALNIARSWLFLRS |
| | QEYKKYQTNKTTGNTDKRAFVETWQSFYRKKLKEVWKPAV (SEQ ID NO: |
| | 117) |
| 279 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 118) |
| 280 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 119) |
| 285 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 120) |
| 286 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 121) |
| 287 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 122) |
| 288 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTMSSGFACSQCCQPLYVYKLEQVNDKGKPHTNY |
| | FGRCNVSEHERLILLSPHKPEANDELVTYSLGKFGQRALDFYSIHVTRESNH |
| | PVKPLEQIGGNSCASGPVGKALSDACMGAVASFLTKYQDIIIEHQKVIKKNE |
| | KRLANLKDIASANGLAFPKITLPPQPHTKEGIEAYNNVVAQIVIWVNLNLWQ |
| | KLKIGRDEAKPLQRLKGFPSFPLVERQANEVDWWDMVCNVKKLINEKKEDGK |
| | VFWQNLAGYKRQEALRPYLSSEEDRKKGKKFARYQFGDLLLHLEKKHGEDWG |
| | KVYDEAWERIDKKVEGLSKHIKLEEERRSEDAQSKAALTDWLRAKASFVIEG |
| | LKEADKDEFCRCELKLQKWYGDLRGKPFAIEAENSILDISGFSKQYNCAFIW |
| | QKDGVKKLNLYLIINYFKGGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGIDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 123) |
| 290 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 124) |
| 291 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 125) |
| 293 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 126) |
| 300 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 127) |
| 492 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 128) |
| 493 | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 129) |
| 387: | QEIKRINKIRRRLVKDSNTKKAGKTGPMKTLLVRVMTPDLRERLENLRKKPE |
| NT SB swap | NIPQPISNTSRANLNKLLTDYTEMKKAILHVYWEEFQKDPVGLMSRVAQPAS |
| from SEQ ID | KKIDQNKLKPEMDEKGNLTTAGFACSQCGQPLFVYKLEQVSEKGKAYTNYFG |
| NO: 1 | RCNVAEHEKLILLAQLKPEKDSDEAVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 130) |
| 395: | QEIKRINKIRRRLVKDSNTKKAGKTGPMKTLLVRVMTPDLRERLENLRKKPE |
| Helical 1B | NIPQPISNTSRANLNKLLTDYTEMKKAILHVYWEEFQKDPVGLMSRVAQPAP |
| swap from | KNIDQRKLIPVKDGNERLTSSGFACSQCCQPLYVYKLEQVNDKGKPHTNYFG |
| SEQ ID | RCNVSEHERLILLSPHKPEANDELVTYSLGKFGQRALDFYSIHVTKESTHPV |
| NO: 1 | KPLAQIAGNRYASGPVGKALSDACMGTIASFLSKYQDIIIEHQKVVKGNQKR |
| | LESLRELAGKENLEYPSVTLPPQPHTKEGVDAYNEVIARVRMWVNLNLWQKL |
| | KLSRDDAKPLLRLKGFPSFPLVERQANEVDWWDMVCNVKKLINEKKEDGKVF |
| | WQNLAGYKRQEALRPYLSSEEDRKKGKKFARYQFGDLLLHLEKKHGEDWGKV |
| | YDEAWERIDKKVEGLSKHIKLEEERRSEDAQSKAALTDWLRAKASFVIEGLK |
| | EADKDEFCRCELKLQKWYGDLRGKPFAIEAENSILDISGFSKQYNCAFIWQK |
| | DGVKKLNLYLIINYFKGGKLRFKKIKPEAFEANRFYTVINKKSGEIVPMEVN |
| | FNFDDPNLIILPLAFGKRQGREFIWNDLLSLETGSLKLANGRVIEKTLYNRR |
| | TRQDEPALFVALTFERREVLDSSNIKPMNLIGIDRGENIPAVIALTDPEGCP |
| | LSRFKDSLGNPTHILRIGESYKEKQRTIQAKKEVEQRRAGGYSRKYASKAKN |
| | LADDMVRNTARDLLYYAVTQDAMLIFENLSRGFGRQGKRTFMAERQYTRMED |
| | WLTAKLAYEGLSKTYLSKTLAQYTSKTCSNCGFTITSADYDRVLEKLKKTAT |
| | GWMTTINGKELKVEGQITYYNRYKRQNVVKDLSVELDRLSEESVNNDISSWT |
| | KGRSGEALSLLKKRFSHRPVQEKFVCLNCGFETHADEQAALNIARSWLFLRS |
| | QEYKKYQTNKTTGNTDKRAFVETWQSFYRKKLKEVWKPAV (SEQ ID NO: |
| | 131) |
| 485: | QEIKRINKIRRRLVKDSNTKKAGKTGPMKTLLVRVMTPDLRERLENLRKKPE |
| Helical 1B | NIPQPISNTSRANLNKLLTDYTEMKKAILHVYWEEFQKDPVGLMSRVAQPAP |
| swap from | KNIDQRKLIPVKDGNERLTSSGFACSQCCQPLYVYKLEQVNDKGKPHTNYFG |
| SEQ ID | RCNVSEHERLILLSPHKPEANDELVTYSLGKFGQRALDFYSIHVTKESTHPV |
| NO: 1 | KPLAQIAGNRYASGPVGKALSDACMGTIASFLSKYQDIIIEHQKVVKGNQKR |
| | LESLRELAGKENLEYPSVTLPPQPHTKEGVDAYNEVIARVRMWVNLNLWQKL |
| | KLSRDDAKPLLRLKGFPSFPLVERQANEVDWWDMVCNVKKLINEKKEDGKVF |
| | WQNLAGYKRQEALRPYLSSEEDRKKGKKFARYQLGDLLLHLEKKHGEDWGKV |
| | YDEAWERIDKKVEGLSKHIKLEEERRSEDAQSKAALTDWLRAKASFVIEGLK |
| | EADKDEFCRCELKLQKWYGDLRGKPFAIEAENSILDISGFSKQYNCAFIWQK |
| | DGVKKLNLYLIINYFKGGKLRFKKIKPEAFEANRFYTVINKKSGEIVPMEVN |
| | FNFDDPNLIILPLAFGKRQGREFIWNDLLSLETGSLKLANGRVIEKTLYNRR |
| | TRQDEPALFVALTFERREVLDSSNIKPMNLIGVDRGENIPAVIALTDPEGCP |
| | LSRFKDSLGNPTHILRIGESYKEKQRTIQAKKEVEQRRAGGYSRKYASKAKN |
| | LADDMVRNTARDLLYYAVTQDAMLIFENLSRGFGRQGKRTFMAERQYTRMED |
| | WLTAKLAYEGLSKTYLSKTLAQYTSKTCSNCGFTITSADYDRVLEKLKKTAT |
| | GWMTTINGKELKVEGQITYYNRRKRQNVVKDLSVELDRLSEESVNNDISSWT |
| | KGRSGEALSLLKKRFSHRPVQEKFVCLNCGFETHADEQAALNIARSWLFLRS |
| | QEYKKYQTNKTTGNTDKRAFVETWQSFYRKKLKEVWKPAV (SEQ ID NO: |
| | 132) |
| 486: | QEIKRINKIRRRLVKDSNTKKAGKTGPMKTLLVRVMTPDLRERLENLRKKPE |
| Helical 1B | NIPQPISNTSRANLNKLLTDYTEMKKAILHVYWEEFQKDPVGLMSRVAQPAP |
| swap from | KNIDQRKLIPVKDGNERLTSSGFACSQCCQPLYVYKLEQVNDKGKPHTNYFG |
| SEQ ID | RCNVSEHERLILLSPHKPEANDELVTYSLGKFGQRALDFYSIHVTKESTHPV |
| NO: 1 | KPLAQIAGNRYASGPVGKALSDACMGTIASFLSKYQDIIIEHQKVVKGNQKR |
| | LESLRELAGKENLEYPSVTLPPQPHTKEGVDAYNEVIARVRMWVNLNLWQKL |
| | KLSRDDAKPLLRLKGFPSFPLVERQANEVDWWDMVCNVKKLINEKKEDGKVF |
| | WQNLAGYKRQEALRPYLSSEEDRKKGKKFARYQLGDLLKHLEKKHGEDWGKV |
| | YDEAWERIDKKVEGLSKHIKLEEERRSEDAQSKAALTDWLRAKASFVIEGLK |
| | EADKDEFCRCELKLQKWYGDLRGKPFAIEAENSILDISGFSKQYNCAFIWQK |
| | DGVKKLNLYLIINYFKGGKLRFKKIKPEAFEANRFYTVINKKSGEIVPMEVN |
| | FNFDDPNLIILPLAFGKRQGREFIWNDLLSLETGSLKLANGRVIEKTLYNRR |
| | TRQDEPALFVALTFERREVLDSSNIKPMNLIGVDRGENIPAVIALTDPEGCP |
| | LSRFKDSLGNPTHILRIGESYKEKQRTIQAKKEVEQRRAGGYSRKYASKAKN |
| | LADDMVRNTARDLLYYAVTQDAMLIFENLSRGFGRQGKRTFMAERQYTRMED |
| | WLTAKLAYEGLSKTYLSKTLAQYTSKTCSNCGFTITSADYDRVLEKLKKTAT |
| | GWMTTINGKELKVEGQITYYNRRKRQNVVKDLSVELDRLSEESVNNDISSWT |
| | KGRSGEALSLLKKRFSHRPVQEKFVCLNCGFETHADEQAALNIARSWLFLRS |
| | QEYKKYQTNKTTGNTDKRAFVETWQSFYRKKLKEVWKPAV (SEQ ID NO: |
| | 133) |
| 487: | QEIKRINKIRRRLVKDSNTKKAGKTGPMKTLLVRVMTPDLRERLENLRKKPE |
| Helical 1B | NIPQPISNTSRANLNKLLTDYTEMKKAILHVYWEEFQKDPVGLMSRVAQPAP |
| swap from | KNIDQRKLIPVKDGNERLTSSGFACSQCCQPLYVYKLEQVNDKGKPHTNYFG |
| SEQ ID | RCNVSEHERLILLSPHKPEANDELVTYSLGKFGQRALDFYSIHVTKESTHPV |
| NO: 1 | KPLAQIAGNRYASGPVGKALSDACMGTIASFLSKYQDIIIEHQKVVKGNQKR |
| | LESLRELAGKENLEYPSVTLPPQPHTKEGVDAYNEVIARVRMWVNLNLWQKL |
| | KLSRDDAKPLLRLKGFPSFPLVERQANEVDWWDMVCNVKKLINEKKEDGKVF |
| | WQNLAGYKRQEALRPYLSSEEDRKKGKKFARYQLGDLLLHLEKKHGEDWGKV |
| | YDEAWERIDKKVEGLSKHIKLEEERRSEDAQSKAALTDWLRAKASFVIEGLK |
| | EADKDEFCRCELKLQKWYGDLRGKPFAIEAENSILDISGFSKQYNCAFIWQK |
| | DGVKKLNLYLIINYFKGGKLRFKKIKPEAFEANRFYTVINKKSGEIVPMEVN |
| | FNFDDPNLIILPLAFGKRQGREFIWNDLLSLETGSLKLANGRVIEKTLYNRR |
| | TRQDEPALFVALTFERREVLDSSNIKPMNLIGVDRGENIPAVIALTDPEGCP |
| | LSRFKDSLGNPTHILRIGESYKEKQRTIQAKKEVEQRRAGGYSRKYASKAKN |
| | LADDMVRNTARDLLYYAVTQDAMLIFENLSRGFGRQGKRTFMAERQYTRMED |
| | WLTAKLAYEGLSKTYLSKTLAQYTSKTCSNCGFTITSADYDRVLEKLKKTAT |
| | GWMTTINGKELKVEGQITYYNRYKRQNVVKDLSVELDRLSEESVNNDISSWT |
| | KGRSGEALSLLKKRFSHRPVQEKFVCLNCGFETHADEQAALNIARSWLFLRS |
| | QEYKKYQTNKTTGNTDKRAFVETWQSFYRKKLKEVWKPAV (SEQ ID NO: |
| | 134) |
| 488: | QEIKRINKIRRRLVKDSNTKKAGKTGPMKTLLVRVMTPDLRERLENLRKKPE |
| NTSB and | NIPQPISNTSRANLNKLLTDYTEMKKAILHVYWEEFQKDPVGLMSRVAQPAS |
| Helical 1B | KKIDQNKLKPEMDEKGNLTTAGFACSQCGQPLFVYKLEQVSEKGKAYTNYFG |
| swap from | RCNVAEHEKLILLAQLKPEKDSDEAVTYSLGKFGQRALDFYSIHVTKESTHP |
| SEQ ID | VKPLAQIAGNRYASGPVGKALSDACMGTIASFLSKYQDIIIEHQKVVKGNQK |
| NO: 1 | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 135) |
| 489: | QEIKRINKIRRRLVKDSNTKKAGKTGPMKTLLVRVMTPDLRERLENLRKKPE |
| NTSB and | NIPQPISNTSRANLNKLLTDYTEMKKAILHVYWEEFQKDPVGLMSRVAQPAS |
| Helical 1B | KKIDQNKLKPEMDEKGNLTTAGFACSQCGQPLFVYKLEQVSEKGKAYTNYFG |
| swap from | RCNVAEHEKLILLAQLKPEKDSDEAVTYSLGKFGQRALDFYSIHVTKESTHP |
| SEQ ID | VKPLAQIAGNRYASGPVGKALSDACMGTIASFLSKYQDIIIEHQKVVKGNQK |
| NO: 1 | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 136) |
| 490: | QEIKRINKIRRRLVKDSNTKKAGKTGPMKTLLVRVMTPDLRERLENLRKKPE |
| NTSB and | NIPQPISNTSRANLNKLLTDYTEMKKAILHVYWEEFQKDPVGLMSRVAQPAS |
| Helical 1B | KKIDQNKLKPEMDEKGNLTTAGFACSQCGQPLFVYKLEQVSEKGKAYTNYFG |
| swap from | RCNVAEHEKLILLAQLKPEKDSDEAVTYSLGKFGQRALDFYSIHVTKESTHP |
| SEQ ID | VKPLAQIAGNRYASGPVGKALSDACMGTIASFLSKYQDIIIEHQKVVKGNQK |
| NO: 1 | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLKHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRRKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 137) |
| 491: | QEIKRINKIRRRLVKDSNTKKAGKTGPMKTLLVRVMTPDLRERLENLRKKPE |
| NTSB and | NIPQPISNTSRANLNKLLTDYTEMKKAILHVYWEEFQKDPVGLMSRVAQPAS |
| Helical 1B | KKIDQNKLKPEMDEKGNLTTAGFACSQCGQPLFVYKLEQVSEKGKAYTNYFG |
| swap from | RCNVAEHEKLILLAQLKPEKDSDEAVTYSLGKFGQRALDFYSIHVTKESTHP |
| SEQ ID | VKPLAQIAGNRYASGPVGKALSDACMGTIASFLSKYQDIIIEHQKVVKGNQK |
| NO: 1 | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 138) |
| 494: | QEIKRINKIRRRLVKDSNTKKAGKTGPMKTLLVRVMTPDLRERLENLRKKPE |
| NTSB swap | NIPQPISNTSRANLNKLLTDYTEMKKAILHVYWEEFQKDPVGLMSRVAQPAS |
| from SEQ ID | KKIDQNKLKPEMDEKGNLTTAGFACSQCGQPLFVYKLEQVSEKGKAYTNYFG |
| NO: 1 | RCNVAEHEKLILLAQLKPEKDSDEAVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 139) |
| 328: S867G | MQEIKRINKIRRRLVKDSNTKKAGKTGPMKTLLVRVMTPDLRERLENLRKKP |
| | ENIPQPISNTSRANLNKLLTDYTEMKKAILHVYWEEFQKDPVGLMSRVAQPA |
| | PKNIDQRKLIPVKDGNERLTSSGFACSQCCQPLYVYKLEQVNDKGKPHTNYF |
| | GRCNVSEHERLILLSPHKPEANDELVTYSLGKFGQRALDFYSIHVTRESNHP |
| | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALLPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLGVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 140) |
| 388: | MQEIKRINKIRRRLVKDSNTKKAGKTGPMKTLLVRVMTPDLRERLENLRKKP |
| L379R + A70 | ENIPQPISNTSRANLNKLLTDYTEMKKAILHVYWEEFQKDPVGLMSRVAQPA |
| 8K [P793] | PKNIDQRKLIPVKDGNERLTSSGFACSQCCQPLYVYKLEQVNDKGKPHTNYF |
| + X1 | GRCNVSEHERLILLSPHKPEANDELVTYSLGKFGQRALDFYSIHVTRESNHP |
| Helical2 | VKPLEQIGGNSCASGPVGKALSDACMGAVASFLTKYQDIIIEHQKVIKKNEK |
| swap | RLANLKDIASANGLAFPKITLPPQPHTKEGIEAYNNVVAQIVIWVNLNLWQK |
| | LKIGRDEAKPLQRLKGFPSFPVVERRENEVDWWNTINEVKKLIDAKRDMGRV |
| | FWSGVTAEKRNTILEGYNYLPNENDHKKREGSLENPKKPAKRQFGDLLLYLE |
| | KKYAGDWGKVFDEAWERIDKKIAGLTSHIEREEARNAEDAQSKAVLTDWLRA |
| | KASFVLERLKEMDEKEFYACEIQLQKWYGDLRGNPFAVEAENSILDISGFSK |
| | QYNCAFIWQKDGVKKLNLYLIINYFKGGKLRFKKIKPEAFEANRFYTVINKK |
| | SGEIVPMEVNFNFDDPNLIILPLAFGKRQGREFIWNDLLSLETGSLKLANGR |
| | VIEKTLYNRRTRQDEPALFVALTFERREVLDSSNIKPMNLIGIDRGENIPAV |
| | IALTDPEGCPLSRFKDSLGNPTHILRIGESYKEKQRTIQAKKEVEQRRAGGY |
| | SRKYASKAKNLADDMVRNTARDLLYYAVTQDAMLIFENLSRGFGRQGKRTFM |
| | AERQYTRMEDWLTAKLAYEGLSKTYLSKTLAQYTSKTCSNCGFTITSADYDR |
| | VLEKLKKTATGWMTTINGKELKVEGQITYYNRYKRQNVVKDLSVELDRLSEE |
| | SVNNDISSWTKGRSGEALSLLKKRFSHRPVQEKFVCLNCGFETHADEQAALN |
| | IARSWLFLRSQEYKKYQTNKTTGNTDKRAFVETWQSFYRKKLKEVWKPAV |
| | (SEQ ID NO: 141) |
| 389: | MQEIKRINKIRRRLVKDSNTKKAGKTGPMKTLLVRVMTPDLRERLENLRKKP |
| L379R + A70 | ENIPQPISNTSRANLNKLLTDYTEMKKAILHVYWEEFQKDPVGLMSRVAQPA |
| 8K [P793] | PKNIDQRKLIPVKDGNERLTSSGFACSQCCQPLYVYKLEQVNDKGKPHTNYF |
| + X1 RuvC1 | GRCNVSEHERLILLSPHKPEANDELVTYSLGKFGQRALDFYSIHVTRESNHP |
| swap | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPVNLIGVDRGENIPAVIALTDPEGC |
| | PLPEFKDSSGGPTDILRIGEGYKEKQRAIQAAKEVEQRRAGGYSRKFASKSR |
| | NLADDMVRNSARDLFYHAVTHDAVLVFENLSRGFGRQGKRTFMTERQYTKME |
| | DWLTAKLAYEGLTSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 142) |
| 390: | MQEIKRINKIRRRLVKDSNTKKAGKTGPMKTLLVRVMTPDLRERLENLRKKP |
| L379R + A70 | ENIPQPISNTSRANLNKLLTDYTEMKKAILHVYWEEFQKDPVGLMSRVAQPA |
| 8K + [P793] | PKNIDQRKLIPVKDGNERLTSSGFACSQCCQPLYVYKLEQVNDKGKPHTNYF |
| + X1 RuvC2 | GRCNVSEHERLILLSPHKPEANDELVTYSLGKFGQRALDFYSIHVTRESNHP |
| swap | VKPLEQIGGNSCASGPVGKALSDACMGAVASFLTKYQDIIIEHQKVIKKNEK |
| | RLANLKDIASANGLAFPKITLPPQPHTKEGIEAYNNVVAQIVIWVNLNLWQK |
| | LKIGRDEAKPLQRLKGFPSFPLVERQANEVDWWDMVCNVKKLINEKKEDGKV |
| | FWQNLAGYKRQEALRPYLSSEEDRKKGKKFARYQFGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGIDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLN |
| | SNSTEFKSYKSGKQPFVGAWQAFYKRRLKEVWKPNA (SEQ ID NO: |
| | 143) |
| 514: | QEIKRINKIRRRLVKDSNTKKAGKTGPMKTLLVRVMTPDLRERLENLRKKPE |
| H817 in 491 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTIHTSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 144) |
| 515: | QEIKRINKIRRRLVKDSNTKKAGKTGPMKTLLVRVMTPDLRERLENLRKKPE |
| \hat{ }P793 in 491 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 145) |
| 516: | QEIKRINKIRRRLVKDSNTKKAGKTGPMKTLLVRVMTPDLRERLENLRKKPE |
| L307H in | NIPQPISNTSRANLNKLLTDYTEMKKAILHVYWEEFQKDPVGLMSRVAQPAS |
| 491 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNHNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 146) |
| 517: | QEIKRINKIRRRLVKDSNTKKAGKTGPMKTLLVRVMTPDLRERLENLRKKPE |
| \hat{ }A224 in 491 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGAPVGKALSDACMGTIASFLSKYQDIIIEHQKVVKGNQ |
| | KRLESLRELAGKENLEYPSVTLPPQPHTKEGVDAYNEVIARVRMWVNLNLWQ |
| | KLKLSRDDAKPLLRLKGFPSFPLVERQANEVDWWDMVCNVKKLINEKKEDGK |
| | VFWQNLAGYKRQEALRPYLSSEEDRKKGKKFARYQLGDLLLHLEKKHGEDWG |
| | KVYDEAWERIDKKVEGLSKHIKLEEERRSEDAQSKAALTDWLRAKASFVIEG |
| | LKEADKDEFCRCELKLQKWYGDLRGKPFAIEAENSILDISGFSKQYNCAFIW |
| | QKDGVKKLNLYLIINYFKGGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGVDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 147) |
| 518: | RQEIKRINKIRRRLVKDSNTKKAGKTGPMKTLLVRVMTPDLRERLENLRKKP |
| \hat{ }R1 in 491 | ENIPQPISNTSRANLNKLLTDYTEMKKAILHVYWEEFQKDPVGLMSRVAQPA |
| | SKKIDQNKLKPEMDEKGNLTTAGFACSQCGQPLFVYKLEQVSEKGKAYTNYF |
| | GRCNVAEHEKLILLAQLKPEKDSDEAVTYSLGKFGQRALDFYSIHVTKESTH |
| | PVKPLAQIAGNRYASGAPVGKALSDACMGTIASFLSKYQDIIIEHQKVVKGN |
| | QKRLESLRELAGKENLEYPSVTLPPQPHTKEGVDAYNEVIARVRMWVNLNLW |
| | QKLKLSRDDAKPLLRLKGFPSFPLVERQANEVDWWDMVCNVKKLINEKKEDG |
| | KVFWQNLAGYKRQEALRPYLSSEEDRKKGKKFARYQLGDLLLHLEKKHGEDW |
| | GKVYDEAWERIDKKVEGLSKHIKLEEERRSEDAQSKAALTDWLRAKASFVIE |
| | GLKEADKDEFCRCELKLQKWYGDLRGKPFAIEAENSILDISGFSKQYNCAFI |
| | WQKDGVKKLNLYLIINYFKGGKLRFKKIKPEAFEANRFYTVINKKSGEIVPM |
| | EVNFNFDDPNLIILPLAFGKRQGREFIWNDLLSLETGSLKLANGRVIEKTLY |
| | NRRTRQDEPALFVALTFERREVLDSSNIKPMNLIGVDRGENIPAVIALTDPE |
| | GCPLSRFKDSLGNPTHILRIGESYKEKQRTIQAKKEVEQRRAGGYSRKYASK |
| | AKNLADDMVRNTARDLLYYAVTQDAMLIFENLSRGFGRQGKRTFMAERQYTR |
| | MEDWLTAKLAYEGLSKTYLSKTLAQYTSKTCSNCGFTITSADYDRVLEKLKK |
| | TATGWMTTINGKELKVEGQITYYNRYKRQNVVKDLSVELDRLSEESVNNDIS |
| | SWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID |
| | NO: 148) |
| 519: | QEIKRINKIRRRLVKDSNTKKAGKTGPMKTLLVRVMTPDLRERLENLRKKPE |
| \hat{ }Q692 in 491 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHIQLRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 149) |
| 520: | QEIKRINKIRRRLVKDSNTKKAGKTGPMKTLLVRVMTPDLRERLENLRKKPE |
| I705T in 491 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTTQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 150) |
| 522: | QEIKRINKIRRRLVKDSNTKKAGKTGPMKTLLVRVMTPDLRERLENLRKKPE |
| D683R in | NIPQPISNTSRANLNKLLTDYTEMKKAILHVYWEEFQKDPVGLMSRVAQPAS |
| 491 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKRSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 151) |
| 523: | QEIKRINKIRRRLVKDSNTKKAGKTYPMKTLLVRVMTPDLRERLENLRKKPE |
| G26Y in 491 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 152) |
| 524: | QEIKRINKIRRRLVKDSNTKKAGKTGPMKTLLVRVMTPDLRERLENLRKKPE |
| T817H in | NIPQPISNTSRANLNKLLTDYTEMKKAILHVYWEEFQKDPVGLMSRVAQPAS |
| 491 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTIHSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 153) |
| 525: | QEIKRINKIRRRLVKDSNTKKAGKTGPMKTLLVRVMTPDLRERLENLRKKPE |
| V746A in | NIPQPISNTSRANLNKLLTDYTEMKKAILHVYWEEFQKDPVGLMSRVAQPAS |
| 491 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAATQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 154) |
| 526: | QEIKRINKIRRRLVKDSNTKKAGKTGPMKTLLVRVMTPDLRERLENLRKKPE |
| K708A in | NIPQPISNTSRANLNKLLTDYTEMKKAILHVYWEEFQKDPVGLMSRVAQPAS |
| 491 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAAKEVEQRRAGGYSRKYASKAK |
| | NLADDMVRNTARDLLYYAVTQDAMLIFENLSRGFGRQGKRTFMAERQYTRME |
| | DWLTAKLAYEGLSKTYLSKTLAQYTSKTCSNCGFTITSADYDRVLEKLKKTA |
| | TGWMTTINGKELKVEGQITYYNRYKRQNVVKDLSVELDRLSEESVNNDISSW |
| | TKGRSGEALSLLKKRFSHRPVQEKFVCLNCGFETHADEQAALNIARSWLFLR |
| | SQEYKKYQTNKTTGNTDKRAFVETWQSFYRKKLKEVWKPAV (SEQ ID |
| | NO: 155) |
| 527: | QEIKRINKIRRRLVKDSNTKKAGKTRGPMKTLLVRVMTPDLRERLENLRKKP |
| \hat{ }R26 in 491 | ENIPQPISNTSRANLNKLLTDYTEMKKAILHVYWEEFQKDPVGLMSRVAQPA |
| | SKKIDQNKLKPEMDEKGNLTTAGFACSQCGQPLFVYKLEQVSEKGKAYTNYF |
| | GRCNVAEHEKLILLAQLKPEKDSDEAVTYSLGKFGQRALDFYSIHVTKESTH |
| | PVKPLAQIAGNRYASGPVGKALSDACMGTIASFLSKYQDIIIEHQKVVKGNQ |
| | KRLESLRELAGKENLEYPSVTLPPQPHTKEGVDAYNEVIARVRMWVNLNLWQ |
| | KLKLSRDDAKPLLRLKGFPSFPLVERQANEVDWWDMVCNVKKLINEKKEDGK |
| | VFWQNLAGYKRQEALRPYLSSEEDRKKGKKFARYQLGDLLLHLEKKHGEDWG |
| | KVYDEAWERIDKKVEGLSKHIKLEEERRSEDAQSKAALTDWLRAKASFVIEG |
| | LKEADKDEFCRCELKLQKWYGDLRGKPFAIEAENSILDISGFSKQYNCAFIW |
| | QKDGVKKLNLYLIINYFKGGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGVDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 156) |
| 528: | QEIKRINKIRRRLVKDSNTKKAGKTGPMKTLLVRVMTPDLRERLENLRKKPE |
| G223Y in | NIPQPISNTSRANLNKLLTDYTEMKKAILHVYWEEFQKDPVGLMSRVAQPAS |
| 515 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASYPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 157) |
| 529: | QEIKRINKIRRRLVKDSNTKKAGKTGPMKTLLVRVMTPDLRERLENLRKKPE |
| G223N in | NIPQPISNTSRANLNKLLTDYTEMKKAILHVYWEEFQKDPVGLMSRVAQPAS |
| 515 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASNPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGGKLRFKKIKPEAFEANRFYTVINKKSGEIVPMEV |
| | NFNFDDPNLIILPLAFGKRQGREFIWNDLLSLETGSLKLANGRVIEKTLYNR |
| | RTRQDEPALFVALTFERREVLDSSNIKPMNLIGVDRGENIPAVIALTDPEGC |
| | PLSRFKDSLGNPTHILRIGESYKEKQRTIQAKKEVEQRRAGGYSRKYASKAK |
| | NLADDMVRNTARDLLYYAVTQDAMLIFENLSRGFGRQGKRTFMAERQYTRME |
| | DWLTAKLAYEGLPSKTYLSKTLAQYTSKTCSNCGFTITSADYDRVLEKLKKT |
| | ATGWMTTINGKELKVEGQITYYNRYKRQNVVKDLSVELDRLSEESVNNDISS |
| | WTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAV (SEQ ID |
| | NO: 158) |
| 530: | QEIKRINKIRRRLVKDSNTKKAGKTGPMKTLLVRVMTPDLRERLENLRKKPE |
| \hat{ }W539 in | NIPQPISNTSRANLNKLLTDYTEMKKAILHVYWEEFQKDPVGLMSRVAQPAS |
| 515 | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGWGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGVDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLPSKTYLSKTLAQYTSKTCSNCGFTITSADYDRVLEKLKK |
| | TATGWMTTINGKELKVEGQITYYNRYKRQNVVKDLSVELDRLSEESVNNDIS |
| | SWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID |
| | NO: 159) |
| 531: | QEIKRINKIRRRLVKDSNTKKAGKTGPMKTLLVRVMTPDLRERLENLRKKPE |
| \hat{ }Y539 in515 | NIPQPISNTSRANLNKLLTDYTEMKKAILHVYWEEFQKDPVGLMSRVAQPAS |
| | KKIDQNKLKPEMDEKGNLTTAGFACSQCGQPLFVYKLEQVSEKGKAYTNYFG |
| | RCNVAEHEKLILLAQLKPEKDSDEAVTYSLGKFGQRALDFYSIHVTKESTHP |
| | VKPLAQIAGNRYASGPVGKALSDACMGTIASFLSKYQDIIIEHQKVVKGNQK |
| | RLESLRELAGKENLEYPSVTLPPQPHTKEGVDAYNEVIARVRMWVNLNLWQK |
| | LKLSRDDAKPLLRLKGFPSFPLVERQANEVDWWDMVCNVKKLINEKKEDGKV |
| | FWQNLAGYKRQEALRPYLSSEEDRKKGKKFARYQLGDLLLHLEKKHGEDWGK |
| | VYDEAWERIDKKVEGLSKHIKLEEERRSEDAQSKAALTDWLRAKASFVIEGL |
| | KEADKDEFCRCELKLQKWYGDLRGKPFAIEAENSILDISGFSKQYNCAFIWQ |
| | KDGVKKLNLYLIINYFKGYGKLRFKKIKPEAFEANRFYTVINKKSGEIVPME |
| | VNFNFDDPNLIILPLAFGKRQGREFIWNDLLSLETGSLKLANGRVIEKTLYN |
| | RRTRQDEPALFVALTFERREVLDSSNIKPMNLIGVDRGENIPAVIALTDPEG |
| | CPLSRFKDSLGNPTHILRIGESYKEKQRTIQAKKEVEQRRAGGYSRKYASKA |
| | KNLADDMVRNTARDLLYYAVTQDAMLIFENLSRGFGRQGKRTFMAERQYTRM |
| | EDWLTAKLAYEGLPSKTYLSKTLAQYTSKTCSNCGFTITSADYDRVLEKLKK |
| | TATGWMTTINGKELKVEGQITYYNRYKRQNVVKDLSVELDRLSEESVNNDIS |
| | SWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAV (SEQ ID |
| | NO: 160) |

In some embodiments, the CasX variant protein comprises a sequence selected from the group consisting of SEQ ID NOS: 49-160, 439, 441, 443, 445, 447-460, 472, 474, 476, 478, 480, 482, 484, 486, 488, and 490, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto. In some embodiments, the CasX variant protein comprises a sequence selected from the group consisting of SEQ ID NOS: 49-160, 439, 441, 443, 445, 447-460, 472, 474, 476, 478, 480, 482, 484, 486, 488, and 490. In some embodiments, the CasX variant protein comprises a sequence selected from the group consisting of SEQ ID NOs: 49-160, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto. In some embodiments, the CasX variant protein comprises a sequence selected from the group consisting of SEQ ID NOs: 49-160.

In some embodiments, the CasX variant protein has one or more improved characteristics of the CasX protein when compared to a reference CasX protein, for example a reference protein of SEQ ID NO: 1, SEQ ID NO: 2 or SEQ ID NO: 3. In some embodiments, the at least one improved characteristic of the CasX variant is at least about 1.1 to about 100,000-fold improved relative to the reference protein. In some embodiments, the at least one improved characteristic of the CasX variant is at least about 1.1 to about 10,000-fold improved, at least about 1.1 to about 1,000-fold improved, at least about 1.1 to about 500-fold improved, at least about 1.1 to about 400-fold improved, at least about 1.1 to about 300-fold improved, at least about 1.1 to about 200-fold improved, at least about 1.1 to about 100-fold improved, at least about 1.1 to about 50-fold improved, at least about 1.1 to about 40-fold improved, at least about 1.1 to about 30-fold improved, at least about 1.1 to about 20-fold improved, at least about 1.1 to about 10-fold improved, at least about 1.1 to about 9-fold improved, at least about 1.1 to about 8-fold improved, at least about 1.1 to about 7-fold improved, at least about 1.1 to about 6-fold improved, at least about 1.1 to about 5-fold improved, at least about 1.1 to about 4-fold improved, at least about 1.1 to about 3-fold improved, at least about 1.1 to about 2-fold improved, at least about 1.1 to about 1.5-fold improved, at least about 1.5 to about 3-fold improved, at least about 1.5 to about 4-fold improved, at least about 1.5 to about 5-fold improved, at least about 1.5 to about 10-fold improved, at least about 5 to about 10-fold improved, at least about 10 to about 20-fold improved, at least 10 to about 30-fold improved, at least 10 to about 50-fold improved or at least 10 to about 100-fold improved than the reference CasX protein. In some embodiments, the at least one improved characteristic of the CasX variant is at least about 10 to about 1000-fold improved relative to the reference CasX protein.

In some embodiments, the at least one improved characteristic of the CasX variant protein is at least about 5, at least about 10, at least about 20, at least about 30, at least about 40, at least about 50, at least about 60, at least about 70, at least about 80, at least about 90, at least about 100, at least about 250, at least about 500, at least about 1000, at least about 5,000 or at least about 10,000-fold improved relative to a reference CasX protein. In some embodiments, a CasX variant protein is at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.5, at least about 4, at least about 4.5, at least about 5, at least about 5.5, at least about 6, at least about 6.5, at least about 7.0, at least about 7.5, at least about 8, at least about 8.5, at least about 9, at least about 9.5, at least about 10, at least about 11, at least about 12, at least about 13, at least about 14, at least about 15, at least about 20, at least about 30, at least about 40, at least about 50, at least about 60, at least about 70, at least about 80, at least about 90 at least about 100, at least about 500, at least about 1,000 or at least about 10,000-fold improved relative to a reference CasX protein. Exemplary characteristics that can be improved in CasX variant proteins relative to the same characteristics in reference CasX proteins include, but are not limited to, improved folding of the variant, improved binding affinity to the gNA, improved binding affinity to the target DNA, altered binding affinity to one or more PAM sequences, improved unwinding of the target DNA, increased activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved target nucleic acid sequence cleavage rate, improved protein stability, improved protein:gNA complex stability, improved protein solubility, improved ribonuclear protein complex (RNP) formation, higher percentage of cleavage-competent RNP, improved protein:gNA complex (RNP) solubility, improved protein yield, improved protein expression, and improved fusion characteristics. In some embodiments, the variant comprises at least one improved characteristic. In other embodiments, the variant comprises at least two improved characteristics. In further embodiments, the variant comprises at least three improved characteristics. In some embodiments, the variant comprises at least four improved characteristics. In still further embodiments, the variant comprises at least five, at least six, at least seven, at least eight, at least nine, at least ten, at least eleven, at least twelve, at least thirteen, or more improved characteristics. These improved characteristics are described in more detail below.

j. Protein Stability

In some embodiments, the disclosure provides a CasX variant protein with improved stability relative to a reference CasX protein. In some embodiments, improved stability of the CasX variant protein results in expression of a higher steady state of protein, which improves editing efficiency. In some embodiments, improved stability of the CasX variant protein results in a larger fraction of CasX protein that remains folded in a functional conformation and improves editing efficiency or improves purifiability for manufacturing purposes. As used herein, a “functional conformation” refers to a CasX protein that is in a conformation where the protein is capable of binding a gNA and target DNA. In embodiments wherein the CasX variant does not carry one or more mutations rendering it catalytically dead, the CasX variant is capable of cleaving, nicking, or otherwise modifying the target DNA. For example, a functional CasX variant can, in some embodiments, be used for gene-editing, and a functional conformation refers to an “editing-competent” conformation. In some exemplary embodiments, including those embodiments where the CasX variant protein results in a larger fraction of CasX protein that remains folded in a functional conformation, a lower concentration of CasX variant is needed for applications such as gene editing compared to a reference CasX protein. Thus, in some embodiments, the CasX variant with improved stability has improved efficiency compared to a reference CasX in one or more gene editing contexts.

In some embodiments, the disclosure provides a CasX variant protein having improved thermostability relative to a reference CasX protein. In some embodiments, the CasX variant protein has improved thermostability of the CasX variant protein at a particular temperature range. Without wishing to be bound by any theory, some reference CasX proteins natively function in organisms with niches in groundwater and sediment; thus, some reference CasX proteins may have evolved to exhibit optimal function at lower or higher temperatures that may be desirable for certain applications. For example, one application of CasX variant proteins is gene editing of mammalian cells, which is typically carried out at about 37° C. In some embodiments, a CasX variant protein as described herein has improved thermostability compared to a reference CasX protein at a temperature of at least 16° C., at least 18° C., at least 20° C., at least 22° C., at least 24° C., at least 26° C., at least 28° C., at least 30° C., at least 32° C., at least 34° C., at least 35° C., at least 36° C., at least 37° C., at least 38° C., at least 39° C., at least 40° C., at least 41° C., at least 42° C., at least 44° C., at least 46° C., at least 48° C., at least 50° C., at least 52° C., or greater. In some embodiments, a CasX variant protein has improved thermostability and functionality compared to a reference CasX protein that results in improved gene editing functionality, such as mammalian gene editing applications, which may include human gene editing applications.

In some embodiments, the disclosure provides a CasX variant protein having improved stability of the CasX variant protein:gNA complex relative to the reference CasX protein:gNA complex such that the RNP remains in a functional form. Stability improvements can include increased thermostability, resistance to proteolytic degradation, enhanced pharmacokinetic properties, stability across a range of pH conditions, salt conditions, and tonicity. Improved stability of the complex may, in some embodiments, lead to improved editing efficiency.

In some embodiments, the disclosure provides a CasX variant protein having improved thermostability of the CasX variant protein:gNA complex relative to the reference CasX protein:gNA complex. In some embodiments, a CasX variant protein has improved thermostability relative to a reference CasX protein. In some embodiments, the CasX variant protein:gNA complex has improved thermostability relative to a complex comprising a reference CasX protein at temperatures of at least 16° C., at least 18° C., at least 20° C., at least 22° C., at least 24° C., at least 26° C., at least 28° C., at least 30° C., at least 32° C., at least 34° C., at least 35° C., at least 36° C., at least 37° C., at least 38° C., at least 39° C., at least 40° C., at least 41° C., at least 42° C., at least 44° C., at least 46° C., at least 48° C., at least 50° C., at least 52° C., or greater. In some embodiments, a CasX variant protein has improved thermostability of the CasX variant protein:gNA complex compared to a reference CasX protein:gNA complex, which results in improved function for gene editing applications, such as mammalian gene editing applications, which may include human gene editing applications.

In some embodiments, the improved stability and/or thermostability of the CasX variant protein comprises faster folding kinetics of the CasX variant protein relative to a reference CasX protein, slower unfolding kinetics of the CasX variant protein relative to a reference CasX protein, a larger free energy release upon folding of the CasX variant protein relative to a reference CasX protein, a higher temperature at which 50% of the CasX variant protein is unfolded (Tm) relative to a reference CasX protein, or any combination thereof. These characteristics may be improved by a wide range of values; for example, at least 1.1, at least 1.5, at least 10, at least 50, at least 100, at least 500, at least 1,000, at least 5,000, or at least a 10,000-fold improved, as compared to a reference CasX protein. In some embodiments, improved thermostability of the CasX variant protein comprises a higher Tm of the CasX variant protein relative to a reference CasX protein. In some embodiments, the Tm of the CasX variant protein is between about 20° C. to about 30° C., between about 30° C. to about 40° C., between about 40° C. to about 50° C., between about 50° C. to about 60° C., between about 60° C. to about 70° C., between about 70° C. to about 80° C., between about 80° C. to about 90° C. or between about 90° C. to about 100° C. Thermal stability is determined by measuring the “melting temperature” (Tm), which is defined as the temperature at which half of the molecules are denatured. Methods of measuring characteristics of protein stability such as Tm and the free energy of unfolding are known to persons of ordinary skill in the art, and can be measured using standard biochemical techniques in vitro. For example, Tm may be measured using Differential Scanning calorimetry, a thermo-analytical technique in which the difference in the amount of heat required to increase the temperature of a sample and a reference is measured as a function of temperature (Chen et al (2003) Pharm Res 20:1952-60; Ghirlando et al (1999) Immunol Lett 68:47-52). Alternatively, or in addition, CasX variant protein Tm may be measured using commercially available methods such as the ThermoFisher Protein Thermal Shift system. Alternatively, or in addition, circular dichroism may be used to measure the kinetics of folding and unfolding, as well as the Tm (Murray et al. (2002) J. Chromatogr Sci 40:343-9). Circular dichroism (CD) relies on the unequal absorption of left-handed and right-handed circularly polarized light by asymmetric molecules such as proteins. Certain structures of proteins, for example alpha-helices and beta-sheets, have characteristic CD spectra. Accordingly, in some embodiments, CD may be used to determine the secondary structure of a CasX variant protein.

In some embodiments, improved stability and/or thermostability of the CasX variant protein comprises improved folding kinetics of the CasX variant protein relative to a reference CasX protein. In some embodiments, folding kinetics of the CasX variant protein are improved relative to a reference CasX protein by at least about 5, at least about 10, at least about 50, at least about 100, at least about 500, at least about 1,000, at least about 2,000, at least about 3,000, at least about 4,000, at least about 5,000, or at least about a 10,000-fold improvement. In some embodiments, folding kinetics of the CasX variant protein are improved relative to a reference CasX protein by at least about 1 kJ/mol, at least about 5 kJ/mol, at least about 10 kJ/mol, at least about 20 kJ/mol, at least about 30 kJ/mol, at least about 40 kJ/mol, at least about 50 kJ/mol, at least about 60 kJ/mol, at least about 70 kJ/mol, at least about 80 kJ/mol, at least about 90 kJ/mol, at least about 100 kJ/mol, at least about 150 kJ/mol, at least about 200 kJ/mol, at least about 250 kJ/mol, at least about 300 kJ/mol, at least about 350 kJ/mol, at least about 400 kJ/mol, at least about 450 kJ/mol, or at least about 500 kJ/mol.

Exemplary amino acid changes that can increase the stability of a CasX variant protein relative to a reference CasX protein may include, but are not limited to, amino acid changes that increase the number of hydrogen bonds within the CasX variant protein, increase the number of disulfide bridges within the CasX variant protein, increase the number of salt bridges within the CasX variant protein, strengthen interactions between parts of the CasX variant protein, increase the buried hydrophobic surface area of the CasX variant protein, or any combinations thereof.

k. Protein Yield

In some embodiments, the disclosure provides a CasX variant protein having improved yield during expression and purification relative to a reference CasX protein. In some embodiments, the yield of CasX variant proteins purified from bacterial or eukaryotic host cells is improved relative to a reference CasX protein. In some embodiments, the bacterial host cells are Escherichia coli cells. In some embodiments, the eukaryotic cells are yeast, plant (e.g. tobacco), insect (e.g. Spodoptera frugiperda sf9 cells), mouse, rat, hamster, guinea pig, monkey, or human cells. In some embodiments, the eukaryotic host cells are mammalian cells, including, but not limited to HEK293 cells, BHK cells, NS0 cells, SP2/0 cells, YO myeloma cells, P3X63 mouse myeloma cells, PER cells, PER.C6 cells, hybridoma cells, NIH3T3 cells, COS, HeLa, or CHO cells.

In some embodiments, improved yield of the CasX variant protein is achieved through codon optimization. Cells use 64 different codons, 61 of which encode the 20 standard amino acids, while another 3 function as stop codons. In some cases, a single amino acid is encoded by more than one codon. Different organisms exhibit bias towards use of different codons for the same naturally occurring amino acid. Therefore, the choice of codons in a protein coding sequence, and matching codon choice to the organism in which the protein will be expressed, can, in some cases, significantly affect protein translation and therefore protein expression levels. In some embodiments, the CasX variant protein is encoded by a nucleic acid that has been codon optimized. In some embodiments, the nucleic acid encoding the CasX variant protein has been codon optimized for expression in a bacterial cell, a yeast cell, an insect cell, a plant cell, or a mammalian cell. In some embodiments, the mammal cell is a mouse, a rat, a hamster, a guinea pig, a monkey, or a human. In some embodiments, the CasX variant protein is encoded by a nucleic acid that has been codon optimized for expression in a human cell. In some embodiments, the CasX variant protein is encoded by a nucleic acid from which nucleotide sequences that reduce translation rates in prokaryotes and eukaryotes have been removed. For example, runs of greater than three thymine residues in a row can reduce translation rates in certain organisms or internal polyadenylation signals can reduce translation.

In some embodiments, improvements in solubility and stability, as described herein, result in improved yield of the CasX variant protein relative to a reference CasX protein.

Improved protein yield during expression and purification can be evaluated by methods known in the art. For example, the amount of CasX variant protein can be determined by running the protein on an SDS-page gel, and comparing the CasX variant protein to a either a control whose amount or concentration is known in advance to determine an absolute level of protein. Alternatively, or in addition, a purified CasX variant protein can be run on an SDS-page gel next to a reference CasX protein undergoing the same purification process to determine relative improvements in CasX variant protein yield. Alternatively, or in addition, levels of protein can be measured using immunohistochemical methods such as Western blot or ELISA with an antibody to CasX, or by HPLC. For proteins in solution, concentration can be determined by measuring of the protein's intrinsic UV absorbance, or by methods which use protein-dependent color changes such as the Lowry assay, the Smith copper/bicinchoninic assay or the Bradford dye assay. Such methods can be used to calculate the total protein (such as, for example, total soluble protein) yield obtained by expression under certain conditions. This can be compared, for example, to the protein yield of a reference CasX protein under similar expression conditions.

l. Protein Solubility

In some embodiments, a CasX variant protein has improved solubility relative to a reference CasX protein. In some embodiments, a CasX variant protein has improved solubility of the CasX:gNA ribonucleoprotein complex variant relative to a ribonucleoprotein complex comprising a reference CasX protein.

In some embodiments, an improvement in protein solubility leads to higher yield of protein from protein purification techniques such as purification from E. coli. Improved solubility of CasX variant proteins may, in some embodiments, enable more efficient activity in cells, as a more soluble protein may be less likely to aggregate in cells. Protein aggregates can in certain embodiments be toxic or burdensome on cells, and, without wishing to be bound by any theory, increased solubility of a CasX variant protein may ameliorate this result of protein aggregation. Further, improved solubility of CasX variant proteins may allow for enhanced formulations permitting the delivery of a higher effective dose of functional protein, for example in a desired gene editing application. In some embodiments, improved solubility of a CasX variant protein relative to a reference CasX protein results in improved yield of the CasX variant protein during purification of at least about 5, at least about 10, at least about 20, at least about 30, at least about 40, at least about 50, at least about 60, at least about 70, at least about 80, at least about 90, at least about 100, at least about 250, at least about 500, or at least about 1000-fold greater yield. In some embodiments, improved solubility of a CasX variant protein relative to a reference CasX protein improves activity of the CasX variant protein in cells by at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.5, at least about 4, at least about 4.5, at least about 5, at least about 5.5, at least about 6, at least about 6.5, at least about 7.0, at least about 7.5, at least about 8, at least about 8.5, at least about 9, at least about 9.5, at least about 10, at least about 11, at least about 12, at least about 13, at least about 14, or at least about 15-fold greater activity.

Methods of measuring CasX protein solubility, and improvements thereof in CasX variant proteins, will be readily apparent to the person of ordinary skill in the art. For example, CasX variant protein solubility can in some embodiments be measured by taking densitometry readings on a gel of the soluble fraction of lysed E. coli. Alternatively, or addition, improvements in CasX variant protein solubility can be measured by measuring the maintenance of soluble protein product through the course of a full protein purification. For example, soluble protein product can be measured at one or more steps of gel affinity purification, tag cleavage, cation exchange purification, running the protein on a size exclusion chromatography (SEC) column. In some embodiments, the densitometry of every band of protein on a gel is read after each step in the purification process. CasX variant proteins with improved solubility may, in some embodiments, maintain a higher concentration at one or more steps in the protein purification process when compared to the reference CasX protein, while an insoluble protein variant may be lost at one or more steps due to buffer exchanges, filtration steps, interactions with a purification column, and the like.

In some embodiments, improving the solubility of CasX variant proteins results in a higher yield in terms of mg/L of protein during protein purification when compared to a reference CasX protein.

In some embodiments, improving the solubility of CasX variant proteins enables a greater amount of editing events compared to a less soluble protein when assessed in editing assays such as the EGFP disruption assays described herein.

m. Protein Affinity for the gNA

In some embodiments, a CasX variant protein has improved affinity for the gNA relative to a reference CasX protein, leading to the formation of the ribonucleoprotein complex. Increased affinity of the CasX variant protein for the gNA may, for example, result in a lower Kd for the generation of a RNP complex, which can, in some cases, result in a more stable ribonucleoprotein complex formation. In some embodiments, the Kd of a CasX variant protein for a gNA is increased relative to a reference CasX protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the CasX variant has about 1.1 to about 10-fold increased binding affinity to the gNA compared to the reference CasX protein of SEQ ID NO: 2.

In some embodiments, increased affinity of the CasX variant protein for the gNA results in increased stability of the ribonucleoprotein complex when delivered to mammalian cells, including in vivo delivery to a subject. This increased stability can affect the function and utility of the complex in the cells of a subject, as well as result in improved pharmacokinetic properties in blood, when delivered to a subject. In some embodiments, increased affinity of the CasX variant protein, and the resulting increased stability of the ribonucleoprotein complex, allows for a lower dose of the CasX variant protein to be delivered to the subject or cells while still having the desired activity; for example in vivo or in vitro gene editing. The increased ability to form RNP and keep them in stable form can be assessed using assays such as the in vitro cleavage assays described in the Examples herein. In some embodiments, RNP comprising the CasX variants of the disclosure are able to achieve a K_{cleave} rate when complexed as an RNP that is at last 2-fold, at least 5-fold, or at least 10-fold higher compared to RNP comprising a reference CasX of SEQ ID NOS: 1-3.

In some embodiments, a higher affinity (tighter binding) of a CasX variant protein to a gNA allows for a greater amount of editing events when both the CasX variant protein and the gNA remain in an RNP complex. Increased editing events can be assessed using editing assays such as the EGFP disruption assay described herein.

Without wishing to be bound by theory, in some embodiments amino acid changes in the Helical I domain can increase the binding affinity of the CasX variant protein with the gNA targeting sequence, while changes in the Helical II domain can increase the binding affinity of the CasX variant protein with the gNA scaffold stem loop, and changes in the oligonucleotide binding domain (OBD) increase the binding affinity of the CasX variant protein with the gRNA triplex.

Methods of measuring CasX protein binding affinity for a CasX gNA include in vitro methods using purified CasX protein and gNA. The binding affinity for reference CasX and variant proteins can be measured by fluorescence polarization if the gNA or CasX protein is tagged with a fluorophore. Alternatively, or in addition, binding affinity can be measured by biolayer interferometry, electrophoretic mobility shift assays (EMSAs), or filter binding. Additional standard techniques to quantify absolute affinities of RNA binding proteins such as the reference CasX and variant proteins of the disclosure for specific gNAs such as reference gNAs and variants thereof include, but are not limited to, isothermal calorimetry (ITC), and surface plasmon resonance (SPR), as well as the methods of the Examples.

n. Affinity for Target Nucleic Acid

In some embodiments, a CasX variant protein has improved binding affinity for a target nucleic acid relative to the affinity of a reference CasX protein for a target nucleic acid. CasX variants with higher affinity for their target nucleic acid may, in some embodiments, cleave the target nucleic acid sequence more rapidly than a reference CasX protein that does not have increased affinity for the target nucleic acid.

In some embodiments, the improved affinity for the target nucleic acid comprises improved affinity for the target sequence or protospacer sequence of the target nucleic acid, improved affinity for the PAM sequence, an improved ability to search DNA for the target sequence, or any combinations thereof. Without wishing to be bound by theory, it is thought that CRISPR/Cas system proteins such as CasX may find their target sequences by one-dimension diffusion along a DNA molecule. The process is thought to include (1) binding of the ribonucleoprotein to the DNA molecule followed by (2) stalling at the target sequence, either of which may be, in some embodiments, affected by improved affinity of CasX proteins for a target nucleic acid sequence, thereby improving function of the CasX variant protein compared to a reference CasX protein.

In some embodiments, a CasX variant protein with improved target nucleic acid affinity has increased overall affinity for DNA. In some embodiments, a CasX variant protein with improved target nucleic acid affinity has increased affinity for or the ability to utilize specific PAM sequences other than the canonical TTC PAM recognized by the reference CasX protein of SEQ ID NO: 2, including PAM sequences selected from the group consisting of TTC, ATC, GTC, and CTC, thereby increasing the amount of target DNA that can be edited compared to wild-type CasX nucleases. Without wishing to be bound by theory, it is possible that these protein variants may interact more strongly with DNA overall and may have an increased ability to access and edit sequences within the target DNA due to the ability to utilize additional PAM sequences beyond those of wild-type reference CasX, thereby allowing for a more efficient search process of the CasX protein for the target sequence. A higher overall affinity for DNA also, in some embodiments, can increase the frequency at which a CasX protein can effectively start and finish a binding and unwinding step, thereby facilitating target strand invasion and R-loop formation, and ultimately the cleavage of a target nucleic acid sequence.

Without wishing to be bound by theory, it is possible that amino acid changes in the NTSBD that increase the efficiency of unwinding, or capture, of a non-target DNA strand in the unwound state, can increase the affinity of CasX variant proteins for target DNA. Alternatively, or in addition, amino acid changes in the NTSBD that increase the ability of the NTSBD to stabilize DNA during unwinding can increase the affinity of CasX variant proteins for target DNA. Alternatively, or in addition, amino acid changes in the OBD may increase the affinity of CasX variant protein binding to the protospacer adjacent motif (PAM), thereby increasing affinity of the CasX variant protein for the target nucleic acid sequence. Alternatively, or in addition, amino acid changes in the Helical I and/or II, RuvC and TSL domains that increase the affinity of the CasX variant protein for the target nucleic acid strand can increase the affinity of the CasX variant protein for the target nucleic acid sequence.

In some embodiments, the CasX variant protein has increased binding affinity to the target nucleic acid sequence compared to the reference protein of SEQ ID NO: 1, SEQ ID NO: 2, or SEQ ID NO: 3. In some embodiments, affinity of a CasX variant protein of the disclosure for a target nucleic acid molecule is increased relative to a reference CasX protein of SEQ ID NO: 1, SEQ ID NO: 2, or SEQ ID NO: 3 by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100.

In some embodiments, a CasX variant protein has improved binding affinity for the non-target strand of the target nucleic acid. As used herein, the term “non-target strand” refers to the strand of the DNA target nucleic acid sequence that does not form Watson and Crick base pairs with the targeting sequence in the gNA, and is complementary to the target DNA strand. In some embodiments, the CasX variant protein has about 1.1 to about 100-fold increased binding affinity to the non-target stand of the target nucleic acid compared to the reference protein of SEQ ID NO: 1, SEQ ID NO: 2, or SEQ ID NO: 3.

Methods of measuring CasX protein (such as reference or variant) affinity for a target nucleic acid molecule may include electrophoretic mobility shift assays (EMSAs), filter binding, isothermal calorimetry (ITC), and surface plasmon resonance (SPR), fluorescence polarization and biolayer interferometry (BLI). Further methods of measuring CasX protein affinity for a target include in vitro biochemical assays that measure DNA cleavage events over time.

o. Improved Specificity for a Target Site

In some embodiments, a CasX variant protein has improved specificity for a target nucleic acid sequence relative to a reference CasX protein of SEQ ID NOS: 1-3. As used herein, “specificity,” interchangeably referred to as “target specificity,” refers to the degree to which a CRISPR/Cas system ribonucleoprotein complex cleaves off-target sequences that are similar, but not identical to the target nucleic acid sequence; e.g., a CasX variant RNP with a higher degree of specificity would exhibit reduced off-target cleavage of sequences relative to a reference CasX protein. The specificity, and the reduction of potentially deleterious off-target effects, of CRISPR/Cas system proteins can be vitally important in order to achieve an acceptable therapeutic index for use in mammalian subjects.

In some embodiments, a CasX variant protein has improved specificity for a target site within the target sequence that is complementary to the targeting sequence of the gNA relative to a reference CasX protein of SEQ ID NOS: 1-3. Without wishing to be bound by theory, it is possible that amino acid changes in the helical I and II domains that increase the specificity of the CasX variant protein for the target nucleic acid strand can increase the specificity of the CasX variant protein for the target nucleic acid overall. In some embodiments, amino acid changes that increase specificity of CasX variant proteins for target nucleic acid may also result in decreased affinity of CasX variant proteins for DNA.

Methods of testing CasX protein (such as variant or reference) target specificity may include guide and Circularization for In vitro Reporting of Cleavage Effects by Sequencing (CIRCLE-seq), or similar methods. In brief, in CIRCLE-seq techniques, genomic DNA is sheared and circularized by ligation of stem-loop adapters, which are nicked in the stem-loop regions to expose 4 nucleotide palindromic overhangs. This is followed by intramolecular ligation and degradation of remaining linear DNA. Circular DNA molecules containing a CasX cleavage site are subsequently linearized with CasX, and adapter adapters are ligated to the exposed ends followed by high-throughput sequencing to generate paired end reads that contain information about the off-target site. Additional assays that can be used to detect off-target events, and therefore CasX protein specificity include assays used to detect and quantify indels (insertions and deletions) formed at those selected off-target sites such as mismatch-detection nuclease assays and next generation sequencing (NGS). Exemplary mismatch-detection assays include nuclease assays, in which genomic DNA from cells treated with CasX and sgNA is PCR amplified, denatured and rehybridized to form hetero-duplex DNA, containing one wild type strand and one strand with an indel. Mismatches are recognized and cleaved by mismatch detection nucleases, such as Surveyor nuclease or T7 endonuclease I.

p. Protospacer and PAM Sequences

Herein, the protospacer is defined as the DNA sequence complementary to the targeting sequence of the guide RNA and the DNA complementary to that sequence, referred to as the target strand and non-target strand, respectively. As used herein, the PAM is a nucleotide sequence located 5′ proximal to the protospacer that, in conjunction with the targeting sequence of the gNA, helps the orientation and positioning of the CasX for the potential cleavage of the protospacer strand(s).

PAM sequences may be degenerate, and specific RNP constructs may have different preferred and tolerated PAM sequences that support different efficiencies of cleavage. Following convention, unless stated otherwise, the disclosure refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with a single nucleotide separating the PAM from the first nucleotide of the protospacer. Thus, in the case of reference CasX, a TTC PAM should be understood to mean a sequence following the formula 5′- . . . NNTTCN(protospacer) . . . 3′ (SEQ ID NO: 218) where ‘N’ is any DNA nucleotide and ‘(protospacer)’ is a DNA sequence having identity with the targeting sequence of the guide RNA. In the case of a CasX variant with expanded PAM recognition, a TTC, CTC, GTC, or ATC PAM should be understood to mean a sequence following the formulae:

5′- . . . NNTTCN(protospacer) . . . 3′ (SEQ ID NO: 218);

5′- . . . NNCTCN(protospacer) . . . 3′ (SEQ ID NO: 219);

5′- . . . NNGTCN(protospacer) . . . 3′ (SEQ ID NO: 220); or

5′- . . . NNATCN(protospacer) . . . 3′ (SEQ ID NO: 221). Alternatively, a TC PAM should be understood to mean a sequence following the formula:

(SEQ ID NO: 222)
5′-. . . NNNTCN(protospacer)NNNNNN . . . 3′.

In some embodiments, a CasX variant has improved editing of a PAM sequence exhibits greater editing efficiency and/or binding of a target sequence in the target DNA when any one of the PAM sequences TTC, ATC, GTC, or CTC is located 1 nucleotide 5′ to the non-target strand of the protospacer having identity with the targeting sequence of the gNA in a cellular assay system compared to the editing efficiency and/or binding of an RNP comprising a reference CasX protein in a comparable assay system. In some embodiments, the PAM sequence is TTC. In some embodiments, the PAM sequence is ATC. In some embodiments, the PAM sequence is CTC. In some embodiments, the PAM sequence is GTC.

q. Unwinding of DNA

In some embodiments, a CasX variant protein has improved ability of unwinding DNA relative to a reference CasX protein. Poor dsDNA unwinding has been shown previously to impair or prevent the ability of CRISPR/Cas system proteins AnaCas9 or Cas14s to cleave DNA. Therefore, without wishing to be bound by any theory, it is likely that increased DNA cleavage activity by some CasX variant proteins of the disclosure is due, at least in part, to an increased ability to find and unwind the dsDNA at a target site. Methods of measuring the ability of CasX proteins (such as variant or reference) to unwind DNA include, but are not limited to, in vitro assays that observe increased on rates of dsDNA targets in fluorescence polarization or biolayer interferometry.

Without wishing to be bound by theory, it is thought that amino acid changes in the NTSB domain may produce CasX variant proteins with increased DNA unwinding characteristics. Alternatively, or in addition, amino acid changes in the OBD or the helical domain regions that interact with the PAM may also produce CasX variant proteins with increased DNA unwinding characteristics.

r. Catalytic Activity

The ribonucleoprotein complex of the CasX:gNA systems disclosed herein comprise a CasX variant protein that binds to a target nucleic acid sequence and cleaves the target nucleic acid sequence. In some embodiments, a CasX variant protein has improved catalytic activity relative to a reference CasX protein. Without wishing to be bound by theory, it is thought that in some cases cleavage of the target strand can be a limiting factor for Cas12-like molecules in creating a dsDNA break. In some embodiments, CasX variant proteins improve bending of the target strand of DNA and cleavage of this strand, resulting in an improvement in the overall efficiency of dsDNA cleavage by the CasX ribonucleoprotein complex.

In some embodiments, a CasX variant protein has increased nuclease activity compared to a reference CasX protein. Variants with increased nuclease activity can be generated, for example, through amino acid changes in the RuvC nuclease domain. In some embodiments, the CasX variant comprises a nuclease domain having nickase activity. In the foregoing, the CasX nickase of a CasX:gNA system generates a single-stranded break within 10-18 nucleotides 3′ of a PAM site in the non-target strand. In other embodiments, the CasX variant comprises a nuclease domain having double-stranded cleavage activity. In the foregoing, the CasX of the CasX:gNA system generates a double-stranded break within 18-26 nucleotides 5′ of a PAM site on the target strand and 10-18 nucleotides 3′ on the non-target strand. Nuclease activity can be assayed by a variety of methods, including those of the Examples. In some embodiments, a CasX variant has a Kcleave constant that is at least 2-fold, or at least 3-fold, or at least 4-fold, or at least 5-fold, or at least 6-fold, or at least 7-fold, or at least 8-fold, or at least 9-fold, or at least 10-fold greater compared to a reference CasX.

In some embodiments, a CasX variant protein has the improved characteristic of forming RNP with gNA that result in a higher percentage of cleavage-competent RNP compared to an RNP of a reference CasX protein of SEQ ID NO: 1, SEQ ID NO: 2, or SEQ ID NO: 3 and the gNA. By cleavage competent, it is meant that the RNP that is formed has the ability to cleave the target nucleic acid. In some embodiments, the RNP of the CasX variant and the gNA exhibit at least a 2-fold, or at least a 3-fold, or at least a 4-fold, or at least a 5-fold, or at least a 10-fold cleavage rate compared to an RNP of a reference CasX protein of SEQ ID NO: 1, SEQ ID NO: 2, or SEQ ID NO: 3 and the gNA.

In some embodiments, a CasX variant protein has increased target strand loading for double strand cleavage compared to a reference CasX. Variants with increased target strand loading activity can be generated, for example, through amino acid changes in the TLS domain.

Without wishing to be bound by theory, amino acid changes in the TSL domain may result in CasX variant proteins with improved catalytic activity. Alternatively, or in addition, amino acid changes around the binding channel for the RNA:DNA duplex may also improve catalytic activity of the CasX variant protein.

In some embodiments, a CasX variant protein has increased collateral cleavage activity compared to a reference CasX protein. As used herein, “collateral cleavage activity” refers to additional, non-targeted cleavage of nucleic acids following recognition and cleavage of a target nucleic acid sequence. In some embodiments, a CasX variant protein has decreased collateral cleavage activity compared to a reference CasX protein.

In some embodiments, for example those embodiments encompassing applications where cleavage of the target nucleic acid sequence is not a desired outcome, improving the catalytic activity of a CasX variant protein comprises altering, reducing, or abolishing the catalytic activity of the CasX variant protein. In some embodiments, a ribonucleoprotein complex comprising a dCasX variant protein binds to a target nucleic acid sequence and does not cleave the target nucleic acid.

In some embodiments, the CasX ribonucleoprotein complex comprising a CasX variant protein binds a target DNA but generates a single stranded nick in the target DNA. In some embodiments, particularly those embodiments wherein the CasX protein is a nickase, a CasX variant protein has decreased target strand loading for single strand nicking. Variants with decreased target strand loading may be generated, for example, through amino acid changes in the TSL domain.

Exemplary methods for characterizing the catalytic activity of CasX proteins may include, but are not limited to, in vitro cleavage assays, including those of the Examples, below. In some embodiments, electrophoresis of DNA products on agarose gels can interrogate the kinetics of strand cleavage.

s. Affinity for PCSK9 Target RNA

In some embodiments, a ribonucleoprotein complex comprising a reference CasX protein or variant thereof binds to a target PCSK9 DNA and cleaves the target nucleic acid sequence. In some embodiments, variants of a reference CasX protein increase the specificity of the CasX variant protein for a target PCSK9 RNA, and increase the activity of the CasX variant protein with respect to a target RNA when compared to the reference CasX protein. For example, CasX variant proteins can display increased binding affinity for target RNAs, or increased cleavage of target RNAs, when compared to reference CasX proteins. In some embodiments, a ribonucleoprotein complex comprising a CasX variant protein binds to a target RNA and/or cleaves the target RNA. In some embodiments, a CasX variant has at least about two-fold to about 10-fold increased binding affinity to the PCSK9 target RNA compared to the reference protein of SEQ ID NO: 1, SEQ ID NO: 2, or SEQ ID NO: 3.

t. Combinations of Mutations

The present disclosure provides Cas X variants that are a combination of mutations from separate CasX variant proteins. In some embodiments, any variant to any domain described herein can be combined with other variants described herein. In some embodiments, any variant within any domain described herein can be combined with other variants described herein, in the same domain. Combinations of different amino acid changes may in some embodiments produce new optimized variants whose function is further improved by the combination of amino acid changes. In some embodiments, the effect of combining amino acid changes on CasX protein function is linear. As used herein, a combination that is linear refers to a combination whose effect on function is equal to the sum of the effects of each individual amino acid change when assayed in isolation. In some embodiments, the effect of combining amino acid changes on CasX protein function is synergistic. As used herein, a combination of variants that is synergistic refers to a combination whose effect on function is greater than the sum of the effects of each individual amino acid change when assayed in isolation. In some embodiments, combining amino acid changes produces CasX variant proteins in which more than one function of the CasX protein has been improved relative to the reference CasX protein.

u. CasX Fusion Proteins

In some embodiments, the disclosure provides CasX proteins comprising a heterologous protein fused to the CasX. In other cases, the CasX is a CasX variant of any of the embodiments described herein.

In some embodiments, the CasX variant protein comprises any one of SEQ ID NOS: 49-160, 439, 441, 443, 445, 447-460, 472, 474, 476, 478, 480, 482, 484, 486, 488, or 490 fused to one or more proteins or domains thereof that has a different activity of interest, resulting in a fusion protein. For example, in some embodiments, the CasX variant protein is fused to a protein (or domain thereof) that inhibits transcription, modifies a target nucleic acid sequence, or modifies a polypeptide associated with a nucleic acid (e.g., histone modification).

In some embodiments, a heterologous polypeptide (or heterologous amino acid such as a cysteine residue or a non-natural amino acid) can be inserted at one or more positions within a CasX protein to generate a CasX fusion protein. In other embodiments, a cysteine residue can be inserted at one or more positions within a CasX protein followed by conjugation of a heterologous polypeptide described below. In some alternative embodiments, a heterologous polypeptide or heterologous amino acid can be added at the N- or C-terminus of the CasX variant protein. In other embodiments, a heterologous polypeptide or heterologous amino acid can be inserted internally within the sequence of the CasX protein.

In some embodiments, the CasX variant fusion protein retains RNA-guided sequence specific target nucleic acid binding and cleavage activity. In some cases, the CasX variant fusion protein has (retains) 50% or more of the activity (e.g., cleavage and/or binding activity) of the corresponding CasX variant protein that does not have the insertion of the heterologous protein. In some cases, the CasX variant fusion protein retains at least about 60%, or at least about 70% or more, at least about 80%, or at least about 90%, or at least about 92%, or at least about 95%, or at least about 98%, or at least about 100% of the activity (e.g., cleavage and/or binding activity) of the corresponding CasX protein that does not have the insertion of the heterologous protein.

In some cases, the CasX variant fusion protein retains (has) target nucleic acid binding activity relative to the activity of the CasX protein without the inserted heterologous amino acid or heterologous polypeptide. In some cases, the CasX variant fusion protein retains at least about 60%, or at least about 70% or more, at least about 80%, or at least about 90%, or at least about 92%, or at least about 95%, or at least about 98%, or at least about 100% of the binding activity of the corresponding CasX protein that does not have the insertion of the heterologous protein.

In some cases, the CasX variant fusion protein retains (has) target nucleic acid binding and/or cleavage activity relative to the activity of the parent CasX protein without the inserted heterologous amino acid or heterologous polypeptide. For example, in some cases, the CasX variant fusion protein has (retains) 50% or more of the binding and/or cleavage activity of the corresponding parent CasX protein (the CasX protein that does not have the insertion). For example, in some cases, the CasX variant fusion protein has (retains) 60% or more (70% or more, 80% or more, 90% or more, 92% or more, 95% or more, 98% or more, or 100%) of the binding and/or cleavage activity of the corresponding CasX parent protein (the CasX protein that does not have the insertion). Methods of measuring cleaving and/or binding activity of a CasX protein and/or a CasX fusion protein will be known to one of ordinary skill in the art and any convenient method can be used.

A variety of heterologous polypeptides are suitable for inclusion in a reference CasX or CasX variant fusion protein of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some embodiments, a CasX variant comprises any one of SEQ ID NOS: 49-160, 439, 441, 443, 445, 447-460, 472, 474, 476, 478, 480, 482, 484, 486, 488, or 490 and a polypeptide with methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity.

In some embodiments, a CasX variant comprises any one of SEQ ID NOS: 49-160, 439, 441, 443, 445, 447-460, 472, 474, 476, 478, 480, 482, 484, 486, 488, or 490 and a fusion partner having enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCNS, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET 1, DME, DML1, DML2, ROS1, and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the fusion partner to a CasX variant comprises of any one of SEQ ID NOS: 49-160, 439, 441, 443, 445, 447-460, 472, 474, 476, 478, 480, 482, 484, 486, 488, or 490 has enzymatic activity that modifies the target nucleic acid sequence (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme, e.g., an APOBEC protein such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, a CasX variant protein of the present disclosure is fused to a polypeptide selected from a domain for increasing transcription (e.g., a VP16 domain, a VP64 domain), a domain for decreasing transcription (e.g., a KRAB domain, e.g., from the Kox1 protein), a core catalytic domain of a histone acetyltransferase (e.g., histone acetyltransferase p300), a protein/domain that provides a detectable signal (e.g., a fluorescent protein such as GFP), a nuclease domain (e.g., a Fokl nuclease), or a base editor (e.g., cytidine deaminase such as APOBEC1).

In some embodiments, a CasX variant comprises any one of SEQ ID NOS: 49-160, 439, 441, 443, 445, 447-460, 472, 474, 476, 478, 480, 482, 484, 486, 488, or 490 and a fusion partner having enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB 1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCNS, PCAF, CBP, TAF1, TIP60/PLIP, MOZ/MYST3, MORF/MYST4, HB01/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

Additional examples of suitable fusion partners for a CasX variant are (i) a dihydrofolate reductase (DHFR) destabilization domain (e.g., to generate a chemically controllable subject RNA-guided polypeptide or a conditionally active RNA-guided polypeptide), and (ii) a chloroplast transit peptide.

In some embodiments, a CasX variant comprises any one of SEQ ID NOS: 49-160, 439, 441, 443, 445, 447-460, 472, 474, 476, 478, 480, 482, 484, 486, 488, or 490 and a chloroplast transit peptide including, but are not limited to:

(SEQ ID NO: 31)
MASMISSSAVTTVSRASRGQSAAMAPFGGLKSMTGFPVRKVNTDITSIT
SNGGRVKCMQVWPPIGKKKFETLSYLPPLTRDSRA;
(SEQ ID NO: 304)
MASMISSSAVTTVSRASRGQSAAMAPFGGLKSMTGFPVRKVNTDITSIT
SNGGRVKS;
(SEQ ID NO: 13863)
MASSMLSSATMVASPAQATMVAPFNGLKSSAAFPATRKANNDITSITSN
GGRVNCMQVWPPIEKKKFETLSYLPDLTDSGGRVNC;
(SEQ ID NO: 305)
MAQVSRICNGVQNPSLISNLSKSSQRKSPLSVSLKTQQHPRAYPISSSW
GLKKSGMTLIGSELRPLKVMSSVSTAC;
(SEQ ID NO: 306)
MAQVSRICNGVWNPSLISNLSKSSQRKSPLSVSLKTQQHPRAYPISSSW
GLKKSGMTLIGSELRPLKVMSSVSTAC;
(SEQ ID NO: 307)
MAQINNMAQGIQTLNPNSNFHKPQVPKSSSFLVFGSKKLKNSANSMLVL
KKDSIFMQLFCSFRISASVATAC;
(SEQ ID NO: 308)
MAALVTSQLATSGTVLSVTDRFRRPGFQGLRPRNPADAALGMRTVGASA
APKQSRKPHRFDRRCLSMVV;
(SEQ ID NO: 309)
MAALTTSQLATSATGFGIADRSAPSSLLRHGFQGLKPRSPAGGDATSLS
VTTSARATPKQQRSVQRGSRRFPSVVVC;
(SEQ ID NO: 310)
MASSVLSSAAVATRSNVAQANMVAPFTGLKSAASFPVSRKQNLDITSIA
SNGGRVQC;
(SEQ ID NO: 13864)
MESLAATSVFAPSRVAVPAARALVRAGTVVPTRRTSSTSGTSGVKCSAA
VTPQASPVISRSAAAA;
and
(SEQ ID NO: 311)
MGAAATSMQSLKFSNRLVPPSRRLSPVPNNVTCNNLPKSAAPVRTVKCCA
SSWNSTINGAAATTNGASAASS.

In some cases, a CasX variant protein of the present disclosure can include an endosomal escape peptide. In some cases, an endosomal escape polypeptide comprises the amino acid sequence GLFXALLXLLXSLWXLLLXA (SEQ ID NO: 312), wherein each X is independently selected from lysine, histidine, and arginine. In some cases, an endosomal escape polypeptide comprises the amino acid sequence GLFHALLHLLHSLWHLLLHA (SEQ ID NO: 313), or HHHHHHHHH (SEQ ID NO: 314).

Non-limiting examples of fusion partners for use with CasX variant proteins when targeting ssRNA target nucleic acid sequences include (but are not limited to): splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; RNA-binding proteins; and the like. It is understood that a heterologous polypeptide can include the entire protein or in some cases can include a fragment of the protein (e.g., a functional domain).

In some embodiments, a CasX variant comprises any one of SEQ ID NOS: 49-160, 439, 441, 443, 445, 447-460, 472, 474, 476, 478, 480, 482, 484, 486, 488, or 490 comprises a fusion partner of any domain capable of interacting with ssRNA (which, for the purposes of this disclosure, includes intramolecular and/or intermolecular secondary structures, e.g., double-stranded RNA duplexes such as hairpins, stem-loops, etc.), whether transiently or irreversibly, directly or indirectly, including but not limited to an effector domain selected from the group comprising; endonucleases (for example RNase III, the CRR22 DYW domain, Dicer, and PIN (PilT N-terminus) domains from proteins such as SMGS and SMG6); proteins and protein domains responsible for stimulating RNA cleavage (for example CPSF, CstF, CFIm and CFIIm); exonucleases (for example XRN-1 or Exonuclease T); deadenylases (for example HNT3); proteins and protein domains responsible for nonsense mediated RNA decay (for example UPF1, UPF2, UPF3, UPF3b, RNP SI, Y14, DEK, REF2, and SRm160); proteins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI Dl and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP Al); proteins and protein domains responsible for stimulation of RNA splicing (for example serine/arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable heterologous polypeptide is a PUF RNA-binding domain, which is described in more detail in WO2012068627, which is hereby incorporated by reference in its entirety.

Some RNA splicing factors that can be used (in whole or as fragments thereof) as a fusion partner with a CasX variant have modular organization, with separate sequence-specific RNA binding modules and splicing effector domains. For example, members of the serine/arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP Al can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived post mitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cc-elements that are located in either the core exon region or the exon extension region (i.e., between the two alternative 5′ splice sites). For more examples, see WO2010075303, which is hereby incorporated by reference in its entirety.

Further suitable fusion partners for use with a CasX variant include, but are not limited to proteins (or fragments thereof) that are boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In some cases, a heterologous polypeptide (a fusion partner) for use with a CasX variant provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a CasX variant protein includes (is fused to) a nuclear localization signal (NLS). In some cases, a CasX variant protein is fused to 2 or more, 3 or more, 4 or more, or 5 or more 6 or more, 7 or more, 8 or more NLSs. In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus of the CasX variant. In some cases, one or more NLSs (2 or more, 3 or more, 4 or more, or 5 or more NLSs) are positioned at or near (e.g., within 50 amino acids of) the C-terminus of the CasX variant. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus. In some cases, a reference or CasX variant protein includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a reference or CasX variant protein includes (is fused to) between 2 and 5 NLSs (e.g., 2-4, or 2-3 NLSs).

Non-limiting examples of NLSs include sequences derived from: the NLS of the SV40 virus large T-antigen, having the amino acid sequence PKKKRKV (SEQ ID NO: 217); the NLS from nucleoplasmin (e.g., the nucleoplasmin bipartite NLS with the sequence KRPAATKKAGQAKKKK (SEQ ID NO: 223); the c-myc NLS having the amino acid sequence PAAKRVKLD (SEQ ID NO: 224) or RQRRNELKRSP (SEQ ID NO: 161); the hRNPA1 M9 NLS having the sequence NQSSNFGPMKGGNFGGRSSGPYGGGGQYFAKPRNQGGY (SEQ ID NO: 162); the sequence RMRIZFKNKGKDTAELRRRRVEVSVELRKAKKDEQILKRRNV (SEQ ID NO: 163) of the IBB domain from importin-alpha; the sequences VSRKRPRP (SEQ ID NO: 164) and PPKKARED (SEQ ID NO: 165) of the myoma T protein; the sequence PQPKKKPL (SEQ ID NO: 166) of human p53; the sequence SALIKKKKKMAP (SEQ ID NO: 167) of mouse c-abl IV; the sequences DRLRR (SEQ ID NO: 168) and PKQKKRK (SEQ ID NO: 169) of the influenza virus NS1; the sequence RKLKKKIKKL (SEQ ID NO: 170) of the Hepatitis virus delta antigen; the sequence REKKKFLKRR (SEQ ID NO: 171) of the mouse Mxl protein; the sequence KRKGDEVDGVDEVAKKKSKK (SEQ ID NO: 172) of the human poly(ADP-ribose) polymerase; the sequence RKCLQAGMNLEARKTKK (SEQ ID NO: 173) of the steroid hormone receptors (human) glucocorticoid; the sequence PRPRKIPR (SEQ ID NO: 174) of Borna disease virus P protein (BDV-P1); the sequence PPRKKRTVV (SEQ ID NO: 175) of hepatitis C virus nonstructural protein (HCV-NS5A); the sequence NLSKKKKRKREK (SEQ ID NO: 176) of LEF1; the sequence RRPSRPFRKP (SEQ ID NO: 177) of ORF57 simirae; the sequence KRPRSPSS (SEQ ID NO: 178) of EBV LANA; the sequence KRGINDRNFWRGENERKTR (SEQ ID NO: 179) of Influenza A protein; the sequence PRPPKMARYDN (SEQ ID NO: 180) of human RNA helicase A (RHA); the sequence KRSFSKAF (SEQ ID NO: 181) of nucleolar RNA helicase II; the sequence KLKIKRPVK (SEQ ID NO: 182) of TUS-protein; the sequence PKKKRKVPPPPAAKRVKLD (SEQ ID NO: 183) associated with importin-alpha; the sequence PKTRRRPRRSQRKRPPT (SEQ ID NO: 184) from the Rex protein in HTLV-1; the sequence MSRRRKANPTKLSENAKKLAKEVEN (SEQ ID NO: 185) from the EGL-13 protein of Caenorhabditis elegans; and the sequences KTRRRPRRSQRKRPPT (SEQ ID NO: 186), RRKKRRPRRKKRR (SEQ ID NO: 187), PKKKSRKPKKKSRK (SEQ ID NO: 188), HKKKHPDASVNFSEFSK (SEQ ID NO: 189), QRPGPYDRPQRPGPYDRP (SEQ ID NO: 190), LSPSLSPLLSPSLSPL (SEQ ID NO: 191), RGKGGKGLGKGGAKRHRK (SEQ ID NO: 192), PKRGRGRPKRGRGR (SEQ ID NO: 193), PKKKRKVPPPPAAKRVKLD (SEQ ID NO: 183) and PKKKRKVPPPPKKKRKV (SEQ ID NO: 194). In general, NLS (or multiple NLSs) are of sufficient strength to drive accumulation of a reference or CasX variant fusion protein in the nucleus of a eukaryotic cell. Detection of accumulation in the nucleus may be performed by any suitable technique. For example, a detectable marker may be fused to a reference or CasX variant fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined.

In some cases, a CasX variant fusion protein includes a “Protein Transduction Domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of a CasX variant fusion protein. In some embodiments, a PTD is covalently linked to the carboxyl terminus of a CasX variant fusion protein. In some cases, the PTD is inserted internally in the sequence of a CasX variant fusion protein at a suitable insertion site. In some cases, a CasX variant fusion protein includes (is conjugated to, is fused to) one or more PTDs (e.g., two or more, three or more, four or more PTDs). In some cases, a PTD includes one or more nuclear localization signals (NLS). Examples of PTDs include but are not limited to peptide transduction domain of HIV TAT comprising YGRKKRRQRRR (SEQ ID NO: 195), RKKRRQRR (SEQ ID NO: 196); YARAAARQARA (SEQ ID NO: 197); THRLPRRRRRR (SEQ ID NO: 198); and GGRRARRRRRR (SEQ ID NO: 199); a polyarginine sequence comprising a number of arginines sufficient to direct entry into a cell (e.g., 3, 4, 5, 6, 7, 8, 9, 10, or 10-50 arginines (SEQ ID NO: 200)); a VP22 domain (Zender et al. (2002) Cancer Gene Ther. 9(6):489-96); an Drosophila Antennapedia protein transduction domain (Noguchi et al. (2003) Diabetes 52(7): 1732-1737); a truncated human calcitonin peptide (Trehin et al. (2004) Pharm. Research 21 :1248-1256); polylysine (Wender et al. (2000) Proc. Natl. Acad. Sci. USA 97: 13003-13008); RRQRRTSKLMKR (SEQ ID NO: 201); Transportan GWTLNSAGYLLGKINLKALAALAKKIL (SEQ ID NO: 202); KALAWEAKLAKALAKALAKHLAKALAKALKCEA (SEQ ID NO: 203); and RQIKIWFQNRRMKWKK (SEQ ID NO: 204). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a CasX variant fusion protein can include a CasX protein that is linked to an internally inserted heterologous amino acid or heterologous polypeptide (a heterologous amino acid sequence) via a linker polypeptide (e.g., one or more linker polypeptides). In some embodiments, a CasX variant fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Example linker polypeptides include glycine polymers (G)n, glycine-serine polymers (including, for example, (GS)n, GSGGSn (SEQ ID NO: 205), GGSGGSn (SEQ ID NO: 206), and GGGSn (SEQ ID NO: 207), where n is an integer of at least one), glycine-alanine polymers, alanine-serine polymers, glycine-proline polymers, proline polymers and proline-alanine polymers. Example linkers can comprise amino acid sequences including, but not limited to, GGSG (SEQ ID NO: 208), GGSGG (SEQ ID NO: 209), GSGSG (SEQ ID NO: 210), GSGGG (SEQ ID NO: 211), GGGSG (SEQ ID NO: 212), GSSSG (SEQ ID NO: 213), GPGP (SEQ ID NO: 214), GGP, PPP, PPAPPA (SEQ ID NO: 215), PPPGPPP (SEQ ID NO: 216) and the like. The ordinarily skilled artisan will recognize that design of a peptide conjugated to any elements described above can include linkers that are all or partially flexible, such that the linker can include a flexible linker as well as one or more portions that confer less flexible structure.

### V. Systems and Methods for Modification of PCSK9 Genes

The CRISPR proteins, guide nucleic acids, and variants thereof provided herein are useful for various applications, including as therapeutics, diagnostics, and for research. In some embodiments, to effect the methods of the disclosure for gene editing, provided herein are programmable CasX:gNA systems. The programmable nature of the CasX:gNA systems provided herein allows for the precise targeting to achieve the desired effect (nicking, cleaving, repairing, etc.) at one or more regions of predetermined interest in the target nucleic acid sequence of the PCSK9 gene. In some embodiments, it may be desirable to knock-down or knock-out expression of the PCSK9 protein in the subject comprising mutations, for example dominant mutations leading to hypercholesterolemia or autosomal dominant hypercholesterolemia. The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant or heterologous sequence. The term “knock-down” as used herein refers to reduction in the expression of a gene or its gene product(s). As a result of a gene knock-down, the protein activity or function may be attenuated or the protein levels may be reduced or eliminated. In such embodiments, gNAs having targeting sequences specific for a portion of the gene encoding the PCSK9 protein or the PCSK9 regulatory regions may be used. Depending on the CasX protein and gNA used, the event may be a cleavage event, allowing for knock-down/knock-out of expression. In some embodiments, PCSK9 gene expression may be disrupted or eliminated by introducing random insertions or deletions (indels), for example by utilizing the imprecise non-homologous DNA end joining (NHEJ) repair pathway. In such embodiments, the targeted region of the PCSK9 includes coding sequences (exons) of the PCSK9 gene, as inserting or deleting nucleotides within coding sequences can generate a frame shift mutation. This approach can also be used in non-coding regions such as introns, or regulatory regions to disturb expression of the PCSK9 gene. In other embodiments, the disclosure provides systems and methods for correcting mutations in the PCSK9 gene wherein a corrective sequence is knocked-in by introducing insertions or deletions at select locations by design of the targeting sequence of the gNA or by introduction of a donor template, described more fully, below.

In some embodiments, the CasX:gNA systems provided herein for modification of the PCSK9 target nucleic acid comprise a CasX variant of SEQ ID NOS: 49-160, 439, 441, 443, 445, 447-460, 472, 474, 476, 478, 480, 482, 484, 486, 488, or 490 as set forth in Tables 3, 5, 6, 7, or 9 or a variant sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the gNA scaffold comprises a sequence of Table 2 or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the gNA comprises a targeting sequence of SEQ ID NOS: 247-303, 315-436, 612-2100, or 2286-13861 or a sequence at least 65% identical, at least 70% identical, at least 75% identical, at least 80% identical, at least 85% identical, at least 90% identical, or at least 95% identical thereto and having between 15 and 30 amino acids.

In other embodiments, the disclosure provides one or more polynucleotides encoding the foregoing CasX variant proteins and gNAs. In some cases, the CasX:gNA system further comprises a donor template nucleic acid, wherein the donor template can be inserted by HDR or HITI repair mechanisms of the host cell. In the embodiments, the donor template can comprise a nucleic acid comprising at least a portion of a PCSK9 gene selected from the group consisting of a PCSK9 exon, a PCSK9 intron, a PCSK9 intron-exon junction, and a PCSK9 regulatory element and combinations thereof. In the embodiments, the donor template can comprise a sequence encoding all or a portion of SEQ ID NO:33. In some embodiments, e.g. for knock-down/knock-out modifications, the donor template sequence will have at least about 60%, 70%, 80%, 90%, 95%, 98%, 99%, or 99.9% sequence identity to the PCSK9 genomic sequence with which recombination is desired, such that upon insertion, the expression of the PCSK9 gene product is reduced or eliminated such that expression of the non-functional PCSK9 protein is decreased by at least about 10%, at least about 20%, at least about 30%, at least about 40%, at least about 50%, at least about 60%, at least about 70%, at least about 80%, or at least about 90% in comparison to a cell where the PCSK9 gene has not been modified. In some embodiments, the donor template comprises a sequence to correct the mutation(s) of the PCSK9 gene, wherein upon insertion, the expression of functional PCSK9 protein by the cells of the population is increased by at least about 10%, at least about 20%, at least about 30%, at least about 40%, at least about 50%, at least about 60%, at least about 70%, at least about 80%, or at least about 90% in comparison to a cell where the PCSK9 gene has not been modified. In other embodiments, the insertion of the corrective donor template modifies the PCSK9 gene of the cells such that at least about 50%, at least about 60%, at least about 70%, at least about 75%, at least about 80%, at least about 85%, at least about 90%, or at least about 95% of the modified cells express a detectable level of functional PCSK9. In some embodiments, the insertion of the corrective donor template modifies the PCSK9 gene of the cells such that at least about 10%, at least about 20%, at least about 30%, at least about 40%, at least about 50%, at least about 60%, at least about 70%, at least about 80%, or at least about 90% of the modified cells do not express a detectable level of non-functional PCSK9 protein.

In other embodiments, the donor template comprises a sequence to abridge the mutant exons to be excised from the PCSK9 gene; e.g., two or more consecutive exons, which can further comprise the intervening introns between the two or more consecutive exons, or a cDNA comprising the exons and a shortened synthetic intron. The donor template can be a short single-stranded or double-stranded oligonucleotide, or a long single-stranded or double-stranded oligonucleotide. The donor template sequence comprises a sequence flanked by two regions of homology (“homologous arms”) to the 5′ and 3′ sides of the break site(s) such that homology-directed repair between the target DNA region and the two flanking sequences results in insertion of the donor template at the target region. In those cases where the PCSK9 mutation spans multiple exons, the methods of the disclosure contemplate use a donor template of sufficient length that may also be optimized to contain synthetic intron sequences of shortened length (relative to the genomic intron) between the exons in the donor template to ensure proper expression and processing of the PCSK9 locus. In some embodiments, the donor polynucleotide comprises at least about 10, at least about 50, at least about 100, or at least about 200, or at least about 300, or at least about 400, or at least about 500, or at least about 600, or at least about 700, or at least about 800, or at least about 900, or at least about 1000, or at least about 10,000, or at least about 15,000 nucleotides. In other embodiments, the donor polynucleotide comprises at least about 10 to about 15,000 nucleotides, or at least about 100 to about 10,000 nucleotides, or at least about 400 to about 8,000 nucleotides, or at least about 600 to about 5000 nucleotides, or at least about 1000 to about 2000 nucleotides. The donor template sequence may comprise certain sequence differences as compared to the genomic sequence, e.g., restriction sites, nucleotide polymorphisms, selectable markers (e.g., drug resistance genes, fluorescent proteins, enzymes etc.), etc., which may be used to assess for successful insertion of the donor nucleic acid at the cleavage site or in some cases may be used for other purposes (e.g., to signify expression at the targeted genomic locus). Alternatively, these sequence differences may include flanking recombination sequences such as FLPs, loxP sequences, or the like, that can be activated at a later time for removal of the marker sequence.

A variety of strategies and methods can be employed to modify the target nucleic acid sequence in a cell using the CasX:gNA systems provided herein. As used herein “modifying” includes but is not limited to cleaving, nicking, editing, deleting, knocking in, knocking out, repairing/correcting, exon-skipping and the like. Depending on the CasX protein and gNA utilized, the editing event may be a cleavage event followed by introducing random insertions or deletions (indels) or other mutations (e.g., a substitution, duplication, or inversion of one or more nucleotides), for example by utilizing the imprecise non-homologous DNA end joining (NHEJ) repair pathway, which may generate, for example, a frame shift mutation. Alternatively, the editing event may be a cleavage event followed by homology-directed repair (HDR), homology-independent targeted integration (HITI), micro-homology mediated end joining (MMEJ), single strand annealing (SSA) or base excision repair (BER), resulting in modification of the target nucleic acid sequence.

In one embodiment, the disclosure provides for a method of modifying a target nucleic acid sequence of a PCSK9 gene comprising one or more mutations in a population of cells, the method comprising introducing into each cell of the population: i) a CasX:gNA system comprising a CasX and a gNA of any one of the embodiments described herein; ii) a CasX:gNA system comprising a CasX, a gNA, and a donor template of any one of the embodiments described herein; iii) a nucleic acid encoding the CasX and the gNA, and optionally comprising the donor template; iv) a vector selected from the group consisting of a retroviral vector, a lentiviral vector, an adenoviral vector, an adeno-associated viral (AAV) vector, and a herpes simplex virus (HSV) vector, and comprising the nucleic acid of (iii), above; v) a VLP comprising the CasX:gNA system of any one of the embodiments described herein; or vi) combinations of two or more of (i) to (v), wherein the target nucleic acid sequence of the cells targeted by the gNA is modified by the CasX protein. In some embodiments of the method, the PCSK9 target nucleic acid of at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60% or more of the cells of the population is modified. In some embodiments of the method, the PCSK9 gene in the cells of the population is modified such that expression of non-functional PCSK9 protein is decreased by at least about 10%, at least about 20%, at least about 30%, at least about 40%, at least about 50%, at least about 60%, at least about 70%, at least about 80%, or at least about 90% in comparison to a cell where the PCSK9 gene has not been modified. In other embodiments of the method, the PCSK9 gene of the cells of the population is modified such that at least about 10%, at least about 20%, at least about 30%, at least about 40%, at least about 50%, at least about 60%, at least about 70%, at least about 80%, or at least about 90% of the modified cells do not express a detectable level of non-functional PCSK9 protein.

In one embodiment of the method, the CasX and gNA of the CasX:gNA system is introduced into the cells as an RNP. The polynucleotide can be introduced into the cells to be modified by a vector as described herein, or as a plasmid using conventional methods known in the art; e.g. electroporation, microinjection, or chemically. In some embodiments of the method, the cells to be modified are selected from the group consisting of rodent cells, mouse cells, rat cells, and non-human primate cells. In other embodiments of the method, the cells to be modified are human cells. In some embodiments of the method, the modification of the population of cells occurs in vivo in a subject, wherein the subject is selected from the group consisting of a rodent, a mouse, a rat, a non-human primate, and a human. In other embodiments of the method, the modification of the population of cells occurs ex vivo. In some embodiments of the method, the cells of the population to be modified are selected from the group consisting of progenitor cells, hematopoietic stem cells, and pluripotent stem cells. In other embodiments of the method, the cells are induced pluripotent stem cells. In some embodiments of the methods, the modified cell is a hepatocyte, or a cell of the intestine, the kidney, the central nervous system, a smooth muscle cell, macrophage or a cell of arterial walls such as the endothelium. In some embodiments, the cells of the population are autologous with respect to a subject to be administered said cell. In other embodiments of the method, the cells of the population are allogeneic with respect to a subject to be administered said cell.

In some embodiments of the method, the targeting sequence of the gNA is complementary to a sequence comprising one or more single nucleotide polymorphisms (SNPs) of the PCSK9 gene. In other embodiments, the targeting sequence of the gNA is complementary to a sequence of an exonic splicing enhancer of the PCSK9 gene.

In some embodiments of the method of modifying a target nucleic acid sequence, the target nucleic acid sequence comprises all or a portion of the PCSK9 gene. In some embodiments, the PCSK9 gene to be modified comprises a wild type sequence corresponding to a polynucleotide encoding all or a portion of the sequence of SEQ ID NO:33 or comprises a polynucleotide sequence that spans chr1:55,039,476-55,064,853 of the human genome (GRCh38/hg38) (the notation refers to the chromosome 1 (chr1), starting at the 55,039,476 bp to 55,064,853 bp on chromosome 1 (Homo sapiens Updated Annotation Release 109.20190905, GRCh38.p13) (NCBI). In some embodiments of the method, the targeting sequence of the gNA is complementary to a sequence of a PCSK9 exon selected from the group consisting of exon 1, exon 2, exon 3, exon 4, exon 5, exon 6, exon 7, exon 8, exon 9, exon 10, exon 11, and exon 12.

In some embodiments of the methods of modifying a target nucleic acid sequence, modifying the target nucleic acid sequence comprises nicking the target nucleic acid to introduce a single-stranded break in the target nucleic acid sequence, wherein the modification of the PCSK9 gene comprises introducing a mutation, an insertion, or a deletion. In some embodiments, the modifying comprises cleaving the target nucleic acid sequence to introduce a double-stranded break in the target nucleic acid, wherein the modification of the PCSK9 gene comprises introducing a mutation, an insertion, or a deletion of one or more nucleotides as compared to the wild-type sequence. In some embodiments, the mutation to be corrected by the method is a gain of function mutation. In other embodiments, the mutation to be corrected by the method is a loss of function mutation. In some cases, the PCSK9 protein to be modified comprises a mutation that disrupts the function of the PCSK9 protein. In some embodiments of the method to correct the one or more mutations, the modifying results in a correction or compensation of the mutation of the PCSK9 gene in the cells of the population such that functional PCSK9 protein is expressed by the cells. In some embodiments of the method, expression of the functional PCSK9 protein by the cells of the population is increased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

In some embodiments of the method of modifying a target nucleic acid sequence, modifying the PCSK9 gene comprises binding of the CasX:gNA complex to the target nucleic acid sequence. In some embodiments, the CasX is a catalytically inactive CasX (dCasX) protein that retains the ability to bind to the gNA and the target nucleic acid sequence. For example, the target nucleic acid sequence comprises a PCSK9 sequence comprising a mutation, and binding of the dCasX:gRNA complex to the target sequence interferes with or represses transcription of mutant PCSK9 allele. In some embodiments, the dCasX comprises a mutation at residues D672, E769, and/or D935 corresponding to the CasX protein of SEQ ID NO:1 or D659, E756 and/or D922 corresponding to the CasX protein of SEQ ID NO: 2. In some embodiments of the foregoing, the mutation in the CasX reference protein is a substitution of alanine or glycine for the residue.

Methods of introducing a nucleic acid (e.g., a nucleic acid comprising a donor polynucleotide sequence, one or more nucleic acids encoding a CasX protein and/or gNA, or a vector comprising same) into a cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, nucleofection, electroporation, direct addition by cell penetrating CasX proteins that are fused to or recruit donor DNA, cell squeezing, calcium phosphate precipitation, direct microinjection, nanoparticle-mediated nucleic acid delivery, and the like.

In some embodiments of the method, a CasX can be provided as an RNA sequence. The RNA can be provided by direct chemical synthesis, or may be transcribed in vitro from a DNA (e.g., a DNA encoding an mRNA comprising a sequence encoding the CasX protein variant). Once synthesized, the RNA may, for example, be introduced into a cell by any of the well-known techniques for introducing nucleic acids into cells, including, but not limited to microinjection, electroporation, and transfection, for translation into the CasX protein.

Nucleic acids may be introduced into the cells using well-developed transfection techniques, and the commercially available TransMessenger® reagents from Qiagen, Stemfect™ RNA Transfection Kit from Stemgent, and TransIT®-mRNA Transfection Kit from Minis Bio LLC, Lonza nucleofection, Maxagen electroporation and the like.

Introducing recombinant expression vectors comprising sequences encoding the CasX:gNA systems (and, optionally, the donor template sequences) of the disclosure into cells under in vitro conditions can occur in any suitable culture media and under any suitable culture conditions that promote the survival of the cells and production of the CasX:gNA. Introducing recombinant expression vectors into a target cell can be carried out in vivo, in vitro or ex vivo. In some embodiments of the method, vectors may be provided directly to a target host cell. For example, cells may be contacted with vectors having nucleic acids encoding the CasX and gNA of any of the embodiments described herein and, optionally, having a donor template sequence such that the vectors are taken up by the cells. Methods for contacting cells with nucleic acid vectors that are plasmids include electroporation, calcium chloride transfection, microinjection, transduction and lipofection are well known in the art. For viral vector delivery, cells can be contacted with viral particles comprising the subject viral expression vectors and the nucleic acid encoding the CasX and gNA and, optionally, the donor template. In some embodiments, the vector is an Adeno-Associated Viral (AAV) vector, wherein the AAV is selected from AAV1, AAV2, AAV3, AAV4, AAV5, AAV6, AAV7, AAV8, AAV9, AAV10, AAV11, AAV12, AAV 44.9, AAV-Rh74, or AAVRh10. In other embodiments, the vector is a lentiviral vector. Retroviruses, for example, lentiviruses, may be suitable for use in methods of the present disclosure. Commonly used retroviral vectors are “defective”, e.g., are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, and this envelope protein determines the specificity or tropism of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines, and of collecting the viral particles that are generated by the packaging lines, are well known in the art, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Nucleic acids can also be introduced by direct micro-injection (e.g., injection of RNA).

In some embodiments, the vector is administered to a subject at a therapeutically effective dose. In the foregoing, the subject is selected from the group consisting of mouse, rat, pig, non-human primate, and human. In particular embodiments, the subject is a human. In some embodiments of the method, the vector is administered to a subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg. In other embodiments of the method, the VLP is administered to a subject at a dose of at least about 1×10⁵ particles/kg, at least about 1×10⁶ particles/kg, at least about 1×10⁷ particles/kg, at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, or at least about 1×10¹⁶ particles/kg.

The vector or VLP can be administered by a route of administration selected from the group consisting of intravenous, intraportal vein injection, intraperitoneal, intramuscular, subcutaneous, intraocular, and oral routes. In some embodiments, the vector is an AAV vector comprising a CasX:gNA system of the disclosure, and is delivered via intraocular injection to one or both eyes of the subject.

In other embodiments, the disclosure provides methods of modifying target nucleic acid sequences using the CasX:gNA systems of any of the embodiments described herein, and the methods further comprise contacting the target nucleic acid sequence with an additional CRISPR protein, or a polynucleotide encoding the additional CRISPR protein. In some embodiments, the additional CRISPR protein is a CasX protein having a sequence different from the CasX of the CasX:gNA system. In some embodiments, the additional CRISPR protein is not a CasX protein; e.g., the additional CRISPR protein can be Cpf1, Cas9, Cas12a, or Cas13a.

The CasX:gNA systems and methods described herein can be used to engineer a variety of cells in which mutations in PCSK9 are associated with disease, e.g., cells of the liver, the intestine, the kidney, the central nervous system, smooth muscle cells, macrophages or cells of arterial walls such as the endothelium, to produce a cell or cells in which the PCSK9 comprising mutations is corrected or knocked-out. This approach, therefore, could be used to modify cells for applications in a subject with a PCSK9-related disorder such as, but not limited to autosomal dominant hypercholesterolemia (ADH), hypercholesterolemia, elevated total cholesterol levels, hyperlipidemia, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, age-related macular degeneration (AMD), or a combination thereof.

### VI. Polynucleotides and Vectors

In another aspect, the present disclosure relates to polynucleotides encoding the Class2, Type V nucleases and gNA that have utility in the editing of the PCSK9 gene comprising one or more mutations. In additional embodiments, the disclosure provides donor template polynucleotides encoding portions or all of a PCSK9 gene. In some cases, the PCSK9 gene of the donor template comprises a mutation or a heterologous sequence for knocking down or knocking out the PCSK9 gene in the target nucleic acid. In other cases, the donor template comprises a corrective sequence for knocking in a functional PCSK9 gene or portion thereof. In yet further embodiments, the disclosure provides vectors comprising polynucleotides encoding the CasX proteins and the CasX gNAs described herein, as well as the donor templates of the embodiments.

In some embodiments, the disclosure provides polynucleotide sequences encoding the reference CasX of SEQ ID NOS: 1-3. In other embodiments, the disclosure provides polynucleotide sequences encoding the CasX variants of any of the embodiments described herein, including the CasX protein variants of SEQ ID NOS: 49-160, 439, 441, 443, 445, 447-460, 472, 474, 476, 478, 480, 482, 484, 486, 488, and 490 as set forth in Tables 3, 5, 6, 7 and 9, or sequences having at least about 50%, at least about 60%, at least about 70%, at least about 80%, at least about 90%, at least about 95%, at least about 96%, at least about 97%, at least about 98%, or at least about 99% sequence identity to a sequence of SEQ ID NOS: 49-160, 439, 441, 443, 445, 447-460, 472, 474, 476, 478, 480, 482, 484, 486, 488, and 490 as set forth in Tables 3, 5, 6, 7 and 9. In some embodiments, the disclosure provides an isolated polynucleotide sequence encoding a gNA sequence of any of the embodiments described herein. In some embodiments, the disclosure provides polynucleotides encoding a gNA scaffold sequence of SEQ ID NOS: 4-16 or 2101-2285 as set forth in Table 1 or Table 2, or a sequence having at least about 50%, at least about 60%, at least about 70%, at least about 80%, at least about 90%, at least about 95%, at least about 96%, at least about 97%, at least about 98%, at least about 99% sequence identity thereto. In some embodiments, the polynucleotide encodes a gNA scaffold sequence selected from the group consisting of SEQ ID NOS:2101-2285, or a sequence having at least about 50%, at least about 60%, at least about 70%, at least about 80%, at least about 90%, at least about 95%, at least about 96%, at least about 97%, at least about 98%, at least about 99% sequence identity thereto. In other embodiments, the disclosure provides gNAs comprising targeting sequence polynucleotides of SEQ ID NOS: 247-303, 315-436, 612-2100, or 2286-13861, or a sequences having at least about 65%, at least about 75%, at least about 85%, or at least about 95% identity thereto, as well as DNA encoding the targeting sequences. In some embodiments, the polynucleotide encoding the scaffold sequence further comprises the sequence encoding the targeting sequence such that a gNA capable of binding the CasX and the target sequence can be expressed as a sgNA or dgNA. In other embodiments, the disclosure provides an isolated polynucleotide sequence encoding a gNA sequence having a scaffold and targeting sequence that hybridizes with the PCSK9 gene comprising one or more mutations. In some cases, the polynucleotide sequence encodes a gNA of a scaffold and targeting sequence that hybridizes with a PCSK9 gene exon selected from the group consisting of exon 1, exon 2, exon 3, exon 4, exon 5, exon 6, exon 7, exon 8, exon 9, exon 10, exon 11, and exon 12. In other embodiments, the polynucleotide sequence encodes a gNA comprising a targeting sequence that hybridizes with a PCSK9 intron. In other embodiments, the polynucleotide sequence encodes a gNA comprising a targeting sequence that hybridizes with a PCSK9 intron-exon junction. In other embodiments, the polynucleotide sequence encodes a gNA comprising a targeting sequence that hybridizes with an intergenic region of the PCSK9 gene. In other embodiments, the polynucleotide sequence encodes a gNA comprising a targeting sequence that hybridizes with a PCSK9 regulatory region. In some cases, the PCSK9 regulatory region is a PCSK9 promoter or enhancer. In some cases, the PCSK9 regulatory region is located 5′ of the PCSK9 transcription start site, 3′ of the PCSK9 transcription start, or in a PCSK9 intron. In some cases, the PCSK9 regulatory region is in an intron of the PCSK9 gene. In other cases, the PCSK9 regulatory region comprises the 5′ UTR of the PCSK9 gene. In still other cases, the PCSK9 regulatory region comprises the 3′UTR of the PCSK9 gene. In some cases, the PCSK9 sequence is a wild-type sequence. In other cases, the PCSK9 sequence comprises one or more mutations.

In other embodiments, the disclosure provides donor template nucleic acids wherein the donor template comprises a nucleotide sequence having homology but not complete identity to a target sequence of the target nucleic acid for which gene editing is intended. The donor template sequence is typically not identical to the genomic sequence that it replaces and may contain one or more single base changes, insertions, deletions, inversions or rearrangements with respect to the genomic sequence, provided that there is sufficient homology with the target sequence to support homology-directed repair, or the donor template has homologous arms, whereupon insertion can result in splicing out of exons comprising mutations such that the reading frame of the PCSK9 gene is restored, or the donor template comprises wild-type sequence such that upon insertions, the mutation is corrected. In some embodiments, the donor template has a sequence that hybridizes with the protein target nucleic acid and is inserted at the break sites introduced by the CasX, effecting a modification of the gene sequence. In those cases where the PCSK9 mutation spans multiple exons, the disclosure contemplates a donor template of sufficient length that may also be optimized to contain synthetic intron sequences of shortened length (relative to the genomic intron) between the exons in the donor template to ensure proper expression and processing of the PCSK9 locus. In some embodiments, the donor polynucleotide comprises at least about 10, at least about 50, at least about 100, or at least about 200, or at least about 300, or at least about 400, or at least about 500, or at least about 600, or at least about 700, or at least about 800, or at least about 900, or at least about 1000, or at least about 10,000, or at least about 15,000 nucleotides. In other embodiments, the donor polynucleotide comprises at least about 10 to about 15,000 nucleotides, or at least about 100 to about 10,000 nucleotides, or at least about 400 to about 8,000 nucleotides, or at least about 600 to about 5000 nucleotides, or at least about 1000 to about 2000 nucleotides. In some embodiments, the donor template is a single stranded DNA template or a single stranded RNA template. In other embodiments, the donor template is a double stranded DNA template.

In some embodiments, the disclosure relates to methods to produce polynucleotide sequences encoding the reference CasX, the CasX variants, or the gNA of any of the embodiments described herein, including variants thereof, as well as methods to express the proteins expressed or RNA transcribed by the polynucleotide sequences. In general, the methods include producing a polynucleotide sequence coding for the CasX or the gNA of any of the embodiments described herein and incorporating the encoding gene into an expression vector appropriate for a host cell. For production of the encoded CasX or the gNA of any of the embodiments described herein, the methods include transforming an appropriate host cell with an expression vector comprising the encoding polynucleotide, and culturing the host cell under conditions causing or permitting the resulting CasX or the gNA of any of the embodiments described herein to be expressed or transcribed in the transformed host cell, thereby producing the CasX or the gNA, which are recovered by methods described herein (e.g., in the Examples, below) or by standard purification methods known in the art. Standard recombinant techniques in molecular biology are used to make the polynucleotides and expression vectors of the present disclosure.

In accordance with the disclosure, nucleic acid sequences that encode the reference CasX, the CasX variants, or the gNA of any of the embodiments described herein are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the reference CasX, the CasX variants, or the gNA that is used to transform a host cell for expression of the composition.

In one approach, a construct is first prepared containing the DNA sequence encoding a reference CasX, a CasX variant, or a gNA. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the CasX, or the gNA. Where desired, the host cell is an E. coli. In other embodiments, the host cell is a eukaryotic cell. The eukaryotic host cell can be selected from BHK cells, HEK293 cells, HEK293T cells, Lenti-X HEK293 cells, NS0 cells, SP2/0 cells, YO myeloma cells, P3X63 mouse myeloma cells, PER cells, PER.C6 cells, hybridoma cells, NIH3T3 cells, COS, HeLa, CHO, yeast cells, or other eukaryotic cells known in the art suitable for the production of recombinant products. Exemplary methods for the creation of expression vectors, the transformation of host cells and the expression and recovery of reference CasX, the CasX variants, or the gNA are described in the Examples.

The gene encoding the reference CasX, the CasX variant, or the gNA construct can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., CasX and gNA) genes of a desired sequence. Genes encoding polypeptide compositions are assembled from oligonucleotides using standard techniques of gene synthesis.

In some embodiments, the nucleotide sequence encoding a CasX protein is codon optimized. This type of optimization can entail a mutation of an encoding nucleotide sequence to mimic the codon preferences of the intended host organism or cell while encoding the same CasX protein. Thus, the codons can be changed, but the encoded protein remains unchanged. For example, if the intended target cell of the CasX protein was a human cell, a human codon-optimized CasX-encoding nucleotide sequence could be used. As another non-limiting example, if the intended host cell were a mouse cell, then a mouse codon-optimized CasX-encoding nucleotide sequence could be generated. As another non-limiting example, if the intended host cell were a plant cell, then a plant codon-optimized CasX protein variant-encoding nucleotide sequence could be generated. As another non-limiting example, if the intended host cell were an insect cell, then an insect codon-optimized CasX protein-encoding nucleotide sequence could be generated. The gene design can be performed using algorithms that optimize codon usage and amino acid composition appropriate for the host cell utilized in the production of the reference CasX, the CasX variants, or the gNA. In one method of the disclosure, a library of polynucleotides encoding the components of the constructs is created and then assembled, as described above. The resulting genes are then assembled and the resulting genes used to transform a host cell and produce and recover the reference CasX, the CasX variants, or the gNA compositions for evaluation of its properties, as described herein.

In some embodiments, a nucleotide sequence encoding a gNA is operably linked to a control element, e.g., a transcriptional control element, such as a promoter. In some embodiments, a nucleotide sequence encoding a CasX protein is operably linked to a control element, e.g., a transcriptional control element, such as a promoter. In other cases, the nucleotide encoding the CasX and gNA are linked and are operably linked to a single control element. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., neurons, spinal motor neurons, oligodendrocytes, or glial cells. Non-limiting examples of eukaryotic promoters (promoters functional in a eukaryotic cell) include EF1alpha, EF1alpha core promoter, those from cytomegalovirus (CMV) immediate early, herpes simplex virus (HSV) thymidine kinase, early and late SV40, long terminal repeats (LTRs) from retrovirus, and mouse metallothionein-I. Further non-limiting examples of eukaryotic promoters include the CMV promoter full-length promoter, the minimal CMV promoter, the chicken β-actin promoter, the hPGK promoter, the HSV TK promoter, the Mini-TK promoter, the human synapsin I promoter which confers neuron-specific expression, the Mecp2 promoter for selective expression in neurons, the minimal IL-2 promoter, the Rous sarcoma virus enhancer/promoter (single), the spleen focus-forming virus long terminal repeat (LTR) promoter, the SV40 promoter, the SV40 enhancer and early promoter, the TBG promoter: promoter from the human thyroxine-binding globulin gene (Liver specific), the PGK promoter, the human ubiquitin C promoter, the UCOE promoter (Promoter of HNRPA2B1-CBX3), the Histone H2 promoter, the Histone H3 promoter, the U1a1 small nuclear RNA promoter (226 nt), the U1b2 small nuclear RNA promoter (246 nt) 26, the TTR minimal enhancer/promoter, the b-kinesin promoter, the human eIF4A1 promoter, the ROSA26 promoter and the glyceraldehyde 3-phosphate dehydrogenase (GAPDH) promoter.

Selection of the appropriate vector and promoter is well within the level of ordinary skill in the art, as it related to controlling expression, e.g., for modifying a PCSK9 gene. The expression vector may also contain a ribosome binding site for translation initiation, and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

In some embodiments, a nucleotide sequence encoding each of a gNA variant or a CasX protein is operably linked to an inducible promoter, a constitutively active promoter, a spatially restricted promoter (i.e., transcriptional control element, enhancer, tissue specific promoter, cell type specific promoter, etc.), or a temporally restricted promoter. In other embodiments, individual nucleotide sequences encoding the gNA or the CasX are linked to one of the foregoing categories of promoters, which are then introduced into the cells to be modified by conventional methods, described below.

In certain embodiments, suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human HI promoter (HI), a POL1 promoter, a 7SK promoter, tRNA promoters and the like.

In some embodiments, one or more nucleotide sequences encoding a CasX and gNA and, optionally, comprising a donor template, are each operably linked to (under the control of) a promoter operable in a eukaryotic cell. Examples of inducible promoters may include, but are not limited to, T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore, in some embodiments, be regulated by molecules including, but not limited to, doxycycline; estrogen and/or an estrogen analog; IPTG; etc.

In certain embodiments, inducible promoters suitable for use may include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR, etc.), tetracycline regulated promoters, (e.g., promoter systems including Tet Activators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Recombinant expression vectors of the disclosure can also comprise elements that facilitate robust expression of CasX proteins and the gNAs of the disclosure. For example, recombinant expression vectors can include one or more of a polyadenylation signal (poly(A)), an intronic sequence or a post-transcriptional regulatory element such as a woodchuck hepatitis post-transcriptional regulatory element (WPRE). Exemplary poly(A) sequences include hGH poly(A) signal (short), HSV TK poly(A) signal, synthetic polyadenylation signals, SV40 poly(A) signal, β-globin poly(A) signal and the like. A person of ordinary skill in the art will be able to select suitable elements to include in the recombinant expression vectors described herein.

The polynucleotides encoding the reference CasX, the CasX variants, or the gNA sequences can be individually cloned into an expression vector. Vectors include bacterial plasmids, viral vectors, and the like. In some embodiments, the vector is a recombinant expression vector that comprises a nucleotide sequence encoding a CasX protein. In other embodiments, the disclosure provides a recombinant expression vector comprising a nucleotide sequence encoding a CasX protein and a nucleotide sequence encoding a CasX gNA. In some cases, the nucleotide sequence encoding the CasX protein variant and/or the nucleotide sequence encoding the CasX gNA are operably linked to a promoter that is operable in a cell type of choice. In other embodiments, the nucleotide sequence encoding the CasX protein variant and the nucleotide sequence encoding the CasX gNA are provided in separate vectors.

In some embodiments, provided herein are one or more recombinant expression vectors comprising sequences such as (i) a nucleotide sequence of a donor template nucleic acid where the donor template comprises a nucleotide sequence having homology to a PCSK9 sequence of a target nucleic acid sequence (e.g., a target genome); (ii) a nucleotide sequence that encodes a CasX gNA (e.g., gRNA), that hybridizes to a sequence of the target PCSK9 locus of the targeted genome (e.g., configured as a single or dual guide RNA) operably linked to a promoter that is operable in a target cell such as a eukaryotic cell; and (iii) a nucleotide sequence encoding a CasX protein operably linked to a promoter that is operable in a target cell such as a eukaryotic cell. In some embodiments, the sequences comprising the donor template and encoding the CasX gNA and the CasX proteins are in different recombinant expression vectors, and in other embodiments one, two or all three polynucleotide sequences (for the donor template, CasX and gNA) are in the same recombinant expression vector.

The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the CasX PCSK9 editing system can be determined using any nucleic acid or protein assay known in the art. For example, the presence of transcribed mRNA of reference CasX or the CasX variants can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of CasX polynucleotide.

The disclosure provides for the use of plasmid expression vectors containing replication and control sequences that are compatible with and recognized by the host cell and are operably linked to the gene encoding the polypeptide for controlled expression of the polypeptide or transcription of the RNA. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired. The control sequences of the vector include a promoter to effect transcription, an optional operator sequence to control such transcription, a sequence encoding suitable mRNA ribosome binding sites, and sequences that control termination of transcription and translation. The promoter may be any DNA sequence, which shows transcriptional activity in the host cell of choice and may be derived from genes encoding proteins either homologous or heterologous to the host cell.

The recombinant expression vectors can be delivered to the target host cells by a variety of methods, as described more fully, below. Such methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, nucleofection, electroporation, direct addition by cell penetrating CasX proteins that are fused to or recruit donor DNA, cell squeezing, calcium phosphate precipitation, direct microinjection, nanoparticle-mediated nucleic acid delivery, and the like.

A recombinant expression vector sequence can be packaged into a virus or virus-like particle (also referred to herein as a “particle” or “virion”) for subsequent infection and transformation of a cell, ex vivo, in vitro or in vivo. Such particles or virions will typically include proteins that encapsidate or package the vector genome. Suitable expression vectors may include viral expression vectors based on vaccinia virus; poliovirus; adenovirus; a retroviral vector (e.g., Murine Leukemia Virus), spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus; and the like.

In some embodiments, a recombinant expression vector of the present disclosure is a recombinant adeno-associated virus (AAV) vector. In some embodiments, a recombinant expression vector of the present disclosure is a recombinant lentivirus vector. In some embodiments, a recombinant expression vector of the present disclosure is a recombinant retroviral vector.

AAV is a small (20 nm), nonpathogenic virus that is useful in treating human diseases in situations that employ a viral vector for delivery to a cell such as a eukaryotic cell, either in vivo or ex vivo for cells to be prepared for administering to a subject. A construct is generated, for example a construct encoding any of the CasX proteins and/or CasX gNA embodiments as described herein, and is flanked with AAV inverted terminal repeat (ITR) sequences, thereby enabling packaging of the AAV vector into an AAV viral particle.

An “AAV” vector may refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are many known serotypes of primate AAVs. In some embodiments, the AAV vector is selected from AAV1, AAV2, AAV3, AAV4, AAV5, AAV6, AAV7, AAV8, AAV9, AAV10, AAV11, AAV12, AAV 44.9, AAV-Rh74 (Rhesus macaque-derived AAV), and AAVRh10, and modified capsids of these serotypes. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped recombinant AAV (rAAV) are produced using standard techniques described in the art. As used herein, for example, rAAV1 may be used to refer an AAV having both capsid proteins and 5′-3′ ITRs from the same serotype or it may refer to an AAV having capsid proteins from serotype 1 and 5′-3′ ITRs from a different AAV serotype, e.g., AAV serotype 2. For each example illustrated herein the description of the vector design and production describes the serotype of the capsid and 5′-3′ ITR sequences.

An “AAV virus” or “AAV viral particle” refers to a viral particle composed of at least one AAV capsid protein (preferably by all of the capsid proteins of a wild-type AAV) and an encapsidated polynucleotide. If the particle additionally comprises a heterologous polynucleotide (i.e., a polynucleotide other than a wild-type AAV genome to be delivered to a mammalian cell), it is typically referred to as “rAAV”. An exemplary heterologous polynucleotide is a polynucleotide comprising a CasX protein and/or sgRNA and, optionally, a donor template of any of the embodiments described herein.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, for example Kotin, R. M. (1994) Human Gene Therapy 5:793-801; Berns, K. I. “Parvoviridae and their Replication” in Fundamental Virology, 2^{nd} Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV-Rh74, and AAVRh10, and modified capsids of these serotypes. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein.)

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, AAV capsids utilized for delivery of the encoding sequences for the CasX and gNA, and, optionally, the PCSK9 donor template nucleotides to a host cell can be derived from any of several AAV serotypes, including without limitation, AAV1, AAV2, AAV3, AAV4, AAV5, AAV6, AAV7, AAV8, AAV9, AAV10, AAV11, AAV12, AAV 44.9, AAV-Rh74 (Rhesus macaque-derived AAV), and AAVRh10, and the AAV ITRs are derived from AAV serotype 2.

In order to produce rAAV viral particles, an AAV expression vector is introduced into a suitable host cell using known techniques, such as by transfection. Packaging cells are typically used to form virus particles; such cells include HEK293 cells (and other cells known in the art), which package adenovirus. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector.

In other embodiments, retroviruses, for example, lentiviruses, may be suitable for use as vectors for delivery of the encoding nucleic acids of the CasX:gNA systems of the present disclosure. Commonly used retroviral vectors are “defective”, e.g. unable to produce viral proteins required for productive infection, and may be referred to a virus-like particles (VLP). Rather, replication of the vector requires growth in a packaging cell line. To generate viral particles comprising nucleic acids of interest, the retroviral nucleic acids comprising the nucleic acid are packaged into VLP capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art.

In other embodiments, the disclosure provides VLPs produced in vitro that comprise a CasX:gNA RNP complex of the CasX and gNA of any of the embodiments described herein and, optionally, a donor template. Combinations of structural proteins from different viruses can be used to create VLPs, including components from virus families including Parvoviridae (e.g., adeno-associated virus), Retroviridae (e.g., HIV), Flaviviridae (e.g., Hepatitis C virus), Paramyxoviridae (e.g., Nipah) and bacteriophages (e.g., Qβ, AP205). In some embodiments, the disclosure provides VLP systems designed using components of retrovirus, including lentiviruses such as HIV, in which individual plasmids comprising nucleic acids encoding the various components are introduced into a packaging cell that, in turn, produces the VLP. In some embodiments, the VLP retroviral components can be derived from any of the Retroviridae family, including Othoretrovirinae (Lentivirus, Alpharetrovirus, Betaretrovirus, Deltaretrovirus, Epsilonretrovirus, Gammaretrovirus), and Spumaretrovirinae. Exemplary VLP comprising CasX editing systems are described in PCT/US2020/063488,filed on Dec. 4, 2020, the contents of which are incorporated by reference in their entirety herein. In some embodiments, the disclosure provides VLP having an retroviral capsid that contains a CasX:gNA RNP wherein upon administration and entry into a target cell, the RNP molecule free to be transported into the nucleus of the cell. The foregoing offers advantages over other vectors in the art in that viral transduction to dividing and non-dividing cells is efficient and that the VLP delivers potent and short-lived RNP that escape a subject's immune surveillance mechanisms that would otherwise detect a foreign protein.

In some embodiments, a VLP system comprises a) a first nucleic acid comprising a sequence encoding a fusion polypeptide that comprises: i) one or more components of a Gag polyprotein; ii) a CasX protein of any of the embodiments described herein; and optionally iii) a protease cleavage site, wherein the protease cleavage site is located between the gag polyprotein component and the CasX protein of the fusion protein; b) a second nucleic acid comprising a sequence encoding a guide NA of any of the embodiments described herein; and c) a third nucleic acid comprising a sequence encoding a lentiviral pol polyprotein comprising a protease capable of cleaving the protease cleavage site between the CasX protein and the gag polyprotein. In the foregoing embodiment, the one or more components of the Gag polyprotein are selected from the group consisting of matrix protein (MA), nucleocapsid protein (NC), capsid protein (CA), p 1-p6 protein, a PP21/24 peptide, a P12/P3/P8 peptide, a p2 peptide, a P10 peptide, a p68 Gag polypeptide, a p3 Gag polypeptide. In some embodiments of the foregoing, the VLP system further comprises a fourth nucleic acid, comprising a sequence encoding a pseudotyping viral envelope protein or glycoprotein that provides for binding to a target cell or, in the alternative, the nucleic acid encodes an antibody fragment that provides for binding to a target cell, or comprises both the pseudotyping viral envelope protein or glycoprotein and the antibody fragment. The envelope protein or glycoprotein can be derived from any enveloped viruses known in the art to confer tropism to VLP, including but not limited to the group consisting of influenza A, influenza B, influenza C virus, hepatitis A virus, hepatitis B virus, hepatitis C virus, hepatitis D virus, hepatitis E virus, rotavirus, Norwalk virus, enteric adenovirus, parvovirus, Dengue fever virus, monkey pox, Mononegavirales, rabies virus, Lagos bat virus, Mokola virus, Duvenhage virus, European bat virus 1, European bat virus 2, Australian bat virus, Ephemerovirus, Vesiculovirus, vesicular stomatitis virus (VSV), herpes simplex virus type 1, herpes simplex virus type 2, varicella zoster, cytomegalovirus, Epstein-Bar virus (EBV), human herpesvirus (HHV), human herpesvirus type 6, human herpesvirus type 8, human immunodeficiency virus (HIV), papilloma virus, murine gammaherpesvirus, Argentine hemorrhagic fever virus, Bolivian hemorrhagic fever virus, Sabia-associated hemorrhagic fever virus, Venezuelan hemorrhagic fever virus, Lassa fever virus, Machupo virus, lymphocytic choriomeningitis virus (LCMV), Crimean-Congo hemorrhagic fever virus, Hantavirus, Rift Valley fever virus, Ebola hemorrhagic fever virus, Marburg hemorrhagic fever virus, Kaysanur Forest disease virus, Omsk hemorrhagic fever virus, tick-borne encephalitis causing virus, Hendra virus, Nipah virus, variola major virus, variola minor virus, Venezuelan equine encephalitis virus, eastern equine encephalitis virus, western equine encephalitis virus, SARS-associated coronavirus (SARS-CoV), and West Nile virus. In some embodiments, the packaging cell used for the production of VLP is selected from the group consisting of HEK293 cells, Lenti-X 293T cells, BHK cells, HepG2, Saos-2, HuH7, NS0 cells, SP2/0 cells, YO myeloma cells, A549 cells, P3X63 mouse myeloma cells, PER cells, PER.C6 cells, hybridoma cells, VERO, NIH3T3 cells, COS, WI38, MRCS, A549, HeLa cells (e.g., B-50), CHO cells, and HT1080 cells. Upon production and recovery of the VLP comprising the CasX:gNA RNP of any of the embodiments described herein, the VLP can be used in methods to edit target cells of subjects by the administering of such VLP, as described more fully, below.

### VII. Therapeutic Methods

The present disclosure provides methods of treating a PCSK9-related disorder in a subject in need thereof, including but not limited to autosomal dominant hypercholesterolemia (ADH), hypercholesterolemia, elevated total cholesterol levels, elevated low-density lipoprotein (LDL) levels, reduced high-density lipoprotein levels, liver steatosis, atherosclerotic cardiovascular disease, and coronary artery disease, ischemia, stroke, peripheral vascular disease, thrombosis, type 2 diabetes, high elevated blood pressure, obesity, Alzheimer's disease, neurodegeneration, age-related macular degeneration (AMD), or a combination thereof. In some embodiments, the methods of the disclosure can prevent, treat and/or ameliorate a PCSK9-related disorder of a subject by the administering to the subject of a composition of the disclosure. In some embodiments, the composition administered to the subject further comprises pharmaceutically acceptable carrier, diluent or excipient.

In some cases, one or both alleles of the PCSK9 gene of the subject comprises a mutation. In some cases, the PCSK9-related disorder mutation is a gain of function mutation, including, but not limited to mutations encoding amino acid substitutions selected from the group consisting of S127R, D129G, F216L, D374H, and D374Y relative to the sequence of SEQ ID NO:33. In other cases, the PCSK9-related disorder mutation is a loss of function mutation including, but not limited to mutations encoding amino acid substitutions selected from the group consisting of R46L, G106R, Y142X, N157K, R237W and C679X relative to the sequence of SEQ ID NO: 33. In other cases, the PCSK9-related disorder mutation comprises a PCKS9 allele disclosed in Table B. In other cases, the PCSK9 gene encodes a mutation that alters the function or expression of the PCSK9 protein such as, but not limited to, substitutions, deletions or insertions of one or more nucleotides as compared to the wild-type sequence.

In some embodiments, the disclosure provides methods of treating a PCSK9 or related disorder in a subject in need thereof comprising modifying a PCSK9 gene in a cell of the subject, the modifying comprising contacting said cells with a therapeutically effective dose of i) a composition comprising a CasX and a gNA of any of the embodiments described herein; ii) a composition comprising a CasX, a gNA, and a donor template of any of the embodiments described herein; iii) one or more nucleic acids encoding or comprising the compositions of (i) or (ii); iv) a vector selected from the group consisting of a retroviral vector, a lentiviral vector, an adenoviral vector, an adeno-associated viral (AAV) vector, a herpes simplex virus (HSV) vector and comprising the nucleic acids of (iii); v) a VLP comprising the composition of (i) or (ii); or vi) combinations of two or more of (i)-(v), wherein the PCSK9 gene of the cells is modified by the CasX protein and, optionally, the donor template such that a wild-type or a functional PCSK9 protein is expressed. In some embodiments of the method, a second gNA having a scaffold of any of the embodiments described herein is utilized, wherein the second gNA has a targeting sequence complementary to a different or overlapping portion of the target nucleic acid compared to the first gNA, resulting in an additional break in the PCSK9 target nucleic acid of the cells of the subject. In the foregoing, the gene can be modified by the NHEJ host repair mechanisms, or utilized in conjunction with a donor template that is inserted by HDR or HITI mechanisms to either excise or correct the mutation, resulting in the expression of a functional PCSK9 protein. The modified cell of the treated subject can be a eukaryotic cell selected from the group consisting of a rodent cell, a mouse cell, a rat cell, a primate cell, a non-human primate cell, and a human cell. In some embodiments, the eukaryotic cell of the treated subject is a human cell. In some embodiments, the cell is a cell involved in the production of LDL, including but not limited to a hepatocyte, or a cell of the intestine, the kidney, the central nervous system, a smooth muscle cell, macrophage, a retinal cell, or cell of arterial walls such as the endothelium. In some embodiments, the cell is an eye cell. In some embodiments, the cell comprises at least one modified allele of a PCSK9 gene in a cell wherein the modification is used to correct a mutation in the subject. In some cases, the mutation of the subject is a gain of function mutation. In other cases, the mutation of the subject is a loss of function mutation.

In some embodiments of the method of treatment, the method comprises administering to the subject a therapeutically effective dose of a vector of any of the embodiments described herein comprising or encoding the CasX protein and the gNA and, optionally, the donor template (described supra), wherein the contacting of the cells of the subject with the vector results in modification of the target nucleic acid of the cells by the CasX:gNA complex. In some embodiments, the method comprises administration of the vector comprising or encoding a CasX and a plurality of gNAs targeted to different locations in the PCSK9 gene, wherein the contacting of the cells of the subject with the CasX:gNA complexes results in modification of the target nucleic acid of the cells. In one particular embodiment, the vector is an AAV selected from the group consisting of AAV1, AAV2, AAV3, AAV4, AAV5, AAV6, AAV7, AAV8, AAV9, AAV10, AAV11, AAV12, AAV 44.9, AAV-Rh74, or AAVRh10. The vector of the embodiments are administered to the subject at a therapeutically effective dose. In some embodiments, the vector is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg. In other embodiments of the method, the VLP is administered to a subject at a dose of at least about 1×10⁵ particles/kg, at least about 1×10⁶ particles/kg, at least about 1×10⁷ particles/kg, at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, or at least about 1×10¹⁶ particles/kg. The vector or VLP can be administered by a route of administration selected from the group consisting of intravenous, intraportal vein injection, intraperitoneal, intramuscular, subcutaneous, intraocular, and oral routes. In some embodiments of the methods of treating a PCSK9-related disorder in a subject, the subject is selected from the group consisting of mouse, rat, pig, non-human primate, and human.

In other embodiments of the methods of treatment, the methods comprises further administering an additional CRISPR protein, or a polynucleotide encoding the additional CRISPR protein to the subject. In the foregoing embodiment, the additional CRISPR protein has a sequence different from the first CasX protein of the method. In some embodiments, the additional CRISPR protein is not a CasX protein; i.e., is a Cpf1, Cas9, Cas10, Cas12a, or Cas13a. In some cases, the gNA used in the method of treatment is a single-molecule gNA (sgNA). In other cases, the gNA is a dual-molecule gNA (dgNA). In still other cases, the method comprises contacting the target nucleic acid sequence with a plurality of gNAs targeted to different or overlapping sequences of the PCSK9 gene.

A number of therapeutic strategies have been used to design the compositions for use in the methods of treatment of a subject with a PCSK9-related disorder. In some embodiments, the invention provides a method of treatment of a subject having a PCSK9-related disorder, the method comprising administering to the subject a CasX:gNA composition or a vector of any of the embodiments disclosed herein according to a treatment regimen comprising one or more consecutive doses using a therapeutically effective dose. In some embodiments of the treatment regimen, the therapeutically effective dose of the composition or vector is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regiment, the effective doses are administered by a route selected from the group consisting of intravenous, intraportal vein injection, intraperitoneal, intramuscular, subcutaneous, intraocular, and oral routes.

In some embodiments of the method of treatment of a subject with a PCSK9-related disorder, the method comprises administering to the subject a CasX:gNA composition as an RNP within a VLP disclosed herein according to a treatment regimen comprising one or more consecutive doses using a therapeutically effective dose.

In some embodiments, the administering of the therapeutically effective amount of a CasX:gNA modality, including a vector comprising a polynucleotide encoding a CasX protein and a guide nucleic acid, or the administering of a CasX-gNA composition disclosed herein, to knock down or knock out expression of PCSK9 to a subject with a PCSK9-related disorder leads to the prevention or amelioration of the underlying PCSK9-related disorder such that an improvement is observed in the subject, notwithstanding that the subject may still be afflicted with the underlying disorder. In some embodiments, the administration of the therapeutically effective amount of the CasX-gNA modality leads to an improvement in at least one clinically-relevant endpoint including, but not limited to percent change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina, or visual acuity. In some embodiments, the administration of the therapeutically effective amount of the CasX-gNA modality leads to an improvement in at least two clinically-relevant endpoints. In some embodiments, the subject is selected from mouse, rat, pig, dog, non-human primate, and human.

In some embodiments, the methods of treatment further comprise administering a chemotherapeutic agent wherein the agent is effective in lowering LDL levels. Such agents include, but are not limited to, statins, niacin, fibrates, or anti-PCSK9 antibody drugs.

Methods of obtaining samples from treated subjects for analysis to determine the effectiveness of the treatment, such as body fluids or tissues, and methods of preparation of the samples to allow for analysis are well known to those skilled in the art. Methods for analysis of RNA and protein levels are discussed above and are well known to those skilled in the art. The effects of treatment can also be assessed by measuring biomarkers associated with the target gene expression in the aforementioned fluids, tissues or organs, collected from an animal contacted with one or more compounds of the invention, by routine clinical methods known in the art. Biomarkers of PCSK9 disorders include, but are not limited to, PCSK9 levels, low-density lipoprotein (LDL-cholesterol), apolipoprotein B, non-HDL cholesterol, triglycerides and lipoprotein a, soluble CD40 ligand, osteopontin (OPN), osteoprotegerin (OPG), matrix metalloproteinases (MMP) and myeloperoxidase (MPOP), wherein the concentration of the marker is compared to concentrations known to be physiologically normal or in subjects not having a PCSK9 disorder.

Several mouse models expressing mutant forms of PCSK9 exist and are suitable for evaluating the methods of treatment. Transgenic mouse models of PCSK9-related disorders include knock-in mouse models having hPCSK9 (Carreras, A. In vivo genome and base editing of a human PCSK9 knock-in hypercholesterolemic mouse model. MC Biology 17:4 (2019); Herbert B., et al. Increased secretion of lipoproteins in transgenic mice expressing human D374Y PCSK9 under physiological genetic control. Arterioscler Thromb Vasc Biol. 30(7):1333 (2010)).

### VIII. Pharmaceutical Compositions, Kits, and Articles of Manufacture

In some embodiments, the disclosure provides pharmaceutical compositions comprising: i) a CasX protein and one or a plurality of gNA of any of the embodiments of the disclosure comprising a targeting sequence specific for a PCSK9 gene; ii) one or more nucleic acids encoding the CasX and the gNA of (i); iii) a vector comprising the one or more nucleic acids of (ii); or iv) a VLP comprising an RNP of the CasX and gNA of (i); together with one or more pharmaceutically suitable excipients. In some embodiments, the pharmaceutical composition is formulated for a route of administration selected from the group consisting of intravenous, intraportal vein injection, intraperitoneal, intramuscular, subcutaneous, intraocular, and oral routes. In one embodiment, the pharmaceutical composition is in a liquid form or a frozen form. In another embodiment, the pharmaceutical composition is in a pre-filled syringe for a single injection. In another embodiment, the pharmaceutical composition is in solid form, for example the pharmaceutical composition is lyophilized.

In other embodiments, provided herein are kits comprising a CasX protein and one or a plurality of CasX gNA of any of the embodiments of the disclosure comprising a targeting sequence specific for a PCSK9 gene and a suitable container (for example a tube, vial or plate). In exemplary embodiments, a kit of the disclosure comprises a CasX variant of any one of SEQ ID NOS: 49-160, 439, 441, 443, 445, 447-460, 472, 474, 476, 478, 480, 482, 484, 486, 488, 490.

In some embodiments, the kit comprises a gNA or a vector encoding a gNA, wherein the gNA comprises a sequence selected from the group consisting of SEQ ID NOS: 247-303, 315-436, 612-2100, or 2286-13861. In some embodiments, the gNA comprises a sequence selected from the group consisting of SEQ ID NOS: 2101-2285. In some embodiments, the gNA comprises a sequence selected from the group consisting of SEQ ID NOS: 2236, 2237, 2238, 2241, 2244, 2248, 2249, and 2259-2285.

In certain embodiments, provided herein are kits comprising a CasX protein and gNA editing pair comprising a CasX variant protein of SEQ ID NOS: 49-160, 439, 441, 443, 445, 447-460, 472, 474, 476, 478, 480, 482, 484, 486, 488, 490 as set forth in Tables 3, 5, 6, 7 and 9 and a gNA variant as described herein (e.g., SEQ ID NOs: 2101-2285). In exemplary embodiments, a kit of the disclosure comprises a CasX and gNA editing pair, wherein the CasX variant comprises of any one of SEQ ID NOS: 49-160, 439, 441, 443, 445, 447-460, 472, 474, 476, 478, 480, 482, 484, 486, 488, 490. In some embodiments, the gNA of the gene editing pair comprises any one of SEQ ID NOS: 247-303, 315-436, 612-2100, or 2286-13861. In some embodiments, the gNA of the gene editing pair comprises a scaffold sequence of any one of SEQ ID NOS: 2101-2285 and a targeting sequence of any one of SEQ ID NOS: 247-303, 315-436, 612-2100, or 2286-13861. In some embodiments, the gNA of the gene editing pair comprises a scaffold sequence of any one of SEQ ID NOS: 2236, 2237, 2238, 2241, 2244, 2248, 2249, or 2259-2285 and a targeting sequence of any one of SEQ ID NOS: 247-303, 315-436, 612-2100, or 2286-13861.

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

In some embodiments, the kit comprises a vector comprising a sequence encoding a CasX protein of the disclosure, a CasX gNA of the disclosure, optionally a donor template, or a combination thereof.

### IX. Enumerated Embodiments

The present description sets forth numerous exemplary configurations, methods, parameters, and the like. It should be recognized, however, that such description is not intended as a limitation on the scope of the present disclosure, but is instead provided as a description of exemplary embodiments. Embodiments of the present subject matter described above may be beneficial alone or in combination, with one or more other aspects or embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

The inventions may be defined by reference to the following enumerated, illustrative embodiments:

Embodiment 1. A CasX:gNA system comprising a CasX protein and a guide nucleic acid (gNA), wherein the gNA comprises a targeting sequence complementary to a target nucleic acid sequence comprising a proprotein convertase subtilisin/kexin Type 9 (PCSK9) gene.

Embodiment 2. The CasX:gNA system of Embodiment 1, wherein the PCSK9 gene comprises one or more mutations.

Embodiment 3. The CasX:gNA system of Embodiment 1 or Embodiment 2, wherein the PCSK9 gene encodes a PCSK9 protein comprising one or more mutations.

Embodiment 4. The CasX:gNA system of Embodiment 3, wherein the one or more mutations comprise amino acid substitutions selected from the group consisting of S127R, D129G, F216L, D374H, and D374Y relative to the sequence of SEQ ID NO: 33.

Embodiment 5. The CasX:gNA system of any one of Embodiments 2-4, wherein the mutation is a gain-of-function mutation.

Embodiment 6. The CasX:gNA system of any one of the preceding Embodiments, wherein the gNA is a guide RNA (gRNA).

Embodiment 7. The CasX:gNA system of any one of Embodiments 1-6, wherein the gNA is a guide DNA (gDNA).

Embodiment 8. The CasX:gNA system of any one of Embodiments 1-6, wherein the gNA is a chimera comprising DNA and RNA.

Embodiment 9. The CasX:gNA system of any one of Embodiments 1-8, wherein the gNA is a single-molecule gNA (sgNA).

Embodiment 10. The CasX:gNA system of any one of Embodiments 1-8, wherein the gNA is a dual-molecule gNA (dgNA).

Embodiment 11. The CasX:gNA system of any one of Embodiments 1-10, wherein the targeting sequence of the gNA is complementary to a sequence comprising one or more single nucleotide polymorphisms (SNPs) of the PCSK9 gene.

Embodiment 12. The CasX:gNA system of any one of Embodiments 1-10, wherein the targeting sequence of the gNA comprises a sequence selected from the group consisting of SEQ ID NOS: 247-303, 315-436, 612-2100, and 2286-13861.

Embodiment 13. The CasX:gNA system of any one of Embodiments 1-10, wherein the targeting sequence of the gNA comprises a sequence of SEQ ID NOS: 247-303, 315-436, 612-2100, or 2286-13861 with a single nucleotide removed from the 3′ end of the sequence.

Embodiment 14. The CasX:gNA system of any one of Embodiments 1-10, wherein the targeting sequence of the gNA comprises a sequence of SEQ ID NOS: 247-303, 315-436, 612-2100, or 2286-13861 with two nucleotides removed from the 3′ end of the sequence.

Embodiment 15. The CasX:gNA system of any one of Embodiments 1-10, wherein the targeting sequence of the gNA comprises a sequence of SEQ ID NOS: 247-303, 315-436, 612-2100, or 2286-13861 with three nucleotides removed from the 3′ end of the sequence.

Embodiment 16. The CasX:gNA system of any one of Embodiments 1-10, wherein the targeting sequence of the gNA comprises a sequence of SEQ ID NOS: 247-303, 315-436, 612-2100, or 2286-13861 with four nucleotides removed from the 3′ end of the sequence.

Embodiment 17. The CasX:gNA system of any one of Embodiments 1-10, wherein the targeting sequence of the gNA comprises a sequence of SEQ ID NOS: 247-303, 315-436, 612-2100, or 2286-13861 with five nucleotides removed from the 3′ end of the sequence.

Embodiment 18. The CasX:gNA system of any one of Embodiments 1-10, wherein the targeting sequence of the gNA comprises a sequence having at least about 65%, at least about 75%, at least about 85%, or at least about 95% identity to a sequence selected from the group consisting of SEQ ID NOS: 247-303, 315-436, 612-2100, and 2286-13861.

Embodiment 19. The CasX:gNA system of any one of Embodiments 1-10, wherein the targeting sequence of the gNA comprises a sequence having one or more single nucleotide polymorphisms (SNP) relative to a sequence of SEQ ID NOS: 247-303, 315-436, 612-2100, or 2286-13861.

Embodiment 20. The CasX:gNA system of any one of Embodiments 1-19, wherein the targeting sequence of the gNA is complementary to a non-coding region of the PCSK9 gene.

Embodiment 21. The CasX:gNA system of any one of Embodiments 1-19, wherein the targeting sequence of the gNA is complementary to a coding region of the PCSK9 gene.

Embodiment 22. The CasX:gNA system of any one of Embodiments 1-19, wherein the targeting sequence of the gNA is complementary to a sequence of a PCSK9 exon.

Embodiment 23. The CasX:gNA system of any one of Embodiments 1-19, wherein the targeting sequence of the gNA is complementary to a sequence of a PCSK9 intron.

Embodiment 24. The CasX:gNA system of any one of Embodiments 1-19, wherein the targeting sequence of the gNA is complementary to a sequence of a PCSK9 intron-exon junction.

Embodiment 25. The CasX:gNA system of any one of Embodiments 1-19, wherein the targeting sequence of the gNA is complementary to a sequence of a PCSK9 regulatory region.

Embodiment 26. The CasX:gNA system of any one of Embodiments 1-19, wherein the targeting sequence of the gNA is complementary to a sequence of an intergenic region of the PCSK9 gene.

Embodiment 27. The CasX:gNA system of any one of Embodiments 1-26, further comprising a second gNA, wherein the second gNA has a targeting sequence complementary to a different or overlapping portion of the target nucleic acid sequence compared to the targeting sequence of the gNA of any one of the preceding Embodiments.

Embodiment 28. The CasX:gNA system of any one of Embodiments 1-27, wherein the gNA has a scaffold comprising a sequence selected from the group consisting of SEQ ID NOS: 4-16 and 2101-2285, or a sequence having at least about 50%, at least about 60%, at least about 70%, at least about 80%, at least about 90%, at least about 95%, at least about 95%, at least about 96%, at least about 97%, at least about 98%, or at least about 99% sequence identity thereto.

Embodiment 29. The CasX:gNA system of any one of Embodiments 1-28, wherein the gNA has a scaffold comprising a sequence having at least one modification relative to a reference gNA sequence selected from the group consisting of the sequences of SEQ ID NOS: 4-16.

Embodiment 30. The CasX:gNA system of Embodiment 29, wherein the at least one modification of the reference gNA comprises at least one substitution, deletion, or insertion of a nucleotide of the gNA sequence.

Embodiment 31. The CasX:gNA system of any one of Embodiments 1-30, wherein the gNA is chemically modified.

Embodiment 32. The CasX:gNA system of any one of Embodiments 1-31, wherein the CasX protein comprises a reference CasX protein having a sequence of any one of SEQ ID NOS: 1-3, a CasX variant protein having a sequence of SEQ ID NOS: 49-160, 439, 441, 443, 445, 447-460, 472, 474, 476, 478, 480, 482, 484, 486, 488, or 490, or a sequence having at least about 50%, at least about 60%, at least about 70%, at least about 80%, at least about 90%, at least about 95%, at least about 96%, at least about 97%, at least about 98%, or at least about 99% sequence identity thereto.

Embodiment 33. The CasX:gNA system of Embodiment 32, wherein the CasX protein has binding affinity for a protospacer adjacent motif (PAM) sequence selected from the group consisting of TTC, ATC, GTC, and CTC.

Embodiment 34. The CasX:gNA system of Embodiment 32 or Embodiment 33, wherein the CasX variant protein comprises at least one modification relative to a reference CasX protein having a sequence selected from SEQ ID NOS:1-3.

Embodiment 35. The CasX:gNA system of Embodiment 34, wherein the at least one modification comprises at least one amino acid substitution, deletion, or insertion in a domain of the CasX variant protein relative to the reference CasX protein.

Embodiment 36. The CasX:gNA system of Embodiment 35, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

Embodiment 37. The CasX:gNA system of any one of Embodiments 32-36, wherein the CasX protein further comprises one or more nuclear localization signals (NLS).

Embodiment 38. The CasX:gNA system of Embodiment 37, wherein the one or more NLS are selected from the group of sequences consisting of PKKKRKV (SEQ ID NO: 217), KRPAATKKAGQAKKKK (SEQ ID NO: 223), PAAKRVKLD (SEQ ID NO: 224), RQRRNELKRSP (SEQ ID NO: 161), NQSSNFGPMKGGNFGGRSSGPYGGGGQYFAKPRNQGGY (SEQ ID NO: 162), RMRIZFKNKGKDTAELRRRRVEVSVELRKAKKDEQILKRRNV (SEQ ID NO: 163), VSRKRPRP (SEQ ID NO: 164), PPKKARED (SEQ ID NO: 165), PQPKKKPL (SEQ ID NO: 166), SALIKKKKKMAP (SEQ ID NO: 167), DRLRR (SEQ ID NO: 168), PKQKKRK (SEQ ID NO: 169), RKLKKKIKKL (SEQ ID NO: 170), REKKKFLKRR (SEQ ID NO: 171), KRKGDEVDGVDEVAKKKSKK (SEQ ID NO: 172), RKCLQAGMNLEARKTKK (SEQ ID NO: 173), PRPRKIPR (SEQ ID NO: 174), PPRKKRTVV (SEQ ID NO: 175), NLSKKKKRKREK (SEQ ID NO: 176), RRPSRPFRKP (SEQ ID NO: 177), KRPRSPSS (SEQ ID NO: 178), KRGINDRNFWRGENERKTR (SEQ ID NO: 179), PRPPKMARYDN (SEQ ID NO: 180), KRSFSKAF (SEQ ID NO: 181), KLKIKRPVK (SEQ ID NO: 182), PKTRRRPRRSQRKRPPT (SEQ ID NO: 184), RRKKRRPRRKKRR (SEQ ID NO: 187), PKKKSRKPKKKSRK (SEQ ID NO: 188), HKKKHPDASVNFSEFSK (SEQ ID NO: 189), QRPGPYDRPQRPGPYDRP (SEQ ID NO: 190), LSPSLSPLLSPSLSPL (SEQ ID NO: 191), RGKGGKGLGKGGAKRHRK (SEQ ID NO: 192), PKRGRGRPKRGRGR (SEQ ID NO: 193), MSRRRKANPTKLSENAKKLAKEVEN (SEQ ID NO: 185), PKKKRKVPPPPAAKRVKLD (SEQ ID NO: 183), and PKKKRKVPPPPKKKRKV (SEQ ID NO: 194).

Embodiment 39. The CasX:gNA system of Embodiment 37 or Embodiment 38, wherein the one or more NLS are at the C-terminus of the CasX protein.

Embodiment 40. The CasX:gNA system of Embodiment 37 or Embodiment 38, wherein the one or more NLS are at the N-terminus of the CasX protein.

Embodiment 41. The CasX:gNA system of Embodiment 37 or Embodiment 38, wherein the one or more NLS are at the N-terminus and C-terminus of the CasX protein.

Embodiment 42. The CasX:gNA system of any one of Embodiments 32-41, wherein the CasX variant protein and the gNA exhibit at least one or more improved characteristics as compared to a reference CasX protein and the gNA.

Embodiment 43. The CasX:gNA system of Embodiment 42, wherein the improved characteristic is selected from the group consisting of improved folding of the CasX protein, improved binding affinity of the CasX protein to the gNA, improved ribonuclear protein complex (RNP) formation, higher percentage of cleavage-competent RNP, improved binding affinity to the target nucleic acid sequence, altered binding affinity to one or more PAM sequences, improved unwinding of the target nucleic acid sequence, increased activity, increased target nucleic acid sequence cleavage rate, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved CasX protein stability, improved protein:guide RNA complex stability, improved protein solubility, improved protein:gNA complex solubility, improved protein yield, improved protein expression, and improved fusion characteristics.

Embodiment 44. The CasX:gNA system of Embodiment 42 or Embodiment 43, wherein the improved characteristic of the CasX variant protein is at least about 1.1 to about 100,000-fold improved relative to the reference CasX protein of SEQ ID NO: 1, SEQ ID NO: 2, or SEQ ID NO: 3.

Embodiment 45. The CasX:gNA system of Embodiment 42 or Embodiment 43, wherein the improved characteristic of the CasX variant protein is at least about 10-fold, at least about 100-fold, at least about 1,000-fold, or at least about 10,000-fold improved relative to the reference CasX protein of SEQ ID NO: 1, SEQ ID NO: 2, or SEQ ID NO: 3.

Embodiment 46. The CasX:gNA system of any one of Embodiments 43-45, wherein the improved characteristic is improved binding affinity to the target nucleic acid sequence.

Embodiment 47. The CasX:gNA system of any one of Embodiments 43-45, wherein the improved characteristic is increased target nucleic acid sequence cleavage rate.

Embodiment 48. The CasX:gNA system of any one of Embodiments 43-45, wherein the improved characteristic is increased binding affinity to one or more PAM sequences wherein the one or more PAM sequences are selected from the group consisting of TTC, ATC, GTC, and CTC.

Embodiment 49. The CasX:gNA system of any one of the preceding Embodiments, wherein the CasX variant protein and the gNA are associated together in an RNP.

Embodiment 50. The CasX:gNA system of Embodiment 49, wherein the RNP has a higher percentage of cleavage-competent RNP compared to an RNP of a reference CasX and the gNA.

Embodiment 51. The CasX:gNA system of any one of Embodiments 32-50, wherein the CasX variant protein comprises a nuclease domain having nickase activity.

Embodiment 52. The CasX:gNA system of Embodiment 51, wherein the CasX variant can cleave only one strand of a double-stranded target nucleic acid molecule.

Embodiment 53. The CasX:gNA system of any one of Embodiments 1-50, wherein the CasX variant protein comprises a nuclease domain having double-stranded cleavage activity.

Embodiment 54. The CasX:gNA system of any one of Embodiments 1-41, wherein the CasX protein is a catalytically inactive CasX (dCasX) protein, and wherein the dCasX and the gNA retain the ability to bind to the target nucleic acid sequence.

Embodiment 55. The CasX:gNA system of Embodiment 54, wherein the dCasX comprises a mutation at residues:

a) D672, E769, and/or D935 corresponding to the reference CasX protein of SEQ ID NO:1; or

b) D659, E756 and/or D922 corresponding to the reference CasX protein of SEQ ID NO: 2.

Embodiment 56. The CasX:gNA system of Embodiment 55, wherein the mutation is a substitution of alanine for the residue.

Embodiment 57. The CasX:gNA system of any one of Embodiments 1-53, further comprising a donor template nucleic acid.

Embodiment 58. The CasX:gNA system of Embodiment 57, wherein the donor template comprises a nucleic acid comprising at least a portion of the PCSK9 gene, wherein the PCSK9 gene portion is selected from the group consisting of a PCSK9 exon, a PCSK9 intron, a PCSK9 intron-exon junction, the PCSK9 regulatory region, or a combination thereof.

Embodiment 59. The CasX:gNA system of Embodiment 57 or Embodiment 58, wherein the donor template comprises homologous arms complementary to sequences flanking a cleavage site in the target nucleic acid.

Embodiment 60. The CasX:gNA system of Embodiment 57-59, wherein the donor template ranges in size from 10-15,000 nucleotides.

Embodiment 61. The CasX:gNA system of any one of Embodiments 57-60, wherein the donor template is a single-stranded DNA template or a single stranded RNA template.

Embodiment 62. The CasX:gNA system of any one of Embodiments 57-60, wherein the donor template is a double-stranded DNA template.

Embodiment 63. The CasX:gNA system of any one of Embodiments 57-62, wherein the donor template comprises one or more mutations compared to a wild-type PCSK9 gene.

Embodiment 64. The CasX:gNA system of any one of Embodiments 57-62, wherein the donor template comprises a heterologous sequence compared to a wild-type PCSK9 gene.

Embodiment 65. The CasX:gNA system of any one of Embodiments 57-62, wherein the donor template comprises all or a portion of a wild-type PCSK9 gene.

Embodiment 66. A nucleic acid comprising a sequence that encodes the CasX:gNA system of any one of Embodiments 1-56.

Embodiment 67. The nucleic acid of Embodiment 66, wherein the sequence encoding the CasX protein is codon optimized for expression in a eukaryotic cell.

Embodiment 68. A vector comprising the nucleic acid of Embodiment 66 or Embodiment 67.

Embodiment 69. The vector of Embodiment 68, wherein the vector further comprises a promoter.

Embodiment 70. A vector comprising a donor template, wherein the donor template comprises a nucleic acid comprising at least a portion of a PCSK9 gene, wherein the PCSK9 gene portion is selected from the group consisting of a PCSK9 exon, a PCSK9 intron, a PCSK9 intron-exon junction, and a PCSK9 regulatory region.

Embodiment 71. The vector of Embodiment 70, wherein the donor template comprises one or more mutations compared to a wild-type PCSK9 gene.

Embodiment 72. The vector of Embodiment 70 or Embodiment 71, further comprising the nucleic acid of Embodiment 66 or Embodiment 67.

Embodiment 73. The vector of any one of Embodiments 68-70, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a plasmid, a minicircle, a nanoplasmid, and an RNA vector.

Embodiment 74. The vector of Embodiment 73, wherein the vector is an AAV vector.

Embodiment 75. The vector of Embodiment 74, wherein the AAV vector is selected from AAV1, AAV2, AAV3, AAV4, AAV5, AAV6, AAV7, AAV8, AAV9, AAV10, AAV-Rh74, or AAVRh10.

Embodiment 76. The vector of Embodiment 73, wherein the vector is a retroviral vector.

Embodiment 77. The vector of Embodiment 73, wherein the vector encoding the VLP comprises one or more nucleic acids encoding a gag polyprotein, the CasX protein of any one of Embodiments 32-56, and the gNA of any one of Embodiments 1-31.

Embodiment 78. A virus-like particle (VLP) comprising the CasX protein of any one of Embodiments 32-56, and the gNA of any one of Embodiments 1-31.

Embodiment 79. The VLP of Embodiment 78, wherein the CasX protein and the gNA are associated together in an RNP.

Embodiment 80. A method of modifying a PCSK9 target nucleic acid sequence, the method comprising contacting the target nucleic acid sequence with a CasX protein and a guide nucleic acid (gNA) comprising a targeting sequence wherein said contacting comprises introducing into a cell:

a) the CasX:gNA system of any one of Embodiments 1-65;

b) the nucleic acid of Embodiment 66 or Embodiment 67;

c) the vector as in any one of Embodiments 68-77;

d) the VLP of Embodiment 78 or Embodiment 79; or

e) combinations thereof,

Embodiment 81. wherein said contacting results in modification of the PCSK9 target nucleic acid sequence by the CasX protein.

Embodiment 82. The method of Embodiment 80, wherein the CasX protein and the gNA are associated together in a ribonuclear protein complex (RNP).

Embodiment 83. The method of Embodiment 80 or Embodiment 81, further comprising a second gNA or a nucleic acid encoding the second gNA, wherein the second gNA has a targeting sequence complementary to a different portion of the target nucleic acid sequence or its complement.

Embodiment 84. The method any one of Embodiments 80-82, wherein the PCSK9 gene comprises a mutation.

Embodiment 85. The method of Embodiment 83, wherein the mutation is a gain of function mutation.

Embodiment 86. The method of any one of Embodiments 80-82, wherein the PCSK9 gene comprises a wild-type sequence.

Embodiment 87. The method of any one of Embodiments 80-85, wherein the modifying comprises introducing a single-stranded break in the target nucleic acid sequence.

Embodiment 88. The method of any one of Embodiments 80-85, wherein the modifying comprises introducing a double-stranded break in the target nucleic acid sequence.

Embodiment 89. The method of any one of Embodiments 80-87, wherein the modifying comprises introducing an insertion, deletion, substitution, duplication, or inversion of one or more nucleotides in the target nucleic acid sequence as compared to the wild-type sequence.

Embodiment 90. The method of any one of Embodiments 80-88, wherein the modifying of the target nucleic acid sequence occurs inside of a cell.

Embodiment 91. The method of any one of Embodiments 80-89, wherein the modifying of the target nucleic acid sequence occurs in vivo.

Embodiment 92. The method of any one of Embodiments 80-90, wherein the cell is a eukaryotic cell.

Embodiment 93. The method of Embodiment 91, wherein the eukaryotic cell is selected from the group consisting of a rodent cell, a mouse cell, a rat cell, a pig cell, a primate cell, a non-human primate cell, and a human cell.

Embodiment 94. The method of Embodiment 92, wherein the eukaryotic cell is a human cell.

Embodiment 95. The method of any one of Embodiments 80-94, wherein the cell is selected from the group consisting of a hepatocyte, a cell of the intestine, a cell of the kidney, a cell of the central nervous system, a smooth muscle cell, a macrophage, and an arterial endothelial cell.

Embodiment 96. The method of any one of Embodiments 80-94, wherein the method further comprises contacting the target nucleic acid sequence with a donor template complementary to at least a portion of a PCSK9 gene, wherein the donor template is inserted into the target nucleic acid sequence to replace all or a portion of the target nucleic acid sequence.

Embodiment 97. The method of Embodiment 96, wherein the donor template comprises one or more mutations compared to the wild-type PCSK9 gene sequence, and wherein the insertion results in a knock-down or knock-out of the PCSK9 gene.

Embodiment 98. The method of Embodiment 96, wherein the donor template comprises all or a portion of a wild-type PCSK9 gene sequence, wherein the insertion corrects one or more mutation(s) of the PCSK9 gene.

Embodiment 99. The method of any one of Embodiments 96-98, wherein the donor template ranges in size from 10-15,000 nucleotides.

Embodiment 100. The method of any one of Embodiments 96-98, wherein the donor template ranges in size from 100-1,000 nucleotides.

Embodiment 101. The method of any one of Embodiments 96-100, wherein the donor template is a single-stranded DNA template or a single stranded RNA template.

Embodiment 102. The method of any one of Embodiments 96-100, wherein the donor template is a double-stranded DNA template.

Embodiment 103. The method of any one of Embodiments 96-102, wherein the donor template is inserted by homology directed repair (HDR).

Embodiment 104. The method of any one of Embodiments 80-103, wherein the vector is administered to a subject at a therapeutically effective dose.

Embodiment 105. The method of Embodiment 104, wherein the subject is selected from the group consisting of mouse, rat, pig, non-human primate, and human.

Embodiment 106. The method of Embodiment 104, wherein the subject is a human.

Embodiment 107. The method of any one of Embodiments 80-106, wherein the vector is administered at a dose of at least about 1×1010 vector genomes (vg), or at least about 1×10¹¹ vg, or at least about 1×10¹² vg, or at least about 1×10¹³ vg, or at least about 1×10¹⁴ vg, or at least about 1×10¹⁵ vg, or at least about 1×10¹⁶ vg.

Embodiment 108. The method of any one of Embodiments 80-106, wherein the vector is administered by a route of administration selected from the group consisting of intravenous, intraportal vein injection, intraperitoneal, intramuscular, subcutaneous, and oral routes.

Embodiment 109. The method of any one of Embodiments 80-108, comprising further contacting the target nucleic acid sequence with an additional CRISPR protein, or a polynucleotide encoding the additional CRISPR protein.

Embodiment 110. The method of Embodiment 109, wherein the additional CRISPR protein is a CasX protein having a sequence different from the CasX protein of any of the preceding Embodiments.

Embodiment 111. The method of Embodiment 109, wherein the additional CRISPR protein is not a CasX protein.

Embodiment 112. A method of altering a PCSK9 target nucleic acid sequence of a cell, comprising contacting said cell with:

a) the CasX:gNA system of any one of Embodiments 1-65;

b) the nucleic acid of Embodiment 66 or Embodiment 67;

c) the vector of any one of Embodiments 68-77;

d) the VLP of Embodiment 78 or Embodiment 79; or

e) combinations thereof.

Embodiment 113. The method of Embodiment 112, wherein the cell has been modified such that expression of the PCSK9 protein is reduced by at least about 10%, at least about 20%, at least about 30%, at least about 40%, at least about 50%, at least about 60%, at least about 70%, at least about 80%, or at least about 90% in comparison to a cell that has not been modified.

Embodiment 114. The method of Embodiment 112 or Embodiment 113, wherein the cell has been modified such that the cell does not express a detectable level of the PCSK9 protein.

Embodiment 115. A population of cells modified by the method of Embodiment 112 or Embodiment 113, wherein the cells have been modified such that at least 10%, at least about 20%, at least about 30%, at least about 40%, at least about 50%, at least about 60%, at least about 70%, at least about 80%, or at least about 90% of the modified cells do not express a detectable level of PCSK9 protein.

Embodiment 116. The population of cells of Embodiment 115, wherein the cell is a non-primate mammalian cell, a non-human primate cell, or a human cell.

Embodiment 117. The population of cells of Embodiment 115 or Embodiment 116, wherein the cells are selected from the group consisting of hepatocytes, cells of the intestine, cells of the kidney, cells of the central nervous system, smooth muscle cells, macrophages, and arterial endothelial cells.

Embodiment 118. A method of treating a PCSK9-related disorder in a subject in need thereof, comprising modifying a PCSK9 gene in a cell of the subject, the modifying comprising either contacting said cell with;

a) CasX:gNA system of any one of Embodiments 1-65;

b) the nucleic acid of Embodiment 66 or Embodiment 67;

c) the vector as in any one of Embodiments 68-77;

d) the VLP of Embodiment 78 or Embodiment 79; or

e) combinations thereof.

Embodiment 119. The method of Embodiment 118, wherein the PCSK9-related disorder is selected from the group consisting of autosomal dominant hypercholesterolemia (ADH), hypercholesterolemia, elevated LDL, atherosclerotic cardiovascular disease, and coronary artery disease.

Embodiment 120. The method of Embodiment 118 or Embodiment 119, further comprising a second gNA or a nucleic acid encoding the second gNA, wherein the second gNA has a targeting sequence complementary to a different or overlapping portion of the target nucleic acid sequence.

Embodiment 121. The method of any one of Embodiments 118-120, wherein the modifying introduces one or more mutations in the PCSK9 gene, or wherein expression of the PCSK9 protein is inhibited or suppressed.

Embodiment 122. The method of any one of Embodiments 118-121, wherein the method comprises contacting the cell with the donor template of any one of Embodiments 57-65.

Embodiment 123. The method of any one of Embodiments 118-122, wherein the cell is selected from the group consisting of a hepatocyte, a cell of the intestine, a cell of the kidney, a cell of the central nervous system, a smooth muscle cell, a macrophage, and arterial endothelial cell.

Embodiment 124. The method of any one of Embodiments 118-123, wherein the vector is administered to a subject at a therapeutically effective dose.

Embodiment 125. The method of Embodiment 124, wherein the subject is selected from the group consisting of mouse, rat, pig, non-human primate, and human.

Embodiment 126. The method of Embodiment 124, wherein the subject is a human.

Embodiment 127. The method of any one of Embodiments 118-126, wherein the vector is administered to the subject at a dose of at least about 1×10¹⁰ vector genomes (vg), or at least about 1×10¹¹ vg, or at least about 1×10¹² vg, or at least about 1×10¹³ vg, or at least about 1×10¹⁴ vg, or at least about 1×10¹⁵ vg, or at least about 1×10¹⁶ vg.

Embodiment 128. The method of any one of Embodiments 118-127, wherein the vector is administered by a route of administration selected from the group consisting of intravenous, intraportal vein injection, intraperitoneal, intramuscular, subcutaneous, and oral routes.

Embodiment 129. The method of any one of Embodiments 118-128, comprising further contacting the target nucleic acid sequence with an additional CRISPR protein, or a polynucleotide encoding the additional CRISPR protein.

Embodiment 130. The method of Embodiment 129, wherein the additional CRISPR protein is a CasX protein having a sequence different from the CasX of any of the preceding Embodiments.

Embodiment 131. The method of Embodiment 130, wherein the additional CRISPR protein is not a CasX protein.

Embodiment 132. The method of any one of Embodiments 118-131, wherein the method further comprises administering a chemotherapeutic agent.

Embodiment 133. The method of any one of Embodiments 118-132, wherein the method results in improvement in at least one clinically-relevant endpoint selected from the group consisting of percent change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, and unstable angina.

Embodiment 134. The method of any one of Embodiments 118-132, wherein the method results in improvement in at least two clinically-relevant endpoints selected from the group consisting of percent change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, or unstable angina.

The following Examples are merely illustrative and are not meant to limit any aspects of the present disclosure in any way.

### EXAMPLES

### Example 1: Creation, Expression and Purification of CasX Stx2

### 1. Growth and Expression

An expression construct for CasX Stx2 (also referred to herein as CasX2), derived from Planctomycetes (comprising the CasX amino acid sequence of SEQ ID NO: 2 and encoded by the sequence of SEQ ID NO: 437, below), was constructed from gene fragments (Twist Biosciences) that were codon optimized for E. coli. The assembled construct contains a TEV-cleavable, C-terminal, TwinStrep tag and was cloned into a pBR322-derivative plasmid backbone containing an ampicillin resistance gene. The expression construct was transformed into chemically competent BL21* (DE3) E. coli and a starter culture was grown overnight in LB broth supplemented with carbenicillin at 37° C., 200 RPM, in UltraYield Flasks (Thomson Instrument Company). The following day, this culture was used to seed expression cultures at a 1:100 ratio (starter culture:expression culture). Expression cultures were Terrific Broth (Novagen) supplemented with carbenicillin and grown in UltraYield flasks at 37° C., 200 RPM. Once the cultures reached an optical density (OD) of 2, they were chilled to 16° C. and IPTG (isopropyl β-D-1-thiogalactopyranoside) was added to a final concentration of 1 mM, from a 1 M stock. The cultures were induced at 16° C., 200 RPM for 20 hours before being harvested by centrifugation at 4,000×g for 15 minutes, 4° C. The cell paste was weighed and resuspended in lysis buffer (50 mM HEPES-NaOH, 250 mM NaCl, 5 mM MgCl₂, 1 mM TCEP, 1 mM benzamidine-HCL, 1 mM PMSF, 0.5% CHAPS, 10% glycerol, pH 8) at a ratio of 5 mL of lysis buffer per gram of cell paste. Once resuspended, the sample was frozen at −80° C. until purification.

TABLE 4
DNA sequence of CasX Stx2 construct

| Construct | DNA Sequence |
|---|---|
| SV40 NLS-CasX-SV40 | ATGGCTCCGAAGAAGAAGCGAAAGGTCAGCCAGGAAATTAAACGCATCAA |
| NLS-TEV | CAAGATCCGCCGTCGTCTGGTAAAAGACAGCAATACGAAAAAAGCCGGAA |
| cleavage site- | AAACCGGTCCGATGAAAACGCTGCTGGTGCGCGTGATGACGCCGGATCTC |
| TwinStrep tag | CGCGAACGTCTTGAGAATTTGCGTAAGAAACCTGAAAATATTCCGCAACC |
| | GATTTCTAACACCTCGCGCGCCAATCTGAATAAACTGCTGACCGATTACA |
| | CCGAAATGAAGAAAGCGATTCTGCACGTTTACTGGGAAGAGTTCCAGAAA |
| | GACCCGGTCGGTCTGATGAGCCGCGTTGCGCAACCTGCGCCGAAAAATAT |
| | CGATCAGCGCAAGTTAATCCCGGTTAAAGATGGTAATGAACGTTTAACCT |
| | CCAGCGGCTTTGCCTGCAGTCAGTGCTGCCAGCCACTTTATGTTTATAAA |
| | CTTGAACAGGTTAACGATAAAGGGAAACCCCATACCAATTATTTCGGCCG |
| | CTGCAATGTCAGCGAACATGAACGCCTGATTTTGTTAAGCCCGCATAAAC |
| | CGGAAGCGAATGACGAACTGGTGACCTATTCCCTGGGTAAATTTGGTCAG |
| | CGGGCGCTGGATTTTTACAGCATTCATGTGACGCGGGAAAGTAACCATCC |
| | GGTAAAGCCACTGGAACAAATCGGCGGTAACAGCTGCGCCTCTGGCCCGG |
| | TTGGCAAAGCGCTTAGCGATGCCTGTATGGGCGCGGTGGCGAGCTTTCTG |
| | ACAAAATACCAGGATATTATCCTGGAGCATCAGAAGGTGATCAAAAAGAA |
| | CGAGAAACGTCTGGCAAATTTAAAGGATATTGCCTCCGCTAACGGCCTGG |
| | CGTTCCCGAAGATTACCTTACCGCCGCAGCCGCACACCAAAGAAGGTATC |
| | GAAGCGTATAACAACGTTGTTGCCCAGATCGTCATCTGGGTGAATCTCAA |
| | CCTGTGGCAAAAACTGAAAATTGGTCGTGATGAAGCAAAACCGTTGCAGC |
| | GACTGAAAGGATTCCCGTCGTTTCCGCTGGTTGAACGACAGGCGAACGAA |
| | GTGGATTGGTGGGATATGGTTTGTAACGTCAAAAAATTGATCAACGAAAA |
| | AAAGGAAGATGGCAAAGTTTTCTGGCAAAATCTGGCGGGTTACAAACGTC |
| | AGGAGGCGTTGCTTCCGTATCTCTCTTCAGAAGAAGATCGCAAAAAAGGC |
| | AAGAAGTTTGCTCGCTATCAGTTTGGCGATTTATTACTGCATCTGGAAAA |
| | AAAACACGGCGAAGACTGGGGCAAAGTGTACGATGAAGCCTGGGAGCGTA |
| | TCGACAAAAAAGTGGAAGGTTTGTCGAAACATATTAAACTCGAAGAAGAG |
| | CGCCGCAGTGAAGATGCGCAGTCAAAAGCAGCGCTGACGGACTGGTTACG |
| | TGCGAAAGCCAGTTTTGTGATTGAAGGATTAAAAGAAGCTGATAAAGATG |
| | AATTTTGCCGTTGCGAACTGAAACTGCAAAAATGGTATGGCGACCTGCGC |
| | GGCAAACCGTTCGCCATTGAGGCAGAAAATAGCATCCTTGATATCTCCGG |
| | TTTCAGCAAACAATATAACTGCGCGTTTATTTGGCAGAAAGACGGCGTGA |
| | AAAAGCTTAACCTGTATCTGATCATTAACTATTTTAAAGGCGGGAAACTG |
| | CGTTTCAAGAAAATCAAGCCGGAAGCATTTGAAGCCAATCGTTTTTATAC |
| | CGTTATTAATAAAAAAAGCGGTGAAATCGTGCCGATGGAAGTTAATTTTA |
| | ACTTTGATGATCCGAACTTGATTATTCTGCCGCTGGCATTCGGTAAACGG |
| | CAGGGCCGTGAGTTTATCTGGAACGACCTGTTATCGCTGGAAACGGGCAG |
| | CCTGAAATTAGCCAACGGTCGCGTCATTGAAAAAACGCTCTACAACCGCC |
| | GCACCCGCCAGGATGAGCCGGCACTGTTTGTCGCGCTGACCTTTGAACGG |
| | CGTGAAGTCCTCGATAGCAGCAACATCAAACCAATGAACCTTATCGGTAT |
| | TGATCGTGGTGAAAACATTCCTGCCGTTATCGCCCTGACTGATCCAGAAG |
| | GCTGCCCGCTTTCTCGCTTCAAAGATTCACTGGGCAACCCGACCCATATC |
| | CTCCGTATTGGCGAGAGCTACAAAGAGAAACAGCGTACCATTCAGGCAGC |
| | CAAAGAAGTGGAGCAGCGTCGCGCGGGCGGCTATAGCCGTAAATATGCCA |
| | GCAAAGCTAAAAACCTGGCGGATGACATGGTGCGTAACACGGCGCGCGAT |
| | TTGCTGTACTACGCCGTCACCCAGGACGCGATGCTGATTTTTGAGAACCT |
| | CTCCCGCGGTTTTGGGCGTCAGGGTAAACGCACGTTTATGGCGGAACGCC |
| | AGTATACGCGTATGGAGGACTGGCTGACCGCGAAGCTGGCCTATGAAGGC |
| | TTGCCGTCTAAAACTTACCTGAGCAAGACCCTGGCTCAGTACACCAGTAA |
| | AACCTGTAGTAATTGCGGCTTTACCATCACCAGCGCCGATTATGACCGCG |
| | TGCTGGAAAAGCTGAAGAAAACCGCCACCGGCTGGATGACCACCATCAAT |
| | GGTAAAGAGCTTAAAGTCGAAGGGCAGATTACTTATTACAACCGTTATAA |
| | GCGGCAAAACGTGGTGAAAGATCTGTCGGTTGAGCTGGACCGTTTGTCTG |
| | AAGAAAGCGTGAACAATGATATCAGCTCCTGGACCAAAGGTCGTTCCGGC |
| | GAAGCGTTAAGTCTGTTGAAAAAGCGCTTTAGCCATCGCCCGGTGCAGGA |
| | AAAATTCGTTTGCCTGAACTGTGGCTTCGAAACCCACGCCGACGAGCAAG |
| | CGGCGCTCAATATTGCGCGTAGCTGGCTGTTCCTGCGCAGCCAGGAATAT |
| | AAAAAATATCAAACCAACAAAACAACTGGCAATACCGAGAAGCGTGCCTT |
| | TGTTGAAACCTGGCAGAGCTTCTATCGCAAAAAACTGAAAGAGGTCTGGA |
| | AACCGGCGGTAGCGCCAAAGAAAAAACGCAAAGTGAGCGAAAATCTTTAT |
| | TTTCAAGGTAGCGCATGGAGTCATCCTCAATTCGAGAAAGGTGGAGGTTC |
| | TGGCGGTGGATCGGGAGGTTCAGCGTGGAGCCACCCGCAGTTCGAAAAAG |
| | GAAGGGGATCCGGCTGCTAA (SEQ ID NO: 437) |

### 2. Purification

Frozen samples were thawed overnight at 4° C. with magnetic stirring. The viscosity of the resulting lysate was reduced by sonication and lysis was completed by homogenization in three passes at 17 k PSI using an Emulsiflex C3 (Avestin). Lysate was clarified by centrifugation at 50,000×g, 4° C., for 30 minutes and the supernatant was collected. The clarified supernatant was applied to a Heparin 6 Fast Flow column (GE Life Sciences) by gravity flow. The column was washed with 5 CV of Heparin Buffer A (50 mM HEPES-NaOH, 250 mM NaCl, 5 mM MgCl₂, 1 mM TCEP, 10% glycerol, pH 8), then with 5 CV of Heparin Buffer B (Buffer A with the NaCl concentration adjusted to 500 mM). Protein was eluted with 5 CV of Heparin Buffer C (Buffer A with the NaCl concentration adjusted to 1 M), collected in fractions. Fractions were assayed for protein by Bradford Assay and protein-containing fractions were pooled. The pooled heparin eluate was applied to a Strep-Tactin XT Superflow column (IBA Life Sciences) by gravity flow. The column was washed with 5 CV of Strep Buffer (50 mM HEPES-NaOH, 500 mM NaCl, 5 mM MgCl₂, 1 mM TCEP, 10% glycerol, pH 8). Protein was eluted from the column using 5 CV of Strep Buffer with 50 mM D-Biotin added and collected in fractions. CasX-containing fractions were pooled, concentrated at 4° C. using a 30 kDa cut-off spin concentrator, and purified by size exclusion chromatography on a Superdex 200 pg column (GE Life Sciences). The column was equilibrated with SEC Buffer (25 mM sodium phosphate, 300 mM NaCl, 1 mM TCEP, 10% glycerol, pH 7.25) operated by an AKTA Pure FPLC system (GE Life Sciences). CasX-containing fractions that eluted at the appropriate molecular weight were pooled, concentrated at 4° C. using a 30 kDa cut-off spin concentrator, aliquoted, and snap-frozen in liquid nitrogen before being stored at −80° C.

### 3. Results

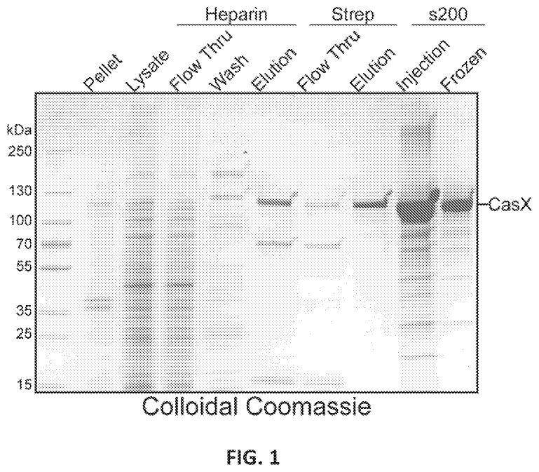

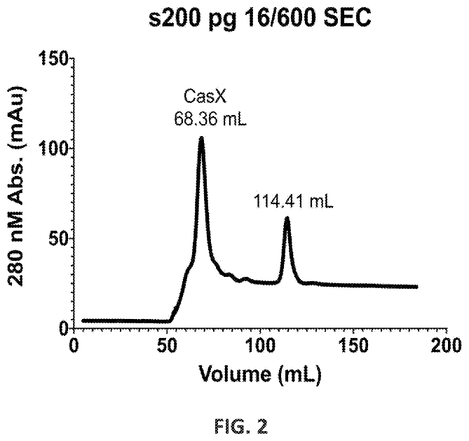

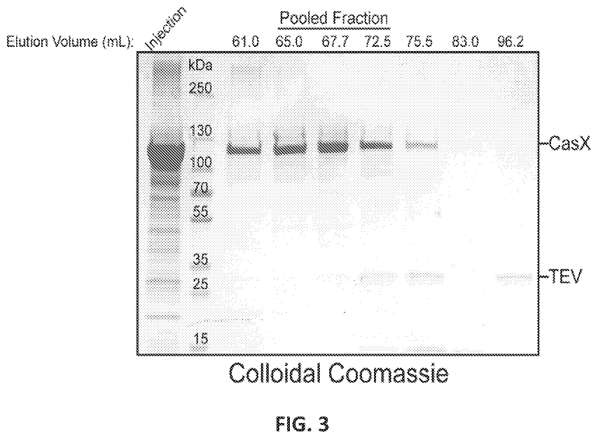

Samples from throughout the purification were resolved by SDS-PAGE and visualized by colloidal Coomassie staining, as shown in FIG. 1 and FIG. 3. In FIG. 1, the lanes, from left to right, are: molecular weight standards, Pellet: insoluble portion following cell lysis, Lysate: soluble portion following cell lysis, Flow Thru: protein that did not bind the Heparin column, Wash: protein that eluted from the column in wash buffer, Elution: protein eluted from the heparin column with elution buffer, Flow Thru: Protein that did not bind the StrepTactinXT column, Elution: protein eluted from the StrepTactin XT column with elution buffer, Injection: concentrated protein injected onto the s200 gel filtration column, Frozen: pooled fractions from the s200 elution that have been concentrated and frozen. In FIG. 3, the lanes from right to left, are the injection (sample of protein injected onto the gel filtration column,) molecular weight markers, lanes 3-9 are samples from the indicated elution volumes. Results from the gel filtration are shown in FIG. 2. The 68.36 mL peak corresponds to the apparent molecular weight of CasX and contained the majority of CasX protein. The average yield was 0.75 mg of purified CasX protein per liter of culture, with 75% purity, as evaluated by colloidal Coomassie staining.

### Example 2: CasX Construct 119, 438 and 457

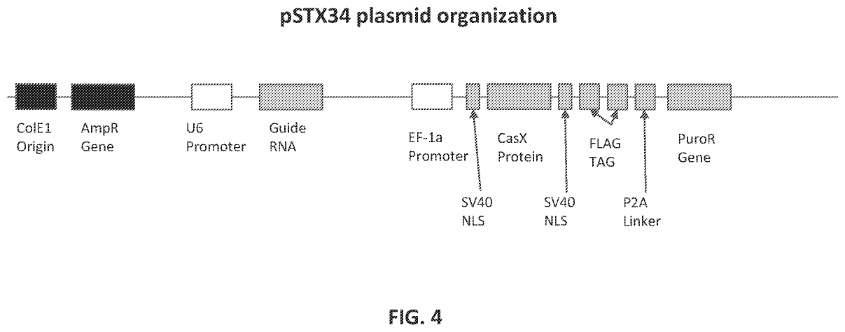

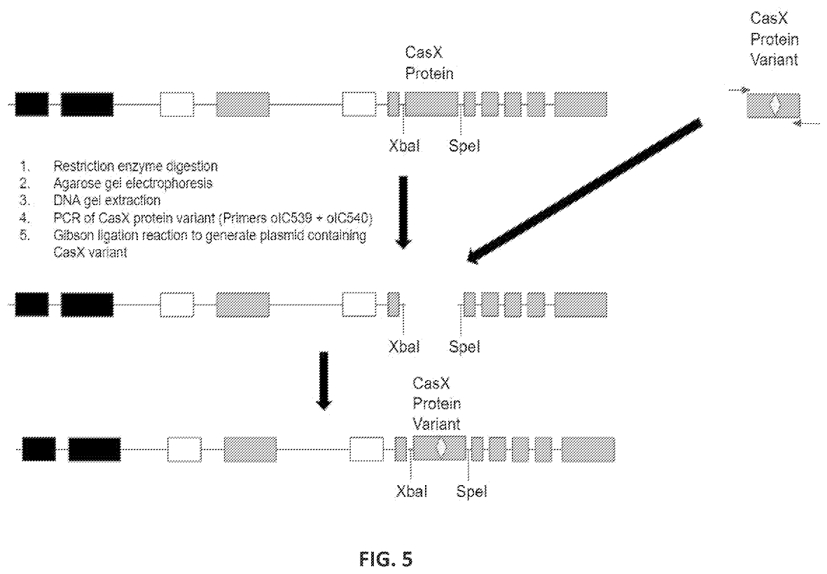

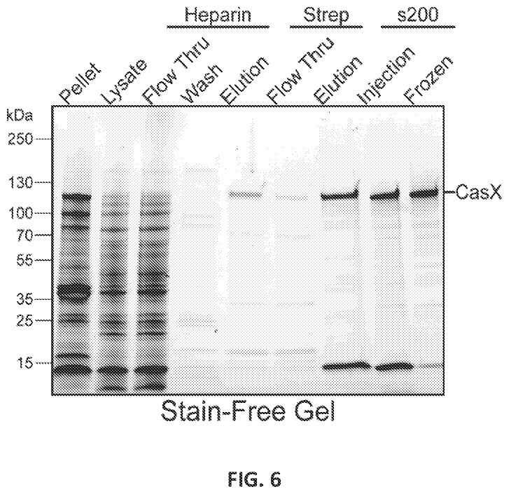

In order to generate the CasX 119, 438, and 457 constructs (sequences in Table 5), the codon-optimized CasX 37 construct (based on the Stx2 construct of Example 1, encoding Planctomycetes CasX SEQ ID NO:2, with a A708K substitution and a [P793] deletion with fused NLS, and linked guide and non-targeting sequences) was cloned into a mammalian expression plasmid (pStX; see FIG. 4) using standard cloning methods. To build CasX 119, the CasX 37 construct DNA was PCR amplified in two reactions using Q5 DNA polymerase (New England BioLabs Cat #M0491L) using primers oIC539 and oIC88 as well as oIC87 and oIC540 respectively (see FIG. 5). To build CasX 457, the CasX 365 construct DNA was PCR amplified in four reactions using Q5 DNA polymerase using primers oIC539 and oIC212, oIC211 and oIC376, oIC375 and oIC551, and oIC550 and oIC540 respectively. To build CasX 438, the CasX 119 construct DNA was PCR amplified in four reactions using Q5 DNA polymerase using primers oIC539 and oIC689, oIC688 and oIC376, oIC375 and oIC551, and oIC550 and oIC540 respectively. The resulting PCR amplification products were then purified using Zymoclean DNA clean and concentrator (Zymo Research Cat #4014) and the pStX backbone was digested using XbaI and SpeI. The digested backbone fragment was purified by gel extraction from a 1% agarose gel (Gold Bio Cat #A-201-500) using Zymoclean Gel DNA Recovery Kit (Zymo Research Cat #D4002) and the three fragments were then pieced together using Gibson assembly (New England BioLabs Cat #E2621S). Assembled products in the pStx34 were transformed into chemically-competent or electro-competent Turbo Competent E. coli bacterial cells, plated on LB-Agar plates (LB: Teknova Cat #L9315, Agar: Quartzy Cat #214510) containing carbenicillin. Individual colonies were picked and miniprepped using Qiagen Qiaprep spin Miniprep Kit (Qiagen Cat #27104) following the manufacturer's protocol. The resultant plasmids were sequenced using Sanger sequencing. Sequences encoding the targeting sequences that target the gene of interest were designed based on CasX PAM locations. Targeting sequence DNA was ordered as single-stranded DNA (ssDNA) oligos (Integrated DNA Technologies) consisting of the targeting sequence and the reverse complement of this sequence. These two oligos were annealed together and cloned into pStX individually or in bulk by Golden Gate assembly using T4 DNA Ligase (New England BioLabs Cat #M0202L) and an appropriate restriction enzyme for the plasmid. Golden Gate products were transformed into chemically or electro-competent cells such as NEB Turbo competent E. coli (NEB Cat #C29841), plated on LB-Agar plates (LB: Teknova Cat #L9315, Agar: Quartzy Cat #214510) containing carbenicillin. Individual colonies were picked and miniprepped using Qiagen Qiaprep spin Miniprep Kit (Qiagen Cat #27104) and the resultant plasmids were sequenced using Sanger sequencing. SaCas9 and SpyCas9 control plasmids were prepared similarly to pStX plasmids described above, with the protein and guide regions of pStX exchanged for the respective protein and guide. The expression and recovery of the CasX 119 and 457 proteins was performed using the general methodologies of Example 1 (however the DNA sequences were codon optimized for expression in E. coli). The results of analytical assays for CasX 119 are shown in FIGS. 6-8. The average yield of the CasX 119 was 1.56 mg of purified CasX protein per liter of culture at 75% purity, as evaluated by colloidal Coomassie staining. FIG. 6 shows an SDS-PAGE gel of purification samples, visualized on a Bio-Rad Stain-Free™ gel, as described. The lanes, from left to right, are: Pellet: insoluble portion following cell lysis, Lysate: soluble portion following cell lysis, Flow Thru: protein that did not bind the Heparin column, Wash: protein that eluted from the column in wash buffer, Elution: protein eluted from the heparin column with elution buffer, Flow Thru: Protein that did not bind the StrepTactinXT column, Elution: protein eluted from the StrepTactin XT column with elution buffer, Injection: concentrated protein injected onto the s200 gel filtration column, Frozen: pooled fractions from the s200 elution that have been concentrated and frozen.

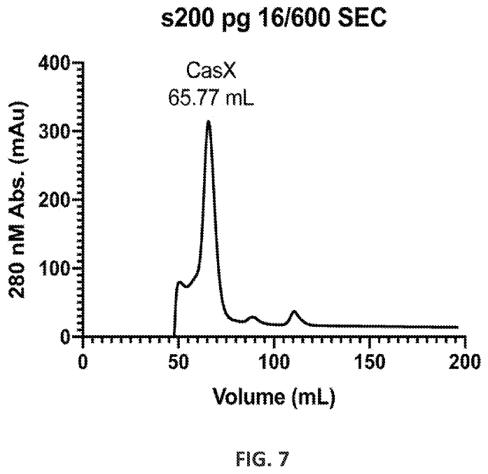

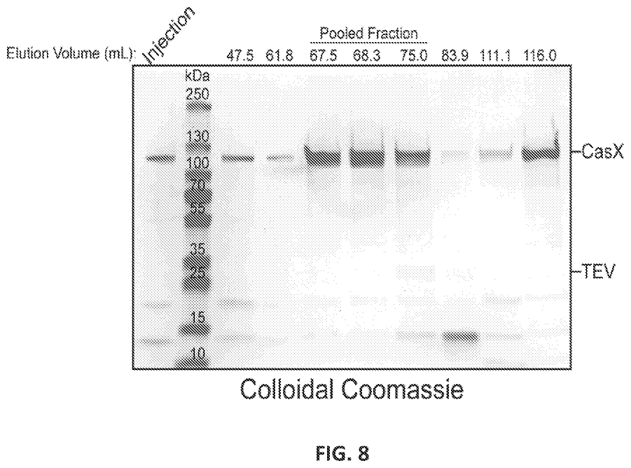

FIG. 7 shows the chromatogram of Superdex 200 16/600 pg Gel Filtration, as described. Gel filtration run of CasX variant 119 protein plotted as 280 nm absorbance against elution volume. The 65.77 mL peak corresponds to the apparent molecular weight of CasX variant 119 and contained the majority of CasX variant 119 protein. FIG. 8 shows an SDS-PAGE gel of gel filtration samples, stained with colloidal Coomassie, as described. Samples from the indicated fractions were resolved by SDS-PAGE and stained with colloidal Coomassie. From right to left, Injection: sample of protein injected onto the gel filtration column, molecular weight markers, lanes 3-10: samples from the indicated elution volumes.

TABLE 5
Sequences of CasX 119, 438 and 457

| Construct | DNA Sequence | Amino Acid Sequence |
|---|---|---|
| CasX 119 | (SEQ ID NO: 438) | QEIKRINKIRRRLVKDSNTKKAGKTGPMKTLLVRVMTPDLRERLEN |
| | | LRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYS |
| | | LGKFGQRALDFYSIHVTRESNHPVKPLEQIGGNSCASGPVGKALSD |
| | | ACMGAVASFLTKYQDIILEHQKVIKKNEKRLANLKDIASANGLAFP |
| | | KITLPPQPHTKEGIEAYNNVVAQIVIWVNLNLWQKLKIGRDEAKPL |
| | | QRLKGFPSFPLVERQANEVDWWDMVCNVKKLINEKKEDGKVFWQNL |
| | | AGYKRQEALRPYLSSEEDRKKGKKFARYQFGDLLLHLEKKHGEDWG |
| | | KVYDEAWERIDKKVEGLSKHIKLEEERRSEDAQSKAALTDWLRAKA |
| | | SFVIEGLKEADKDEFCRCELKLQKWYGDLRGKPFAIEAENSILDIS |
| | | GFSKQYNCAFIWQKDGVKKLNLYLIINYFKGGKLRFKKIKPEAFEA |
| | | NRFYTVINKKSGEIVPMEVNFNFDDPNLIILPLAFGKRQGREFIWN |
| | | DLLSLETGSLKLANGRVIEKTLYNRRTRQDEPALFVALTFERREVL |
| | | DSSNIKPMNLIGIDRGENIPAVIALTDPEGCPLSRFKDSLGNPTHI |
| | | LRIGESYKEKQRTIQAKKEVEQRRAGGYSRKYASKAKNLADDMVRN |
| | | TARDLLYYAVTQDAMLIFENLSRGFGRQGKRTFMAERQYTRMEDWL |
| | | TAKLAYEGLSKTYLSKTLAQYTSKTCSNCGFTITSADYDRVLEKLK |
| | | KTATGWMTTINGKELKVEGQITYYNRYKRQNVVKDLSVELDRLSEE |
| | | SVNNDISSWTKGRSGEALSLLKKRFSHRPVQEKFVCLNCGFETHAD |
| | | EQAALNIARSWLFLRSQEYKKYQTNKTTGNTDKRAFVETWQSFYRK |
| | | KLKEVWKPAV (SEQ ID NO: 439) |
| CasX 457 | (SEQ ID NO: 440) | QEIKRINKIRRRLVKDSNTKKAGKTGPMKTLLVRVMTPDLRERLEN |
| | | LRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYS |
| | | LGKFGQRALDFYSIHVTRESNHPVKPLEQIGGNSCASGPVGKALSD |
| | | ACMGAVASFLTKYQDIILEHKKVIKKNEKRLANLKDIASANGLAFP |
| | | KITLPPQPHTKEGIEAYNNVVAQIVIWVNLNLWQKLKIGRDEAKPL |
| | | QRLKGFPSFPLVERQANEVDWWDMVCNVKKLINEKKEDGKVFWQNL |
| | | AGYKRQEALRPYLSSPEDRKKGKKFARYQLGDLLLHLEKKHGEDWG |
| | | KVYDEAWERIDKKVEGLSKHIKLEEERRSEDAQSKAALTDWLRAKA |
| | | SFVIEGLKEADKDEFCRCELKLQKWYGDLRGKPFAIEAENSILDIS |
| | | GFSKQYNCAFIWQKDGVKKLNLYLIINYFKGGKLRFKKIKPEAFEA |
| | | NRFYTVINKKSGEIVPMEVNFNFDDPNLIILPLAFGKRQGREFIWN |
| | | DLLSLETGSLKLANGRVIEKPLYNRRTRQDEPALFVALTFERREVL |
| | | DSSNIKPMNLIGVDRGENIPAVIALTDPEGCPLSRFKDSLGNPTHI |
| | | LRIGESYKEKQRTIQAKKEVEQRRAGGYSRKYASKAKNLADDMVRN |
| | | TARDLLYYAVTQDAMLIFENLSRGFGRQGKRTFMAERQYTRMEDWL |
| | | TAKLAYEGLSKTYLSKTLAQYTSKTCSNCGFTITSADYDRVLEKLK |
| | | KTATGWMTTINGKELKVEGQITYYNRRKRQNVVKDLSVELDRLSEE |
| | | SVNNDISSWTKGRSGEALSLLKKRFSHRPVQEKFVCLNCGFETHAD |
| | | EQAALNIARSWLFLRSQEYKKYQTNKTTGNTDKRAFVETWQSFYRK |
| | | KLKEVWKPAV (SEQ ID NO: 441) |
| CasX 438 | (SEQ ID NO: 442) | QEIKRINKIRRRLVKDSNTKKAGKTGPMKTLLVRVMTPDLRERLEN |
| | | LRKKPENIPQPISNTSRANLNKLLTDYTEMKKAILHVYWEEFQKDP |
| | | VGLMSRVAQPAPKNIDQRKLIPVKDGNERLTSSGFACSQCCQPLYV |
| | | YKLEQVNDKGKPHTNYFGRCNVSEHERLILLSPHKPEANDELVTYS |
| | | LGKFGQRALDFYSIHVTRESNHPVKPLEQIGGNSCASGPVGKALSD |
| | | ACMGAVASFLTKYQDIILEHQKVIKKNEKRLANLKDIASANGLAFP |
| | | KITLPPQPHTKEGIEAYNNVVAQIVIWVNLNLWQKLKIGRDEAKPL |
| | | QRLKGFPSFPLVERQANEVDWWDMVCNVKKLINEKKEDGKVFWQNL |
| | | AGYKRQEALRPYLSSEEDRKKGKKFARYQLGDLLKHLEKKHGEDWG |
| | | KVYDEAWERIDKKVEGLSKHIKLEEERRSEDAQSKAALTDWLRAKA |
| | | SFVIEGLKEADKDEFCRCELKLQKWYGDLRGKPFAIEAENSILDIS |
| | | GFSKQYNCAFIWQKDGVKKLNLYLIINYFKGGKLRFKKIKPEAFEA |
| | | NRFYTVINKKSGEIVPMEVNFNFDDPNLIILPLAFGKRQGREFIWN |
| | | DLLSLETGSLKLANGRVIEKTLYNRRTRQDEPALFVALTFERREVL |
| | | DSSNIKPMNLIGVDRGENIPAVIALTDPEGCPLSRFKDSLGNPTHI |
| | | LRIGESYKEKQRTIQAKKEVEQRRAGGYSRKYASKAKNLADDMVRN |
| | | TARDLLYYAVTQDAMLIFENLSRGFGRQGKRTFMAERQYTRMEDWL |
| | | TAKLAYEGLSKTYLSKTLAQYTSKTCSNCGFTITSADYDRVLEKLK |
| | | KTATGWMTTINGKELKVEGQITYYNRRKRQNVVKDLSVELDRLSEE |
| | | SVNNDISSWTKGRSGEALSLLKKRFSHRPVQEKFVCLNCGFETHAD |
| | | EQAALNIARSWLFLRSQEYKKYQTNKTTGNTDKRAFVETWQSFYRK |
| | | KLKEVWKPAV (SEQ ID NO: 443) |

### Example 3: CasX Construct 488 and 491

In order to generate the CasX 488 construct (sequences in Table 6), the codon-optimized CasX 119 construct (based on the CasX Stx2 construct of Example 1, encoding Planctomycetes CasX SEQ ID NO:2, with a A708K substitution, a L379R substitution, and a [P793] deletion with fused NLS, and linked guide and non-targeting sequences) was utilized as the starting construct, and was generated using the methodologies of Example 2. In order to generate CasX 491 (sequences in Table 6), the CasX 484 construct DNA was PCR amplified using Q5 DNA polymerase (New England BioLabs Cat #M0491L) was utilized as the starting construct, and was generated using the methodologies of Example 2 (see FIG. 5). The resultant plasmids were sequenced using Sanger sequencing. Sequences encoding the targeting sequences that target the gene of interest were designed based on CasX PAM locations, as described in Example 2. SaCas9 and SpyCas9 control plasmids were prepared similarly to pStX plasmids described above, with the protein and guide regions of pStX exchanged for the respective protein and guide. Targeting sequences for SaCas9 and SpyCas9 were either obtained from the literature or were rationally designed according to established methods. The expression and recovery of the CasX constructs was performed using the general methodologies of Example 1 and Example 2, with similar results obtained.

TABLE 6
Sequences of CasX 488 and 491

| Construct | DNA Sequence | Amino Acid Sequence |
|---|---|---|
| CasX 488 | (SEQ ID NO: 444) | QEIKRINKIRRRLVKDSNTKKAGKTGPMKTLLVRVMTPDLRERLE |
| | | NLRKKPENIPQPISNTSRANLNKLLTDYTEMKKAILHVYWEEFQK |
| | | DPVGLMSRVAQPASKKIDQNKLKPEMDEKGNLTTAGFACSQCGQP |
| | | LFVYKLEQVSEKGKAYTNYFGRCNVAEHEKLILLAQLKPEKDSDE |
| | | AVTYSLGKFGQRALDFYSIHVTKESTHPVKPLAQIAGNRYASGPV |
| | | GKALSDACMGTIASELSKYQD111EHQKVVKGNQKRLESLRELAG |
| | | KENLEYPSVTLPPQPHTKEGVDAYNEVIARVRMWVNLNLWQKLKL |
| | | SRDDAKPLLRLKGFPSFPLVERQANEVDWWDMVCNVKKLINEKKE |
| | | DGKVFWQNLAGYKRQEALRPYLSSEEDRKKGKKFARYQFGDLLLH |
| | | LEKKHGEDWGKVYDEAWERIDKKVEGLSKHIKLEEERRSEDAQSK |
| | | AALTDWLRAKASFVIEGLKEADKDEFCRCELKLQKWYGDLRGKPF |
| | | AIEAENSILDISGFSKQYNCAFIWQKDGVKKLNLYLIINYFKGGK |
| | | LRFKKIKPEAFEANRFYTVINKKSGEIVPMEVNFNFDDPNLIILP |
| | | LAFGKRQGREFIWNDLLSLETGSLKLANGRVIEKTLYNRRTRQDE |
| | | PALFVALTFERREVLDSSNIKPMNLIGIDRGENIPAVIALTDPEG |
| | | CPLSRFKDSLGNPTHILRIGESYKEKQRTIQAKKEVEQRRAGGYS |
| | | RKYASKAKNLADDMVRNTARDLLYYAVTQDAMLIFENLSRGFGRQ |
| | | GKRTFMAERQYTRMEDWLTAKLAYEGLSKTYLSKTLAQYTSKTCS |
| | | NCGFTITSADYDRVLEKLKKTATGWMTTINGKELKVEGQITYYNR |
| | | YKRQNVVKDLSVELDRLSEESVNNDISSWTKGRSGEALSLLKKRF |
| | | SHRPVQEKFVCLNCGFETHADEQAALNIARSWLFLRSQEYKKYQT |
| | | NKTTGNTDKRAFVETWQSFYRKKLKEVWKPAV |
| | | (SEQ ID NO: 445) |
| CasX 491 | (SEQ ID NO: 446) | QEIKRINKIRRRLVKDSNTKKAGKTGPMKTLLVRVMTPDLRERLE |
| | | NLRKKPENIPQPISNTSRANLNKLLTDYTEMKKAILHVYWEEFQK |
| | | DPVGLMSRVAQPASKKIDQNKLKPEMDEKGNLTTAGFACSQCGQP |
| | | LFVYKLEQVSEKGKAYTNYFGRCNVAEHEKLILLAQLKPEKDSDE |
| | | AVTYSLGKFGQRALDFYSIHVTKESTHPVKPLAQIAGNRYASGPV |
| | | GKALSDACMGTIASELSKYQD111EHQKVVKGNQKRLESLRELAG |
| | | KENLEYPSVTLPPQPHTKEGVDAYNEVIARVRMWVNLNLWQKLKL |
| | | SRDDAKPLLRLKGFPSFPLVERQANEVDWWDMVCNVKKLINEKKE |
| | | DGKVFWQNLAGYKRQEALRPYLSSEEDRKKGKKFARYQLGDLLLH |
| | | LEKKHGEDWGKVYDEAWERIDKKVEGLSKHIKLEEERRSEDAQSK |
| | | AALTDWLRAKASFVIEGLKEADKDEFCRCELKLQKWYGDLRGKPF |
| | | AIEAENSILDISGFSKQYNCAFIWQKDGVKKLNLYLIINYFKGGK |
| | | LRFKKIKPEAFEANRFYTVINKKSGEIVPMEVNFNFDDPNLIILP |
| | | LAFGKRQGREFIWNDLLSLETGSLKLANGRVIEKTLYNRRTRQDE |
| | | PALEVALTEERREVLDSSNIKPMNLIGVDRGENIPAVIALTDPEG |
| | | CPLSRFKDSLGNPTHILRIGESYKEKQRTIQAKKEVEQRRAGGYS |
| | | RKYASKAKNLADDMVRNTARDLLYYAVTQDAMLIFENLSRGFGRQ |
| | | GKRTFMAERQYTRMEDWLTAKLAYEGLSKTYLSKTLAQYTSKTCS |
| | | NCGFTITSADYDRVLEKLKKTATGWMTTINGKELKVEGQITYYNR |
| | | YKRQNVVKDLSVELDRLSEESVNNDISSWTKGRSGEALSLLKKRF |
| | | SHRPVQEKFVCLNCGFETHADEQAALNIARSWLFLRSQEYKKYQT |
| | | NKTTGNTDKRAFVETWQSFYRKKLKEVWKPAV |
| | | (SEQ ID NO: 447) |

### Example 4: Design and Generation of CasX Constructs 278-280, 285-288, 290, 291, 293, 300, 492, and 493

In order to generate the CasX 278-280, 285-288, 290, 291, 293, 300, 492, and 493 constructs (sequences in Table 7), the N- and C-termini of the codon-optimized CasX 119 construct (based on the CasX Stx37 construct of Example 2) in a mammalian expression vector were manipulated to delete or add NLS sequences (sequences in Table 8). Constructs 278, 279, and 280 were manipulations of the N- and C-termini using only an SV40 NLS sequence. Construct 280 had no NLS on the N-terminus and added two SV40 NLS' on the C-terminus with a triple proline linker in between the two SV40 NLS sequences. In order to generate constructs 492 and 493, constructs 280 and 291 were used as the starting constructs. Cloning methods were performed as described in Example 2. The resultant plasmids were sequenced using Sanger sequencing. Sequences encoding the targeting sequences that target the gene of interest were designed based on CasX PAM locations, and was prepared as described in Example 2. The plasmids were used to produce and recover CasX protein utilizing the general methodologies of Examples 1 and 2. The resultant plasmids were sequenced using Sanger sequencing. Sequences encoding the targeting spacer sequences that target the gene of interest were designed based on CasX PAM locations. The expression and recovery of the CasX constructs was performed using the general methodologies of Example 1 and Example 2, with similar results obtained.

TABLE 7
CasX 278-280, 285-288, 290, 291, 293, 300, 492, and 493 sequences

| Construct | Amino Acid Sequence |
|---|---|
| 278 | MAPKKKRKVSRQEIKRINKIRRRLVKDSNTKKAGKTGPMKTLLVRVMTPDLRERL |
| | ENLRKKPENIPQPISNTSRANLNKLLTDYTEMKKAILHVYWEEFQKDPVGLMSRV |
| | AQPAPKNIDQRKLIPVKDGNERLTSSGFACSQCCQPLYVYKLEQVNDKGKPHTNY |
| | FGRCNVSEHERLILLSPHKPEANDELVTYSLGKFGQRALDFYSIHVTRESNHPVK |
| | PLEQIGGNSCASGPVGKALSDACMGAVASFLTKYQDIILEHQKVIKKNEKRLANL |
| | KDIASANGLAFPKITLPPQPHTKEGIEAYNNVVAQIVIWVNLNLWQKLKIGRDEA |
| | KPLQRLKGFPSFPLVERQANEVDWWDMVCNVKKLINEKKEDGKVFWQNLAGYKRQ |
| | EALRPYLSSEEDRKKGKKFARYQFGDLLLHLEKKHGEDWGKVYDEAWERIDKKVE |
| | GLSKHIKLEEERRSEDAQSKAALTDWLRAKASFVIEGLKEADKDEFCRCELKLQK |
| | WYGDLRGKPFAIEAENSILDISGFSKQYNCAFIWQKDGVKKLNLYLIINYFKGGK |
| | LRFKKIKPEAFEANRFYTVINKKSGEIVPMEVNFNFDDPNLIILPLAFGKRQGRE |
| | FIWNDLLSLETGSLKLANGRVIEKTLYNRRTRQDEPALFVALTFERREVLDSSNI |
| | KPMNLIGIDRGENIPAVIALTDPEGCPLSRFKDSLGNPTHILRIGESYKEKQRTI |
| | QAKKEVEQRRAGGYSRKYASKAKNLADDMVRNTARDLLYYAVTQDAMLIFENLSR |
| | GFGRQGKRTFMAERQYTRMEDWLTAKLAYEGLSKTYLSKTLAQYTSKTCSNCGFT |
| | ITSADYDRVLEKLKKTATGWMTTINGKELKVEGQITYYNRYKRQNVVKDLSVELD |
| | RLSEESVNNDISSWTKGRSGEALSLLKKRFSHRPVQEKFVCLNCGFETHADEQAA |
| | LNIARSWLFLRSQEYKKYQTNKTTGNTDKRAFVETWQSFYRKKLKEVWKPAV |
| | (SEQ ID NO: 448) |
| 279 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKV |
| | (SEQ ID NO: 449) |
| 280 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANREYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPP |
| | KKKRKV (SEQ ID NO: 450) |
| 285 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANREYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPH |
| | KKKHPDASVNFSEFSK (SEQ ID NO: 451) |
| 286 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPQ |
| | RPGPYDRPQRPGPYDRP (SEQ ID NO: 452) |
| 287 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPL |
| | SPSLSPLLSPSLSPL (SEQ ID NO: 453) |
| 288 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTMSSGFACSQCCQPLYVYKLEQVNDKGKPHTNYFGRCNVSEH |
| | ERLILLSPHKPEANDELVTYSLGKFGQRALDFYSIHVTRESNHPVKPLEQIGGNS |
| | CASGPVGKALSDACMGAVASFLTKYQDIILEHQKVIKKNEKRLANLKDIASANGL |
| | AFPKITLPPQPHTKEGIEAYNNVVAQIVIWVNLNLWQKLKIGRDEAKPLQRLKGF |
| | PSFPLVERQANEVDWWDMVCNVKKLINEKKEDGKVFWQNLAGYKRQEALRPYLSS |
| | EEDRKKGKKFARYQFGDLLLHLEKKHGEDWGKVYDEAWERIDKKVEGLSKHIKLE |
| | EERRSEDAQSKAALTDWLRAKASFVIEGLKEADKDEFCRCELKLQKWYGDLRGKP |
| | FAIEAENSILDISGFSKQYNCAFIWQKDGVKKLNLYLIINYFKGGKLRFKKIKPE |
| | AFEANRFYTVINKKSGEIVPMEVNFNFDDPNLIILPLAFGKRQGREFIWNDLLSL |
| | ETGSLKLANGRVIEKTLYNRRTRQDEPALFVALTFERREVLDSSNIKPMNLIGID |
| | RGENIPAVIALTDPEGCPLSRFKDSLGNPTHILRIGESYKEKQRTIQAKKEVEQR |
| | RAGGYSRKYASKAKNLADDMVRNTARDLLYYAVTQDAMLIFENLSRGFGRQGKRT |
| | FMAERQYTRMEDWLTAKLAYEGLSKTYLSKTLAQYTSKTCSNCGFTITSADYDRV |
| | LEKLKKTATGWMTTINGKELKVEGQITYYNRYKRQNWKDLSVELDRLSEESVNN |
| | DISSWTKGRSGEALSLLKKRFSHRPVQEKFVCLNCGFETHADEQAALNIARSWLF |
| | LRSQEYKKYQTNKTTGNTDKRAFVETWQSFYRKKLKEVWKPAVTSPKKKRKVPPP |
| | RGKGGKGLGKGGAKRHRK (SEQ ID NO: 454) |
| 290 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANREYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPS |
| | RRRKANPTKLSENAKKLAKEVEN (SEQ ID NO: 455) |
| 291 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPP |
| | AAKRVKLD (SEQ ID NO: 456) |
| 293 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANREYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPK |
| | RSFSKAF (SEQ ID NO: 457) |
| 300 | MQEIKRINKIRRRLVKDSNTKKAGKTGPMKTLLVRVMTPDLRERLENLRKKPENI |
| | PQPISNTSRANLNKLLTDYTEMKKAILHVYWEEFQKDPVGLMSRVAQPAPKNIDQ |
| | RKLIPVKDGNERLTSSGFACSQCCQPLYVYKLEQVNDKGKPHTNYFGRCNVSEHE |
| | RLILLSPHKPEANDELVTYSLGKFGQRALDFYSIHVTRESNHPVKPLEQIGGNSC |
| | ASGPVGKALSDACMGAVASFLTKYQDIILEHQKVIKKNEKRLANLKDIASANGLA |
| | FPKITLPPQPHTKEGIEAYNNVVAQIVIWVNLNLWQKLKIGRDEAKPLQRLKGFP |
| | SFPLVERQANEVDWWDMVCNVKKLINEKKEDGKVFWQNLAGYKRQEALRPYLSSE |
| | EDRKKGKKFARYQFGDLLLHLEKKHGEDWGKVYDEAWERIDKKVEGLSKHIKLEE |
| | ERRSEDAQSKAALTDWLRAKASFVIEGLKEADKDEFCRCELKLQKWYGDLRGKPF |
| | AIEAENSILDISGFSKQYNCAFIWQKDGVKKLNLYLIINYFKGGKLRFKKIKPEA |
| | FEANRFYTVINKKSGEIVPMEVNFNFDDPNLIILPLAFGKRQGREFIWNDLLSLE |
| | TGSLKLANGRVIEKTLYNRRTRQDEPALFVALTFERREVLDSSNIKPMNLIGIDR |
| | GENIPAVIALTDPEGCPLSRFKDSLGNPTHILRIGESYKEKQRTIQAKKEVEQRR |
| | AGGYSRKYASKAKNLADDMVRNTARDLLYYAVTQDAMLIFENLSRGFGRQGKRTF |
| | MAERQYTRMEDWLTAKLAYEGLSKTYLSKTLAQYTSKTCSNCGFTITSADYDRVL |
| | EKLKKTATGWMTTINGKELKVEGQITYYNRYKRQNVVKDLSVELDRLSEESVNND |
| | ISSWTKGRSGEALSLLKKRFSHRPVQEKFVCLNCGFETHADEQAALNIARSWLFL |
| | RSQEYKKYQTNKTTGNTDKRAFVETWQSFYRKKLKEVWKPAVTSPKKKRKVPPPK |
| | RGINDRNFWRGENERKTR (SEQ ID NO: 458) |
| 492 | MAPKKKRKVSRMQEIKRINKIRRRLVKDSNTKKAGKTGPMKTLLVRVMTPDLRER |
| | LENLRKKPENIPQPISNTSRANLNKLLTDYTEMKKAILHVYWEEFQKDPVGLMSR |
| | VAQPAPKNIDQRKLIPVKDGNERLTSSGFACSQCCQPLYVYKLEQVNDKGKPHTN |
| | YFGRCNVSEHERLILLSPHKPEANDELVTYSLGKFGQRALDFYSIHVTRESNHPV |
| | KPLEQIGGNSCASGPVGKALSDACMGAVASFLTKYQDIILEHQKVIKKNEKRLAN |
| | LKDIASANGLAFPKITLPPQPHTKEGIEAYNNVVAQIVIWVNLNLWQKLKIGRDE |
| | AKPLQRLKGFPSFPLVERQANEVDWWDMVCNVKKLINEKKEDGKVFWQNLAGYKR |
| | QEALRPYLSSEEDRKKGKKFARYQFGDLLLHLEKKHGEDWGKVYDEAWERIDKKV |
| | EGLSKHIKLEEERRSEDAQSKAALTDWLRAKASFVIEGLKEADKDEFCRCELKLQ |
| | KWYGDLRGKPFAIEAENSILDISGFSKQYNCAFIWQKDGVKKLNLYLIINYFKGG |
| | KLRFKKIKPEAFEANRFYTVINKKSGEIVPMEVNFNFDDPNLIILPLAFGKRQGR |
| | EFIWNDLLSLETGSLKLANGRVIEKTLYNRRTRQDEPALFVALTFERREVLDSSN |
| | IKPMNLIGIDRGENIPAVIALTDPEGCPLSRFKDSLGNPTHILRIGESYKEKQRT |
| | IQAKKEVEQRRAGGYSRKYASKAKNLADDMVRNTARDLLYYAVTQDAMLIFENLS |
| | RGFGRQGKRTFMAERQYTRMEDWLTAKLAYEGLSKTYLSKTLAQYTSKTCSNCGF |
| | TITSADYDRVLEKLKKTATGWMTTINGKELKVEGQITYYNRYKRQNVVKDLSVEL |
| | DRLSEESVNNDISSWTKGRSGEALSLLKKRFSHRPVQEKFVCLNCGFETHADEQA |
| | ALNIARSWLFLRSQEYKKYQTNKTTGNTDKRAFVETWQSFYRKKLKEVWKPAVTS |
| | PKKKRKVPPPPKKKRKV (SEQ ID NO: 459) |
| 493 | MAPKKKRKVSRMQEIKRINKIRRRLVKDSNTKKAGKTGPMKTLLVRVMTPDLRER |
| | LENLRKKPENIPQPISNTSRANLNKLLTDYTEMKKAILHVYWEEFQKDPVGLMSR |
| | VAQPAPKNIDQRKLIPVKDGNERLTSSGFACSQCCQPLYVYKLEQVNDKGKPHTN |
| | YFGRCNVSEHERLILLSPHKPEANDELVTYSLGKFGQRALDFYSIHVTRESNHPV |
| | KPLEQIGGNSCASGPVGKALSDACMGAVASFLTKYQDIILEHQKVIKKNEKRLAN |
| | LKDIASANGLAFPKITLPPQPHTKEGIEAYNNVVAQIVIWVNLNLWQKLKIGRDE |
| | AKPLQRLKGFPSFPLVERQANEVDWWDMVCNVKKLINEKKEDGKVFWQNLAGYKR |
| | QEALRPYLSSEEDRKKGKKFARYQFGDLLLHLEKKHGEDWGKVYDEAWERIDKKV |
| | EGLSKHIKLEEERRSEDAQSKAALTDWLRAKASFVIEGLKEADKDEFCRCELKLQ |
| | KWYGDLRGKPFAIEAENSILDISGFSKQYNCAFIWQKDGVKKLNLYLIINYFKGG |
| | KLRFKKIKPEAFEANRFYTVINKKSGEIVPMEVNFNFDDPNLIILPLAFGKRQGR |
| | EFIWNDLLSLETGSLKLANGRVIEKTLYNRRTRQDEPALFVALTFERREVLDSSN |
| | IKPMNLIGIDRGENIPAVIALTDPEGCPLSRFKDSLGNPTHILRIGESYKEKQRT |
| | IQAKKEVEQRRAGGYSRKYASKAKNLADDMVRNTARDLLYYAVTQDAMLIFENLS |
| | RGFGRQGKRTFMAERQYTRMEDWLTAKLAYEGLSKTYLSKTLAQYTSKTCSNCGF |
| | TITSADYDRVLEKLKKTATGWMTTINGKELKVEGQITYYNRYKRQNVVKDLSVEL |
| | DRLSEESVNNDISSWTKGRSGEALSLLKKRFSHRPVQEKFVCLNCGFETHADEQA |
| | ALNIARSWLFLRSQEYKKYQTNKTTGNTDKRAFVETWQSFYRKKLKEVWKPAVTS |
| | PKKKRKVPPPPAAKRVKLD (SEQ ID NO: 460) |

TABLE 8
Nuclear localization sequence list

| CasX | NLS | DNA Sequence | Amino Acid Sequence |
|---|---|---|---|
| 278, 279, | SV40 | CCAAAGAAGAAGCGGAAGGTC | PKKKRKV (SEQ ID |
| 280, 492, | | (SEQ ID NO: 461) | NO: 217) |
| 493 | | | |
| 285 | SynthNLS3 | CACAAGAAGAAACATCCAGACGC | HKKKHPDASVNFSEF |
| | | ATCAGTCAACTTTAGCGAGTTCA | SK (SEQ ID NO: |
| | | GTAAA (SEQ ID NO: 462) | 189) |
| 286 | SynthNLS4 | CAGCGCCCTGGGCCTTACGATAG | QRPGPYDRPQRPGPY |
| | | GCCGCAAAGACCCGGACCGTATG | DRP (SEQ ID NO: |
| | | ATCGCCCT (SEQ ID NO: | 190) |
| | | 463) | |
| 287 | SynthNLS5 | CTCAGCCCGAGTCTTAGTCCACT | LSPSLSPLLSPSLSP |
| | | GCTTTCCCCGTCCCTGTCTCCAC | L (SEQ ID NO: |
| | | TG (SEQ ID NO: 464) | 191) |
| 288 | SynthNLS6 | CGGGGCAAGGGTGGCAAGGGGCT | RGKGGKGLGKGGAKR |
| | | TGGCAAGGGGGGGGCAAAGAGGC | HRK (SEQ ID NO: |
| | | ACAGGAAG (SEQ ID NO: | 192) |
| | | 465) | |
| 290 | EGL-13 | AGCCGCCGCAGAAAAGCCAATCC | SRRRKANPTKLSENA |
| | | TACAAAACTGTCAGAAAATGCGA | KKLAKEVEN (SEQ |
| | | AAAAACTTGCTAAGGAGGTGGAA | ID NO: 470) |
| | | AAC (SEQ ID NO: 466) | |
| 291 | c-Myc | CCTGCCGCAAAGCGAGTGAAATT | PAAKRVKLD (SEQ |
| | | GGAC (SEQ ID NO: 467) | ID NO: 224) |
| 293 | Nucleolar RNA | AAGCGGTCCTTCAGTAAGGCCTT | KRSFSKAE (SEQ |
| | Helicase II | T (SEQ ID NO: 468) | ID NO: 181) |
| 300 | Influenza A | AAACGGGGAATAAACGACCGGAA | KRGINDRNFWRGENE |
| | protein | CTTCTGGCGCGGGGAAAACGAGC | RKTR (SEQ ID |
| | | GCAAAACCCGA (SEQ ID NO: | NO: 179) |
| | | 469) | |

### Example 5: Design and Generation of CasX Constructs 387, 395, 485-491, and 494

In order to generate CasX 395, CasX 485, CasX 486, CasX 487, the codon optimized CasX 119 (based on the CasX 37 construct of Example 2) was used as the starting construct. CasX 435, CasX 438, and CasX 484 were similarly based on the CasX 119 construct of Example 2, with Gibson primers designed to amplify the CasX SEQ ID NO: 1 Helical I domain from amino acid 192-331 in its own vector to replace this corresponding region (aa 193-332) on CasX 119, CasX 435, CasX 438, and CasX 484 in pStx1 respectively. In order to generate CasX 488, CasX 489, CasX 490, CasX 435, CasX 438, and CasX 484 and CasX 491 (sequences in Table 9), the codon optimized CasX 119 (based on the CasX 37 construct of Example 2), were cloned respectively into a 4 kb staging and Gibson primers were designed to amplify the CasX Stx1 NTSB domain from amino acid 101-191 and Helical I domain from amino acid 192-331 in its own vector to replace this similar region (aa 103-332) on CasX 119, CasX 435, CasX 438, and CasX 484 in pStx1 respectively. The plasmids were used to produce and recover CasX protein utilizing the general methodologies of Examples 1 and 2. The resultant plasmids were sequenced using Sanger sequencing. Sequences encoding the targeting spacer sequences that target the gene of interest were designed based on CasX PAM locations. The expression and recovery of the CasX constructs was performed using the general methodologies of Example 1 and Example 2, with similar results obtained.

TABLE 9
Sequences of CasX 395 and 485-491

| | DNA | |
|---|---|---|
| Construct | Sequence | Amino Acid Sequence |
| CasX 387 | (SEQ | MAPKKKRKVSRQEIKRINKIRRRLVKDSNTKKAGKTGPMKTLLVRV |
| | ID NO: | MTPDLRERLENLRKKPENIPQPISNTSRANLNKLLTDYTEMKKAIL |
| | 471) | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTRESNHPVKPLEQIGGNS |
| | | CASGPVGKALSDACMGAVASFLTKYQDIILEHQKVIKKNEKRLANL |
| | | KDIASANGLAFPKITLPPQPHTKEGIEAYNNVVAQIVIWVNLNLWQ |
| | | KLKIGRDEAKPLQRLKGFPSFPLVERQANEVDWWDMVCNVKKLINE |
| | | KKEDGKVFWQNLAGYKRQEALRPYLSSEEDRKKGKKFARYQFGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGIDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTFM |
| | | AERQYTRMEDWLTAKLAYEGLSKTYLSKTLAQYTSKTCSNCGFTIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 472) |
| CasX 395 | (SEQ ID | MAPKKKRKVSRQEIKRINKIRRRLVKDSNTKKAGKTGPMKTLLVRV |
| | NO: | MTPDLRERLENLRKKPENIPQPISNTSRANLNKLLTDYTEMKKAIL |
| | 473) | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQFGDLLL |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGIDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTFMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRYKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKVTSPKKKRKV (SEQ |
| | | ID NO: 474) |
| CasX 485 | (SEQ | MAPKKKRKVSRQEIKRINKIRRRLVKDSNTKKAGKTGPMKTLLVRV |
| | ID NO: | MTPDLRERLENLRKKPENIPQPISNTSRANLNKLLTDYTEMKKAIL |
| | 475) | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQLGDLLL |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGVDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTFMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRRKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKV (SEQ ID NO: |
| | | 476) |
| CasX 486 | (SEQ ID | MAPKKKRKVSRQEIKRINKIRRRLVKDSNTKKAGKTGPMKTLLVRV |
| | NO: | MTPDLRERLENLRKKPENIPQPISNTSRANLNKLLTDYTEMKKAIL |
| | 477) | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQLGDLLK |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGVDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTFMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRRKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKV (SEQ ID NO: |
| | | 478) |
| CasX 487 | (SEQ | MAPKKKRKVSRQEIKRINKIRRRLVKDSNTKKAGKTGPMKTLLVRV |
| | ID NO: | MTPDLRERLENLRKKPENIPQPISNTSRANLNKLLTDYTEMKKAIL |
| | 479) | HVYWEEFQKDPVGLMSRVAQPAPKNIDQRKLIPVKDGNERLTSSGF |
| | | ACSQCCQPLYVYKLEQVNDKGKPHTNYFGRCNVSEHERLILLSPHK |
| | | PEANDELVTYSLGKFGQRALDFYSIHVTKESTHPVKPLAQIAGNRY |
| | | ASGPVGKALSDACMGTIASFLSKYQDIIIEHQKVVKGNQKRLESLR |
| | | ELAGKENLEYPSVTLPPQPHTKEGVDAYNEVIARVRMWVNLNLWQK |
| | | LKLSRDDAKPLLRLKGFPSFPLVERQANEVDWWDMVCNVKKLINEK |
| | | KEDGKVFWQNLAGYKRQEALRPYLSSEEDRKKGKKFARYQLGDLLL |
| | | HLEKKHGEDWGKVYDEAWERIDKKVEGLSKHIKLEEERRSEDAQSK |
| | | AALTDWLRAKASFVIEGLKEADKDEFCRCELKLQKWYGDLRGKPFA |
| | | IEAENSILDISGFSKQYNCAFIWQKDGVKKLNLYLIINYFKGGKLR |
| | | FKKIKPEAFEANRFYTVINKKSGEIVPMEVNFNFDDPNLIILPLAF |
| | | GKRQGREFIWNDLLSLETGSLKLANGRVIEKTLYNRRTRQDEPALF |
| | | VALTFERREVLDSSNIKPMNLIGVDRGENIPAVIALTDPEGCPLSR |
| | | FKDSLGNPTHILRIGESYKEKQRTIQAKKEVEQRRAGGYSRKYASK |
| | | AKNLADDMVRNTARDLLYYAVTQDAMLIFENLSRGFGRQGKRTFMA |
| | | ERQYTRMEDWLTAKLAYEGLSKTYLSKTLAQYTSKTCSNCGFTITS |
| | | ADYDRVLEKLKKTATGWMTTINGKELKVEGQITYYNRYKRQNVVKD |
| | | LSVELDRLSEESVNNDISSWTKGRSGEALSLLKKRFSHRPVQEKFV |
| | | CLNCGFETHADEQAALNIARSWLFLRSQEYKKYQTNKTTGNTDKRA |
| | | FVETWQSFYRKKLKEVWKPAVTSPKKKRKV (SEQ ID NO: |
| | | 480) |
| CasX 488 | (SEQ ID | MAPKKKRKVSRQEIKRINKIRRRLVKDSNTKKAGKTGPMKTLLVRV |
| | NO: | MTPDLRERLENLRKKPENIPQPISNTSRANLNKLLTDYTEMKKAIL |
| | 481) | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQFGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGIDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTFM |
| | | AERQYTRMEDWLTAKLAYEGLSKTYLSKTLAQYTSKTCSNCGFTIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 482) |
| CasX 489 | (SEQ ID | MAPKKKRKVSRQEIKRINKIRRRLVKDSNTKKAGKTGPMKTLLVRV |
| | NO: | MTPDLRERLENLRKKPENIPQPISNTSRANLNKLLTDYTEMKKAIL |
| | 483) | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQLGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTFM |
| | | AERQYTRMEDWLTAKLAYEGLSKTYLSKTLAQYTSKTCSNCGFTIT |
| | | SADYDRVLEKLKKTATGWMTTINGKELKVEGQITYYNRRKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 484) |
| CasX 490 | (SEQ ID | MAPKKKRKVSRQEIKRINKIRRRLVKDSNTKKAGKTGPMKTLLVRV |
| | NO: | MTPDLRERLENLRKKPENIPQPISNTSRANLNKLLTDYTEMKKAIL |
| | 485) | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQLGDLL |
| | | KHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTFM |
| | | AERQYTRMEDWLTAKLAYEGLSKTYLSKTLAQYTSKTCSNCGFTIT |
| | | SADYDRVLEKLKKTATGWMTTINGKELKVEGQITYYNRRKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 486) |
| CasX 491 | (SEQ ID | MAPKKKRKVSRQEIKRINKIRRRLVKDSNTKKAGKTGPMKTLLVRV |
| | NO: | MTPDLRERLENLRKKPENIPQPISNTSRANLNKLLTDYTEMKKAIL |
| | 487) | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTKESTHPVKPLAQIAGNR |
| | | YASGPVGKALSDACMGTIASFLSKYQDIIIEHQKVVKGNQKRLESL |
| | | RELAGKENLEYPSVTLPPQPHTKEGVDAYNEVIARVRMWVNLNLWQ |
| | | KLKLSRDDAKPLLRLKGFPSFPLVERQANEVDWWDMVCNVKKLINE |
| | | KKEDGKVFWQNLAGYKRQEALRPYLSSEEDRKKGKKFARYQLGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTFM |
| | | AERQYTRMEDWLTAKLAYEGLSKTYLSKTLAQYTSKTCSNCGFTIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 488) |
| CasX 494 | (SEQ | MAPKKKRKVSRQEIKRINKIRRRLVKDSNTKKAGKTGPMKTLLVRV |
| | ID NO: | MTPDLRERLENLRKKPENIPQPISNTSRANLNKLLTDYTEMKKAIL |
| | 489) | HVYWEEFQKDPVGLMSRVAQPASKKIDQNKLKPEMDEKGNLTTAGF |
| | | ACSQCGQPLFVYKLEQVSEKGKAYTNYFGRCNVAEHEKLILLAQLK |
| | | PEKDSDEAVTYSLGKFGQRALDFYSIHVTRESNHPVKPLEQIGGNS |
| | | CASGPVGKALSDACMGAVASFLTKYQDIILEHQKVIKKNEKRLANL |
| | | KDIASANGLAFPKITLPPQPHTKEGIEAYNNVVAQIVIWVNLNLWO |
| | | KLKIGRDEAKPLQRLKGFPSFPLVERQANEVDWWDMVCNVKKLINE |
| | | KKEDGKVFWQNLAGYKRQEALRPYLSSEEDRKKGKKFARYQLGDLL |
| | | LHLEKKHGEDWGKVYDEAWERIDKKVEGLSKHIKLEEERRSEDAQS |
| | | KAALTDWLRAKASFVIEGLKEADKDEFCRCELKLQKWYGDLRGKPF |
| | | AIEAENSILDISGFSKQYNCAFIWQKDGVKKLNLYLIINYFKGGKL |
| | | RFKKIKPEAFEANRFYTVINKKSGEIVPMEVNFNFDDPNLIILPLA |
| | | FGKRQGREFIWNDLLSLETGSLKLANGRVIEKTLYNRRTRQDEPAL |
| | | FVALTFERREVLDSSNIKPMNLIGVDRGENIPAVIALTDPEGCPLS |
| | | RFKDSLGNPTHILRIGESYKEKQRTIQAKKEVEQRRAGGYSRKYAS |
| | | KAKNLADDMVRNTARDLLYYAVTQDAMLIFENLSRGFGRQGKRTFM |
| | | AERQYTRMEDWLTAKLAYEGLSKTYLSKTLAQYTSKTCSNCGFTIT |
| | | SADYDRVLEKLKKTATGWMTTINGKELKVEGQITYYNRYKRQNVVK |
| | | DLSVELDRLSEESVNNDISSWTKGRSGEALSLLKKRFSHRPVQEKF |
| | | VCLNCGFETHADEQAALNIARSWLFLRSQEYKKYQTNKTTGNTDKR |
| | | AFVETWQSFYRKKLKEVWKPAVTSPKKKRKV (SEQ ID NO: |
| | | 490) |

### Example 6: Generation of RNA Guides

For the generation of RNA single guides and spacers, templates for in vitro transcription were generated by performing PCR with Q5 polymerase (NEB M0491) according to the recommended protocol, with template oligos for each backbone and amplification primers with the T7 promoter and the spacer sequence. The DNA primer sequences for the T7 promoter, guide and spacer for guides and spacers are presented in Table 10, below. The template oligos, labeled “backbone fwd” and “backbone rev” for each scaffold, were included at a final concentration of 20 nM each, and the amplification primers (T7 promoter and the unique spacer primer) were included at a final concentration of 1 uM each. The sg2, sg32, sg64, and sg174 guides correspond to SEQ ID NOS: 5, 2104, 2106, and 2238, respectively, with the exception that sg2, sg32, and sg64 were modified with an additional 5′ G to increase transcription efficiency (compare sequences in Table 10 to Table 2). The 7.37 spacer targets beta2-microglobulin (B2M). Following PCR amplification, templates were cleaned and isolated by phenol-chloroform-isoamyl alcohol extraction followed by ethanol precipitation.

In vitro transcriptions were carried out in buffer containing 50 mM Tris pH 8.0, 30 mM MgCl₂, 0.01% Triton X-100, 2 mM spermidine, 20 mM DTT, 5 mM NTPs, 0.5 μM template, and 100 μg/mL T7 RNA polymerase. Reactions were incubated at 37° C. overnight. 20 units of DNase I (Promega #M6101)) were added per 1 mL of transcription volume and incubated for one hour. RNA products were purified via denaturing PAGE, ethanol precipitated, and resuspended in 1× phosphate buffered saline. To fold the sgRNAs, samples were heated to 70° C. for 5 min and then cooled to room temperature. The reactions were supplemented to 1 mM final MgCl₂ concentration, heated to 50° C. for 5 min and then cooled to room temperature. Final RNA guide products were stored at −80° C.

TABLE 10
Sequences for generation of guide RNA

| Primer | Primer sequence | RNA product |
|---|---|---|
| T7 promoter | GAAATTAATACGACTCACTATA (SEQ ID NO: | Used for all |
| primer | 491) | |
| sg2 backbone | GAAATTAATACGACTCACTATAGGTACTGGCGCTT | GGUACUGGCGCUUUU |
| fwd | TTATCTCATTACTTTGAGAGCCATCACGAGCGACT | ACUAUGUCGUAUGGG |
| | ATGTCGTATGGGTAAAG (SEQ ID NO: 492) | UAAAGCGCUUAUUUA |
| sg2 backbone | CTTTGATGCTTCTTATTTATCGGATTTCTCTCCGA | UCGGAGAGAAAUCCG |
| rev | TAAATAAGCGCTTTACCCATAC GACATAG TCGCTG | AUAAAUAAGAAGCAU |
| | GTGATGGC (SEQ ID NO: 493) | CAAAGGGCCGAGAUG |
| sg2.7.37 | CGGAGCGAGACATCTCGGCCCTTTGATGCTTCTTA | UCUCGCUCCG (SEQ |
| spacer primer | TTTATCGGATTTCTCTCCG (SEQ ID NO: | ID NO: 504) |
| | 494) | |
| sg32 backbone | GAAATTAATACGACTCACTATAGGTACTGGCGCTT | AUCUCAUUACUUUGA |
| fwd | TTATCTCATTACTTTGAGAGCCATCACCAGCGACT | GAGCCAUCACCAGCG |
| | ATGTCGTATGGGTAAAGCGC (SEQ ID NO: | GGUACUGGCGCUUUU |
| | 495) | ACUAUGUCGUAUGGG |
| sg32 backbone | CTTTGATGCTTCCCTCCGAAGAGGGCGCTTTACCC | UAAAGCGCCCUCUUC |
| rev | ATACGACATAG (SEQ ID NO: 496) | GGAGGGAAGCAUCAA |
| | | AGGGCCGAGAUGUCU |
| | | CG (SEQ ID NO: |
| | | 505) |
| sg32.7.37 | CGGAGCGAGACATCTCGGCCCTTTGATGCTTCCCT | AUCUCAUUACUUUGA |
| spacer primer | CCGAAGAG (SEQ ID NO: 497) | GAGCCAUCACCAGCG |
| sg64 backbone | GAAATTAATACGACTCACTATAGGTACTGGCGCCT | GGUACUGGCGCCUUU |
| fwd | TTATCTCATTACTTTGAGAGCCATCACCAGCGACT | ACUAUGUCGUAUGGG |
| | ATGTCGTATGGGTAAAGCGC (SEQ ID NO: | UAAAGCGCUUACGGA |
| | 498) | CUUCGGUCCGUAAGA |
| sg64 backbone | CTTTGATGCTTCTTACGGACCGAAGTCCGTAAGCG | AGCAUCAAAGGGCCG |
| rev | CTTTACCCATACGACATAG (SEQ ID NO: | AGAUGUCUCGCUCCG |
| | 499) | (SEQ ID NO: |
| sg64.7.37 | CGGAGCGAGACATCTCGGCCCTTTGATGCTTCTTA | 506) |
| spacer primer | CGGACCGAAG (SEQ ID NO: 500) | |
| sgl74 | GAAATTAATACGACTCACTATAACTGGCGCTTTTA | AUCUCAUUACUUUGA |
| backbone fwd | TCTGATTACTTTGAGAGCCATCACCAGCGACTATG | GAGCCAUCACCAGCG |
| | TCGTAGTGGGTAAAGCT (SEQ ID NO: 501) | ACUGGCGCUUUUAUC |
| sg174 | CTTTGATGCTCCCTCCGAAGAGGGAGCTTTACCCA | AUGUCGUAgUGGGUA |
| backbone rev | CTACGACATAGTCGC (SEQ ID NO: 502) | AAGCUCCCUCUUCGG |
| sg174.7.37 | CGGAGCGAGACATCTCGGCCCTTTGATGCTCCCTC | AGGGAGCAUCAAAGG |
| spacer primer | C (SEQ ID NO: 503) | UgAUUACUUUGAGAG |
| | | CCAUCACCAGCGACU |
| | | GCCGAGAUGUCUCGC |
| | | UCCG (SEQ ID |
| | | NO: 507) |

### Example 7: RNP Assembly

Purified wild-type and RNP of CasX and single guide RNA (sgRNA) were either prepared immediately before experiments or prepared and snap-frozen in liquid nitrogen and stored at −80° C. for later use. To prepare the RNP complexes, the CasX protein was incubated with sgRNA at 1:1.2 molar ratio. Briefly, sgRNA was added to Buffer #1 (25 mM NaPi, 150 mM NaCl, 200 mM trehalose, 1 mM MgCl2), then the CasX was added to the sgRNA solution, slowly with swirling, and incubated at 37° C. for 10 min to form RNP complexes. RNP complexes were filtered before use through a 0.22 μm Costar 8160 filters that were pre-wet with 200 μl Buffer #1. If needed, the RNP sample was concentrated with a 0.5 ml Ultra 100-Kd cutoff filter, (Millipore part #UFC510096), until the desired volume was obtained. Formation of competent RNP was assessed as described in Example 12.

### Example 8: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 9: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 pM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex. The experiments are expected to demonstrate the improved binding affinity of the RNP comprising a CasX variant and gNA variant compared to an RNP comprising a reference CasX and reference gNA.

### Example 10: Editing of Gene Targets PCSK9, PMP22, TRAC, SOD1, B2M and HTT

The purpose of this study was to evaluate the ability of the CasX variant 119 and gNA variant 174 to edit nucleic acid sequences in six gene targets.

### Materials and Methods

Spacers for all targets except B2M and SOD1 were designed in an unbiased manner based on PAM requirements (TTC or CTC) to target a desired locus of interest. Spacers targeting B2M and SOD1 had been previously identified within targeted exons via lentiviral spacer screens carried out for these genes. Designed spacers for the other targets were ordered from Integrated DNA Technologies (IDT) as single-stranded DNA (ssDNA) oligo pairs. ssDNA spacer pairs were annealed together and cloned via Golden Gate cloning into a base mammalian-expression plasmid construct that contains the following components: codon optimized Cas X 119 protein+NLS under an EF1A promoter, guide scaffold 174 under a U6 promoter, carbenicillin and puromycin resistance genes. Assembled products were transformed into chemically-competent E. coli, plated on Lb-Agar plates (LB: Teknova Cat #L9315, Agar: Quartzy Cat #214510) containing carbenicillin and incubated at 37° C. Individual colonies were picked and miniprepped using Qiagen Qiaprep spin Miniprep Kit (Qiagen Cat #27104) following the manufacturer's protocol. The resulting plasmids were sequenced through the guide scaffold region via Sanger sequencing (Quintara Biosciences) to ensure correct ligation.

HEK 293T cells were grown in Dulbecco's Modified Eagle Medium (DMEM; Corning Cellgro, #10-013-CV) supplemented with 10% fetal bovine serum (FBS; Seradigm, #1500-500), 100 Units/ml penicillin and 100 mg/ml streptomycin (100×-Pen-Strep; GIBCO #15140-122), sodium pyruvate (100×, Thermofisher #11360070), non-essential amino acids (100× Thermofisher #11140050), HEPES buffer (100× Thermofisher #15630080), and 2-mercaptoethanol (1000× Thermofisher #21985023). Cells were passed every 3-5 days using TryplE and maintained in an incubator at 37° C. and 5% CO2.

On day 0, HEK293T cells were seeded in 96-well, flat-bottom plates at 30 k cells/well. On day 1, cells were transfected with 100 ng plasmid DNA using Lipofectamine 3000 according to the manufacturer's protocol. On day 2, cells were switched to FB medium containing puromycin. On day 3, this media was replaced with fresh FB medium containing puromycin. The protocol after this point diverged depending on the gene of interest. Day 4 for PCSK9, PMP22, and TRAC: cells were verified to have completed selection and switched to FB medium without puromycin. Day 4 for B2M, SOD1, and HTT: cells were verified to have completed selection and passed 1:3 using TryplE into new plates containing FB medium without puromycin. Day 7 for PCSK9, PMP22, and TRAC: cells were lifted from the plate, washed in dPBS, counted, and resuspended in Quick Extract (Lucigen, QE09050) at 10,000 cells/W. Genomic DNA was extracted according to the manufacturer's protocol and stored at −20° C. Day 7 for B2M, SOD1, and HTT: cells were lifted from the plate, washed in dPBS, and genomic DNA was extracted with the Quick-DNA Miniprep Plus Kit (Zymo, D4068) according to the manufacturer's protocol and stored at −20° C.

NGS Analysis: Editing in cells from each experimental sample was assayed using next generation sequencing (NGS) analysis. All PCRs were carried out using the KAPA HiFi HotStart ReadyMix PCR Kit (KR0370). The template for genomic DNA sample PCR was 5 μl of genomic DNA in QE at 10 k cells/μL for PCSK9, PMP22, and TRAC. The template for genomic DNA sample PCR was 400 ng of genomic DNA in water for B2M, SOD1, and HTT. Primers were designed specific to the target genomic location of interest to form a target amplicon. These primers contain additional sequence at the 5′ ends to introduce Illumina read and 2 sequences. Further, they contain a 7 nt randomer sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Resultant sequencing reads were aligned to a reference sequence and analyzed for indels. Samples with editing that did not align to the estimated cut location or with unexpected alleles in the spacer region were discarded.

### Results

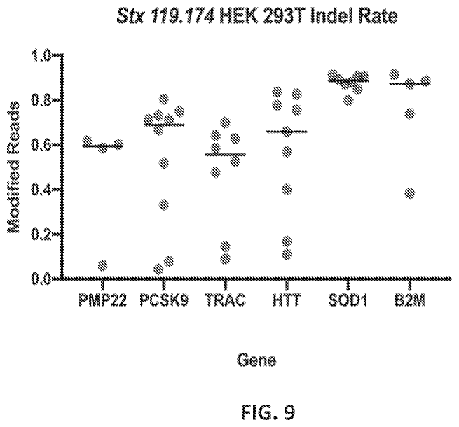

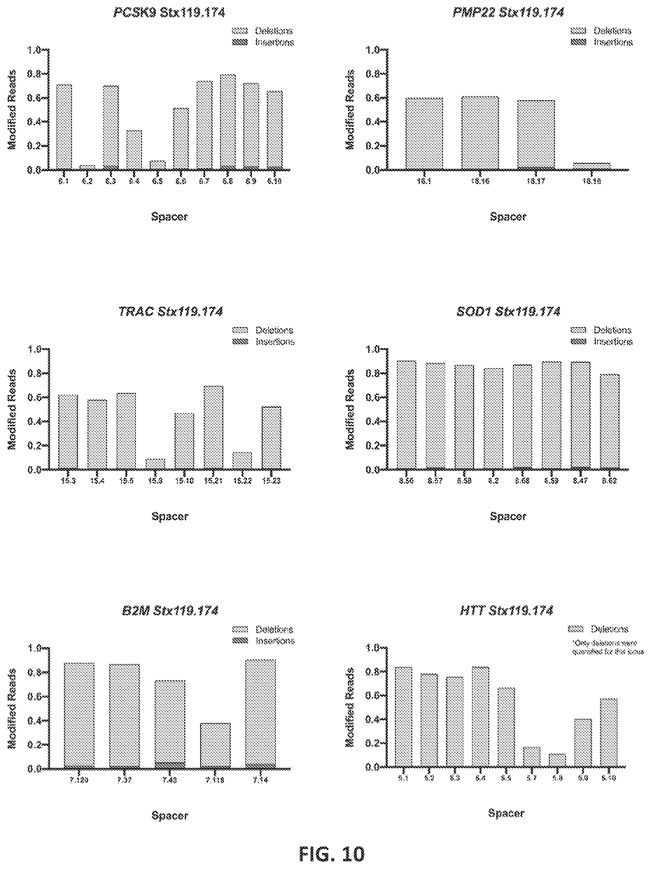

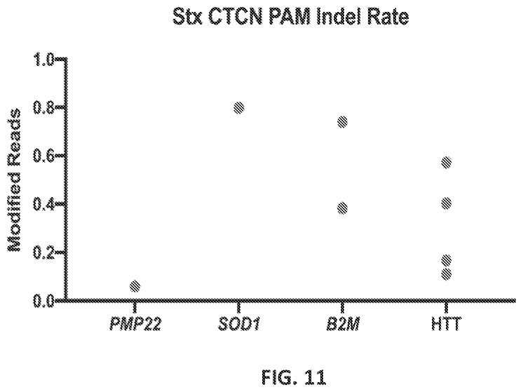

In order to validate the editing effected by the CasX:gNA 119.174 at a variety of genetic loci, a clonal plasmid transfection experiment was performed in HEK 293T cells. Multiple spacers (Table 11, listing the encoding DNA and the RNA sequences of the actual gNA spacers) were designed and cloned into an expression plasmid encoding the CasX 119 nuclease and guide 174 scaffold. HEK 293T cells were transfected with plasmid DNA, selected with puromycin, and harvested for genomic DNA six days post-transfection. Genomic DNA was analyzed via next generation sequencing (NGS) and aligned to a reference DNA sequence for analysis of insertions or deletions (indels). CasX:gNA 119.174 was able to efficiently generate indels across the 6 target genes, as shown in FIGS. 9 and 10. Indel rates varied between spacers, but median editing rates were consistently at 60% or higher, and in some cases, indel rates as high as 91% were observed. Additionally, spacers with non-canonical CTC PAMs were demonstrated to be able to generate indels with all tested target genes (FIG. 11).

The results demonstrate that the CasX variant 119 and gNA variant 174 can consistently and efficiently generate indels at a wide variety of genetic loci in human cells. The unbiased selection of many of the spacers used in the assays shows the overall effectiveness of the 119.174 RNP molecules to edit genetic loci, while the ability to target to spacers with both a TTC and a CTC PAM demonstrates its increased versatility compared to reference CasX that edit only with the TTC PAM.

TABLE 11
Spacer sequences targeting each genetic locus.

| Gene | Spacer | PAM | Spacer DNA Sequence | Spacer RNA Sequence |
|---|---|---|---|---|
| PCSK9 | 6.1 | TTC | GAGGAGGACGGCCTGGCCG | GAGGAGGACGGCCUGGCCGA |
| | | | A (SEQ ID NO: 508) | (SEQ ID NO: 552) |
| PCSK9 | 6.2 | TTC | ACCGCTGCGCCAAGGTGCG | ACCGCUGCGCCAAGGUGCGG |
| | | | G (SEQ ID NO: 509) | (SEQ ID NO: 553) |
| PCSK9 | 6.4 | TTC | GCCAGGCCGTCCTCCTCGG | GCCAGGCCGUCCUCCUCGGA |
| | | | A (SEQ ID NO: 510) | (SEQ ID NO: 554) |
| PCSK9 | 6.5 | TTC | GTGCTCGGGTGCTTCGGCC | GUGCUCGGGUGCUUCGGCCA |
| | | | A (SEQ ID NO: 511) | (SEQ ID NO: 555) |
| PCSK9 | 6.3 | TTC | ATGGCCTTCTTCCTGGCTT | AUGGCCUUCUUCCUGGCUUC |
| | | | C (SEQ ID NO: 512) | (SEQ ID NO: 556) |
| PCSK9 | 6.6 | TTC | GCACCACCACGTAGGTGCC | GCACCACCACGUAGGUGCCA |
| | | | A (SEQ ID NO: 513) | (SEQ ID NO: 557) |
| PCSK9 | 6.7 | TTC | TCCTGGCTTCCTGGTGAAG | UCCUGGCUUCCUGGUGAAGA |
| | | | A (SEQ ID NO: 514) | (SEQ ID NO: 558) |
| PCSK9 | 6.8 | TTC | TGGCTTCCTGGTGAAGATG | UGGCUUCCUGGUGAAGAUGA |
| | | | A (SEQ ID NO: 515) | (SEQ ID NO: 559) |
| PCSK9 | 6.9 | TTC | CCAGGAAGCCAGGAAGAAG | CCAGGAAGCCAGGAAGAAGG |
| | | | G (SEQ ID NO: 516) | (SEQ ID NO: 560) |
| PCSK9 | 6.10 | TTC | TCCTTGCATGGGGCCAGGA | UCCUUGCAUGGGGCCAGGAU |
| | | | T (SEQ ID NO: 517) | (SEQ ID NO: 561) |
| PMP22 | 18.16 | TTC | GGCGGCAAGTTCTGCTCAG | GGCGGCAAGUUCUGCUCAGC |
| | | | C (SEQ ID NO: 518) | (SEQ ID NO: 562) |
| PMP22 | 18.17 | TTC | TCTCCACGATCGTCAGCGT | UCUCCACGAUCGUCAGCGUG |
| | | | G (SEQ ID NO: 519) | (SEQ ID NO: 563) |
| PMP22 | 18.18 | CTC | ACGATCGTCAGCGTGAGTG | ACGAUCGUCAGCGUGAGUGC |
| | | | C (SEQ ID NO: 520) | (SEQ ID NO: 564) |
| PMP22 | 18.1 | TTC | CTCTAGCAATGGATCGTGG | CUCUAGCAAUGGAUCGUGGG |
| | | | G (SEQ ID NO: 521) | (SEQ ID NO: 565) |
| TRAC | 15.3 | TTC | CAAACAAATGTGTCACAAA | CAAACAAAUGUGUCACAAAG |
| | | | G (SEQ ID NO: 522) | (SEQ ID NO: 566) |
| TRAC | 15.4 | TTC | GATGTGTATATCACAGACA | GAUGUGUAUAUCACAGACAA |
| | | | A (SEQ ID NO: 523) | (SEQ ID NO: 567) |
| TRAC | 15.5 | TTC | GGAATAATGCTGTTGTTGA | GGAAUAAUGCUGUUGUUGAA |
| | | | A (SEQ ID NO: 524) | (SEQ ID NO: 568) |
| TRAC | 15.9 | TTC | AAATCCAGTGACAAGTCTG | AAAUCCAGUGACAAGUCUGU |
| | | | T (SEQ ID NO: 525) | (SEQ ID NO: 569) |
| TRAC | 15.10 | TTC | AGGCCACAGCACTGTTGCT | AGGCCACAGCACUGUUGCUC |
| | | | C (SEQ ID NO: 526) | (SEQ ID NO: 570) |
| TRAC | 15.21 | TTC | AGAAGACACCTTCTTCCCC | AGAAGACACCUUCUUCCCCA |
| | | | A (SEQ ID NO: 527) | (SEQ ID NO: 571) |
| TRAC | 15.22 | TTC | TCCCCAGCCCAGGTAAGGG | UCCCCAGCCCAGGUAAGGGC |
| | | | C (SEQ ID NO: 528) | (SEQ ID NO: 572) |
| TRAC | 15.23 | TTC | CCAGCCCAGGTAAGGGCAG | CCAGCCCAGGUAAGGGCAGC |
| | | | C (SEQ ID NO: 529) | (SEQ ID NO: 573) |
| HTT | 5.1 | TTC | AGTCCCTCAAGTCCTTCCA | AGUCCCUCAAGUCCUUCGAG |
| | | | G (SEQ ID NO: 530) | (SEQ ID NO: 574) |
| HTT | 5.2 | TTC | AGCAGCAGCAGCAGCAGCA | AGCAGCAGCAGCAGCAGCAG |
| | | | G (SEQ ID NO: 531) | (SEQ ID NO: 575) |
| HTT | 5.3 | TTC | TCAGCCGCCGCCGCAGGCA | UCAGCCGCCGCCGCAGGCAC |
| | | | C (SEQ ID NO: 532) | (SEQ ID NO: 576) |
| HTT | 5.4 | TTC | AGGGTCGCCATGGCGGTCT | AGGGUCGCCAUGGCGGUCUC |
| | | | C (SEQ ID NO: 533) | (SEQ ID NO: 577) |
| HTT | 5.5 | TTC | TCAGCTTTTCCAGGGTCGC | UCAGCUUUUCCAGGGUCGCC |
| | | | C (SEQ ID NO: 534) | (SEQ ID NO: 578) |
| HTT | 5.7 | CTC | GCCGCAGCCGCCCCCGCCG | GCCGCAGCCGCCCCCGCCGC |
| | | | C (SEQ ID NO: 535) | (SEQ ID NO: 579) |
| HTT | 5.8 | CTC | GCCACAGCCGGGCCGGGTG | GCCACAGCCGGGCCGGGUGG |
| | | | G (SEQ ID NO: 536) | (SEQ ID NO: 580) |
| HTT | 5.9 | CTC | TCAGCCACAGCCGGGCCGG | UCAGCCACAGCCGGGCCGGG |
| | | | G (SEQ ID NO: 537) | (SEQ ID NO: 581) |
| HTT | 5.10 | CTC | CGGTCGGTGCAGCGGCTCC | CGGUCGGUGCAGCGGCUCCU |
| | | | T (SEQ ID NO: 538) | (SEQ ID NO: 582) |
| SOD1 | 8.56 | TTC | CCACACCTTCACTGGTCCA | CCAGACCUUCACUGGUCCAU |
| | | | T (SEQ ID NO: 539) | (SEQ ID NO: 583) |
| SOD1 | 8.57 | TTC | TAAAGGAAAGTAATGGACC | UAAAGGAAAGUAAUGGACCA |
| | | | A (SEQ ID NO: 540) | (SEQ ID NO: 584) |
| SOD1 | 8.58 | TTC | CTGGTCCATTACTTTCCTT | CUGGUCCAUUACUUUCCUUU |
| | | | T (SEQ ID NO: 541) | (SEQ ID NO: 585) |
| SOD1 | 8.2 | TTC | ATGTTCATGAGTTTGGAGA | AUGUUCAUGAGUUUGGAGAU |
| | | | T (SEQ ID NO: 542) | (SEQ ID NO: 586) |
| SOD1 | 8.68 | TTC | TGAGTTTGGAGATAATAGA | UGAGUUUGGAGAUAAUACAG |
| | | | G (SEQ ID NO: 543) | (SEQ ID NO: 587) |
| SOD1 | 8.59 | TTC | ATAGACACATCGGCCACAC | AUAGACACAUCGGCCACACC |
| | | | C (SEQ ID NO: 544) | (SEQ ID NO: 588) |
| SOD1 | 8.47 | TTC | TTATTAGGCATGTTGGAGA | UUAUUAGGCAUGUUGGAGAC |
| | | | C (SEQ ID NO: 545) | (SEQ ID NO: 589) |
| SOD1 | 8.62 | CTC | CAGGAGACCATTGCATCAT | CAGGAGACCAUUGCAUCAUU |
| | | | T (SEQ ID NO: 546) | (SEQ ID NO: 590) |
| B2M | 7.120 | TTC | GGCCTGGAGGCTATCCAGC | GGCCUGGAGGCUAUCCAGCG |
| | | | G (SEQ ID NO: 547) | (SEQ ID NO: 591) |
| B2M | 7.37 | TTC | GGCCGAGATGTCTCGCTCC | GGCCGAGAUGUCUCGCUCCG |
| | | | G (SEQ ID NO: 548) | (SEQ ID NO: 592) |
| B2M | 7.43 | CTC | AGGCCAGAAAGAGAGAGTA | AGGCCAGAAAGAGAGAGUAG |
| | | | G (SEQ ID NO: 549) | (SEQ ID NO: 593) |
| B2M | 7.119 | CTC | CGCTGGATAGCCTCCAGGC | CGCUGGAUAGCCUCCAGGCC |
| | | | C (SEQ ID NO: 550) | (SEQ ID NO: 594) |
| B2M | 7.14 | TTC | TGAAGCTGACAGCATTCGG | UGAAGCUGACAGCAUUCGGG |
| | | | G (SEQ ID NO: 551) | (SEQ ID NO: 595) |

### Example 11: Assessing Differential PAM Recognition In Vitro

Purified wild-type and engineered CasX variants will be complexed with single-guide RNA bearing a fixed targeting sequence. The RNP complexes will be added to buffer containing MgCl2 at a final concentration of 100 nM and incubated with 5′ Cy7.5-labeled double-stranded target DNA at a concentration of 10 nM. Separate reactions will be carried out with different DNA substrates containing different PAMs adjacent to the target nucleic acid sequence. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the rate of cleavage of the non-canonical PAMs by the CasX variants will be determined.

### Example 12: CasX:gNA In Vitro Cleavage Assays

### 1. Determining Cleavage-Competent Fractions for Protein Variants Compared to Wild-Type Reference CasX

The ability of CasX variants to form active RNP compared to reference CasX was determined using an in vitro cleavage assay. The beta-2 microglobulin (B2M) 7.37 target for the cleavage assay was created as follows. DNA oligos with the sequence TGAAGCTGACAGCATTCGGGCCGAGATGTCTCGCTCCGTGGCCTTAGCTGTGCTCGC GCT (non-target strand, NTS (SEQ ID NO: 596)) and TGAAGCTGACAGCATTCGGGCCGAGATGTCTCGCTCCGTGGCCTTAGCTGTGCTCGC GCT (target strand, TS (SEQ ID NO: 597)) were purchased with 5′ fluorescent labels (LI-COR IRDye 700 and 800, respectively). dsDNA targets were formed by mixing the oligos in a 1:1 ratio in 1× cleavage buffer (20 mM Tris HCl pH 7.5, 150 mM NaCl, 1 mM TCEP, 5% glycerol, 10 mM MgCl₂), heating to 95° C. for 10 minutes, and allowing the solution to cool to room temperature.

CasX RNPs were reconstituted with the indicated CasX and guides (see graphs) at a final concentration of 1 μM with 1.5-fold excess of the indicated guide unless otherwise specified in 1× cleavage buffer (20 mM Tris HCl pH 7.5, 150 mM NaCl, 1 mM TCEP, 5% glycerol, 10 mM MgCl₂) at 37° C. for 10 min before being moved to ice until ready to use. The 7.37 target was used, along with sgRNAs having spacers complementary to the 7.37 target.

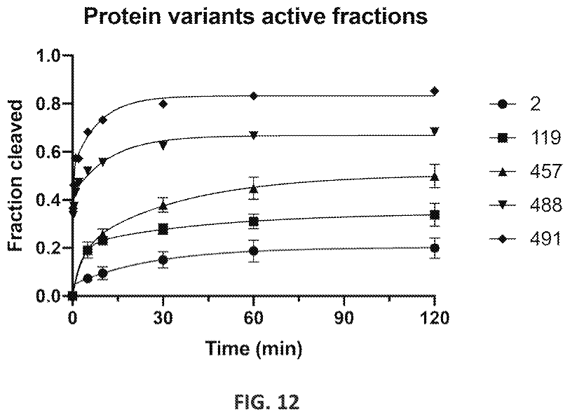

Cleavage reactions were prepared with final RNP concentrations of 100 nM and a final target concentration of 100 nM. Reactions were carried out at 37° C. and initiated by the addition of the 7.37 target DNA. Aliquots were taken at 5, 10, 30, 60, and 120 minutes and quenched by adding to 95% formamide, 20 mM EDTA. Samples were denatured by heating at 95° C. for 10 minutes and run on a 10% urea-PAGE gel. The gels were either imaged with a LI-COR Odyssey CLx and quantified using the LI-COR Image Studio software or imaged with a Cytiva Typhoon and quantified using the Cytiva IQTL software. The resulting data were plotted and analyzed using Prism. We assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. The cleavage traces were fit with a biphasic rate model, as the cleavage reaction clearly deviates from monophasic under this concentration regime, and the plateau was determined for each of three independent replicates. The mean and standard deviation were calculated to determine the active fraction (Table 12). The graphs are shown in FIG. 12.

Apparent active (competent) fractions were determined for RNPs formed for CasX2+guide 174+7.37 spacer, CasX119+guide 174+7.37 spacer, CasX457+guide 174+7.37 spacer, CasX488+guide 174+7.37 spacer, and CasX491+guide 174+7.37 spacer. The determined active fractions are shown in Table 12. All CasX variants had higher active fractions than the wild-type CasX2, indicating that the engineered CasX variants form significantly more active and stable RNP with the identical guide under tested conditions compared to wild-type CasX. This may be due to an increased affinity for the sgRNA, increased stability or solubility in the presence of sgRNA, or greater stability of a cleavage-competent conformation of the engineered CasX: sgRNA complex. An increase in solubility of the RNP was indicated by a notable decrease in the observed precipitate formed when CasX457, CasX488, or CasX491 was added to the sgRNA compared to CasX2.

2. In Vitro Cleavage Assays—Determining k_{cleave} for CasX Variants Compared to Wild-Type Reference CasX

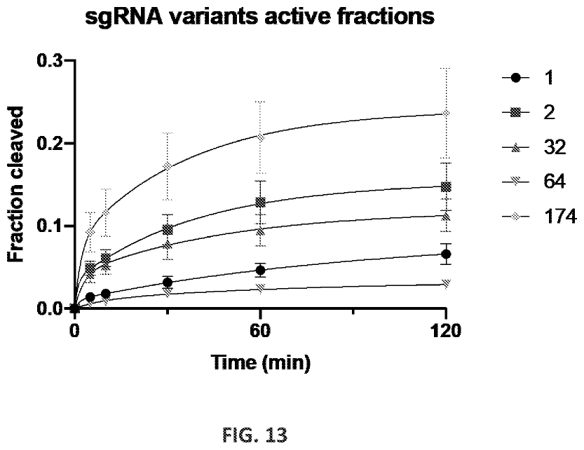

Cleavage-competent fractions were also determined using the same protocol for CasX2.2.7.37, CasX2.32.7.37, CasX2.64.7.37, and CasX2.174.7.37 to be 16±3%, 13±3%, 5±2%, and 22±5%, as shown in FIG. 13 and Table 12.

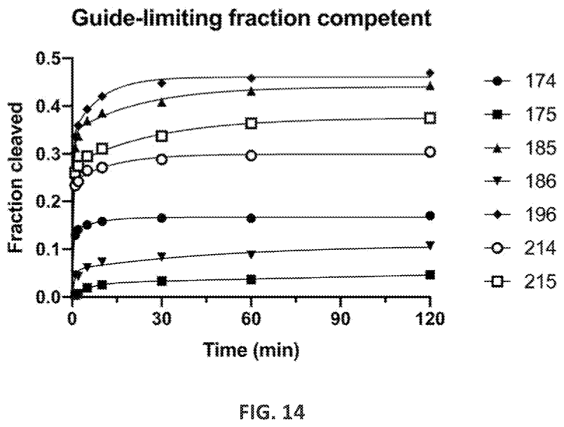

A second set of guides were tested under different conditions to better isolate the contribution of the guide to RNP formation. 174, 175, 185, 186, 196, 214, and 215 guides with 7.37 spacer were mixed with CasX491 at final concentrations of 1 μM for the guide and 1.5 μM for the protein, rather than with excess guide as before. Results are shown in FIG. 14 and Table 12. Many of these guides exhibited additional improvement over 174, with 185 and 196 achieving 44% and 46% competent fractions, respectively, compared with 17% for 174 under these guide-limiting conditions.

The data indicate that both CasX variants and sgRNA variants are able to form a higher degree of active RNP with guide RNA compare to wild-type CasX and wild-type sgRNA.

The apparent cleavage rates of CasX variants 119, 457, 488, and 491 compared to wild-type reference CasX were determined using an in vitro fluorescent assay for cleavage of the target 7.37.

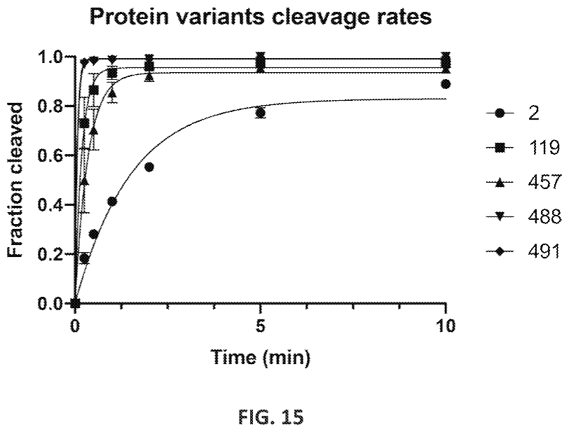

CasX RNPs were reconstituted with the indicated CasX (see FIG. 15) at a final concentration of 1 μM with 1.5-fold excess of the indicated guide in 1× cleavage buffer (20 mM Tris HCl pH 7.5, 150 mM NaCl, 1 mM TCEP, 5% glycerol, 10 mM MgCl₂) at 37° C. for 10 min before being moved to ice until ready to use. Cleavage reactions were set up with a final RNP concentration of 200 nM and a final target concentration of 10 nM. Reactions were carried out at 37° C. except where otherwise noted and initiated by the addition of the target DNA. Aliquots were taken at 0.25, 0.5, 1, 2, 5, and 10 minutes and quenched by adding to 95% formamide, 20 mM EDTA. Samples were denatured by heating at 95° C. for 10 minutes and run on a 10% urea-PAGE gel. The gels were imaged with a LI-COR Odyssey CLx and quantified using the LI-COR Image Studio software or imaged with a Cytiva Typhoon and quantified using the Cytiva IQTL software. The resulting data were plotted and analyzed using Prism, and the apparent first-order rate constant of non-target strand cleavage (k_{cleave}) was determined for each CasX: sgRNA combination replicate individually. The mean and standard deviation of three replicates with independent fits are presented in Table 12, and the cleavage traces are shown in FIG. 15.

Apparent cleavage rate constants were determined for wild-type CasX2, and CasX variants 119, 457, 488, and 491 with guide 174 and spacer 7.37 utilized in each assay (see Table 12 and FIG. 15). All CasX variants had improved cleavage rates relative to the wild-type CasX2. CasX457 cleaved more slowly than 119, despite having a higher competent fraction as determined above. CasX488 and CasX491 had the highest cleavage rates by a large margin; as the target was almost entirely cleaved in the first timepoint, the true cleavage rate exceeds the resolution of this assay, and the reported k_{cleave} should be taken as a lower bound.

The data indicate that the CasX variants have a higher level of activity, with k_{cleave} rates reaching at least 30-fold higher compared to wild-type CasX2.

### 3. In Vitro Cleavage Assays: Comparison of Guide Variants to Wild-Type Guides

Cleavage assays were also performed with wild-type reference CasX2 and reference guide 2 compared to guide variants 32, 64, and 174 to determine whether the variants improved cleavage. The experiments were performed as described above. As many of the resulting RNPs did not approach full cleavage of the target in the time tested, we determined initial reaction velocities (Vo) rather than first-order rate constants. The first two timepoints (15 and 30 seconds) were fit with a line for each CasX:sgRNA combination and replicate. The mean and standard deviation of the slope for three replicates were determined.

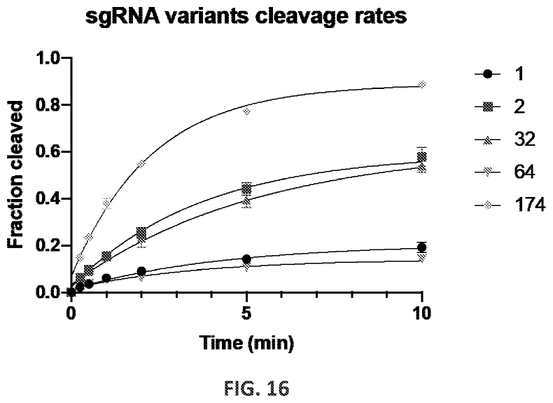

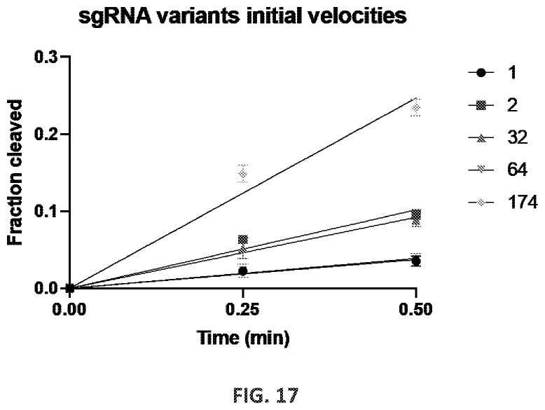

Under the assayed conditions, the Vo for CasX2 with guides 2, 32, 64, and 174 were 20.4±1.4 nM/min, 18.4±2.4 nM/min, 7.8±1.8 nM/min, and 49.3±1.4 nM/min (see Table 12 and FIGS. 16-17). Guide 174 showed substantial improvement in the cleavage rate of the resulting RNP (˜2.5-fold relative to 2, see FIG. 17), while guides 32 and 64 performed similar to or worse than guide 2. Notably, guide 64 supports a cleavage rate lower than that of guide 2 but performs much better in vivo (data not shown). Some of the sequence alterations to generate guide 64 likely improve in vivo transcription at the cost of a nucleotide involved in triplex formation. Improved expression of guide 64 likely explains its improved activity in vivo, while its reduced stability may lead to improper folding in vitro.

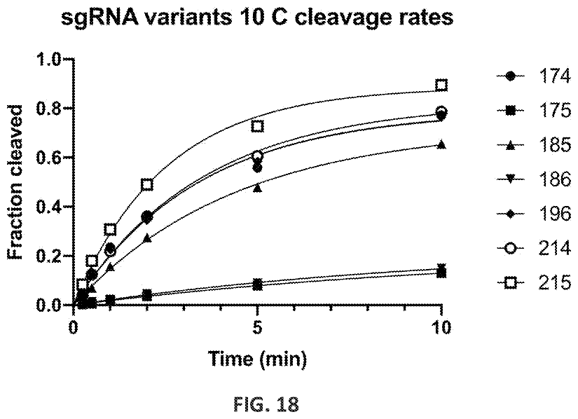

Additional experiments were carried out with guides 174, 175, 185, 186, 196, 214, and 215 with spacer 7.37 and CasX491 to determine relative cleavage rates. To reduce cleavage kinetics to a range measurable with our assay, the cleavage reactions were incubated at 10° C. Results are in FIG. 18 and Table 12. Under these conditions, 215 was the only guide that supported a faster cleavage rate than 174. 196, which exhibited the highest active fraction of RNP under guide-limiting conditions, had kinetics essentially the same as 174, again highlighting that different variants result in improvements of distinct characteristics.

The data support that, under the conditions of the assay, use of the majority of the guide variants with CasX results in RNP with a higher level of activity than one with the wild-type guide, with improvements in initial cleavage velocity ranging from ˜2-fold to >6-fold. Numbers in Table 12 indicate, from left to right, CasX variant, sgRNA scaffold, and spacer sequence of the RNP construct. In the RNP construct names in the table below, CasX protein variant, guide scaffold and spacer are indicated from left to right.

TABLE 12
Results of cleavage and RNP formation assays

| RNP | | | Initial | | |
|---|---|---|---|---|---|
| Construct | k_{cleave}* | | velocity* | | Competent fraction |
| 2.2.7.37 | | | 20.4 ± 1.4 | nM/min | 16 ± 3% |
| 2.32.7.37 | | | 18.4 ± 2.4 | nM/min | 13 ± 3% |
| 2.64.7.37 | | | 7.8 ± 1.8 | nM/min | 5 ± 2% |
| 2.174.7.37 | 0.51 ± 0.01 | min⁻¹ | 49.3 ± 1.4 | nM/min | 22 ± 5% |

| 119.174.7.37 | 6.29 ± 2.11 | min⁻¹ | 35 ± 6% | |
|---|---|---|---|---|
| 457.174.7.37 | 3.01 ± 0.90 | min⁻¹ | 53 ± 7% | |
| 488.174.7.37 | 15.19 | min⁻¹ | 67% | |
| 491.174.7.37 | 16.59 | min⁻¹/ | 83%/17% | (guide-limited) |
| | 0.293 | min⁻¹ (10° C.) | | |
| 491.175.7.37 | 0.089 | min⁻¹ (10° C.) | 5% | (guide-limited) |
| 491.185.7.37 | 0.227 | min⁻¹ (10° C.) | 44% | (guide-limited) |
| 491.186.7.37 | 0.099 | min⁻¹ (10° C.) | 11% | (guide-limited) |
| 491.196.7.37 | 0.292 | min⁻¹ (10° C.) | 46% | (guide-limited) |
| 491.214.7.37 | 0.284 | min⁻¹ (10° C.) | 30% | (guide-limited) |
| 491.215.7.37 | 0.398 | min⁻¹ (10° C.) | 38% | (guide-limited) |

*Mean and standard deviation

### Example 13: CasX:gNA Editing of PCSK9

This example sets forth the parameters to make and test compositions capable of modifying a PCSK9 locus.

### Experimental Design:

### A) PCSK9-Modifying Spacer Selection Process:

20 bp XTC PAM spacers will be designed to target the following regions in the human genome:
(a) PCSK9 cis enhancer elements
(b) PCSK9 proximal non-coding genetic elements highly conserved across vertebrates (UCSC genome browser)
(c) PCSK9 genomic locus. The PCSK9 gene is defined as the sequence that spans chr1:55,039,476-55,064,853 of the human genome (GRCh38/hg38) (the notation refers to the chromosome 1 (chr1), starting at the 55,039,476 bp to 55,064,853 bp on chromosome 1 (Homo sapiens Updated Annotation Release 109.20190905, GRCh38.p13) (NCBI). PCSK9 targeting spacers may be similarly assembled from other genomes.

### B) Methods for Generating PCSK9 Targeting Constructs:

In order to generate PCSK9 targeting constructs, the PCSK9 targeting spacers of Table 11 will be cloned into a base mammalian-expression plasmid construct (pStX) that is comprised of the following components: codon optimized CasX (construct CasX 119 molecule and rRNA guide 174 (119.174); see Tables for sequences)+NLS; and a mammalian selection marker, puromycin. Spacer sequence DNA will be ordered as single-stranded DNA (ssDNA) oligos from Integrated DNA Technologies (IDT) consisting of the spacer sequence and the reverse complement of this sequence. These two oligos will be annealed together and cloned into pStX individually or in bulk by Golden Gate Assembly using T4 DNA Ligase (New England BioLabs Cat #M0202L) and an appropriate restriction enzyme for the plasmid. Assembled products will be transformed into chemically- or electro-competent bacterial cells, plated on Lb-Agar plates (LB: Teknova Cat #L9315, Agar: Quartzy Cat #214510) containing carbenicillin and incubated until colonies appeared. Individual colonies will be picked and miniprepped using a Qiagen Qiaprep spin Miniprep Kit (Qiagen Cat #27104), following the manufacturer's protocol. The resultant plasmids will be sequenced using Sanger sequencing to ensure correct ligation. SaCas9 and SpyCas9 control plasmids, with spacers chosen based on Cas protein-specific PAMs, will be prepared similarly to pStX plasmids described above.

### C) Methods to Generate PCSK9 Reporter Line:

A fluorescent-encoding DNA (e.g., GFP) will be knocked in at the 3′ end of the last PCSK9 exon in a HEPG2 cell line. The modified cells will be expanded by serial passage every 3-5 days and maintained in Fibroblast (FB), consisting of Dulbecco's Modified Eagle Medium (DMEM; Corning Cellgro, #10-013-CV) supplemented with 10% fetal bovine serum (FBS; Seradigm, #1500-500), or other appropriate medium, and 100 Units/ml penicillin and 100 mg/ml streptomycin (100×-Pen-Strep; GIBCO #15140-122), and can additionally include sodium pyruvate (100×, Thermofisher #11360070), non-essential amino acids (100× Thermofisher #11140050), HEPES buffer (100× Thermofisher #15630080), and 2-mercaptoethanol (1000× Thermofisher #21985023). The cells will be incubated at 37° C. and 5% CO2. After 1-2 weeks, single GFP+ cells will be sorted into FB or other appropriate medium. The reporter line clones will be expanded by serial passage every 3-5 days and maintained in FB medium in an incubator at 37° C. and 5% CO2. The lines will be characterized via genomic sequencing, and functional modification of the PCSK9 locus using a PCSK9 targeting molecule. The optimal reporter lines will be identified as ones that i) had a single copy of GFP correctly integrated at the target PCSK9 locus, ii) maintained doubling times equivalent to unmodified cells, iii) resulted in reduction in GFP fluorescence upon disruption of the PCSK9 gene when assayed using the methods described, below.

### D) Methods to Assess PCSK9 Modifying Activity in PCSK9-GFP Reporter Cell Line:

PCSK9 reporter cells will be seeded at 20-40 k cells/well in a 96 well plate in 100 μl of FB (or other appropriate) medium and cultured in a 37° C. incubator with 5% CO2. The following day, confluence of seeded cells will be checked. Ideally, cells should be at ˜75% confluence at time of transfection. If cells will be at the right confluence, transfection will be carried out.

Each CasX construct (CasX 119 and guide 174) with appropriate spacers targeting PCSK9 will be transfected at 100-500 ng per well using Lipofectamine 3000 following the manufacturer's protocol, using 3 wells per construct as replicates. SaCas9 and SpyCas9 targeting PCSK9 will be used as benchmarking controls. For each Cas protein type, a non-targeting plasmid will be used as a negative control.

After 24-48 hours of puromycin selection at 0.3-3 μg/ml, to select for successfully transfected cells, followed by 24-48 hours of recovery in FB or other appropriate medium, fluorescence in transfected cells will be analyzed via flow cytometry. In this process, cells will be gated for the appropriate forward and side scatter, selected for single cells and then gated for reporter expression (Attune Nxt Flow Cytometer, Thermo Fisher Scientific) to quantify the expression levels of fluorophores. At least 10,000 events will be collected for each sample. The data will be used to calculate the percentage of antibody-label negative (edited) cells.

A subset of cells for each sample from the example will be lysed, and genome extracted using a Quick extract solution following the manufacturer's protocol. Editing will be analyzed using a T7E1 assay. Briefly, the genomic locus at the targeted edit site will be amplified using primers (e.g., a 500 bp region around the intended target) using a PCR program on a thermocycler. The PCR amplicon will be then hybridized following a hybridization program on a thermocycler, and then treated with T7 Endonuclease for 30 mins at 37° C. The sample will be then analyzed on a 2% agarose gel, or on a Fragment Analyzer to visualize DNA bands.

### Example 14: Methods to Assess PCSK9 Modifying Activity in HEPG2 or HEK293T Cells

HEPG2 cells or HEK293T cells will be seeded at 20-40 k cells/well in a 96 well plate in 100 μl of FB medium and cultured in a 37° C. incubator with 5% CO2. The following day, confluence of seeded cells will be checked. Cells should be at ˜75% confluence at time of transfection. If cells are at the right confluence, transfection will be carried out.

CasX construct 119 with guide 174 and the spacers of Table 11 targeting PCSK9 will be transfected at 100-500 ng per well using Lipofectamine 3000 following the manufacturer's protocol, and placed into 3 wells per construct as replicates. A non-targeting plasmid will be used as a negative control. After 24-48 hours of puromycin selection at 1-3 μg/ml to select for successfully transfected cells, followed by 24-48 hours of recovery in FB medium, cells will be analyzed for editing by the T7E1 assay as described above or by Western blotting, as described below.

### Example 15: Methods to Package PCSK9 Targeting CasX Constructs in a Lentiviral Vector

Lentiviral particles packaging PCSK9 targeting CasX:gNA constructs (e.g., CasX 119 of Example 2 and guide 174 of Example 6 and the spacers of Table 11 or encoding any one of SEQ ID NOS: 315-436, 612-2100, or 2286-13861) targeting PCSK9 will be produced by transfecting HEK293T at a confluency of 70%-90% using polyethyleneimine based transfection of transgene plasmids encoding CasX, the guide RNA, the lentiviral packaging plasmid, and the VSV-G envelope plasmids. For lentiviral particle production, media will be changed 12 hours post-transfection, and the virus will be harvested at 36-48 hours post-transfection. Viral supernatants will be filtered using 0.45 μm membrane filters, and diluted in FB media (Fibroblast medium, comprised of: DMEM with Glutamax (Gibco 10566-016) supplemented with MEM-NEAA (Thermo 11140050), sodium pyruvate (Thermo 11360070), HEPES (Thermo 15630080), 2-mercaptoethanol (Gibco 21985023), penicillin/streptomycin (Thermo 15140122) with 10% volume fraction of fetal bovine serum (FBS, VWR #97068-085)), if appropriate.

### Example 16: Methods to Assess PCSK9 Modifying Via a Lentiviral Screen

Lentiviral plasmids will be produced following standard cloning procedures such that each lentiviral plasmid has one codon optimized NLS bearing CasX molecule (e.g., construct CasX 119 molecule) and an rRNA guide 174 (119.174) with a spacer targeting PCSK9 (spacer sequences in Table 11 or the DNA counterparts of the sequences of SEQ ID NOS: 315-436, 612-2100, or 2286-13861; i.e., T substituted for U bases) with a puromycin selection marker. The cloning is carried out such that the final titer encompasses the full library size by >100× of all possible PCSK9 spacers targeting all known PAMs and their corresponding spacer regions in the PCSK9 gene and regulatory region. If ˜5,000 is the library size; the libraries evaluated would be >5×10⁵.

Lentiviral particles are produced by transfecting HEK293T at a confluency of 70%-90% using polyethylenimine based transfection of plasmids containing the spacer library, the lentiviral packaging plasmid and the VSV-G envelope plasmids. For particle production, media is changed 12 hr. post-transfection, and virus harvested at 36-48 hr. post-transfection.

Viral supernatants are filtered using 0.45 μm membrane filters, diluted in FB media if appropriate, and added to target cells, in this case the PCSK9-GFP reporter cell line. Supplement polyberene is added at 5-20 μg/ml to enhance transduction efficiency, if necessary. Transduced cells are selected for 24-48 hr. post-transduction using puromycin at 0.3-3 μg/ml in FB medium, and grown for 7-10 days in FB or other appropriate medium in a 37° C. incubator with 5% CO2.

Cells are sorted on a SH-100 or MA900 SONY sorter. In this process, cells are gated for the appropriate forward and side scatter, selected for single cells and then gated for reporter expression. Different cell sorting gates are established based on fluorescence level (OFF=full KO, Med=partial disruption or knockdown (KD), High=no edit, Very High=Enhancer) to distinguish between and collect cells editing by i) highly functional PCSK9 disrupting molecules, ii) molecules that only lower expression, and iii) molecules that increase expression. This assay can also be run to identify allele specific guides if two colors are used in human patient cells. Genomic DNA are collected from each group of sorted cells using Quick Extract (Lucigen Cat #QE09050) solution following manufacturer's recommended protocol.

Spacer libraries from each collected pool are then amplified via PCR directly from the genome and collected for deep sequencing on a Miseq. Analysis of the spacers is done according to gate and abundance for a specific activity; see below for detailed methods for NGS analysis of spacer hits.

Selected guides from each sorted group are then re-cloned and individually validated in reporter cell line and in primary human cell lines for activity by flow cytometry and T7E1 assay and/or Western blotting, and indel spectrum assessed by NGS analysis. Steps followed may be similar to the description provided under Methods to assess PCSK9 modifying activity in reporter cell line.

### Methods for NGS Analysis of Spacer Hits

Data coming from above described lentiviral screen will be analyzed using next generation (NGS) sequencing. Spacers are each assessed for ability to disrupt a PCSK9 gene using next generation sequencing (NGS). NGS libraries are generated through specific amplification of the lentiviral backbone containing the spacer. A different library is generated for each of the sorted populations (GFP high, med, low, etc., corresponding to low, med, high PCSK9 expression), and then assessed with the Illumina Hiseq.

Sequencing reads from the Illumina Hiseq are trimmed for adapter sequences and for regions of low sequencing quality. Paired end reads are merged based on their overlap sequence to form a single consensus sequence per sequenced fragment. Consensus sequences are aligned to the designed spacer sequences using bowtie2. Reads aligning to more than one designed spacer sequence are discarded.

The ‘abundance’ of each spacer sequence is defined as the number of reads aligning to that sequence. The abundance is tabulated for each sequencing library, forming a count table giving the abundance for each spacer sequence across each of the sequencing libraries (i.e., sorted populations). Finally, the numbers of abundances are then normalized to account for the differing sequencing depth of each library by dividing by the overall read count in that library, multiplied by the mean read count across libraries. The normalized count table is used to determine the activity of each spacer in each gate (high, medium, low, etc.).

A PCSK9-GFP reporter line will be constructed by knocking in GFP at the endogenous human PCSK9 locus. A reporter (e.g., GFP reporter) coupled to a gRNA targeting sequence, complementary to the gRNA spacer, is integrated into a reporter cell line. Cells are transformed or transfected with a CasX protein and/or sgRNA variant, with the spacer motif of the sgRNA complementary to and targeting the gRNA target sequence of the reporter. The ability of the CasX:sgRNA ribonucleoprotein complex to cleave the target nucleic acid sequence is assayed by FACS. Cells that lose reporter expression indicate occurrence of CasX:sgRNA ribonucleoprotein complex-mediated cleavage and indel formation. The reporting system is based on reduced GFP fluorescence upon successful modifying (editing) of the PCSK9 locus, detected by flow cytometry.

For screening purposes, the PCSK9 spacers of Table 11 or any one of SEQ ID NOS: 315-436, 612-2100, or 2286-13861 linked to the scaffold of the gNA will be tested. The spacers will be tested with the CasX protein (construct CasX 119 with gNA 174), using SaCas9 and SpyCas9 as controls, in the reporter cell line. The reduction in GFP fluorescence and editing will be evaluated in the PCSK9-GFP reporter cells, selected for successful lipofection using puromycin, and later assayed for GFP disruption via FACS. The CasX 119 and guide 174 are expected to edit at least 5-10% of cells, demonstrating that CasX can modify the endogenous PCSK9 locus, and do so more effectively than the SaCas9 and SpyCas9 system. A T7E1 assay or Western blotting will be performed to assay gene editing in the PCSK9-GFP reporter cell line. CasX 119 and guide 174 with PCSK9 targeting spacers and non-targeting control (NT) will be lipofected into PCSK9-GFP reporter cells, selected for successful lipofection using puromycin, and later assayed for gene editing in the T7E1 assay, demonstrating successful editing of the PCSK9 locus.

### Example 17: Method to Edit the PCSK9 Gene Using CasX Using Lentivirus Construct in an Allele Specific Manner

Experiments are designed and performed to show the ability of CasX to edit the PCSK9 locus. One strategy to permanently treat a PCSK9 related disorder is to specifically disrupt the mutant copy of the gene while sparing the wild-type (WT) allele. HEK293 cells with both wild-type alleles should be editable by a WT CasX spacer of Table 11 or any one of SEQ ID NOS: 315-436, 612-2100, or 2286-13861, but not by a mutant CasX spacer (e.g., a spacer not having sufficient homology to a WT PCSK9 gene sequence to bind). This example will additionally demonstrate the ability of CasX spacers to distinguish between on-target and off-target alleles that differ by a single nucleotide. HEK293 cells are seeded at 20-40 k cells/well in a 96 well plate in 100 μl of FB medium and cultured in a 37° C. incubator with 5% CO2. The following day, confluence of seeded cells are checked to ensure that cells will be at ˜75% confluence at time of transfection. If cells are at the right confluence, transfection is carried out using the viral supernatants of Example 15 (having CasX 119 and guide 174 with the spacers targeting PCSK9, as above), using 3 wells per construct as replicates. SaCas9 and SpyCas9 targeting PCSK9 are used as benchmarking controls. For each Cas protein type, a non-targeting plasmid is used as a negative control. Cells will be selected for successful transfection with puromycin at 0.3-3 μg/ml for 24-48 hours followed by 24-48 hours of recovery in FB medium. A subset of cells for each sample from the experiment will be lysed, and the genome will be extracted using a Quick extract solution following the manufacturer's protocol. Editing will be analyzed using a T7E1 assay. Briefly, the genomic locus at the targeted edit site is amplified using primers (e.g., a 500 bp region around the intended target) using a PCR program on a thermocycler. The PCR amplicon is then hybridized following a hybridization program on a thermocycler, and then treated with T7 Endonuclease for 30 mins at 37° C. The sample is then analyzed on a 2% agarose gel, or on a Fragment Analyzer to visualize the DNA bands.

### Example 18: Method to Demonstrate Allele-Specific Editing in Autosomal Dominant Hypercholesterolemia (ADH) Patient-Derived Cell Lines

Cells derived from ADH patients will be obtained and cultured under supplier recommended conditions. Cells will be transfected with a CasX construct (e.g., an RNP of CasX 119 with guide 174 and a PCSK9 spacer of Table 11 or a spacer of SEQ ID NOS: 247-303) using Lipofectamine 3000 following manufacturer's protocol, or nucleofected using Lonza nucleofector kit according to manufacturer's protocol, and seeded in 96 well plates for incubation and growth. Alternatively, CasX constructs may be packaged in a lentivirus as per Example 15, and used to transduce patient-derived cells. Cells will be selected for successful lipofection or nucleofection, or lentiviral transduction, using medium containing puromycin at 0.3-3 μg/ml for 2-4 days or longer, followed by recovery in puromycin-free medium for 2 days or longer. Editing of the PCSK9 locus may be assessed at the genomic, transcriptomic, and proteomic level. At the end of the selection and recovery period, a subset of cells for each sample from the experiment will be lysed, and genome extracted using a Quick extract (QE) solution following the manufacturer's protocol; another subset of cells will be lysed in RIPA cell lysis buffer for proteomic analysis; another subset of cells may be passaged for analysis at a later point in time. A fraction of the QE treated samples will be used to assess editing using a T7E1 assay. Briefly, the genomic locus at the targeted edit site will be amplified using primers (e.g., a 500 bp region around the intended target) using a PCR program on a thermocycler. The PCR amplicon will then be hybridized following a hybridization program on a thermocycler, and then treated with T7 Endonuclease for 30 mins at 37° C. The sample will be then analyzed on a 2% agarose gel, or on a Fragment Analyzer to visualize DNA bands to confirm that the CasX construct can successfully edit the PCSK9 mutation. Another fraction of the QE treated samples will be used to assess editing at the PCSK9 locus using NGS.

Proteomic analysis will be performed by Western blotting. The samples lysed in RIPA buffer will first be quantified for protein content using a colorimetric protein quantification assay such as BCA (Pierce) or Bradford (BioRad) according to the manufacturer's protocol. Following quantification, the sample will be diluted in beta-mercaptoethanol-supplemented Laemmli buffer to load 2.5-20 μg of total protein per well. Samples will be heat denatured at 95-100° C. for 5-10 minutes, and then cooled to room temperature. Samples will then be loaded onto and run on a polyacrylamide gel. Once the gel has run, proteins will be transferred onto a PVDF membrane, blocked for at least 1 hour at room temperature, and labeled with primary antibodies against PCSK9 and an appropriate loading control. The blot will be washed three times, at five minutes per wash, with PBST (PBS supplemented with 0.1 v/v % Triton X100) on a rocker at room temperature. An appropriate reporter-conjugated secondary antibody will then be used to label the primary antibody for 1 hour at room temperature. The blot will be washed three times, at five minutes per wash, with PBST (PBS supplemented with 0.1 v/v % Triton X100) on a rocker at room temperature. Any necessary substrates will subsequently be added, quenched if required, and imaged on a gel imager. Band intensities will be quantified using appropriate software following manufacturer's protocol.

### Example 19: Method to Deliver PCSK9-Targeting Construct Via AAV: Making and Recovery of AAV with Encoded CasX System

This example describes a typical protocol followed to produce and characterize AAV vectors packaging CasX molecules and guide

### Materials and Methods:

For AAV production, the tri-plasmid transfection method is used and requires three essential plasmids—a pTransgene plasmid carrying the PCSK9 gene of interest to be packaged in AAV, pRC, and pHelper plasmids. DNA encoding CasX and guide RNA are cloned into an AAV transgene cassette, between the ITRs to generate the pTransgene plasmid. The constructed transgene plasmid is verified via full-length plasmid sequencing, restriction digestion, and functional tests including in vitro transfection of mammalian cells. Additional plasmids required for AAV production (pRC plasmid and pHelper plasmid) are purchased from commercial suppliers (Aldevron, Takara).

For AAV production, HEK293 cells are cultured in FB medium in a 37° C. incubator with 5% CO2. 10-40 15 cm dishes of HEK293 cells are used in a single batch of viral production. For a single 15 cm dish, 45-60 μg plasmids are mixed together at 1:1:1 molar ratio together in 4 ml of FB medium, and complexed with Polyethyleneimine (PEI) i.e., at 3 μg PEI/μg of DNA, for 10 mins at room temperature. The ratio of the three plasmids used may be varied to optimize virus production. The PEI-DNA complex is then slowly dripped onto the 15 cm plate of HEK293 cells, and the plate of transfected cells is moved back into the incubator. The next day, the medium may be changed to FB with 2% FBS (instead of 10% FBS, (Fibroblast medium, comprised of: DMEM with Glutamax (Gibco 10566-016) supplemented with MEM-NEAA (Thermo 11140050), sodium pyruvate (Thermo 11360070), HEPES (Thermo 15630080), 2-mercaptoethanol (Gibco 21985023), penicillin/streptomycin (Thermo 15140122) with 10% volume fraction of fetal bovine serum (FBS, VWR #97068-085)), if appropriate). AAV may be harvested from the supernatant, or from the cell pellet, or from a combination of the supernatant and the cell pellet, at any time between 48-120 hours after initial transfection of the plasmids.

If virus is harvested after 72 hours post-transfection, the media from the cells may be collected at this time to increase virus yields. At 2-5 days post-transfection, the medium and cells are collected. The timing of harvest may be varied to optimize virus yield. The cells are pelleted by centrifugation, and the medium collected from the top. Cells are lysed in a buffer with high salt content and high-salt-active nuclease for 1 h at 37° C. The cells may also be lysed using additional methods, such as sequential freeze-thaw, or chemical lysis by detergent. The medium collected at harvest, and any medium collected at earlier time points, are treated with a 1:5 dilution of a solution containing 40% PEG8000 and 2.5M NaCl, and incubated on ice for 2 h, in order to precipitate AAV. The incubation may also be carried out overnight at 4° C. The AAV precipitate from the medium is pelleted by centrifugation, resuspended in high salt content buffer with high-salt-active nuclease and combined with the lysed cell pellet. The combined cell lysate is then clarified by centrifugation and filtration through a 0.45 μm filter, and purified on an AAV Poros affinity resin column (Thermofisher Scientific). The virus is eluted from the column into a neutralizing solution. At this stage, the virus may be taken through additional rounds of purification to increase the quality of the virus preparation. The eluted virus is then titered via qPCR to quantify the virus yield. For titering, a sample of virus is first digested with DNAse to remove any non-packaged viral DNA, the DNAse deactivated, and then viral capsids disrupted with Proteinase K to expose the packaged viral genomes for titering.

It is expected that ˜1×10¹² viral genomes will be obtained from one batch of virus produced using the methods as described here.

### Example 20: In Vivo Evaluation of PCSK9 Editing in Mouse Models

In a first set of experiments, wildtype C57BL/6J mice will be used to test the ability of AAV particles encoding CasX and guide RNA targeted to PCSK9, or XDP comprising RNP of CasX and guide RNA targeted to PCSK9, to edit the mouse PCSK9 gene in vivo. (Carreras et al. BMC Biology 2019 17:4).

### Materials and Methods

AAV (Table 13, utilizing constructs 3A, 36A, and 37A) encoding CasX 491 and gRNA 174, or XDP packaging RNP of CasX 491 and gRNA 174 (Table 15—pXDP0017, pXDP0001, pGP2, pStx42.174.27.5) constructs targeting the mouse PCSK9 gene using spacer sequence 27.5 (spacer sequence GAGGCTAGAGGACTGAGCCA (SEQ ID NO: 225) for AAV and GAGGCUAGAGGACUGAGCCA (SEQ ID NO: 226) for XDP) will be administered into 10 to 14-week old C57BL/6J mice via tail-vein injections. As experimental controls, AAV or XDP encoding or packaging CasX and gRNA constructs targeting a safe harbor site in the mouse genome, e.g., the mRosa26 locus, will also be administered to control groups. 1 month and 3 months after administration of the respective vectors, cholesterol levels in the blood plasma will be assessed using an enzymatic colorimetric assay, and PCSK9 levels in the blood plasma will be assessed by an ELISA or Western blot assay. A subset of mice will be sacrificed at 1 month and 3 months post-administration of vectors, and tissues will be processed for assessment of PCSK9 gene editing at the genomic, transcriptomic and proteomic levels via NGS, qPCR, and immunohistology. Tissues will also be assessed for off-target editing using established tools for off-target analysis; for example GUIDE-Seq. Additionally, expression of CasX will also be measured across tissues of interest by immunohistology. The results are expected to demonstrate the ability to edit the PCSK9 gene in mice, with a concomitant reduction in cholesterol levels.

TABLE 13
Sequence for AAV constructs
targeting the mouse PCSK9 locus

| Construct | DNA SEQUENCE |
|---|---|
| 3A | (SEQ ID NO: 227) |
| 36A | (SEQ ID NO: 228) |
| 37A | (SEQ ID NO: 229) |

A transgenic mouse model of hypercholesterolemia with liver-specific expression of human PCSK9 will be used to test the ability of AAV particles encoding CasX and guide RNA, or XDP packaging RNP of CasX and guide RNA targeting human PCSK9, to edit the PCSK9 gene in vivo. (Carreras et al. BMC Biology 2019 17:4).

AAV (Table 14 with sequences for constructs 3, 36, and 37) encoding CasX 491 and gRNA 174 or XDP packaging RNP of CasX 491 and gRNA 174 constructs (Table 15 with sequences for pXDP0017, pXDP0001, pGP2, pStx42.174.6.8) targeting the human PCSK9 gene (spacer sequence TGGCTTCCTGGTGAAGATGA (SEQ ID NO: 515) for AAV and UGGCUUCCUGGUGAAGAUGA (SEQ ID NO: 559) for XDP) will be administered into 10 to 14-week old transgenic mice via tail-vein injections. As experimental controls, AAV or XDP packaging CasX and gRNA constructs targeting a safe harbor site in the mouse genome, e.g., the mRosa26 locus will be administered. 1 month and 3 months after administration of the respective vectors, cholesterol levels in the blood plasma will be assessed using an enzymatic colorimetric assay, and PCSK9 levels in the blood plasma will be assessed by an ELISA or Western blot assay. A subset of mice will be sacrificed at 1 month and 3 months post-administration of the vectors, and tissues will be processed for assessment of PCSK9 gene editing at the genomic, transcriptomic and proteomic levels via NGS, qPCR, and immunohistology. Tissues will also be assessed for off-target editing using established tools for off-target analysis; for example GUIDE-Seq. Additionally, expression of CasX will also be measured across tissues of interest by immunohistology. The results are expected to demonstrate the ability to edit the human PCSK9 gene in mice, with a concomitant reduction in cholesterol levels.

TABLE 14
Sequence for AAV constructs
targeting the PCSK9 locus

| Construct | DNA SEQUENCE |
|---|---|
| 3 | (SEQ ID NO: 230) |
| 36 | (SEQ ID NO: 231) |
| 37 | (SEQ ID NO: 232) |

TABLE 15
Sequences for XDP constructs targeting
the human PCSK9 locus (spacer 6.8)
and mouse PCSK9 locus (spacer 27.5)

| Construct | DNA SEQUENCE |
|---|---|
| pStx42.174.27.5 | (SEQ ID NO: 25) |
| pStx42.174.6.8 | (SEQ ID NO: 26) |
| pGP2 | (SEQ ID NO: 28) |
| pXDP0017 | (SEQ ID NO: 29) |
| pXDP0001 | (SEQ ID NO: 30) |

### Example 21: Assays Used to Measure sgNA and CasX Protein Activity

Several assays were used to carry out initial screens of CasX protein and sgNA Deep Mutational Evolution (DME) libraries and modified mutants, and to measure the activity of select protein and sgNA variants relative to CasX reference sgNAs and proteins. E. coli CRISPRi screen:

Briefly, biological triplicates of dead CasX DME Libraries on a chloramphenicol (CM) resistant plasmid with a GFP gNA on a carbenicillin (Carb) resistant plasmid were transformed (at >5× library size) into MG1655 with genetically integrated and constitutively expressed GFP and RFP. Cells were grown overnight in EZ-RDM+Carb, CM and Anhydrotetracycline (aTc) inducer. E. coli were FACS sorted based on gates for the top 1% of GFP but not RFP repression, collected, and resorted immediately to further enrich for highly functional CasX molecules. Double sorted libraries were then grown out and DNA was collected for deep sequencing on a highseq. This DNA was also re-transformed onto plates and individual clones were picked for further analysis.

E. coli Toxin Selection:

Briefly carbenicillin resistant plasmid containing an arabinose inducible toxin were transformed into E. coli cells and made electrocompetent. Biological triplicates of CasX DME Libraries with a toxin targeted gNA on a chloramphenicol resistant plasmid were transformed (at >5× library size) into said cells and grown in LB+CM and arabinose inducer. E. coli that cleaved the toxin plasmid survived in the induction media and were grown to mid log and plasmids with functional CasX cleavers were recovered. This selection was repeated as needed. Selected libraries were then grown out and DNA was collected for deep sequencing on a highseq. This DNA was also re-transformed onto plates and individual clones were picked for further analysis and testing.

### Lentiviral Based Screen EGFP Screen:

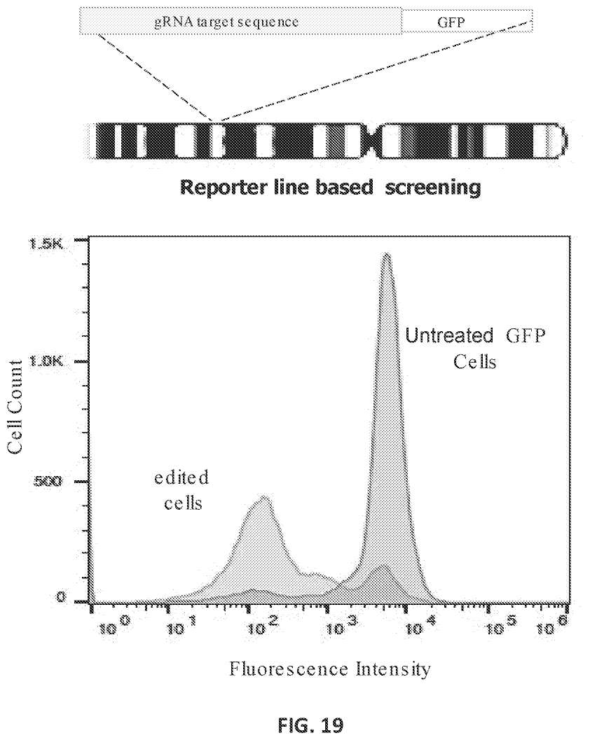

Lentiviral particles were produced in HEK293 cells at a confluency of 70%-90% at time of transfection. Cells were transfected using polyethylenimine based transfection of plasmids containing a CasX DME library. Lentiviral vectors were co-transfected with the lentiviral packaging plasmid and the VSV-G envelope plasmids for particle production. Media was changed 12 hours post-transfection, and virus harvested at 36-48 hours post-transfection. Viral supernatants were filtered using 0.45 mm membrane filters, diluted in cell culture media if appropriate, and added to target cells HEK cells with an Integrated GFP reporter. Polybrene was supplemented to enhance transduction efficiency, if necessary. Transduced cells were selected for 24-48 hours post-transduction using puromycin and grown for 7-10 days. Cells were then sorted for GFP disruption & collected for highly functional CasX sgNA or protein variants (see FIG. 19). Libraries were then Amplified via PCR directly from the genome and collected for deep sequencing on a highseq. This DNA could also be re-cloned and re-transformed onto plates and individual clones were picked for further analysis.

### Example 22: Assaying Editing Efficiency of an HEK EGFP Reporter

To assay the editing efficiency of CasX reference sgNAs and proteins and variants thereof, EGFP HEK293T reporter cells were seeded into 96-well plates and transfected according to the manufacturer's protocol with lipofectamine 3000 (Life Technologies) and 100-200 ng plasmid DNA encoding a reference or CasX variant protein, P2A-puromycin fusion and the reference or variant sgNA. The next day cells were selected with 1.5 μg/ml puromycin for 2 days and analyzed by fluorescence-activated cell sorting (FACS) 7 days after selection to allow for clearance of EGFP protein from the cells. EGFP disruption via editing was traced using an Attune NxT Flow Cytometer and high-throughput autosampler.

### Example 23: Cleavage Efficiency of CasX Reference sgRNA

The reference CasX sgRNA of SEQ ID NO:4 (below) is described in WO 2018064371 and U.S. Ser. No. 10/570,415B2, the contents of which are incorporated herein by reference:

(SEQ ID NO: 4)
ACAUCUGGCGCGUUUAUUCCAUUACUUUGGAGCCAGUCCCAGCGACUAU
GUCGUAUGGACGAAGCGCUUAUUUAUCGGAGAGAAACCGAUAAGUAAAA
CGCAUCAAAG.

It was found that alterations to the sgRNA reference sequence of SEQ ID NO:4, producing SEQ ID NO:5 (below) were able to improve CasX cleavage efficiency. The sequence is:

(SEQ ID NO: 5)
UACUGGCGCUUUUAUCUCAUUACUUUGAGAGCCAUCACCAGCGACUAUG
UCGUAUGGGUAAAGCGCUUAUUUAUCGGAGAGAAAUCCGAUAAAUAAGA
AGCAUCAAAG.

To assay the editing efficiency of CasX reference sgRNAs and variants thereof, EGFP HEK293T reporter cells were seeded into 96-well plates and transfected according to the manufacturer's protocol with lipofectamine 3000 (Life Technologies) and 100-200 ng plasmid DNA encoding a reference CasX protein, P2A-puromycin fusion and the sgRNA. The next day cells were selected with 1.5 μg/ml puromycin for 2 days and analyzed by fluorescence-activated cell sorting (FACS) 7 days after selection to allow for clearance of EGFP protein from the cells. EGFP disruption via editing was traced using an Attune NxT Flow Cytometer and high-throughput autosampler.

When testing cleavage of an EGFP reporter by CasX reference and sgNA variants, the following spacer target sequences were used: When testing cleavage of an EGFP reporter by CasX reference and sgNA variants, the following spacer target sequences were used: E6 (TGTGGTCGGGGTAGCGGCTG (SEQ ID NO: 17)) and E7 (TCAAGTCCGCCATGCCCGAA (SEQ ID NO: 18)).

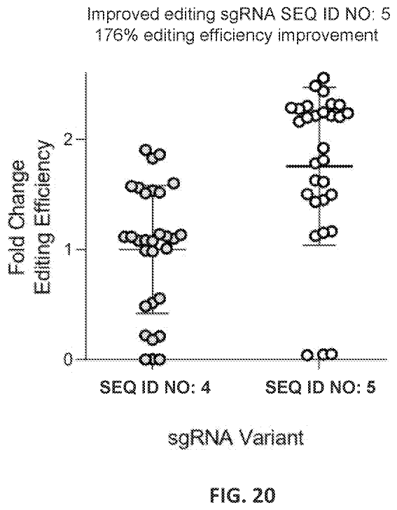

An example of the increased cleavage efficiency of the sgRNA of SEQ ID NO:5 compared to the sgRNA of SEQ ID NO:4 is shown in FIG. 20. Editing efficiency of SEQ ID NO:5 was improved 176% compared to SEQ ID NO: 4. Accordingly, SEQ ID NO: 5 was chosen as reference sgRNA for DME and additional sgNA variant design, described below.

### Example 24: Design, Creation and Evaluation of gNA Variants with Improved Target Cleavage

Guide nucleic acid (gNA) variants were designed and tested in order to assess improvements in cleavage activity relative to reference gNAs. These guides were discovered via DME or rational design and replacement or addition of guide parts such as the extended stem or the addition of ribozymes at the termini, as described herein.

### Experimental Design:

All guides were tested In HEK293T or a HEK293T reporter line as follows. Mammalian cells were maintained in a 37° C. incubator, at 5% CO2. HEK293T Human kidney cells and derivatives thereof were grown in Dulbecco's Modified Eagle Medium (DMEM; Corning Cellgro, #10-013-CV) supplemented with 10% fetal bovine serum (FBS; Seradigm, #1500-500), and 100 Units/ml penicillin and 100 mg/ml streptomycin (100×-Pen-Strep; GIBCO #15140-122), and can additionally include sodium pyruvate (100×, Thermofisher #11360070), Non-essential amino acids (100× Thermofisher #11140050), HEPES buffer (100× Thermofisher #15630080), and 2-mercaptoethanol (1000× Thermofisher #21985023). Cells were seeded at 20-30 thousand cells per well into 96-well plates and transfected using 0.25-1 uL of Lipofectamine 3000 (Thermo Fisher Scientific #L3000008), 50-500 ng of a plasmid containing CasX and the reference or variant CasX guide targeting the reporter or target gene following the manufacturer's protocol. 24-72 hours later the media was changed and 0.3-3.0 ug/ml puromycin (Sigma #P8833) was added to select for transformation. 24-96 hours following selection the cells were analyzed by flow cytometry and gated for the appropriate forward and side scatter, selected for single cells and then gated for green fluorescent protein (GFP) or antibody reporter expression (Attune Nxt Flow Cytometer, Thermo Fisher Scientific) to quantify the expression levels of fluorophores. At least 10,000 events were collected for each sample. For the HEK293T-GFP genome editing reporter cell line, flow cytometry was used to quantify the percentage of GFP-negative (edited) cells and the number of cells with GFP disruption for each variant was compared to the reference guide to generate a fold change measurement.

### Results:

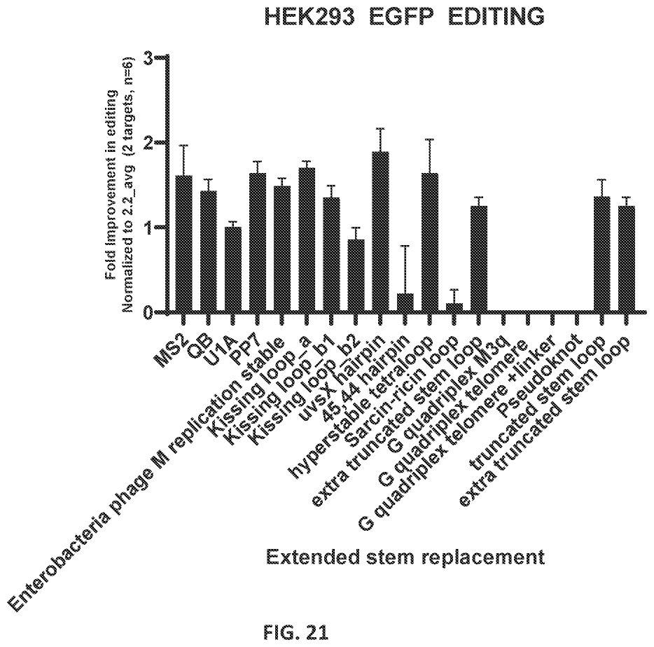

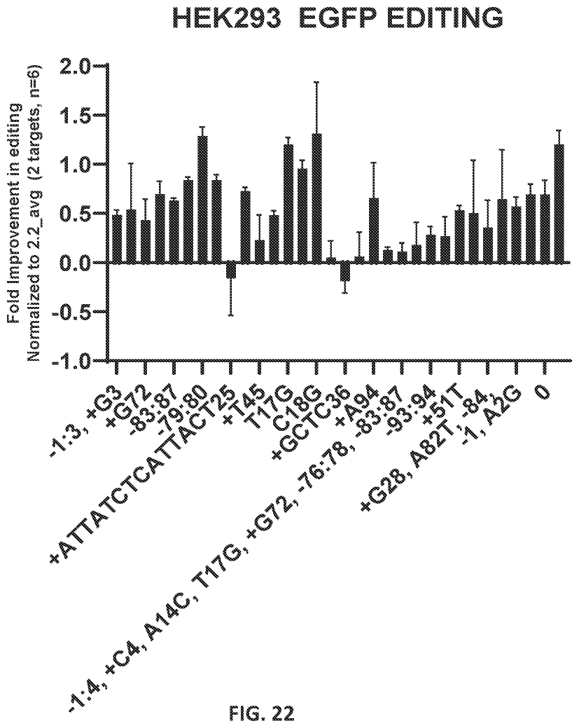

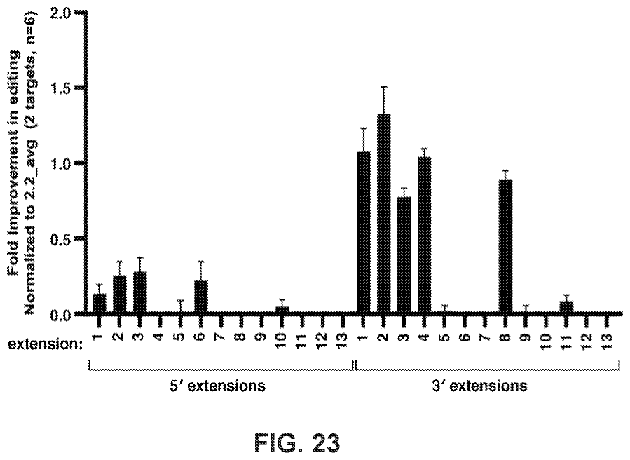

Results from the sgNA variants generated via DME were measured and compared to the reference gNA of SEQ ID NO: 4. These results are presented in FIG. 22, with most variants showing improvements from 0.1 to nearly 1.5-fold compared to the reference gNA. Results of the variants generated via rational design and replacement or addition of guide parts (such as the extended stem or the addition of ribozymes at the termini) are shown in FIGS. 21 and 23 respectively; again showing improvements with many of the constructs. The additions to the variants, along with their encoding sequences, portrayed by number in FIG. 23 are listed in Table 16, below. We observed that single mutations such as the C18G mutation improve guide activity when compared to the reference. Additionally, rationally swapping in different stem loops for the extended stem loop, such as MS2, QB, PP7, UvsX, etc. improved activity when compared to the reference guide, as does truncating the original extended stem loop. Finally, we demonstrate that while most ribozymes disrupt activity, the addition of a 3′ HDV to the reference guide RNA can improve activity up to 20-50%.

TABLE 16
Extensions added to 3′ and 5′ ends of gNA

| Extension | | |
|---|---|---|
| Number | Extension Name | Extension Encoding Sequence |
| 1 | HDV antigenomic | GGGTCGGCATGGCATCTCCACCTCCTCGCGGTCCGACCTGGGC |
| | ribozyme | ATCCGAAGGAGGACGCACGTCCACTCGGATGGCTAAGGGAGAG |
| | | CCA (SEQ ID NO: 598) |
| 2 | HDV genomic | GGCCGGCATGGTCCCAGCCTCCTCGCTGGCGCCGGCTGGGCAA |
| | ribozyme | CATTCCGAGGGGACCGTCCCCTCGGTAATGGCGAATGGGACCC |
| | | (SEQ ID NO: 599) |
| 3 | HDV ribozyme | GATGGCCGGCATGGTCCCAGCCTCCTCGCTGGCGCCGGCTGGG |
| | (v1) | CAACACCTTCGGGTGGCGAATGGGAC (SEQ ID NO: 600) |
| 4 | HDV ribozyme | TTTTGGCCGGCATGGTCCCAGCCTCCTCGCTGGCGCCGGCTGG |
| | (v2) | GCAACATGCTTCGGCATGGCGAATGGGACCCCGGG (SEQ ID |
| | | NO: 601) |
| 5 | Hatchet | CATTCCTCAGAAAATGACAAACCTGTGGGGCGTAAGTAGATCT |
| | | TCGGATCTATGATCGTGCAGACGTTAAAATCAGGT (SQE ID |
| | | NO: 602) |
| 6 | env25 pistol | CGTGGTTAGGGCCACGTTAAATAGTTGCTTAAGCCCTAAGCGT |
| | ribozyme (with | TGATCTTCGGATCAGGTGCAA (SEQ ID NO: 603) |
| | CUUCGGloop) | |
| 7 | HH15 Minimal | GGGAGCCCCGCTGATGAGGTCGGGGAGACCGAAAGGGACTTCG |
| | Hammerhead | GTCCCTACGGGGCTCCC (SEQ ID NO: 604) |
| | ribozyme | |
| 8 | sTRSV WT viral | CCTGTCACCGGATGTGCTTTCCGGTCTGATGAGTCCGTGAGGA |
| | Hammerhead | CGAAACAGG (SEQ ID NO: 605) |
| | ribozyme | |
| 9 | Hammerhead | CGACTACTGATGAGTCCGTGAGGACGAAACGAGTAAGCTCGTC |
| | ribozyme | TAGTCGCGTGTAGCGAAGCA (SEQ ID NO: 606) |
| 10 | Hammerhead | CGACTACTGATGAGTCCGTGAGGACGAAACGAGTAAGCTCGTC |
| | ribozyme, | TAGTCG (SEQ ID NO: 607) |
| | smaller scar | |
| 11 | Hammerhead | CCAGTACTGATGAGTCCGTGAGGACGAAACGAGTAAGCTCGTC |
| | ribozyme, guide | TACTGGCGCTTTTATCTCAT (SEQ ID NO: 608) |
| | scaffold scar | |
| 12 | Twisted | ACCCGCAAGGCCGACGGCATCCGCCGCCGCTGGTGCAAGTCCA |
| | Sister 1 | GCCGCCCCTTCGGGGGCGGGCGCTCATGGGTAAC (SEQ ID |
| | | NO: 609) |
| 13 | Env-9 Twister | GGCAATAAAGCGGTTACAAGCCCGCAAAAATAGCAGAGTAATG |
| | | TCGCGATAGCGCGGCATTAATGCAGCTTTATTG (SEQ ID |
| | | NO: 610) |
| 14 | RBMX recruiting | CCACCCCCACCACCACCCCCACCCCCACCACCACCC (SEQ |
| | motif | ID NO: 611) |

The results support the conclusion that DME and rational design can be used to improve the performance of the gNAs and that many of these variant RNAs can now be used with the targeting sequences as a component of the CasX:gNA systems described herein to edit target nucleic acid sequences.

### Example 25: CasX Molecule 119 and Guide Scaffold 174 Edits PCSK9 Locus in HEK293T Cells

The purpose of the experiments was to demonstrate editing of the PCSK9 locus in HEK293T cells using constructs of CasX 119, guide 174 and spacers targeting the WT sequence, when delivered by plasmid transfection.

### Materials and Methods:

Spacers targeting PCSK9 were chosen manually based on PAM availability without prior knowledge of activity (sequences in Table 11). HEK293T cells were seeded at 20-40 k cells/well in a 96 well plate in 100 μl of FB medium and cultured in a 37° C. incubator with 5% CO2. The following day, confluence of seeded cells was checked to ensure that cells were at ˜75% confluence at time of transfection. If cells were at the right confluence, transfection was carried out. Each CasX and guide construct (e.g., see Table 5 for sequence of CasX 119; see Table 2 for sequence of guide 174; and see Table 11 for PCSK9 spacer sequences) was transfected into the HEK293T cells at 100-500 ng per well using Lipofectamine 3000 following the manufacturer's protocol, using 3 wells per construct as replicates. SaCas9 and SpyCas9 targeting PCSK9 were used as benchmarking controls. For each Cas protein type, a non-targeting plasmid was used as a negative control. Cells were selected for successful transfection with puromycin at 0.3-3 μg/ml for 24-48 hours, followed by 24-96 hours of recovery in FB medium. Cells for each sample from the experiment was lysed, and the genome was extracted following the manufacturer's protocol and standard practices. Editing in cells from each experimental sample were assayed using NGS analysis. Briefly, genomic DNA was amplified via PCR with primers specific to the target genomic location of interest to form a target amplicon. These primers contain additional sequence at the 5′ ends to introduce Illumina reads 1 and 2 sequences. Further, they contain a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions and/or deletions anywhere within this window.

### Results:

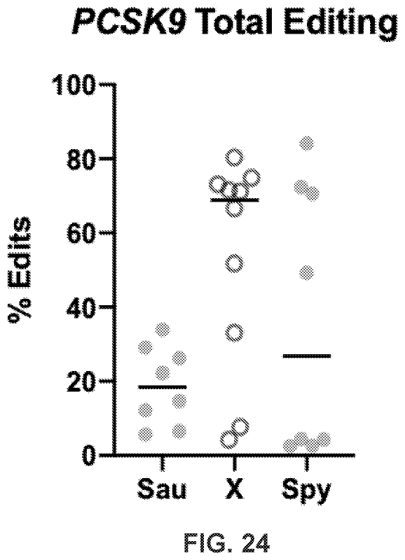

The graph of FIG. 24 shows that constructs utilizing ten different spacers targeted to PCSK9 were able to edit the PCSK9 locus with varying levels of activity, at an average editing of 70%. Each data point is an average measurement of NGS reads of editing outcomes generated by an individual spacer. These results demonstrate that, under the conditions of the assay, CasX with appropriate guides were able to edit the PCSK9 locus, and did so to a greater degree compared to Spy Cas9 (based on mean editing), while exhibiting considerably more editing than Sau Cas9.

### Example 26: CasX 119 and Guide Scaffold 174 Edits the PCSK9 Locus in HepG2 Cells

Experiments were conducted to demonstrate the ability to edit the PCSK9 locus in HepG2 cells using constructs of CasX 119, guide 174 and spacers targeting the WT PCSK9 sequence delivered by lentivirus.

### Materials and Methods:

Lentiviral particles were produced using the methods of Example 15 by transfecting HEK293T at a confluency of 70%-90% using polyethylenimine-based transfection of CasX plasmids containing spacers targeting the PCSK9 locus (sequences 6.7, 6.8, and 6.9 of Table 11), the lentiviral packaging plasmid and the VSV-G envelope plasmids. For particle production, media was changed 12 hr. post-transfection, and virus harvested at 36-48 hours post-transfection. Viral supernatants were filtered using 0.45 μm membrane filters, diluted in media if appropriate, and added to HepG2 target cells cultured in HepG2 medium (EMEM with 10% FBS and 1% penicillin-streptomycin). Supplemental polyberene was added at 5-20 μg/ml to enhance transduction efficiency, if necessary. Transduced cells were selected 24-48 hours post-transduction using puromycin at 0.3-3 μg/ml in HepG2 medium, and grown for 6 days in HepG2 medium in a 37° C. incubator with 5% CO2. Cells were then harvested, and editing was analyzed using NGS. Briefly, genomic DNA was amplified via PCR with primers specific to the target genomic location of interest to form a target amplicon. These primers contained additional sequence at the 5′ ends to introduce Illumina reads 1 and 2 sequences. Further, they contained a 16 nt random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The editing activity of the CasX molecule was quantified as the total percent of reads that contained insertions and/or deletions anywhere within this window.

### Results:

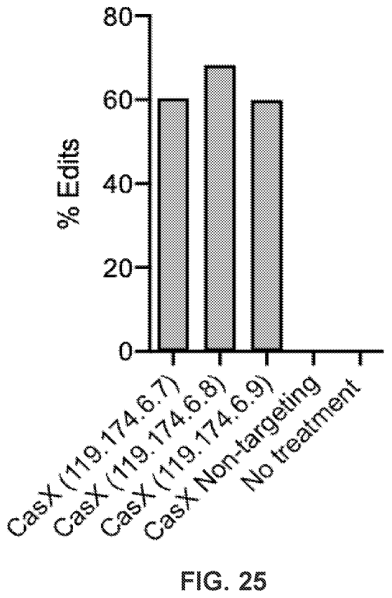

The graph of FIG. 25 shows that constructs with three different spacers targeted to PCSK9 were able to edit the PCSK9 locus with varying levels of activity, at an average editing of 60%. Each data point is an average measurement of NGS reads of editing outcomes generated by an individual spacer.

The results demonstrate that, under the conditions of the assay, CasX with appropriately targeted guides were able to edit the PCSK9 locus in HepG2 cells with a high degree of efficiency.

### Example 27: CasX 491 and Guide Scaffold 174 Edits the PCSK9 Locus in AML12 Cells

Experiments were conducted to demonstrate the ability to edit the wild-type PCSK9 locus in AML12 cells when delivered by transfection.

### Materials and Methods:

Murine hepatocyte cell line AML12 cells were transfected with 1000 ng of plasmid encoding CasX 491 along with gRNA scaffold 174 with spacers 27.1 to 27.7, targeting wild-type murine PCSK9 (sequence in Table 17). Transfected cells are grown for 6 days in AML12 medium (DMEM:F12 supplemented with 10% fetal bovine serum, 10 μg/ml insulin, 5.5 μg/ml transferrin, 5 ng/ml selenium, 40 ng/ml dexamethasone) incubated at 37° C. incubator with 5% CO2. Cells were then harvested and editing analyzed using NGS. Briefly, genomic DNA was amplified via PCR with primers specific to the target genomic location of interest to form a target amplicon. These primers contain additional sequence at the 5′ ends to introduce Illumina read 1 and 2 sequences. Further, contain a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions and/or deletions anywhere within this window.

TABLE 17
Spacer sequences targeting mouse PCSK9 genetic locus

| | | | DNA | | RNA |
|---|---|---|---|---|---|
| | | | SEQ | | SEQ |
| Spacer | PAM | Spacer DNA Sequence | ID NO: | Spacer RNA Sequence | ID NO: |
| 27.1 | TTC | GCCTCGCCCTCCCCAGACAG | 233 | GCCUCGCCCUCCCCAGACAG | 240 |
| 27.2 | TTC | GATGGGGCTCGGGGTGGCGT | 234 | GAUGGGGCUCGGGGUGGCGU | 241 |
| 27.3 | TTC | GGGGTGTGGGTACTGGACGC | 235 | GGGGUGUGGGUACUGGACGC | 242 |
| 27.4 | TTC | CGTGGACGCGCAGGCTGCCG | 236 | CGUGGACGCGCAGGCUGCCG | 243 |
| 27.5 | TTC | GAGGCTAGAGGACTGAGCCA | 237 | GAGGCUAGAGGACUGAGCCA | 244 |
| 27.6 | TTC | CGAGGCCGCGCGCACCTCTC | 238 | CGAGGCCGCGCGCACCUCUC | 245 |
| 27.7 | TTC | TAATCTCCATCCTCGTCCTG | 239 | UAAUCUCCAUCCUCGUCCUG | 246 |

### Results:

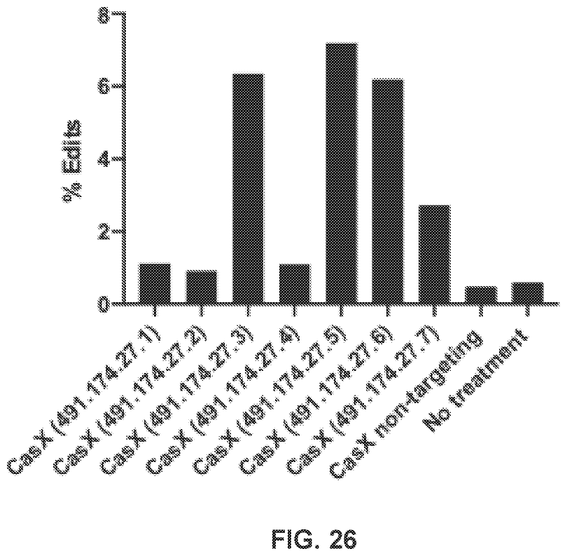

The graph of FIG. 26 shows that constructs with three different spacers were able to edit the PCSK9 locus with at an average editing of at least 6-7%, with other spacers resulting in lower amounts of editing. Each data point is an average measurement of NGS reads of editing outcomes generated by an individual spacer. The results demonstrate that, under the conditions of the assay, CasX with appropriately targeted guides were able to edit the PCSK9 locus in AML12 cells.

What is claimed is:

1. A system comprising a Class 2 Type V CRISPR protein and a first guide nucleic acid (gNA), wherein the gNA comprises a targeting sequence complementary to a proprotein convertase subtilisin/kexin Type 9 (PCSK9) gene target nucleic acid sequence, wherein the PCSK9 gene comprises one or more mutations.

2. The system of claim 1, wherein the PCSK9 gene comprises one or more mutations in a region selected from the group consisting of:
a. a PCSK9 intron;
b. a PCSK9 exon;
c. a PCSK9 intron-exon junction;
d. a PCSK9 regulatory element; and
e. an intergenic region.

3. The system of any one of claim 1 or claim 2, wherein the mutation is an insertion, deletion, substitution, duplication, or inversion of one or more nucleotides as compared to the wild-type PCSK9 gene sequence.

4. The system of any one of claims 1-3, wherein the mutation is a gain of function mutation.

5. The system of claim 3, wherein the one or more mutations comprise amino acid substitutions selected from the group consisting of S127R, D129G, F216L, D374H, and D374Y relative to the sequence of SEQ ID NO: 33.

6. The system of claims 1-5, wherein the targeting sequence of the gNA is complementary to a target nucleic acid sequence encoding the S127R, D129G, F216L, D374H, or D374Y substitution.

7. The system of claim 6, wherein the targeting sequence of the gNA comprises a sequence of selected from the group consisting of AGCAGGUCGCCUCUCAUCUU (SEQ ID NO: 272), CAUCUUCACCAGGAAGCCAG (SEQ ID NO: 273), CCUCUCAUCUUCACCAGGAA (SEQ ID NO: 274), UGGUGAAGAUGAGAGGCGAC (SEQ ID NO: 275), GUGGAGGCGGGUCCCGUCCU (SEQ ID NO: 281), AGCCACUGCAGCACCUGCUU (SEQ ID NO: 287), UUGGUGCCUCCAGCCACUGC (SEQ ID NO: 288), AGCUACUGCAGCACCUGCUU (SEQ ID NO: 289), and UUGGUGCCUCCAGCUACUGC (SEQ ID NO:290).

8. The system of any one of claims 1-3, wherein the mutation is a loss of function mutation.

9. The system of claim 8, wherein the one or more mutations comprise amino acid substitutions selected from the group consisting of R46L, G106R, Y142X, N157K, R237W and C679X relative to the sequence of SEQ ID NO: 33.

10. The system of claim 9, wherein the targeting sequence of the gNA is complementary to a target nucleic acid sequence encoding the R46L, G106R, Y142X, N157K, R237W or C679X substitution.

11. The system of any one of claims 1-10, wherein the PCSK9 gene encodes a non-functional PCSK9 protein.

12. The system of any one of claims 1-11, wherein the gNA is a guide RNA (gRNA).

13. The system of any one of claims 1-11, wherein the gNA is a guide DNA (gDNA).

14. The system of any one of claims 1-11, wherein the gNA is a chimera comprising DNA and RNA.

15. The system of any one of claims 1-14, wherein the gNA is a single-molecule gNA (sgNA).

16. The system of any one of claims 1-14, wherein the gNA is a dual-molecule gNA (dgNA).

17. The system of any one of claims 1-16, wherein the targeting sequence of the gNA comprises a sequence selected from the group consisting of the sequences of SEQ ID NOS: 247-303, 315-436, 612-2100, and 2286-13861, or a sequence having at least about 65%, at least about 75%, at least about 85%, or at least about 95% identity thereto.

18. The system of any one of claims 1-16, wherein the targeting sequence of the gNA comprises a sequence selected from the group consisting of the sequences of SEQ ID NOs: 247-303, 315-436, 612-2100, and 2286-13861.

19. The system of any one of claims 1-16, wherein the targeting sequence of the gNA comprises a sequence of SEQ ID NOs: 247-303, 315-436, 612-2100, or 2286-13861 with a single nucleotide removed from the 3′ end of the sequence.

20. The system of any one of claims 1-16, wherein the targeting sequence of the gNA comprises a sequence of SEQ ID NOs: 247-303, 315-436, 612-2100, or 2286-13861 with two nucleotides removed from the 3′ end of the sequence.

21. The system of any one of claims 1-16, wherein the targeting sequence of the gNA comprises a sequence of SEQ ID NOs: 247-303, 315-436, 612-2100, or 2286-13861 with three nucleotides removed from the 3′ end of the sequence.

22. The system of any one of claims 1-16, wherein the targeting sequence of the gNA comprises a sequence of SEQ ID NOs: 247-303, 315-436, 612-2100, or 2286-13861 with four nucleotides removed from the 3′ end of the sequence.

23. The system of any one of claims 1-16, wherein the targeting sequence of the gNA comprises a sequence of SEQ ID NOs: 247-303, 315-436, 612-2100, or 2286-13861 with five nucleotides removed from the 3′ end of the sequence.

24. The system of any one of claims 17-23, wherein the targeting sequence of the gNA comprises a sequence having one or more single nucleotide polymorphisms (SNP) relative to a sequence of SEQ ID NOS: 247-303, 315-436, 612-2100, or 2286-13861.

25. The system of any one of claims 1-24, wherein the targeting sequence of the gNA is complementary to a sequence of a PCSK9 exon.

26. The system of any one of claims 1-24, wherein the targeting sequence of the gNA is complementary to a sequence of PCSK9 exon 1 or exon 2.

27. The system of any one of claims 1-24, wherein the targeting sequence of the gNA is complementary to a sequence of a PCSK9 intron.

28. The system of any one of claims 1-24, wherein the targeting sequence of the gNA is complementary to a sequence of a PCSK9 intron-exon junction.

29. The system of any one of claims 1-24, wherein the targeting sequence of the gNA is complementary to a sequence of a PCSK9 regulatory element.

30. The system of any one of claims 1-23, wherein the targeting sequence of the gNA is complementary to a sequence comprising one or more single nucleotide polymorphisms (SNPs) of the PCSK9 gene.

31. The system of any one of claims 1-24, wherein the targeting sequence of the gNA is complementary to a sequence of an intergenic region of the PCSK9 gene.

32. The system of any one of claims 1-31, further comprising a second gNA, wherein the second gNA has a targeting sequence complementary to a different or overlapping portion of the PCSK9 target nucleic acid compared to the targeting sequence of the first gNA.

33. The system of claim 32, wherein the second gNA has a targeting sequence complementary to the same exon targeted by the first gNA.

34. The system of claim 32, wherein the second gNA has a targeting sequence complementary to a different exon targeted by the first gNA.

35. The system of claim 32, wherein the second gNA has a targeting sequence complementary to an intron 3′ to the exon targeted by the first gNA.

36. The system of any one of claims 32-35, wherein the targeting sequence of the second gNA comprises a sequence selected from the group consisting of the sequences of SEQ ID NOS: 247-303, 315-436, 612-2100, and 2286-13861, or a sequence having at least about 65%, at least about 75%, at least about 85%, or at least about 95% identity thereto.

37. The system of any one of claims 32-35, wherein the targeting sequence of the second gNA comprises a sequence selected from the group consisting of the sequences of SEQ ID NOs: 247-303, 315-436, 612-2100, and 2286-13861.

38. The system of any one of claims 32-35, wherein the targeting sequence of the second gNA comprises a sequence of SEQ ID NOs: 247-303, 315-436, 612-2100, or 2286-13861 with a single nucleotide removed from the 3′ end of the sequence.

39. The system of any one of claims 32-35, wherein the targeting sequence of the second gNA comprises a sequence of SEQ ID NOs: 247-303, 315-436, 612-2100, or 2286-13861 with two nucleotides removed from the 3′ end of the sequence.

40. The system of any one of claims 32-35, wherein the targeting sequence of the second gNA comprises a sequence of SEQ ID NOs: 247-303, 315-436, 612-2100, or 2286-13861 with three nucleotides removed from the 3′ end of the sequence.

41. The system of any one of claims 32-35, wherein the targeting sequence of the second gNA comprises a sequence of SEQ ID NOs: 247-303, 315-436, 612-2100, or 2286-13861 with four nucleotides removed from the 3′ end of the sequence.

42. The system of any one of claims 32-35, wherein the targeting sequence of the second gNA comprises a sequence of SEQ ID NOs: 247-303, 315-436, 612-2100, or 2286-13861 with five nucleotides removed from the 3′ end of the sequence.

43. The system of any one of claims 1-42, wherein the first and/or second gNA has a scaffold comprising a sequence selected from the group consisting of SEQ ID NOS: 2201-2285, or a sequence having at least about 50%, at least about 60%, at least about 70%, at least about 80%, at least about 90%, at least about 95%, at least about 96%, at least about 97%, at least about 98%, at least about 99% sequence identity thereto.

44. The system of any one of claims 1-42, wherein the first and/or second gNA has a scaffold comprising a sequence selected from the group consisting of SEQ ID NOS: 2201-2285.

45. The system of any one of claims 1-42, wherein the first and/or second gNA has a scaffold consisting of a sequence selected from the group consisting of SEQ ID NOS: 2201-2285.

46. The system of any one of claims 1-45, wherein the first and/or second gNA scaffold comprises a sequence having at least one modification relative to a reference gNA sequence selected from the group consisting of SEQ ID NOS: 4-16.

47. The system of claim 46, wherein the at least one modification of the reference gNA comprises at least one substitution, deletion, or substitution of a nucleotide of the reference gNA sequence.

48. The system of any one of claims 1-47, wherein the first and/or second gNA is chemically modified.

49. The system of any one of claims 1-48, wherein the Class 2 Type V CRISPR protein is a reference CasX protein having a sequence of any one of SEQ ID NOS: 1-3, a CasX variant protein having a sequence of SEQ ID NOS: 49-160, 329, 441, 443, 445, 447-460, 472, 474, 476, 478, 480, 482, 484, 486, 488, or 490, or a sequence having at least about 50%, at least about 60%, at least about 70%, at least about 80%, at least about 90%, or at least about 95%, or at least about 95%, or at least about 96%, or at least about 97%, or at least about 98%, or at least about 99% sequence identity thereto.

50. The system of any one of claims 1-49, wherein the Class 2 Type V CRISPR protein is a CasX variant protein having a sequence of SEQ ID NOS: 49-160, 329, 441, 443, 445, 447-460, 472, 474, 476, 478, 480, 482, 484, 486, 488, or 490.

51. The system of 49, wherein the CasX variant protein comprises at least one modification relative to a reference CasX protein having a sequence selected from SEQ ID NOS:1-3.

52. The system of claim 51, wherein the at least one modification comprises at least one amino acid substitution, deletion, or substitution in a domain of the CasX variant protein relative to the reference CasX protein.

53. The system of claim 52, wherein the domain is selected from the group consisting of a non-target strand binding (NTSB) domain, a target strand loading (TSL) domain, a helical I domain, a helical II domain, an oligonucleotide binding domain (OBD), and a RuvC DNA cleavage domain.

54. The system of any one of claims 49-53, wherein the CasX protein further comprises one or more nuclear localization signals (NLS).

55. The system of claim 54, wherein the one or more NLS are selected from the group of sequences consisting of SEQ ID NOS: 161-194, 217 and 223-224.

56. The system of claim 54 or claim 55, wherein the one or more NLS are expressed at or near the C-terminus of the CasX protein.

57. The system of claim 54 or claim 55, wherein the one or more NLS are expressed at or near the N-terminus of the CasX protein.

58. The system of claim 54 or claim 55, comprising one or more NLS located at or near the N-terminus and at or near the C-terminus of the CasX protein.

59. The system of any one of claims 49-58, wherein the Class 2 Type V CRISPR protein is capable of forming a ribonuclear protein complex (RNP) with the gNA.

60. The system of any one of claims 49-58, wherein the CasX variant is capable of forming a ribonuclear protein complex (RNP) with the gNA.

61. The system of any one of claims 49-58, wherein the CasX variant and the gNA are complexed as an RNP.

62. The system of claim 61, wherein an RNP comprising the CasX variant protein and the gNA exhibit at least one or more improved characteristics as compared to an RNP comprising the reference CasX protein of SEQ ID NO: 1, SEQ ID NO: 2, or SEQ ID NO: 3 and a gNA comprising a sequence of any one of SEQ ID NOS: 4-16.

63. The system of claim 62, wherein the improved characteristic is selected from one or more of the group consisting of improved folding of the CasX variant; improved binding affinity to a guide nucleic acid (gNA); improved binding affinity to a target DNA; improved ability to utilize a greater spectrum of one or more PAM sequences, including ATC, CTC, GTC, or TTC, in the editing of target DNA; improved unwinding of the target DNA; increased editing activity; improved editing efficiency; improved editing specificity; increased nuclease activity; improved target nucleic acid sequence cleavage rate; increased target strand loading for double strand cleavage; decreased target strand loading for single strand nicking; decreased off-target cleavage; improved binding of non-target DNA strand; improved protein stability; improved protein solubility; improved ribonuclear protein complex (RNP) formation; higher percentage of cleavage-competent RNP; improved protein:gNA complex (RNP) stability; improved protein:gNA complex solubility; improved protein yield; improved protein expression; and improved fusion characteristics.

64. The system of claim 62 or claim 63, wherein the improved characteristic of the RNP of the CasX variant protein and the gNA variant is at least about 1.1 to about 100-fold or more improved relative to the RNP of the reference CasX protein of SEQ ID NO: 1, SEQ ID NO: 2, or SEQ ID NO: 3 and the gNA comprising a sequence of any one of SEQ ID NOS: 4-16.

65. The system of claim 62 or claim 63, wherein the improved characteristic of the CasX variant protein is at least about 1.1, at least about 2, at least about 10, at least about 100-fold or more improved relative to the reference CasX protein of SEQ ID NO: 1, SEQ ID NO: 2, or SEQ ID NO: 3 and the gNA comprising a sequence of any one of SEQ ID NOS: 4-16.

66. The system of claim 64 or claim 65, wherein the improved characteristic is improved binding affinity to the target nucleic acid sequence.

67. The system of any one of claims 61-66, wherein the RNP comprising the CasX variant and the gNA variant exhibits greater editing efficiency and/or binding of a target sequence in the target nucleic acid when any one of the PAM sequences TTC, ATC, GTC, or CTC is located 1 nucleotide 5′ to the non-target strand sequence having identity with the targeting sequence of the gNA in a cellular assay system compared to the editing efficiency and/or binding of an RNP comprising a reference CasX protein and a reference gNA in a comparable assay system.

68. The system of claim 67, wherein the PAM sequence is TTC.

69. The system of claim 68, wherein the targeting sequence of the gNA comprises a sequence selected from the group consisting of SEQ ID NOS: 3184-7251.

70. The system of claim 67, wherein the PAM sequence is ATC.

71. The system of claim 70, wherein the targeting sequence of the gNA comprises a sequence selected from the group consisting of SEQ ID NOs: 315-436, 612-2100 and 2286-3183.

72. The system of claim 67, wherein the PAM sequence is CTC.

73. The system of claim 72, wherein the targeting sequence of the gNA comprises a sequence selected from the group consisting of SEQ ID NOs: 7252-11521.

74. The system of claim 67, wherein the PAM sequence is GTC.

75. The system of claim 74, wherein the targeting sequence of the gNA comprises a sequence selected from the group consisting of SEQ ID NOs: 11522-13861.

76. The system of any one of claims 61-75, wherein the RNP has at least a 5%, at least a 10%, at least a 15%, or at least a 20% higher percentage of cleavage-competent RNP compared to an RNP of the reference CasX proteins of SEQ ID NOS: 1-3 and the gNA of SEQ ID NOS: 4-16.

77. The system of any one of claims 60-75, wherein the RNP has at least a 5-fold, at least a 10-fold, or at least a 30-fold increased cleavage rate in an in vitro assay compared to an RNP of the reference CasX proteins of SEQ ID NOS: 1-3.

78. The system of any one of claims 49-77, wherein the CasX variant protein comprises a RuvC DNA cleavage domain having nickase activity.

79. The system of any one of claims 49-77, wherein the CasX variant protein comprises a RuvC DNA cleavage domain having double-stranded cleavage activity.

80. The system of any one of claims 49-75, wherein the CasX protein is a catalytically inactive CasX (dCasX) protein, and wherein an RNP of the dCasX and the gNA retain the ability to bind to the PCSK9 target nucleic acid.

81. The system of claim 80, wherein the dCasX comprises a mutation at residues:
a. D672, E769, and/or D935 corresponding to the CasX protein of SEQ ID NO:1; or
b. D659, E756 and/or D922 corresponding to the CasX protein of SEQ ID NO: 2.

82. The system of claim 81, wherein the mutation is a substitution of alanine for the residue.

83. The system of any one of claims 1-79, further comprising a donor template nucleic acid.

84. The system of claim 83, wherein the donor template comprises a nucleic acid comprising at least a portion of a PCSK9 gene selected from the group consisting of a PCSK9 exon, a PCSK9 intron, a PCSK9 intron-exon junction, and a PCSK9 regulatory element, or a combination thereof.

85. The system of claim 84, wherein the donor template comprises a wild-type nucleic acid sequence.

86. The system of claim 84, wherein the donor template comprises a nucleic acid sequence having one or more mutations relative to the wild-type PCSK9 gene sequence.

87. The system of any one of claims 83-86, wherein the donor template ranges in size from 10-15,000 nucleotides.

88. The system of any one of claims 83-87, wherein the donor template is a single-stranded DNA template or a single stranded RNA template.

89. The system of any one of claims 83-87, wherein the donor template is a double-stranded DNA template.

90. The system of any one of claims 83-89, wherein the donor template comprises homologous arms at or near the 5′ and 3′ ends of the donor template that are complementary to sequences flanking cleavage sites in the PCSK9 target nucleic acid introduced by the Class 2 Type V CRISPR protein.

91. The system of any one of claims 1-90, wherein the target nucleic acid sequence is complementary to a non-target strand sequence located 1 nucleotide 3′ of a protospacer adjacent motif (PAM) sequence.

92. The system of claim 91, wherein the PAM sequence comprises a TC motif.

93. The system of claim 91, wherein the PAM sequence comprises ATC, GTC, CTC or TTC.

94. The system of any one of claims 91-93, wherein the Class 2 Type V CRISPR protein comprises a RuvC domain.

95. The system of claim 94, wherein the RuvC domain generates a staggered double-stranded break in the target nucleic acid sequence.

96. The system of any one of claims 91-95, wherein the Class 2 Type V CRISPR protein does not comprise an HNH nuclease domain.

97. A nucleic acid comprising the donor template of any one of claims 83-90.

98. A nucleic acid comprising a sequence that encodes the CasX of any one of claims 49-82.

99. The nucleic acid of claim 98, wherein the sequence that encodes the CasX protein is codon optimized for expression in a eukaryotic cell.

100. A nucleic acid comprising a sequence that encodes the gNA of any one of claims 1-48.

101. A vector comprising the gNA of any one of claims 1-48, the CasX protein of any one of claims 49-82, or the nucleic acid of any one of claims 97-100.

102. The vector of claim 101, wherein the vector further comprises a promoter.

103. The vector of claim 101 or claim 102, wherein the vector is selected from the group consisting of a retroviral vector, a lentiviral vector, an adenoviral vector, an adeno-associated viral (AAV) vector, a herpes simplex virus (HSV) vector, a virus-like particle (VLP), a plasmid, a minicircle, a nanoplasmid, a DNA vector, and an RNA vector.

104. The vector of claim 103, wherein the vector is an AAV vector.

105. The vector of claim 104, wherein the AAV vector is selected from AAV1, AAV2, AAV3, AAV4, AAV5, AAV6, AAV7, AAV8, AAV9, AAV10, AAV11, AAV12, AAV 44.9, AAV-Rh74, or AAVRh10.

106. The vector of claim 105, wherein the AAV vector is selected from AAV1, AAV2, AAV5, AAV8, or AAV9.

107. The vector of claim 103, wherein the vector is a retroviral vector.

108. The vector of claim 103, wherein the vector is a VLP vector comprising one or more components of a gag polyprotein.

109. The vector of claim 108, wherein the one or more components of the Gag polyprotein are selected from the group consisting of matrix protein (MA), nucleocapsid protein (NC), capsid protein (CA), p1-p6 protein, a PP21/24 peptide, a P12/P3/P8 peptide, a p2 peptide, a P10 peptide, a p68 Gag polypeptide, a p3 Gag polypeptide, and a protease cleavage site.

110. The vector of claim 108 or claim 109, comprising the CasX protein and the gNA.

111. The vector of claim 110, wherein the CasX protein and the gNA are associated together in an RNP.

112. The vector of any one of claims 108-111, further comprising the donor template.

113. The vector of any one of claims 108-112, further comprising a pseudotyping viral envelope glycoprotein or antibody fragment that provides for binding and fusion of the VLP to a target cell.

114. A host cell comprising the vector of any one of claims 101-113.

115. The host cell of claim 114, wherein the host cell is selected from the group consisting of BHK, HEK293, HEK293T, NS0, SP2/0, YO myeloma cells, P3X63 mouse myeloma cells, PER, PER.C6, NIH3T3, COS, HeLa, CHO, and yeast cells.

116. A pharmaceutical composition comprising:
a. the system of any one of claims 1-96;
b. the nucleic acid of any one of claims 97-100; or
c. the vector of any one of claims 101-113,
and one or more pharmaceutically suitable excipients.

117. The pharmaceutical composition of claim 116, wherein the pharmaceutical composition is formulated for a route of administration selected from the group consisting of intravenous, intraportal vein injection, intraperitoneal, intramuscular, subcutaneous, intraocular, and oral routes.

118. The pharmaceutical composition of claim 116, wherein the pharmaceutical composition is in a liquid form or a frozen form.

119. The pharmaceutical composition of any one of claims 116-118, wherein the pharmaceutical composition is in a pre-filled syringe for a single injection.

120. A method of modifying a PCSK9 target nucleic acid sequence in a population of cells, wherein the PCSK9 target nucleic acid comprises one or more mutations, the method comprising introducing into cells of the population:
a. the system of any one of claims 1-96;
b. the nucleic acid of any one of claims 97-100;
c. the vector of any one of claims 101-113;
d. the pharmaceutical composition of any one of claims 116-119; or
e. combinations of two or more of (a)-(d),
wherein the PCSK9 target nucleic acid sequence of the cells targeted by the first gNA is modified by the Class 2 Type V protein.

121. The method of claim 120, wherein the modifying comprises introducing a single-stranded break in the PCSK9 target nucleic acid sequence of the cells of the population.

122. The method of claim 120, wherein the modifying comprises introducing a double-stranded break in the PCSK9 target nucleic acid sequence of the cells of the population.

123. The method of any one of claims 120-122, further comprising introducing into the cells of the population a second gNA or a nucleic acid encoding the second gNA, wherein the second gNA has a targeting sequence complementary to a different or overlapping portion of the PCSK9 target nucleic acid compared to the first gNA, resulting in an additional break in the PCSK9 target nucleic acid of the cells of the population.

124. The method of any one of claims 120-123, wherein the modifying comprises introducing an insertion, deletion, substitution, duplication, or inversion of one or more nucleotides in the PCSK9 target nucleic acid of the cells of the population.

125. The method of any one of claims 120-124, wherein the PCSK9 target nucleic acid of at least about 1%, at least about 2%, at least about 3%, at least about 4%, at least about 5%, at least about 6%, at least about 7%, at least about 8%, at least about 9%, or at least about 10%, at least about 20%, at least about 30%, at least about 40%, at least about 50%, at least about 60% or more of the cells of the population is modified.

126. The method of any one of claims 120-124, wherein the modifying results in a knocking down or knocking out of the PCSK9 gene in the cells of the population such that expression of non-functional PCSK9 protein is decreased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

127. The method of any one of claims 120-126, wherein the PCSK9 gene of the cells of the population is modified such that at least about 10%, at least about 20%, at least about 30%, at least about 40%, at least about 50%, at least about 60%, at least about 70%, at least about 80%, or at least about 90% of the modified cells do not express a detectable level of non-functional PCSK9 protein.

128. The method of any one of claims 120-124, wherein the modifying results in a correction or compensation of the mutation of the PCSK9 gene in the cells of the population such that functional PCSK9 protein is expressed by the cells.

129. The method of any one of claims 120-124 and 128, wherein expression of the functional PCSK9 protein by the cells of the population is increased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

130. The method of any one of claims 120-123, wherein the method comprises insertion of a sequence of the donor template into the break site(s) of the PCSK9 gene target nucleic acid sequence of the cells of the population.

131. The method of claim 130, wherein the insertion of the sequence of the donor template is mediated by homology-directed repair (HDR) or homology-independent targeted integration (HITI).

132. The method of claim 130 or claim 131, wherein insertion of the sequence of the donor template results in a correction or compensation of the PCSK9 gene in the cells of the population such that functional PCSK9 protein is expressed by the cells.

133. The method of any one of claims 130-132, wherein expression of the functional PCSK9 protein by the cells of the population is increased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

134. The method of any one of claims 130-132, wherein the PCSK9 gene of the cells of the population is modified such that at least about 50%, at least about 60%, at least about 70%, at least about 75%, at least about 80%, at least about 85%, at least about 90%, or at least about 95% of the modified cells express a detectable level of functional PCSK9.

135. The method of claim 130 or claim 131, wherein insertion of the sequence of the donor template results in a knocking down or knocking out the PCSK9 gene in the cells of the population such that expression of a non-functional PCSK9 protein is decreased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

136. The method of claim 130 or claim 131, wherein the PCSK9 gene of the cells of the population is modified such that at least about 10%, at least about 20%, at least about 30%, at least about 40%, at least about 50%, at least about 60%, at least about 70%, at least about 80%, or at least about 90% of the modified cells do not express a detectable level of non-functional PCSK9 protein.

137. The method of any one of claims 120-136, wherein the cells are eukaryotic.

138. The method of claim 137, wherein the eukaryotic cells are selected from the group consisting of rodent cells, mouse cells, rat cells, and non-human primate cells.

139. The method of claim 137, wherein the eukaryotic cells are human cells.

140. The method of claim 137-139, wherein the eukaryotic cells are selected from the group consisting of a hepatocyte, a cell of the intestine, a cell of the kidney, a cell of the central nervous system, a smooth muscle cell, a macrophage, a cell of the retina, and an arterial endothelial cell.

141. The method of any one of claim 120-140, wherein the modifying of the PCSK9 gene target nucleic acid sequence of the population of cells occurs in vitro or ex vivo.

142. The method of claims 120-140, wherein the modifying of the PCSK9 gene target nucleic acid sequence of the population of cells occurs in vivo in a subject.

143. The method of claim 142, wherein the subject is selected from the group consisting of a rodent, a mouse, a rat, and a non-human primate.

144. The method of claim 142, wherein the subject is a human.

145. The method of any one of claims 142-144, wherein the method comprises administering a therapeutically effective dose of an AAV vector to the subject.

146. The method of claim 145, wherein the AAV vector is administered to the subject at a dose of at least about 1×10⁵ vector genomes/kg (vg/kg body weight), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

147. The method of claim 145, wherein the AAV vector is administered to the subject at a dose of at least about 1×10⁵ vg/kg to about 1×10¹⁶ vg/kg, at least about 1×10⁶ vg/kg to about 1×10¹⁵ vg/kg, at least about 1×10⁷ vg/kg to about 1×10¹⁴ vg/kg, at least about 1×10⁸ vg/kg to about 1×10¹³ vg/kg, at least about 1×10⁹ vg/kg to about 1×10¹² vg/kg, or at least about 1×10¹⁰ vg/kg to about 1×10¹¹ vg/kg.

148. The method of any one of claims 142-144, wherein the method comprises administering a therapeutically effective dose of a VLP to the subject.

149. The method of claim 148, wherein the VLP is administered to the subject at a dose of at least about 1×10⁵ particles/kg body weight (particles/kg), at least about 1×10⁶ particles/kg, at least about 1×10⁷ particles/kg, at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

150. The method of claim 148, wherein the VLP is administered to the subject at a dose of at least about 1×10⁵ particles/kg to about 1×10¹⁶ particles/kg, at least about 1×10⁶ particles/kg to about 1×10¹⁵ particles/kg, at least about 1×10⁷ particles/kg to about 1×10¹⁴ particles/kg, at least about 1×10⁸ particles/kg to about 1×10¹³ particles/kg, at least about 1×10⁹ particles/kg to about 1×10¹² particles/kg, at least about 1×10¹⁰ particles/kg to about 1×10¹¹ particles/kg.

151. The method of any one of claims 142-150, wherein the vector or VLP is administered to the subject by a route of administration selected from the group consisting of intravenous, intraportal vein injection, intraperitoneal, intramuscular, subcutaneous, intraocular, and oral routes.

152. The method of any one of claims 142-151, further comprising contacting the PCSK9 target nucleic acid sequence of the population of cells with:
a. an additional CRISPR nuclease and a gNA targeting a different or overlapping portion of the PCSK9 target nucleic acid compared to the first gNA;
b. one or more polynucleotides encoding the additional CRISPR nuclease and the gNA of (a);
c. a vector comprising the polynucleotide(s) of (b); or
d. a VLP comprising the additional CRISPR nuclease and the gNA of (a);
wherein the contacting results in modification of the PCSK9 gene at a different location in the sequence compared to the sequence targeted by the first gNA.

153. The method of claim 152, wherein the additional CRISPR nuclease is a CasX protein having a sequence different from the CasX protein of any of the preceding claims.

154. The method of claim 152, wherein the additional CRISPR nuclease is not a CasX protein.

155. The method of claim 154, wherein the additional CRISPR nuclease is selected from the group consisting of Cas9, Cas12a, Cas12b, Cas12c, Cas12d (CasY), Cas12J, Cas13a, Cas13b, Cas13c, Cas13d, CasX, CasY, CasZ, Cas14, Cpf1, C2c1, Csn2, Cas Phi, and sequence variants thereof.

156. A population of cells modified by the method of any one of claims 142-155, wherein the cells have been modified such that at least 70%, at least 75%, at least 80%, at least 85%, at least 90%, or at least 95% of the modified cells do not express a detectable level of non-functional PCSK9 protein.

157. A population of cells modified by the method of any one of claims 142-156, wherein the mutation of the PCSK9 target nucleic acid is corrected or compensated for in the modified cells of the population, resulting in expression of a functional PCSK9 protein by the modified cells.

158. The population of cells of claim 157, wherein the cells have been modified such that expression of a functional PCSK9 protein is increased by at least about 10%, at least about 20%, at least about 30%, at least about 40%, at least about 50%, at least about 60%, at least about 70%, at least about 80%, or at least about 90% in comparison to a cell where the PCSK9 gene has not been modified.

159. The population of cells of any one of claim 156-158, wherein the cells are selected from the group consisting of a hepatocyte, a cell of the intestine, a cell of the kidney, a cell of the central nervous system, a smooth muscle cell, a macrophage, a retinal cell, and an arterial endothelial cell.

160. A method of treating a PCSK9-related disease in a subject in need thereof, the method comprising administering to the subject a therapeutically effective amount of the cells of any one of claims 156-159.

161. The method of claim 160, wherein the PCSK9-related disease is autosomal dominant hypercholesterolemia (ADH), hypercholesterolemia, elevated total cholesterol levels, hyperlipidemia, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, age-related macular degeneration (AMD), or a combination thereof.

162. The method of claim 160 or claim 161, wherein the subject is selected from the group consisting of a rodent, a mouse, a rat, and a non-human primate.

163. The method of any one of claims 160-162, wherein the subject is a human.

164. The method of any one of claims 160-163, wherein the cells are autologous with respect to the subject to be administered the cells.

165. The method of any one of claims 160-163 wherein the cells are allogeneic with respect to the subject to be administered the cells.

166. The method of any one of claims 160-165, wherein the cells are administered by a route of administration selected from the group consisting of intravenous, intraportal vein injection, intraperitoneal, intramuscular, subcutaneous, intraocular, and oral routes.

167. A method of treating a PCSK9-related disease in a subject in need thereof, comprising modifying a PCSK9 gene having one or more mutations in cells of the subject, the modifying comprising contacting said cells with a therapeutically effective dose of:
a. the system of any one of claims 1-96;
b. the nucleic acid of any one of claims 97-100;
c. the vector of any one of claims 101-107;
d. the VLP of any one of claims 108-113;
e. the pharmaceutical composition of any one of claims 116-119; or
f. combinations of two or more of (a)-(e),
wherein the PCSK9 gene of the cells targeted by the first gNA is modified by the CasX protein.

168. The method of claim 167, wherein the modifying comprises introducing a single-stranded break in the PCSK9 gene of the cells.

169. The method of claim 167, wherein the modifying comprises introducing a double-stranded break in the PCSK9 gene of the cells.

170. The method of any one of claims 167-169, further comprising introducing into the cells of the subject a second gNA or a nucleic acid encoding the second gNA, wherein the second gNA has a targeting sequence complementary to a different or overlapping portion of the target nucleic acid compared to the first gNA, resulting in an additional break in the PCSK9 target nucleic acid of the cells of the subject.

171. The method of any one of claims 167-169, wherein the modifying comprises introducing an insertion, deletion, substitution, duplication, or inversion of one or more nucleotides in the PCSK9 gene of the cells.

172. The method of any one of claims 167-170, wherein the modifying comprises insertion of a sequence of the donor template into the break site(s) of the PCSK9 gene target nucleic acid sequence of the cells.

173. The method of claim 172, wherein the insertion of the sequence of the donor template is mediated by homology-directed repair (HDR) or homology-independent targeted integration (HITI).

174. The method of any one of claims 167-173, wherein the modifying results in a correction of or compensation for the mutation(s) in the PCSK9 gene in the modified cells of the subject.

175. The method of claim 174, wherein correction of the mutation results in expression of functional PCSK9 protein by the modified cells of the subject.

176. The method of claim 174 or claim 175, wherein the PCSK9 gene of the modified cells express increased levels of a functional PCSK9 protein, and wherein the increase is at least about 10%, at least about 20%, at least about 30%, at least about 40%, at least about 50%, at least about 60%, at least about 70%, at least about 80%, or at least about 90% in comparison to a cell with a PCSK9 gene that has not been modified.

177. The method of any one of claims 167-173, wherein the modifying results in a knocking down or knocking out the PCSK9 gene in the modified cells of the subject such that at least about 10%, at least about 20%, at least about 30%, at least about 40%, at least about 50%, at least about 60%, at least about 70%, at least about 80%, or at least about 90% of the modified cells do not express a detectable level of non-functional PCSK9 protein.

178. The method of any one of claims 167-173, wherein the modifying results in a knocking down or knocking out the PCSK9 gene in the modified cells of the subject such that expression of non-functional PCSK9 protein in the subject is decreased by at least about 10%, at least about 20%, at least about 30%, at least about 40%, at least about 50%, at least about 60%, at least about 70%, at least about 80%, or at least about 90% in comparison to a subject where the PCSK9 gene has not been modified.

179. The method of any one of claims 167-178, wherein the subject is selected from the group consisting of rodent, mouse, rat, and non-human primate.

180. The method of any one of claims 167-178, wherein the subject is a human.

181. The method of any one of claims 167-180, wherein the cells that are modified are selected from the group consisting of a hepatocyte, a cell of the intestine, a cell of the kidney, a cell of the central nervous system, a smooth muscle cell, a macrophage, a cell of the retina, and an arterial endothelial cell.

182. The method of any one of claims 167-181, wherein the PCSK9-related disease is autosomal dominant hypercholesterolemia (ADH), hypercholesterolemia, elevated total cholesterol levels, hyperlipidemia, elevated low-density lipoprotein (LDL) levels, elevated LDL-cholesterol levels, reduced high-density lipoprotein levels, liver steatosis, coronary heart disease, ischemia, stroke, peripheral vascular disease, thrombosis, type 2 diabetes, high elevated blood pressure, atherosclerosis, obesity, Alzheimer's disease, neurodegeneration, age-related macular degeneration (AMD), or a combination thereof.

183. The method of any one of claims 167-182, wherein the vector is administered to the subject at a therapeutically-effective dose.

184. The method of any one of claims 167-183, wherein the vector is an AAV, and is administered to the subject at a dose of at least about 1×10⁵ vector genomes (vg)/kg, at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

185. The method of any one of claims 167-183, wherein the vector is an AAV, and is administered to the subject at a dose of at least about 1×10⁵ vg/kg to about 1×10¹⁶ vg/kg, at least about 1×10⁶ vg/kg to about 1×10¹⁵ vg/kg, at least about 1×10⁷ vg/kg to about 1×10¹⁴ vg/kg, at least about 1×10⁸ vg/kg to about 1×10¹³ vg/kg, at least about 1×10⁹ vg/kg to about 1×10¹² vg/kg, or at least about 1×10¹⁰ vg/kg to about 1×10¹¹ vg/kg.

186. The method of any one of claims 167-182, wherein the VLP is administered to the subject at a therapeutically-effective dose.

187. The method of claim 186, wherein the VLP is administered to the subject at a dose of at least about 1×10⁵ particles/kg, at least about 1×10⁶ particles/kg, at least about 1×10⁷ particles/kg, at least about 1×10⁸ particles/kg, at least about 1×10⁹ particles/kg, at least about 1×10¹⁰ particles/kg, at least about 1×10¹¹ particles/kg, at least about 1×10¹² particles/kg, at least about 1×10¹³ particles/kg, at least about 1×10¹⁴ particles/kg, at least about 1×10¹⁵ particles/kg, at least about 1×10¹⁶ particles/kg.

188. The method of claim 186, wherein the VLP is administered to the subject at a dose of at least about 1×10⁵ particles/kg to about 1×10¹⁶ particles/kg, at least about 1×10⁶ particles/kg to about 1×10¹⁵ particles/kg, at least about 1×10⁷ particles/kg to about 1×10¹⁴ particles/kg, at least about 1×10⁸ particles/kg to about 1×10¹³ particles/kg, at least about 1×10⁹ particles/kg to about 1×10¹² particles/kg, at least about 1×10¹⁰ particles/kg to about 1×10¹¹ particles/kg.

189. The method of any one of claims 183-188, wherein the vector or VLP is administered by a route of administration selected from the group consisting of intravenous, intraportal vein injection, intraperitoneal, intramuscular, subcutaneous, intraocular, and oral routes.

190. The method of any one of claims 167-189, wherein the method results in improvement in at least one clinically-relevant endpoint selected from the group consisting of change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina, or visual acuity.

191. The method of any one of claims 167-189, wherein the method results in improvement in at least two clinically-relevant endpoints selected from the group consisting of change from baseline in LDL-cholesterol, decrease in plaque atheroma volume, reduction in in coronary plaque, reduction in atherosclerotic cardiovascular disease (ASCVD), cardiovascular death, nonfatal myocardial infarction, ischemic stroke, nonfatal stroke, coronary revascularization, unstable angina or visual acuity.

192. The system of any one of claims 1-96; the nucleic acid of any one of claims 97-100; the vector of any one of claims 101-107; the VLP of any one of claims 108-113; the pharmaceutical composition of any one of claims 116-119; or combinations thereof, for use as a medicament for the treatment of a PCSK9-related disease.
